(19) United States
(12) Patent Application Publication
(10) Pub. No.: US 2022/0162192 A1
(43) Pub. Date: May 26, 2022

(54) INDAZOLES AS LRRK2 INHIBITORS

(71) Applicant: ESCAPE Bio, Inc., South San Francisco, CA (US)

(72) Inventors: Albert W. Garofalo, South San Francisco, CA (US); Stéphane De Lombaert, Brisbane, CA (US); Daniele Andreotti, Verona (IT); Fabio Maria Sabbatini, Verona (IT); Elena Serra, Lecce (IT); Silvia Bernardi, Verona (IT); Marco Migliore, Verona (IT); Federica Budassi, Urbino (IT); Claudia Beato, Verona (IT)

(21) Appl. No.: 17/440,872

(22) PCT Filed: Mar. 20, 2020
(86) PCT No.: PCT/US2020/023763

§ 371 Date: Sep. 20, 2021

Related U.S. Application Data
(60) Provisional application No. 62/937,988, filed on Nov. 20, 2019.
(60) Provisional application No. 62/821,637, filed on Mar. 21, 2019.

(51) Int. Cl. C07D 403/12; C07D 401/14; C07D 471/04; C07D 413/14; C07D 405/14; C07D 417/14; C07D 409/14; C07D 487/04; C07D 495/04; C07D 513/04; A61P 25/16

(52) U.S. Cl. CPC C07D 403/12; C07D 401/14; C07D 471/04; C07D 413/14; C07D 405/14; A61P 25/16; C07D 409/14; C07D 487/04; C07D 495/04; C07D 513/04; C07D 417/14

(57) ABSTRACT

The present invention is directed to indazole compounds which are inhibitors of LRRK2 and are useful in the treatment of CNS disorders.

### FIELD OF THE INVENTION

The present invention is directed to amidoindazole compounds which are inhibitors of LRRK2 and are useful in the treatment of CNS disorders.

### BACKGROUND OF THE INVENTION

Parkinson's disease (“PD”) is the most common form of parkinsonism, a movement disorder, and the second most common, age-related neurodegenerative disease estimated to affect 1-2% of the population over age 65. PD is characterized by tremor, rigidity, postural instability, impaired speech, and bradykinesia. It is a chronic, progressive disease with increasing disability and diminished quality of life. In addition to PD, parkinsonism is exhibited in a range of conditions such as progressive supranuclear palsy, corticobasal degeneration, multiple system atrophy, and dementia with Lewy bodies.

Current therapeutic strategies for PD are primarily palliative and focus on reducing the severity of symptoms using supplemental dopaminergic medications. At present, there is no disease-modifying therapy that addresses the underlying neuropathological cause of the disease, thus constituting a significant unmet medical need.

It has long been known that family members of PD patients have an increased risk of developing the disease compared to the general population. Leucine-rich repeat kinase 2 (“LRRK2,” also known as dardarin) is a 286 kDa multi-domain protein that has been linked to PD by genome-wide association studies. LRRK2 expression in the brain is highest in areas impacted by PD (Eur. J Neurosci. 2006, 23(3):659) and LRRK2 has been found to localize in Lewy Bodies, which are intracellular protein aggregates considered to be a hallmark of the disease. Patients with point mutations in LRRK2 present disease that is indistinguishable from idiopathic patients. While more than 20 LRRK2 mutations have been associated with autosomal-dominantly inherited parkinsonism, the G2019S mutation located within the kinase domain of LRRK2 is by far the most common. This particular mutation is found in >85% of LRRK2-linked PD patients. It has been shown that the G2019S mutation in LRRK2 leads to an enhancement in LRRK2 kinase activity and inhibition of this activity is a therapeutic target for the treatment of PD.

In addition to PD, LRRK2 has been linked to other diseases such as cancer, leprosy, and Crohn's disease (Sci. Signal., 2012, 5(207), pe2). As there are presently limited therapeutic options for treating PD and other disorders associated with aberrant LRRK2 kinase activity, there remains a need for developing LRRK2 inhibitors.

### SUMMARY OF THE INVENTION

The present invention provides a compound of Formula I:

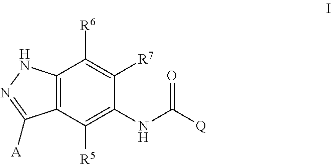

or a pharmaceutically acceptable salt thereof, wherein constituent members are defined herein.

The present invention further provides a pharmaceutical composition comprising a compound of Formula I, or a pharmaceutically acceptable salt thereof, and at least one pharmaceutically acceptable carrier.

The present invention further provides a method of inhibiting LRRK2 activity, comprising contacting a compound of Formula I, or a pharmaceutically acceptable salt thereof, with LRRK2.

The present invention further provides a method of treating a disease or disorder associated with elevated expression or activity of LRRK2, or functional variants thereof, comprising administering to a patient in need thereof a therapeutically effective amount of a compound of Formula I, or a pharmaceutically acceptable salt thereof.

The present invention further provides a method for treating a neurodegenerative disease in a patient, comprising: administering to the patient a therapeutically effective amount of the compound of Formula I, or a pharmaceutically acceptable salt thereof.

The present invention further provides a compound of Formula I, or a pharmaceutically acceptable salt thereof, for use in therapy.

The present invention further provide a compound of Formula I, or a pharmaceutically acceptable salt thereof, for use in the preparation of a medicament for use in therapy.

### DETAILED DESCRIPTION

### Compounds

The present disclosure provides a compound of Formula I:

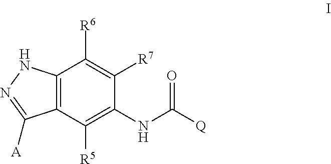

or a pharmaceutically acceptable salt thereof, wherein:

A is C₁₋₆ alkyl, C₁₋₆ haloalkyl, Cy¹, halo, CN, OR^{a}, or NR^{x}R^{y}, wherein said C₁₋₆ alkyl is optionally substituted with Cy¹;

Q is selected from the following groups:

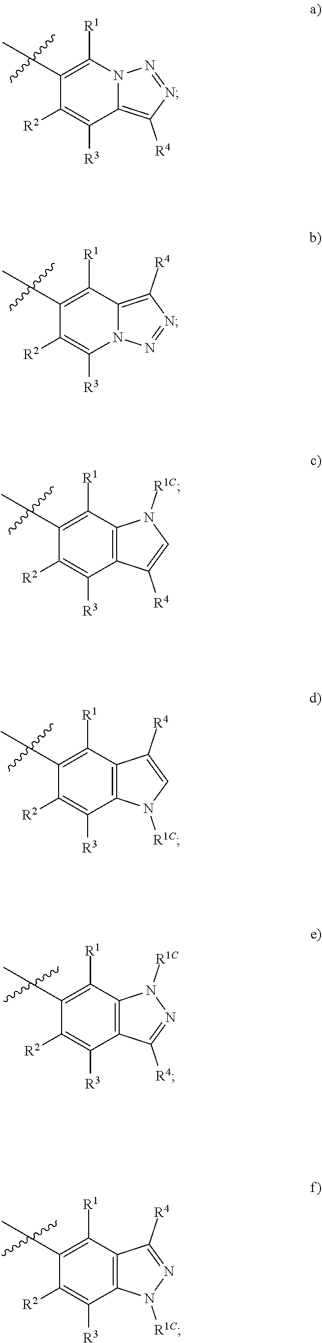

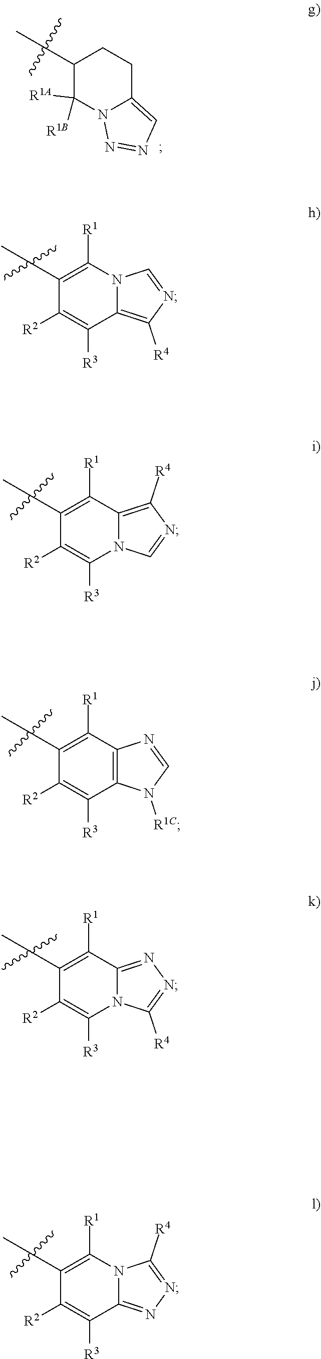

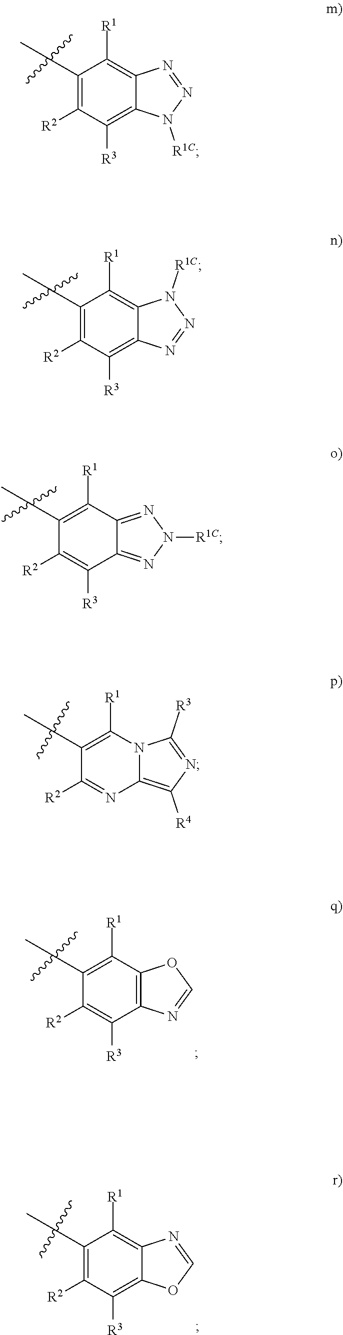

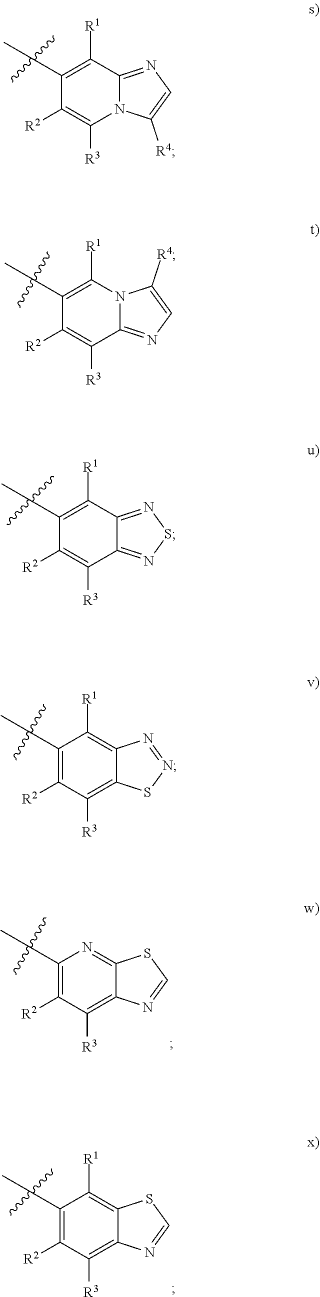

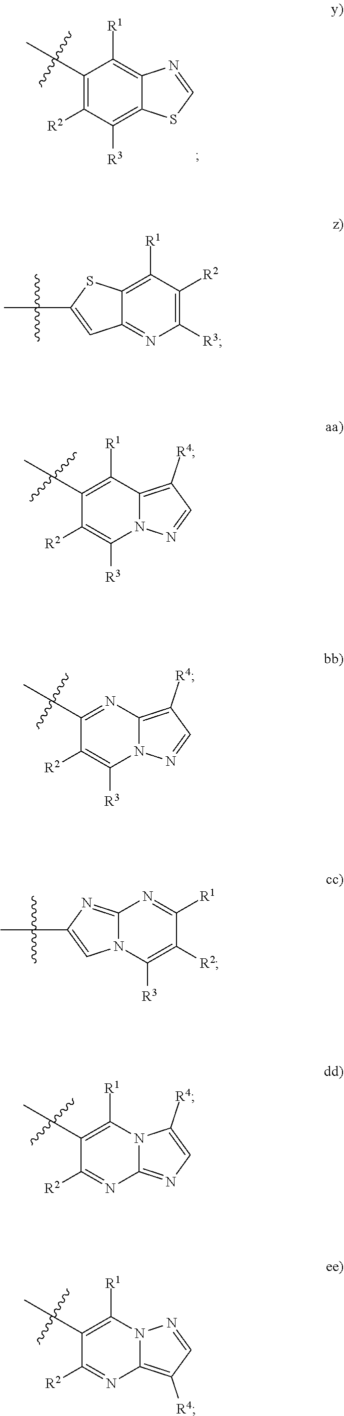

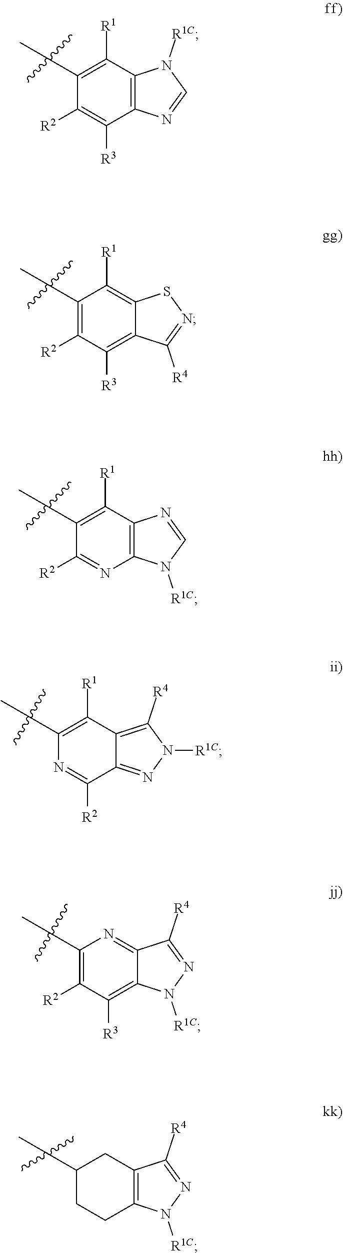

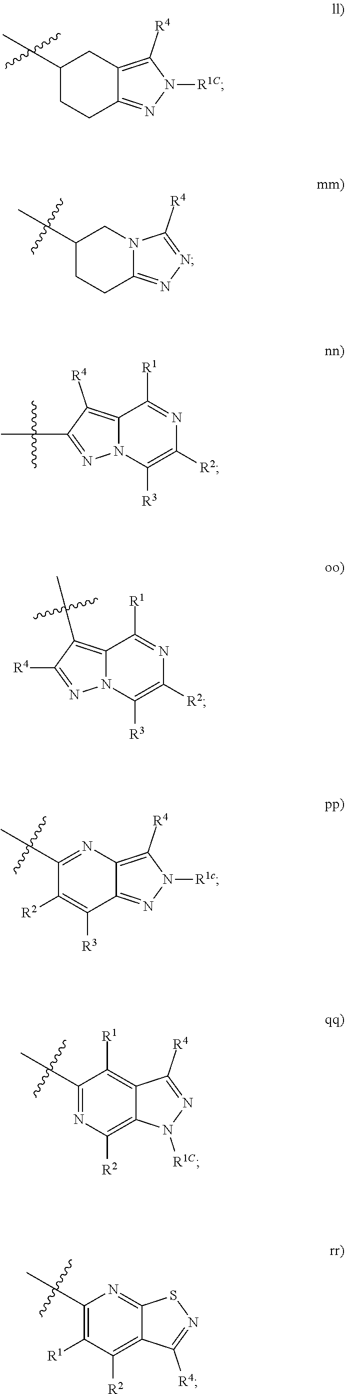

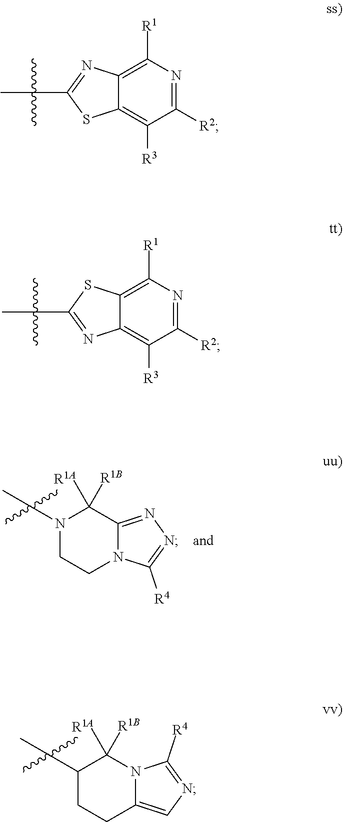

Cy¹ is selected from C₆₋₁₀ aryl, C₃₋₁₀ cycloalkyl, 5-14 membered heteroaryl, and 4-14 membered heterocycloalkyl, each optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d}; R¹, R², R³, R⁴, R⁵, R⁶, and R⁷ are each independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R¹, R², R³, R⁴, R⁵, R⁶, and R⁷ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; R^{1A} and R^{1B} are each independently selected from H, halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{1A} and R^{1B} are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR², SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};

or R^{1A} and R^{1B} together form a C₃₋₇ cycloalkyl or 4-10 membered heterocycloalkyl ring, each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};

R^{1C} is selected from H and C₁₋₆ alkyl;

each R^{a}, R^{b}, R^{c}, R^{d}, R^{a1}, R^{b1}, R^{c1}, R^{d1}, R², R^{b2}, R^{c2}, R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a}, R^{b}, R^{c}, R^{d}, R^{a1}, R^{b1}, R^{c1}, R^{d1}, R^{a2}, R^{b2}, R^{c2}, and R^{d2} is optionally substituted with 1, 2, 3, 4, or 5 substituents independently selected from halo, C₁₋₄ alkyl, C₁₋₄haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b}3, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};

R^{x} is selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{x} is optionally substituted with 1, 2, 3, 4, or 5 substituents independently selected from halo, C₁₋₄ alkyl, C₁₋₄haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};

R^{y} is selected from C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R is optionally substituted with 1, 2, 3, 4, or 5 substituents independently selected from halo, C₁₋₄ alkyl, C₁₋₄haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};

each R^{a3}, R^{b3}, R^{c3}, and R^{d3} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, 4-7 membered heterocycloalkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, and 4-7 membered heterocycloalkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy; and

each R^{e}, R^{e1}, R^{e2}, and R^{e3} is independently selected from H, C₁₋₄ alkyl, and CN.

The present disclosure provides a compound of Formula I:

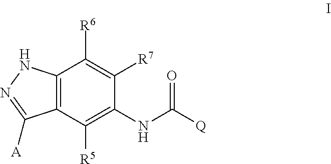

or a pharmaceutically acceptable salt thereof, wherein:

A is C₁₋₆ alkyl, C₁₋₆ haloalkyl, Cy¹, halo, CN, OR^{a}, or NR^{x}R^{y}, wherein said C₁₋₆ alkyl is optionally substituted with Cy¹;

Q is selected from the following groups:

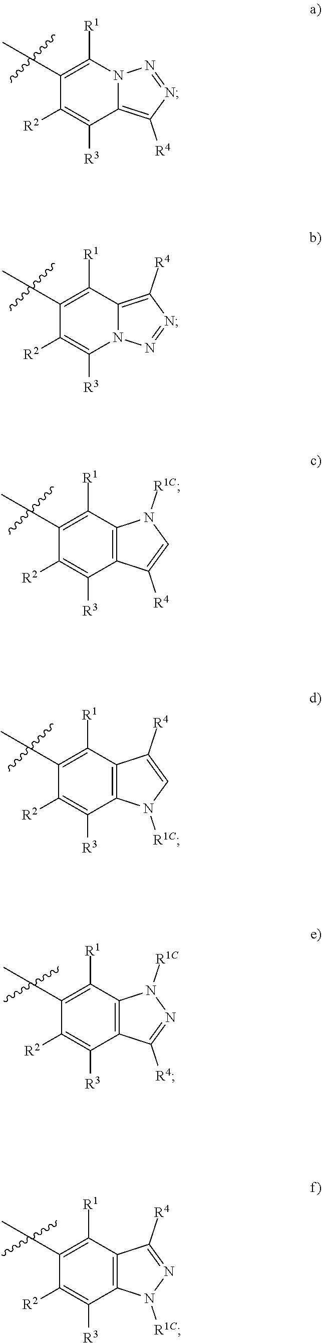

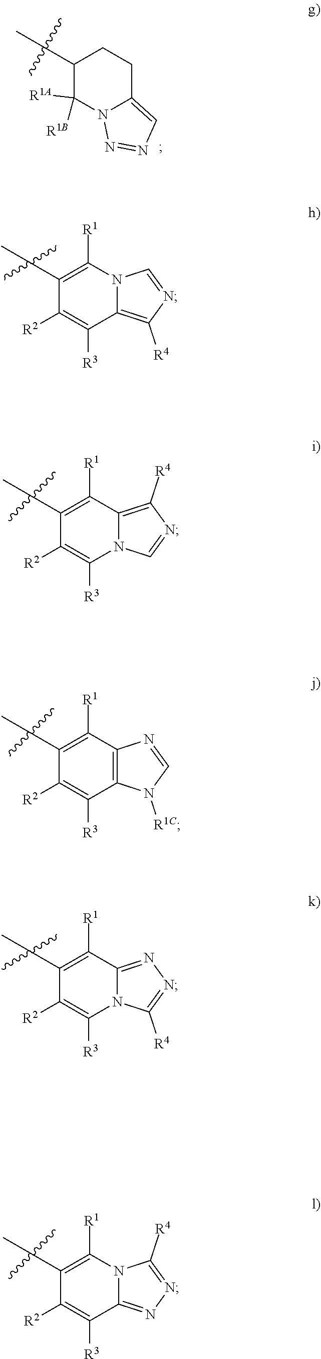

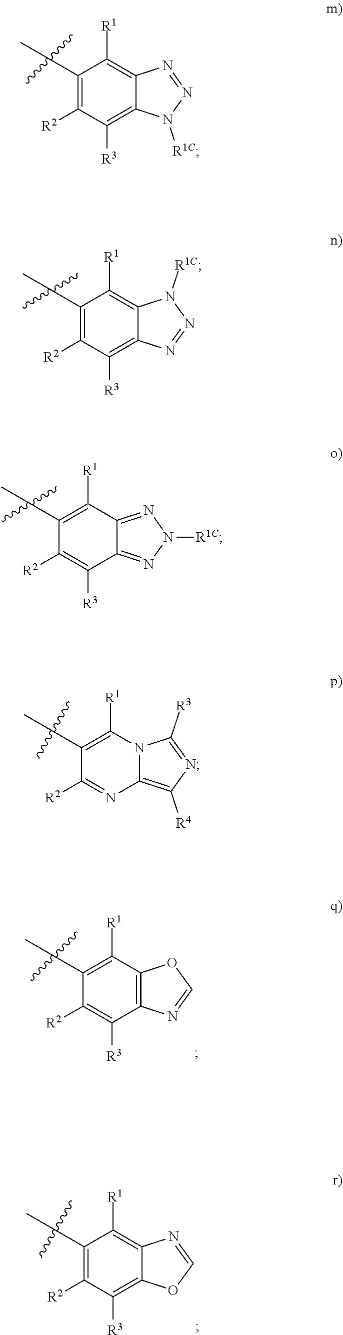

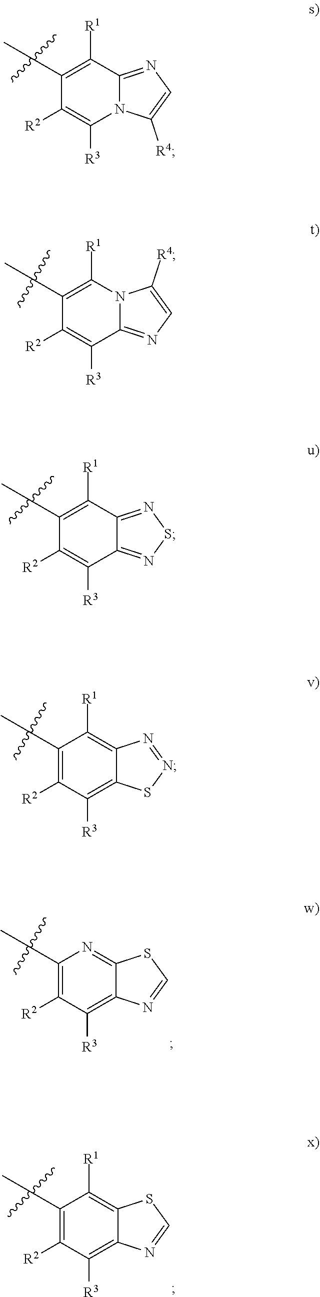

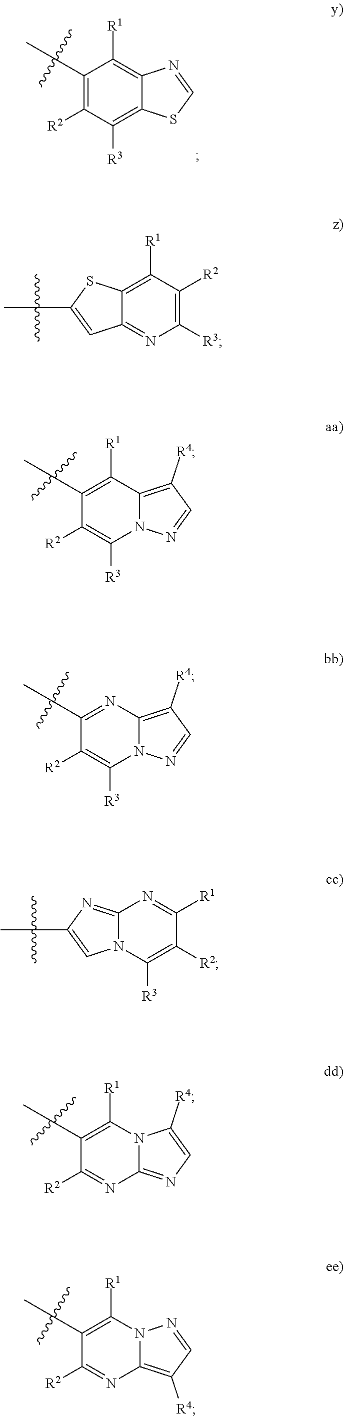

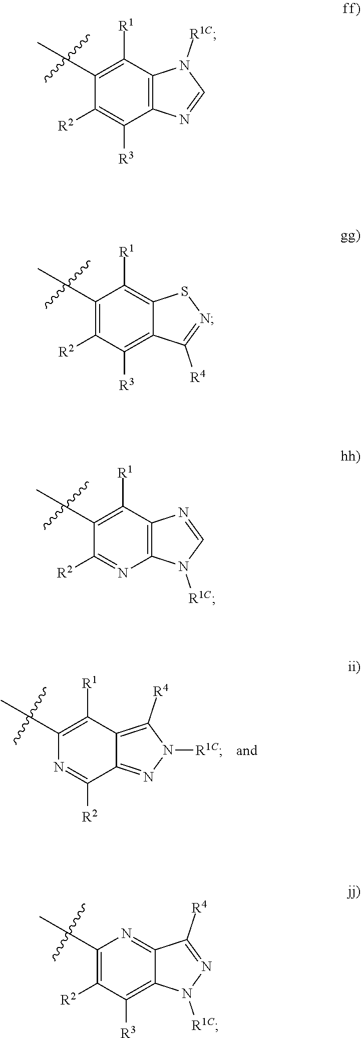

Cy¹ is selected from C₆₋₁₀ aryl, C₃₋₁₀ cycloalkyl, 5-14 membered heteroaryl, and 4-14 membered heterocycloalkyl, each optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};

R¹, R², R³, R⁴, R⁵, R⁶, and R⁷ are each independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R¹, R², R³, R⁴, R⁵, R⁶, and R⁷ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NRC R¹, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

R^{1A} and R^{1B} are each independently selected from H, halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{1A} and R^{1B} are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};

or R^{1A} and R^{1B} together form a C₃₋₇ cycloalkyl or 4-10 membered heterocycloalkyl ring, each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};

R^{1C} is selected from H and C₁₋₆ alkyl;

each R^{a}, R^{b}, R^{c}, R^{d}, R^{a1}, R^{b1}, R^{c1}, R^{d1}, R^{a2}, R^{b2}, R^{c2}, R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a}, R^{b}, R^{c}, R^{d}, R^{a1}, R^{b1}, R^{c1}, R^{d1}, R^{a2}, R^{b2}, R^{c2}, and R^{d2} is optionally substituted with 1, 2, 3, 4, or 5 substituents independently selected from halo, C₁₋₄ alkyl, C₁₋₄haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};

R^{x} is selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{x} is optionally substituted with 1, 2, 3, 4, or 5 substituents independently selected from halo, C₁₋₄ alkyl, C₁₋₄haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};

R^{y} is selected from C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R is optionally substituted with 1, 2, 3, 4, or 5 substituents independently selected from halo, C₁₋₄ alkyl, C₁₋₄haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};

each R^{a3}, R^{b3}, R^{c3}, and R^{d3} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, 4-7 membered heterocycloalkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, and 4-7 membered heterocycloalkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy; and each R^{e}, R^{e1}, R^{e2}, and R^{e3} is independently selected from H, C₁₋₄ alkyl, and CN.

The present disclosure provides a compound of Formula I:

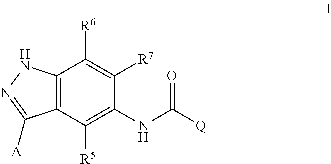

or a pharmaceutically acceptable salt thereof, wherein:

A is C₁₋₆ alkyl, Cy¹, or halo;

Q is selected from the following groups:

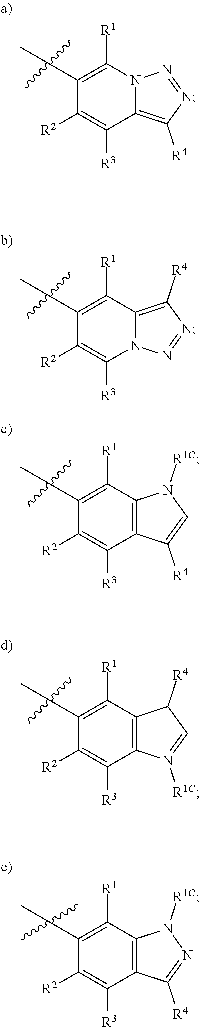

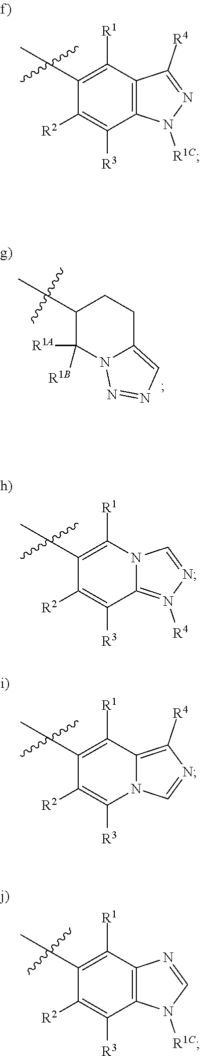

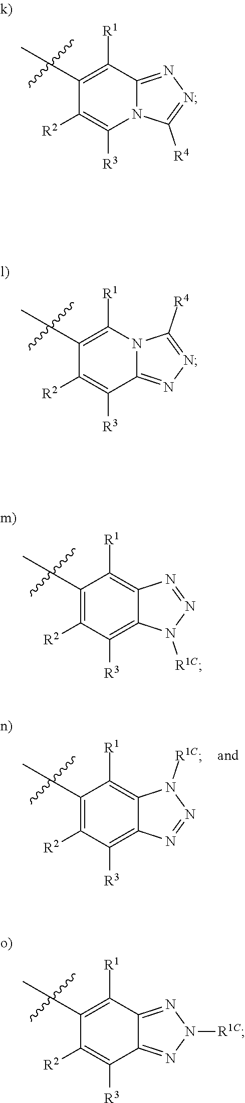

Cy¹ is selected from C₆₋₁₀ aryl, C₃₋₁₀ cycloalkyl, 5-14 membered heteroaryl, and 4-14 membered heterocycloalkyl, each optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a}, SR^{a}, C(O)R^{b}, C(O)NR^{e}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};

R¹, R², R³, R⁴, R⁵, R⁶, and R⁷ are each independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R¹, R², R³, R⁴, R⁵, R⁶, and R⁷ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NRC R¹, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

R^{1A} and R^{1B} are each independently selected from H, halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{1A} and R^{1B} are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};

or R^{1A} and R^{1B} together form a C₃₋₇ cycloalkyl or 4-10 membered heterocycloalkyl ring, each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};

R^{1C} is selected from H and C₁₋₆ alkyl;

each R^{a}, R^{b}, R^{c}, R^{d}, R^{a1}, R^{b1}, R^{c1}, R^{d1}, R^{a2}, R^{b2}, R^{e2}, R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a}, R^{b}, R^{c}, R^{d}, R^{a1}, R^{b1}, R^{c1}, R^{d1}, R^{a2}, R^{b2}, R^{e2}, and R^{d2} is optionally substituted with 1, 2, 3, 4, or 5 substituents independently selected from halo, C₁₋₄ alkyl, C₁₋₄haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b}3, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};

each R^{a3}, R^{b3}, R^{c3}, and R^{d3} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, 4-7 membered heterocycloalkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, and 4-7 membered heterocycloalkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy; and

each R^{e}, R^{e1}, R^{e2}, and R^{e3} is independently selected from H, C₁₋₄ alkyl, and CN.

In some embodiments, provided herein is a compound of Formula I, or a pharmaceutically acceptable salt thereof, wherein:

A is C₁₋₆ alkyl, Cy¹, or halo;

Q is selected from the following groups:

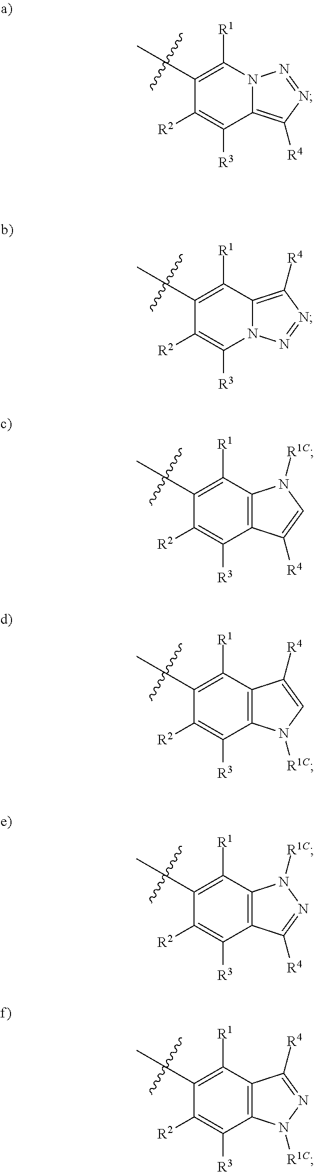

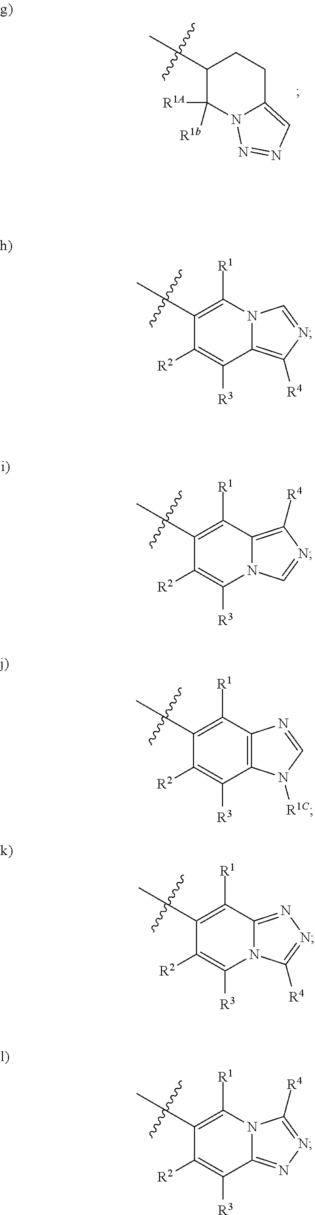

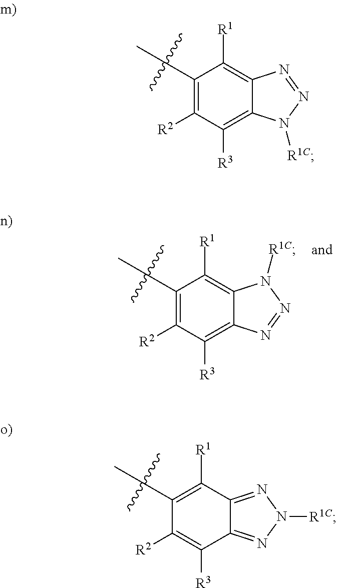

Cy¹ is selected from C₆₋₁₀ aryl and 5-14 membered heteroaryl, each optionally substituted by 1, 2, 3, 4, or 5 substituents independently selected from halo, C₁₋₆ alkyl, C₁₋₆ haloalkyl, C₃₋₁₀ cycloalkyl, 4-14 membered heterocycloalkyl, CN, NO₂, OR^{a}, SR^{a}, NR^{c}R^{d}, NR^{c}C(O)R^{b}, S(O)NR^{c}R^{d}, S(O)₂R^{b}, and S(O)₂NR^{c}R^{d};

R¹, R², R³, R⁴, R⁵, R⁶, and R⁷ are each independently selected from H, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl and C₁₋₆ haloalkyl of R¹, R², R³, R⁴, R⁵, R⁶, and R⁷ are each optionally substituted with 1, 2, 3, 4, or 5 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

R^{1A} and R^{1B} are each independently selected from H, halo, C₁₋₆ alkyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, and NR^{c2}C(O)R^{b2}; wherein said C₁₋₆ alkyl and C₁₋₆ haloalkyl of R^{1A} and R^{1B} are each optionally substituted with 1, 2, 3, 4, or 5 substituents independently selected from CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, and NR^{c2}C(O)R^{b2};

R^{1C} is selected from H and C₁₋₆ alkyl;

each R^{a}, R^{b}, R^{c}, R^{d}, R^{a1}, R^{b1}, R^{c1}, R^{d1}, R^{a2}, R^{b2}, R^{c2}, R^{d2} is independently selected from H, C₁₋₆ alkyl, and C₁₋₆ haloalkyl; and

each R^{e1} is independently selected from H, C₁₋₄ alkyl, and CN.

In some embodiments, provided herein is a compound of Formula I, or a pharmaceutically acceptable salt thereof, wherein:

A is C₁₋₆ alkyl, Cy¹, or halo;

Q is selected from the following groups:

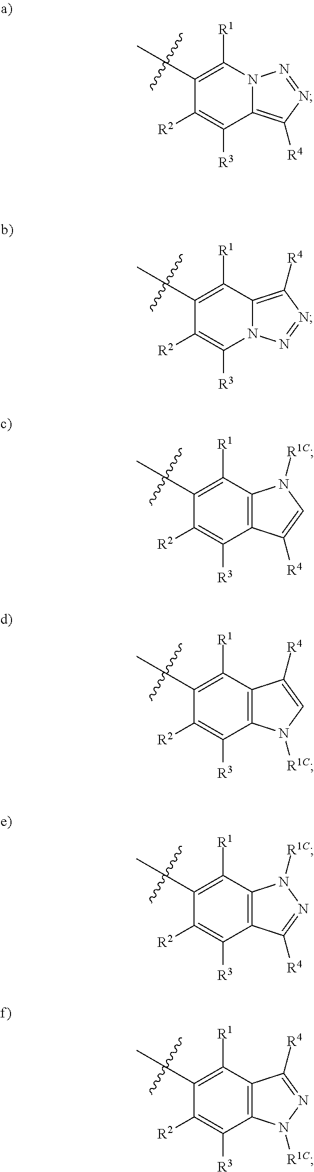

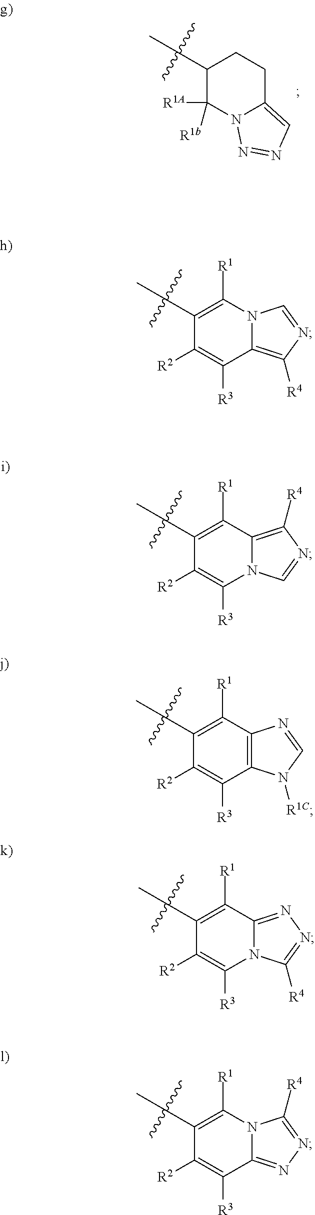

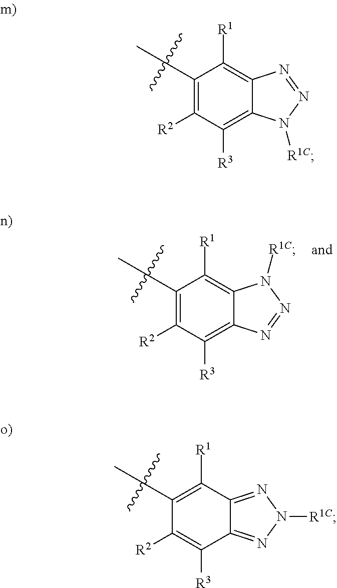

Cy¹ is selected from C₆₋₁₀ aryl and 5-14 membered heteroaryl, each optionally substituted by 1, 2, or 3 substituents independently selected from halo, C₁₋₆ alkyl, 4-14 membered heterocycloalkyl, and OR^{a};

R¹, R², R³, R⁴, R⁵, R⁶, and R⁷ are each independently selected from H, halo, CN, C₁₋₆ alkyl, and C₁₋₆ haloalkyl;

R^{1A} and R^{1B} are each independently selected from H and C₁₋₆ alkyl;

R^{1C} is selected from H and C₁₋₆ alkyl; and

each R^{a} is independently selected from H, C₁₋₆ alkyl, and C₁₋₆ haloalkyl.

In some embodiments, A is C₁₋₆ alkyl, C₁₋₆ haloalkyl, Cy¹, halo, CN, or OR^{a}, wherein said C₁₋₆ alkyl is optionally substituted with Cy¹.

In some embodiments, A is C₁₋₆ alkyl, Cy¹, or halo.

In some embodiments, A is C₁₋₆ alkyl. In some embodiments, A is methyl. In some embodiments, A is methyl or propyl. In some embodiments, A is propyl.

In some embodiments, A is halo. In some embodiments, A is Br.

In some embodiments, A is selected from Cy¹, methyl, propyl, Br, I, CN, methoxy, N(H)CH₂(phenyl), CF₃, and benzyl.

In some embodiments, A is selected from methyl, propyl, Br, I, CN, methoxy, N(H)CH₂(phenyl), CF₃, and benzyl.

In some embodiments, A is Cy¹.

In some embodiments, A is selected from methyl, Br, and Cy¹.

In some embodiments, A is selected from methyl, Br, pyridyl, morpholinophenyl, phenyl, furanyl, oxazolyl, isoxazolyl, methylisoxazolyl, dimethylphenyl, methylfuranyl, pyrimidinyl, methylpyrazolyl, dimethylisoxazolyl, methylpyridinyl, thiazolyl, fluorophenyl, and methoxyphenyl.

In some embodiments, Cy¹ is selected from C₆₋₁₀ aryl and 5-14 membered heteroaryl, each optionally substituted by 1, 2, 3, 4, or 5 substituents independently selected from halo, C₁₋₆ alkyl, C₁₋₆ haloalkyl, C₃₋₁₀ cycloalkyl, 4-14 membered heterocycloalkyl, CN, NO₂, OR^{a}, SR^{a}, NR^{c}R^{d}, NR^{c}(O)R^{b}, S(O)NR^{c}R^{d}, S(O)₂R^{b}, and S(O)₂NR^{c}R^{d}.

In some embodiments, Cy¹ is C₆₋₁₀ aryl, 5-14 membered heteroaryl or 4-14 membered heterocycloalkyl, each of which is optionally substituted with 1, 2, or 3 substituents independently selected from halo, CN, C₁₋₆ alkyl, C₁₋₆ haloalkyl, 4-14 membered heterocycloalkyl, NR^{c}R^{d}, OR^{a}, and C(O)OR^{a}.

In some embodiments, Cy¹ is C₆₋₁₀ aryl or 5-14 membered heteroaryl, each optionally substituted with 1, 2, or 3 substituents independently selected from halo, C₁₋₆ alkyl, 4-14 membered heterocycloalkyl, and OR^{a}.

In some embodiments, Cy¹ is C₆₋₁₀ aryl or 5-14 membered heteroaryl, each optionally substituted with 1, 2, or 3 substituents independently selected from halo, C₁₋₆ alkyl, 4-14 membered heterocycloalkyl, and OCH₃.

In some embodiments, Cy¹ is C₆₋₁₀ aryl, optionally substituted with 1, 2, or 3 substituents independently selected from halo, C₁₋₆ alkyl, 4-14 membered heterocycloalkyl, and OR^{a}.

In some embodiments, Cy¹ is phenyl, optionally substituted with 1, 2, or 3 substituents independently selected from halo, C₁₋₆ alkyl, 4-14 membered heterocycloalkyl, and OR^{a}.

In some embodiments, Cy¹ is phenyl, optionally substituted with 1, 2, or 3 substituents independently selected from halo, C₁₋₆ alkyl, and OR^{a}.

In some embodiments, Cy¹ is 5-14 membered heteroaryl or 4-14 membered heterocycloalkyl, each of which is optionally substituted with 1, 2, or 3 substituents independently selected from halo, CN, C₁₋₆ alkyl, C₁₋₆ haloalkyl, 4-14 membered heterocycloalkyl, NR^{c}R^{d}, OR^{a}, and C(O)OR^{a}.

In some embodiments, Cy¹ is 5-14 membered heteroaryl, optionally substituted with 1, 2, or 3 substituents independently selected from halo, CN, C₁₋₆ alkyl, C₁₋₆ haloalkyl, 4-14 membered heterocycloalkyl, NR^{c}R^{d}, OR^{a}, and C(O)OR^{a}.

In some embodiments, Cy¹ is 5-14 membered heteroaryl, optionally substituted with 1, 2, or 3 substituents independently selected from halo, C₁₋₆ alkyl, and OR^{a}.

In some embodiments, Cy¹ is selected from pyridyl, phenyl, furanyl, oxazolyl, isoxazolyl, pyrimidinyl, pyrazolyl, thiazolyl, dihydrofuranyl, thiophenyl, tetrahydrofuranyl, pyrrolidinyl, isoindolinyl, azetidinyl, and imidazolyl; each of which is optionally substituted with 1, 2, or 3 substituents independently selected from halo, CN, C₁₋₆ alkyl, C₁₋₆ haloalkyl, 4-14 membered heterocycloalkyl, NR^{c}R^{d}, OR^{a} and C(O)OR^{a}.

In some embodiments, Cy¹ is selected from pyridyl, phenyl, furanyl, oxazolyl, isoxazolyl, pyrimidinyl, pyrazolyl, and thiazolyl; each optionally substituted with 1, 2, or 3 substituents independently selected from halo, C₁₋₆ alkyl, and OR^{a}.

In some embodiments, Cy¹ is selected from pyridyl, phenyl, furanyl, oxazolyl, isoxazolyl, pyrimidinyl, pyrazolyl, and thiazolyl; each optionally substituted with 1, 2, or 3 substituents independently selected from halo, C₁₋₆ alkyl, and OCH₃.

In some embodiments, Cy¹ is selected from pyridyl, morpholinophenyl, phenyl, furanyl, oxazolyl, isoxazolyl, methylisoxazolyl, dimethylphenyl, methylfuranyl, pyrimidinyl, methylpyrazolyl, dimethylisoxazolyl, methylpyridinyl, thiazolyl, fluorophenyl, and methoxyphenyl.

In some embodiments, Cy¹ is selected from pyridyl, morpholinophenyl, phenyl, furanyl, oxazolyl, isoxazolyl, methylisoxazolyl, dimethylphenyl, methylfuranyl, pyrimidinyl, methylpyrazolyl, dimethylisoxazolyl, methylpyridinyl, thiazolyl, fluorophenyl, methoxyphenyl, cyanophenyl, hydroxyphenyl, methylphenyl, dimethylpyrazolyl, methoxypyridinyl, dimethylpyridinyl, (difluoromethyl)pyrazolyl, (dimethylamino)phenyl, methoxymethylphenyl, (trifluoromethyl)pyridinyl, methyl(trifluoromethyl)pyrazolyl, (trifluoromethoxy)phenyl, morpholinophenyl, methylthiazolyl, methylthiophenyl, morpholinopyridinyl, thiophenyl, dimethylfuranyl, tetrahydrofuranyl, pyrrolidinyl, isoindolinyl, azetidinyl, pyrazolyl, imidazolyl, and carboxyazetidinyl.

In some embodiments, Cy¹ is furanyl. In some embodiments, Cy¹ is furan-3-yl. In some embodiments, Cy¹ is furan-2-yl.

In some embodiments, Cy¹ is oxazolyl. In some embodiments, Cy¹ is oxazol-5-yl.

In some embodiments, Q is the following group:

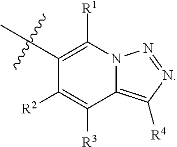

In some embodiments, Q is the following group:

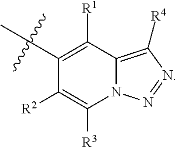

In some embodiments, Q is the following group:

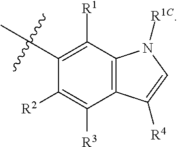

In some embodiments, Q is the following group:

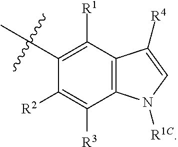

In some embodiments, Q is the following group:

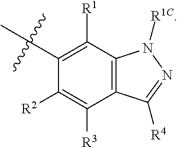

In some embodiments, Q is the following group:

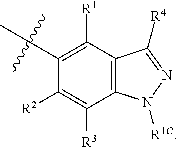

In some embodiments, Q is the following group:

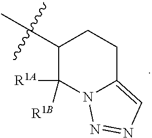

In some embodiments, Q is the following group:

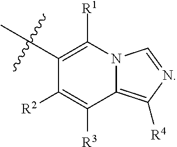

In some embodiments, Q is the following group:

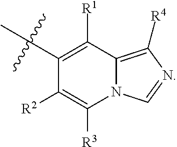

In some embodiments, Q is the following group:

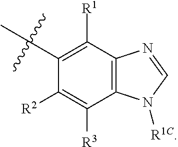

In some embodiments, Q is the following group:

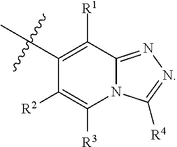

In some embodiments, Q is the following group:

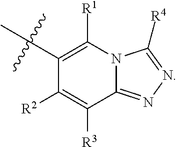

In some embodiments, Q is the following group:

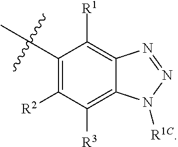

In some embodiments, Q is the following group:

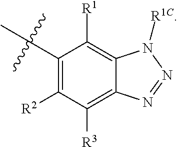

In some embodiments, Q is the following group:

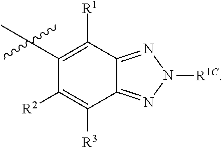

In some embodiments, Q is the following group:

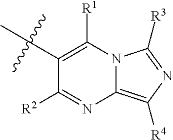

In some embodiments, Q is the following group:

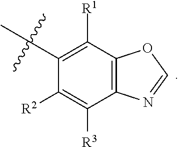

In some embodiments, Q is the following group:

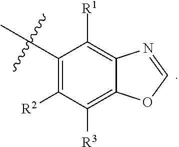

In some embodiments, Q is the following group:

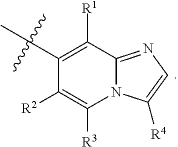

In some embodiments, Q is the following group:

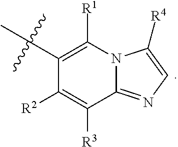

In some embodiments, Q is the following group:

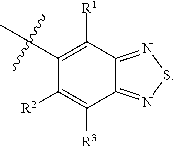

In some embodiments, Q is the following group:

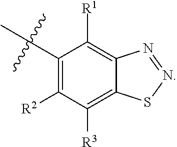

In some embodiments, Q is the following group:

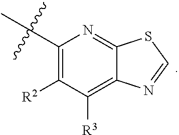

In some embodiments, Q is the following group:

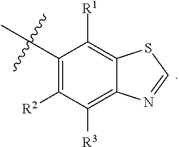

In some embodiments, Q is the following group:

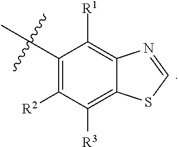

In some embodiments, Q is the following group:

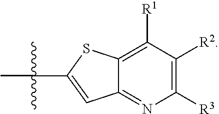

In some embodiments, Q is the following group:

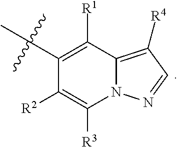

In some embodiments, Q is the following group:

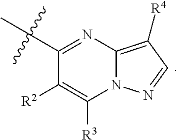

In some embodiments, Q is the following group:

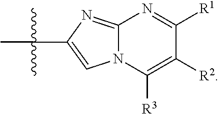

In some embodiments, Q is the following group:

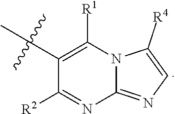

In some embodiments, Q is the following group:

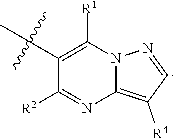

In some embodiments, Q is the following group:

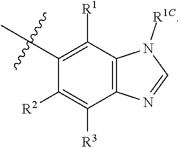

In some embodiments, Q is the following group:

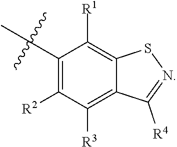

In some embodiments, Q is the following group:

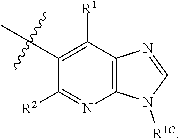

In some embodiments, Q is the following group:

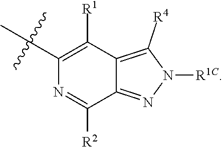

In some embodiments, Q is the following group:

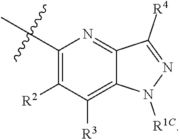

In some embodiments, Q is the following group:

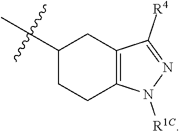

In some embodiments, Q is the following group:

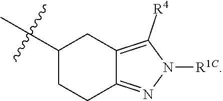

In some embodiments, Q is the following group:

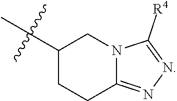

In some embodiments, Q is the following group:

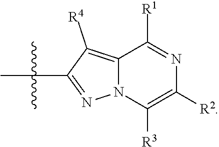

In some embodiments, Q is the following group:

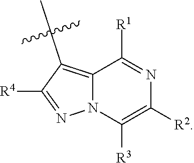

In some embodiments, Q is the following group:

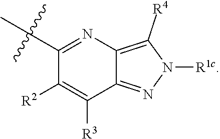

In some embodiments, Q is the following group:

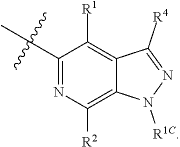

In some embodiments, Q is the following group:

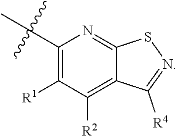

In some embodiments, Q is the following group:

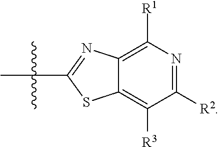

In some embodiments, Q is the following group:

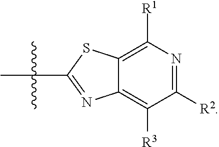

In some embodiments, Q is the following group:

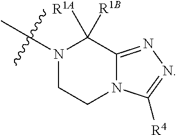

In some embodiments, Q is the following group:

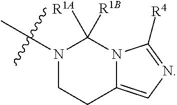

In some embodiments, Q is selected from the following groups:

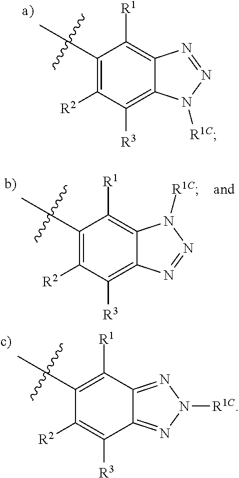

In some embodiments, Q is selected from the following groups:

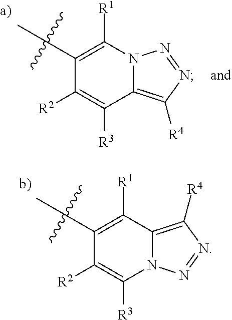

In some embodiments, Q is selected from the following groups:

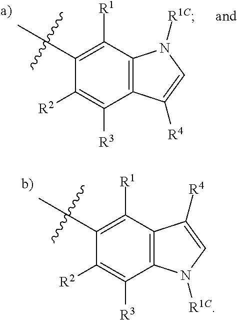

In some embodiments, Q is selected from the following groups:

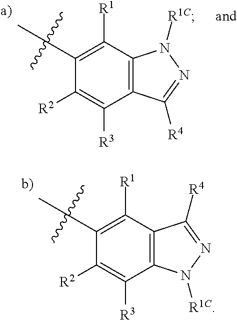

In some embodiments, Q is selected from the following groups:

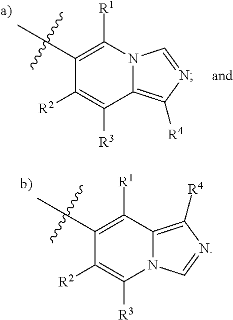

In some embodiments, R¹ is independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R¹ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

In some embodiments, R¹ is selected from H, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl and C₁₋₆ haloalkyl of R¹ are each optionally substituted with 1, 2, 3, 4, or 5 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

In some embodiments, R¹ is selected from halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl and C₁₋₆ haloalkyl of R¹ are each optionally substituted with 1, 2, 3, 4, or 5 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

In some embodiments, R¹ is selected from H, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1}.

In some embodiments, R¹ is selected from halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1}.

In some embodiments, R¹ is H, CN, halo, or C₁₋₆ alkyl. In some embodiments, R¹ is CN, halo, or C₁₋₆ alkyl. In some embodiments, R¹ is H, CN, F, Cl, or methyl. In some embodiments, R¹ is CN, F, Cl, or methyl. In some embodiments, R¹ is H. In some embodiments, R¹ is methyl. In some embodiments, R¹ is F. In some embodiments, R¹ is H, F, or methyl. In some embodiments, R¹ is F or methyl.

In some embodiments, R² is independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R² are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

In some embodiments, R² is selected from H, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl and C₁₋₆ haloalkyl of R² are each optionally substituted with 1, 2, 3, 4, or 5 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

In some embodiments, R² is selected from halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl and C₁₋₆ haloalkyl of R² are each optionally substituted with 1, 2, 3, 4, or 5 substituents independently selected from CN, NO₂, OR^{a}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

In some embodiments, R² is selected from H, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1}.

In some embodiments, R² is selected from halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1}.

In some embodiments, R² is H, halo, C₁₋₆ alkyl, or C₁₋₆ haloalkyl. In some embodiments, R² is halo, C₁₋₆ alkyl, or C₁₋₆ haloalkyl. In some embodiments, R² is H, Br, F, Cl, methyl, or CF₃. In some embodiments, R² is Br, F, Cl, methyl, or CF₃. In some embodiments, R² is H. In some embodiments, R² is methyl. In some embodiments, R² is F. In some embodiments, R² is H, F, or methyl. In some embodiments, R² is F or methyl.

In some embodiments, at least one of R¹ and R² is a group other than H.

In some embodiments, R³ is independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R³ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

In some embodiments, R³ is selected from H, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl and C₁₋₆ haloalkyl of R³ are each optionally substituted with 1, 2, 3, 4, or 5 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

In some embodiments, R³ is selected from H, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1}. In some embodiments, R³ is H or C₁₋₆ alkyl. In some embodiments, R³ is C₁₋₆ alkyl.

In some embodiments, R³ is H or methyl. In some embodiments, R³ is methyl. In some embodiments, R³ is H. In some embodiments, R³ is F. In some embodiments, R³ is H, F, or methyl.

In some embodiments, R¹, R², and R³ are each H. In some embodiments, R¹ and R² are each F, and R³ is H.

In some embodiments, R⁴ is independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R⁴ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

In some embodiments, R⁴ is selected from H, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl and C₁₋₆ haloalkyl of R⁴ are each optionally substituted with 1, 2, 3, 4, or 5 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

In some embodiments, R⁴ is H or C₁₋₆ alkyl. In some embodiments, R⁴ is C₁₋₆ alkyl. In some embodiments, R⁴ is H or methyl. In some embodiments, R⁴ is H. In some embodiments, R⁴ is methyl. In some embodiments, R⁴ is F. In some embodiments, R⁴ is H, F, or methyl.

In some embodiments, R⁵ is independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R⁵ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

In some embodiments, R⁵ is selected from H, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1}.

In some embodiments, R⁵ is selected from H, halo, and C₁₋₆ alkyl. In some embodiments, R⁵ is H. In some embodiments, R⁵ is F. In some embodiments, R⁵ is H or F.

In some embodiments, R⁶ is independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R⁶ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

In some embodiments, R⁶ is selected from H, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1}.

In some embodiments, R⁶ is selected from H, halo, and C₁₋₆ alkyl. In some embodiments, R⁶ is H. In some embodiments, R⁶ is F. In some embodiments, R⁶ is H or F.

In some embodiments, R⁷ is independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R⁷ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

In some embodiments, R⁷ is selected from H, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1}.

In some embodiments, R⁷ is selected from H, halo, and C₁₋₆ alkyl. In some embodiments, R⁷ is H. In some embodiments, R⁷ is F.

In some embodiments, R⁵, R⁶, and R⁷ are each independently selected from H, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1}.

In some embodiments, R⁵, R⁶, and R⁷ are each independently selected from H, halo, and C₁₋₆ alkyl. In some embodiments, R⁵, R⁶, and R⁷ are each H. In some embodiments, R⁵, R⁶, and R⁷ are each independently H or F.

In some embodiments, R^{1A} and R^{1B} are each independently selected from H, halo, C₁₋₆ alkyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, and NR^{c2}C(O)R^{b2}; wherein said C₁₋₆ alkyl and C₁₋₆ haloalkyl of RIA and R^{1B} are each optionally substituted with 1, 2, 3, 4, or 5 substituents independently selected from CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, and NR^{c2}C(O)R^{b2}.

In some embodiments, R^{1A} and R^{1B} are each independently selected from C₁₋₆ alkyl and H. In some embodiments, R^{1A} and R^{1B} are each C₁₋₆ alkyl. In some embodiments, R^{1A} and R^{1B} are each methyl.

In some embodiments, R^{1C} is C₁₋₆ alkyl. In some embodiments, R^{1C} is H or methyl. In some embodiments, R^{1C} is H. In some embodiments, R^{1C} is methyl.

In some embodiments, each R^{a}, R^{b}, R^{c}, R^{d}, R^{a1}, R^{b1}, R^{c1}, R^{d1}, R^{a2}, R^{b2}, R^{e2}, and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl.

In some embodiments, each R^{a}, R^{b}, R^{c}, R^{d}, R^{a1}, R^{b1}, R^{c1}, R^{d1}, R^{a2}, R^{b2}, R^{e2}, and R^{d2} is independently selected from H, C₁₋₆ alkyl, and C₁₋₆ haloalkyl.

In some embodiments, R^{a} is H, C₁₋₆ alkyl, or C₁₋₆ haloalkyl. In some embodiments, R^{a} is methyl. In some embodiments, R^{a} is H.

In some embodiments, R^{x} is H. In some embodiments, R^{x} is selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl.

In some embodiments, R^{y} is C₆₋₁₀ aryl-C₁₋₄ alkyl. In some embodiments, R^{y} is benzyl. In some embodiments, R^{y} is selected from C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl.

In some embodiments, the compound is of Formula Ia:

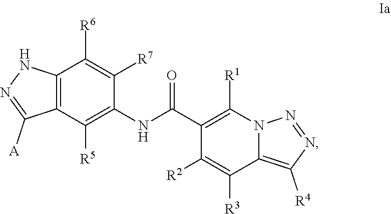

or a pharmaceutically acceptable salt thereof.

In some embodiments, the compound is of Formula Ib:

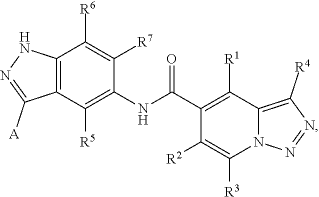

or a pharmaceutically acceptable salt thereof.

In some embodiments, the compound is of Formula Ic:

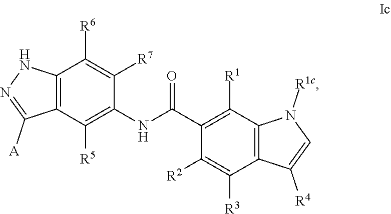

or a pharmaceutically acceptable salt thereof.

In some embodiments, the compound is of Formula Id:

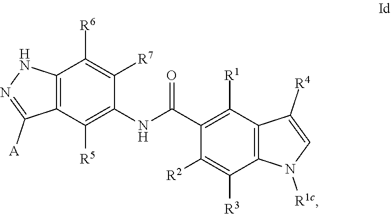

or a pharmaceutically acceptable salt thereof.

In some embodiments, the compound is of Formula Ie:

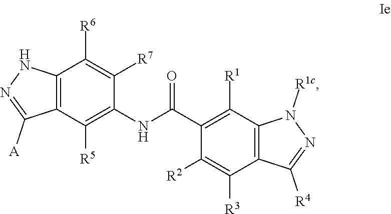

or a pharmaceutically acceptable salt thereof.

In some embodiments, the compound is of Formula If:

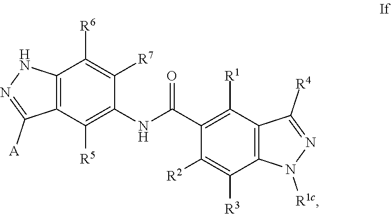

or a pharmaceutically acceptable salt thereof.

In some embodiments, the compound is of Formula Ig:

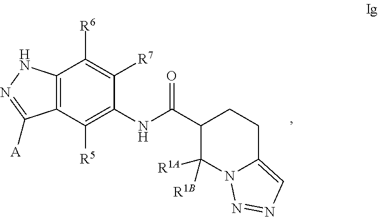

or a pharmaceutically acceptable salt thereof.

In some embodiments, the compound is of Formula Ih:

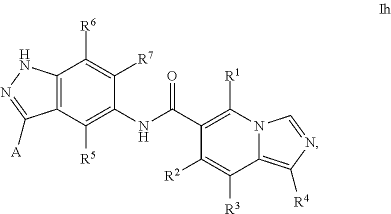

or a pharmaceutically acceptable salt thereof.

In some embodiments, the compound is of Formula Ii:

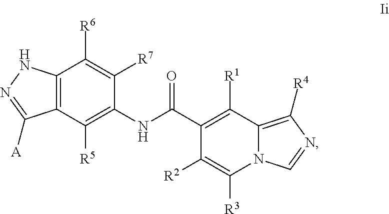

or a pharmaceutically acceptable salt thereof.

In some embodiments, the compound is of Formula Ij:

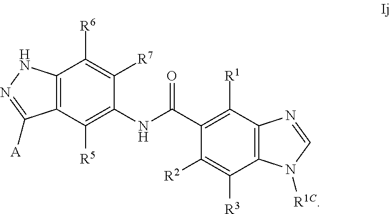

or a pharmaceutically acceptable salt thereof.

In some embodiments, the compound is of Formula Ik:

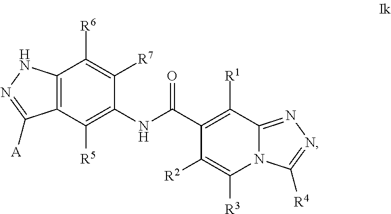

or a pharmaceutically acceptable salt thereof.

In some embodiments, the compound is of Formula Il:

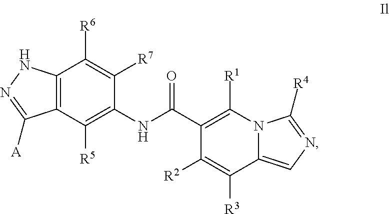

or a pharmaceutically acceptable salt thereof.

In some embodiments, the compound is of Formula Im:

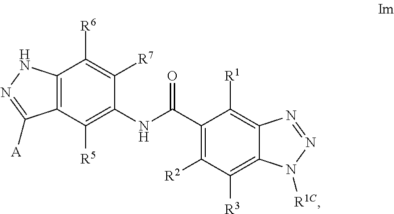

or a pharmaceutically acceptable salt thereof.

In some embodiments, the compound is of Formula In:

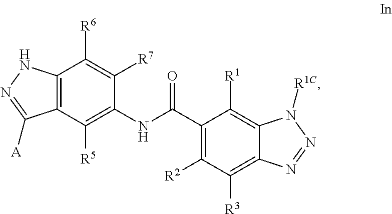

or a pharmaceutically acceptable salt thereof.

In some embodiments, the compound is of Formula Io:

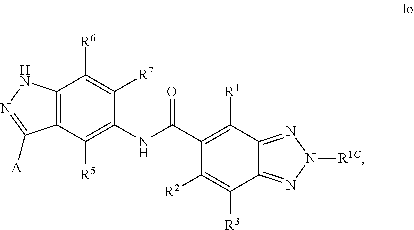

or a pharmaceutically acceptable salt thereof.

In some embodiments, the compound is of Formula IIa:

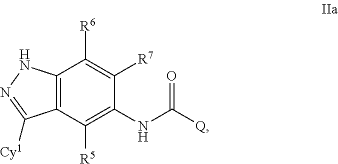

or a pharmaceutically acceptable salt thereof.

In some embodiments, the compound is of Formula IIb:

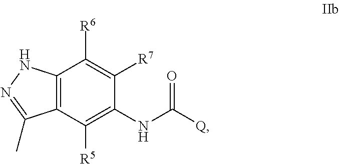

or a pharmaceutically acceptable salt thereof.

In some embodiments, the compound is of Formula IIc:

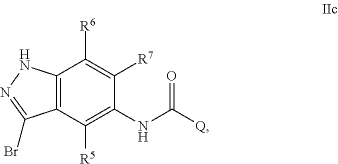

or a pharmaceutically acceptable salt thereof.

In some embodiments, the compound is of Formula III:

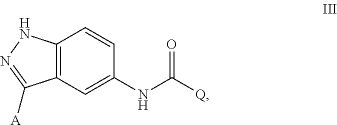

or a pharmaceutically acceptable salt thereof.

In some embodiments, the compound is of Formula IIIa:

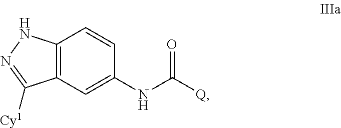

or a pharmaceutically acceptable salt thereof.

In some embodiments, the compound is of Formula IIIb:

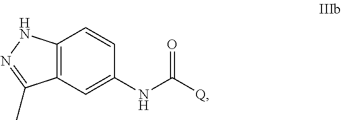

or a pharmaceutically acceptable salt thereof.

In some embodiments, the compound is of Formula IIIc:

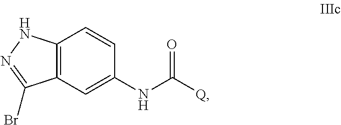

or a pharmaceutically acceptable salt thereof.

In some embodiments, provided herein is a compound selected from:
5-Methyl-N-(3-phenyl-1H-indazol-5-yl)-1H-benzo[d][1,2,3]triazole-6-carboxamide;
5-Methyl-N-(3-(3-morpholinophenyl)-1H-indazol-5-yl)-1H-benzo[d][1,2,3]triazole-6-carboxamide;
2,6-Dimethyl-N-(3-methyl-1H-indazol-5-yl)-2H-benzo[d][1,2,3]triazole-5-carboxamide;
1,6-Dimethyl-N-(3-methyl-1H-indazol-5-yl)-1H-benzo[d][1,2,3]triazole-5-carboxamide;
1,5-dimethyl-N-(3-methyl-1H-indazol-5-yl)-1H-benzo[d][1,2,3]triazole-6-carboxamide;
N-(3-Methyl-1H-indazol-5-yl)-[1,2,4]triazolo[4,3-a]pyridine-7-carboxamide;
3-Methyl-N-(3-methyl-1H-indazol-5-yl)-[1,2,4]triazolo[4,3-a]pyridine-7-carboxamide;
N-(3-Methyl-1H-indazol-5-yl)-[1,2,4]triazolo[4,3-a]pyridine-6-carboxamide;
3-Methyl-N-(3-methyl-1H-indazol-5-yl)-[1,2,4]triazolo[4,3-a]pyridine-6-carboxamide;
N-(3-Methyl-1H-indazol-5-yl)-1H-benzo[d][1,2,3]triazole-6-carboxamide;
N-(3-Methyl-1H-indazol-5-yl)-6-(trifluoromethyl)-1H-benzo[d][1,2,3]triazole-5-carboxamide;
4,6-Dimethyl-N-(3-methyl-1H-indazol-5-yl)-1H-benzo[d][1,2,3]triazole-5-carboxamide;
4-Methyl-N-(3-(pyridin-4-yl)-1H-indazol-5-yl)-1H-benzo[d][1,2,3]triazole-5-carboxamide;
7-Methyl-N-(3-(pyridin-4-yl)-1H-indazol-5-yl)-1H-benzo[d][1,2,3]triazole-5-carboxamide;
4-Cyano-N-(3-methyl-1H-indazol-5-yl)-1H-benzo[d][1,2,3]triazole-5-carboxamide;
N-(3-Bromo-1H-indazol-5-yl)-5-methyl-1H-benzo[d][1,2,3]triazole-6-carboxamide;
5-Methyl-N-(3-methyl-1H-indazol-5-yl)-1H-benzo[d][1,2,3]triazole-6-carboxamide;
6-Methyl-N-(3-phenyl-1H-indazol-5-yl)-1H-benzo[d][1,2,3]triazole-5-carboxamide;
N-(3-Bromo-1H-indazol-5-yl)-6-methyl-1H-benzo[d]imidazole-5-carboxamide;
6-Methyl-N-(3-phenyl-1H-indazol-5-yl)-1H-benzo[d]imidazole-5-carboxamide;
6-Methyl-N-(3-phenyl-1H-indazol-5-yl)-1H-indole-5-carboxamide;
6-Methyl-N-(3-phenyl-1H-indazol-5-yl)-1H-indazole-5-carboxamide;
5-Methyl-N-(3-phenyl-1H-indazol-5-yl)-1H-indole-6-carboxamide;
5-Methyl-N-(3-phenyl-1H-indazol-5-yl)-1H-indazole-6-carboxamide;
5-Bromo-N-(3-phenyl-1H-indazol-5-yl)-1H-indazole-6-carboxamide;
4,6-Difluoro-N-(3-(furan-2-yl)-1H-indazol-5-yl)-1-methyl-1H-indazole-5-carboxamide;
4,6-Difluoro-1-methyl-N-(3-(oxazol-5-yl)-1H-indazol-5-yl)-1H-indazole-5-carboxamide;
4,6-Difluoro-N-(3-(isoxazol-4-yl)-1H-indazol-5-yl)-1-methyl-1H-indazole-5-carboxamide;
4,6-Difluoro-N-(3-(furan-3-yl)-1H-indazol-5-yl)-1-methyl-1H-indazole-5-carboxamide;
4,6-Difluoro-1-methyl-N-(3-(5-methylisoxazol-4-yl)-1H-indazol-5-yl)-1H-indazole-5-carboxamide;
4,6-Difluoro-1-methyl-N-(3-(3-methylisoxazol-4-yl)-1H-indazol-5-yl)-1H-indazole-5-carboxamide;
N-(3-(2,3-Dimethylphenyl)-1H-indazol-5-yl)-4,6-difluoro-1-methyl-1H-indazole-5-carboxamide;
4,6-Difluoro-1-methyl-N-(3-(pyridin-3-yl)-1H-indazol-5-yl)-1H-indazole-5-carboxamide;
4,6-Difluoro-1-methyl-N-(3-(5-methylfuran-2-yl)-1H-indazol-5-yl)-1H-indazole-5-carboxamide;
4,6-Difluoro-1-methyl-N-(3-(pyrimidin-5-yl)-1H-indazol-5-yl)-1H-indazole-5-carboxamide;
4,6-Difluoro-1-methyl-N-(3-(1-methyl-1H-pyrazol-4-yl)-1H-indazol-5-yl)-1H-indazole-5-carboxamide;
N-(3-(3,5-Dimethylisoxazol-4-yl)-1H-indazol-5-yl)-4,6-difluoro-1-methyl-1H-indazole-5-carboxamide;
4,6-Difluoro-1-methyl-N-(3-(2-methylpyridin-4-yl)-1H-indazol-5-yl)-1H-indazole-5-carboxamide;
4,6-Difluoro-1-methyl-N-(3-(4-methylpyridin-3-yl)-1H-indazol-5-yl)-1H-indazole-5-carboxamide;
4,6-Difluoro-1-methyl-N-(3-(2-methylpyridin-3-yl)-1H-indazol-5-yl)-1H-indazole-5-carboxamide;
4,6-Difluoro-1-methyl-N-(3-(3-methylpyridin-4-yl)-1H-indazol-5-yl)-1H-indazole-5-carboxamide;
4,6-Difluoro-1-methyl-N-(3-(pyridin-2-yl)-1H-indazol-5-yl)-1H-indazole-5-carboxamide;
4,6-Difluoro-1-methyl-N-(3-(thiazol-5-yl)-1H-indazol-5-yl)-1H-indazole-5-carboxamide;
5-Methyl-N-(3-phenyl-1H-indazol-5-yl)-[1,2,3]triazolo[1,5-a]pyridine-6-carboxamide;
N-(3-(2-Fluorophenyl)-1H-indazol-5-yl)-5,7-dimethyl-[1,2,3]triazolo[1,5-a]pyridine-6-carboxamide;
N-(3-Bromo-1H-indazol-5-yl)-5,7-dimethyl-[1,2,3]triazolo[1,5-a]pyridine-6-carboxamide;
N-(3-(2-Methoxyphenyl)-1H-indazol-5-yl)-5,7-dimethyl-[1,2,3]triazolo[1,5-a]pyridine-6-carboxamide;
N-(3-(2,3-Dimethylphenyl)-1H-indazol-5-yl)-5,7-dimethyl-[1,2,3]triazolo[1,5-a]pyridine-6-carboxamide;
5,7-Dimethyl-N-(3-phenyl-1H-indazol-5-yl)-[1,2,3]triazolo[1,5-a]pyridine-6-carboxamide;
5,7-Dimethyl-N-(3-methyl-1H-indazol-5-yl)-[1,2,3]triazolo[1,5-a]pyridine-6-carboxamide;
4,6-Difluoro-1-methyl-N-(3-phenyl-1H-indazol-5-yl)-1H-indazole-5-carboxamide;
4,6-Difluoro-1-methyl-N-(3-methyl-1H-indazol-5-yl)-1H-indazole-5-carboxamide;
6-Methyl-N-(3-phenyl-1H-indazol-5-yl)imidazo[1,5-a]pyridine-7-carboxamide;
1-Methyl-N-(3-methyl-1H-indazol-5-yl)-1H-indazole-5-carboxamide;
(R)-7,7-Dimethyl-N-(3-phenyl-1H-indazol-5-yl)-4,5,6,7-tetrahydro-[1,2,3]triazolo[1,5-a]pyridine-6-carboxamide;
5,7-Dimethyl-N-(3-methyl-1H-indazol-5-yl)imidazo[1,5-a]pyridine-6-carboxamide;
6,8-Dichloro-N-(3-methyl-1H-indazol-5-yl)-[1,2,4]triazolo[4,3-a]pyridine-7-carboxamide;
6,8-Dimethyl-N-(3-phenyl-1H-indazol-5-yl)-[1,2,4]triazolo[4,3-a]pyridine-7-carboxamide;
6,8-Dimethyl-N-(3-methyl-1H-indazol-5-yl)-[1,2,4]triazolo[4,3-a]pyridine-7-carboxamide;
6-Chloro-8-methyl-N-(3-phenyl-1H-indazol-5-yl)-[1,2,4]triazolo[4,3-a]pyridine-7-carboxamide;
5,7-Dimethyl-N-(3-phenyl-1H-indazol-5-yl)-[1,2,4]triazolo[4,3-a]pyridine-6-carboxamide;
5,7-Dimethyl-N-(3-methyl-1H-indazol-5-yl)-[1,2,4]triazolo[4,3-a]pyridine-6-carboxamide;
5-Methyl-N-(3-phenyl-1H-indazol-5-yl)imidazo[1,5-a]pyridine-6-carboxamide;
6,8-dichloro-N-(3-phenyl-1H-indazol-5-yl)-[1,2,4]triazolo[4,3-a]pyridine-7-carboxamide;
3,5,7-Trimethyl-N-(3-phenyl-1H-indazol-5-yl)-[1,2,3]triazolo[1,5-a]pyridine-6-carboxamide;
3,5,7-Trimethyl-N-(3-methyl-1H-indazol-5-yl)-[1,2,3]triazolo[1,5-a]pyridine-6-carboxamide;
5,7-Dimethyl-N-(3-(3-morpholinophenyl)-1H-indazol-5-yl)-[1,2,3]triazolo[1,5-a]pyridine-6-carboxamide;
4-Methyl-N-(3-phenyl-1H-indazol-5-yl)-[1,2,3]triazolo[1,5-a]pyridine-5-carboxamide;
N-(3-(Furan-3-yl)-1H-indazol-5-yl)-5,7-dimethyl-[1,2,3]triazolo[1,5-a]pyridine-6-carboxamide; and
5,7-Dimethyl-N-(3-phenyl-1H-indazol-5-yl)imidazo[1,5-a]pyridine-6-carboxamide;

or a pharmaceutically acceptable salt of any of the aforementioned.

In some embodiments, provided herein is a compound selected from:
N-(3-Phenyl-1H-indazol-5-yl)-5-(trifluoromethyl)-1H-indazole-6-carboxamide;
4,6-Difluoro-1-methyl-N-(3-(6-methylpyridin-2-yl)-1H-indazol-5-yl)-1H-indazole-5-carboxamide;
N-(3-(4,5-Dihydrofuran-2-yl)-1H-indazol-5-yl)-4,6-difluoro-1-methyl-1H-indazole-5-carboxamide;
4,6-Difluoro-1-methyl-N-(3-(pyrimidin-4-yl)-1H-indazol-5-yl)-1H-indazole-5-carboxamide;
N-(3-(2-Cyanophenyl)-1H-indazol-5-yl)-4,6-difluoro-1-methyl-1H-indazole-5-carboxamide;
4,6-Difluoro-N-(3-(2-hydroxyphenyl)-1H-indazol-5-yl)-1-methyl-1H-indazole-5-carboxamide;
4,6-Difluoro-1-methyl-N-(3-(m-tolyl)-1H-indazol-5-yl)-1H-indazole-5-carboxamide;
4,6-Difluoro-1-methyl-N-(3-(o-tolyl)-1H-indazol-5-yl)-1H-indazole-5-carboxamide;
4,6-Difluoro-N-(3-(2-fluorophenyl)-1H-indazol-5-yl)-1-methyl-1H-indazole-5-carboxamide;
N-(3-(1,3-Dimethyl-1H-pyrazol-5-yl)-1H-indazol-5-yl)-4,6-difluoro-1-methyl-1H-indazole-5-carboxamide;
N-(3-(1,3-Dimethyl-1H-pyrazol-4-yl)-1H-indazol-5-yl)-4,6-difluoro-1-methyl-1H-indazole-5-carboxamide;
4,6-Difluoro-N-(3-(2-methoxypyridin-3-yl)-1H-indazol-5-yl)-1-methyl-1H-indazole-5-carboxamide;
4,6-Difluoro-N-(3-(5-methoxypyridin-3-yl)-1H-indazol-5-yl)-1-methyl-1H-indazole-5-carboxamide;
N-(3-(2,6-Dimethylpyridin-4-yl)-1H-indazol-5-yl)-4,6-difluoro-1-methyl-1H-indazole-5-carboxamide;
4,6-Difluoro-N-(3-(3-methoxyphenyl)-1H-indazol-5-yl)-1-methyl-1H-indazole-5-carboxamide;
4,6-Difluoro-N-(3-(2-methoxyphenyl)-1H-indazol-5-yl)-1-methyl-1H-indazole-5-carboxamide;
N-(3-(1-(Difluoromethyl)-1H-pyrazol-4-yl)-1H-indazol-5-yl)-4,6-difluoro-1-methyl-1H-indazole-5-carboxamide;
N-(3-(4-(Dimethylamino)phenyl)-1H-indazol-5-yl)-4,6-difluoro-1-methyl-1H-indazole-5-carboxamide;
N-(3-(3-(Dimethylamino)phenyl)-1H-indazol-5-yl)-4,6-difluoro-1-methyl-1H-indazole-5-carboxamide;
4,6-Difluoro-N-(3-(2-methoxy-5-methylphenyl)-1H-indazol-5-yl)-1-methyl-1H-indazole-5-carboxamide;
4,6-Difluoro-N-(3-(5-methoxy-2-methylphenyl)-1H-indazol-5-yl)-1-methyl-1H-indazole-5-carboxamide;
4,6-Difluoro-1-methyl-N-(3-(4-(trifluoromethyl)pyridin-3-yl)-1H-indazol-5-yl)-1H-indazole-5-carboxamide;
4,6-Difluoro-1-methyl-N-(3-(1-methyl-3-(trifluoromethyl)-1H-pyrazol-4-yl)-1H-indazol-5-yl)-1H-indazole-5-carboxamide;
4,6-Difluoro-1-methyl-N-(3-(2-(trifluoromethoxy)phenyl)-1H-indazol-5-yl)-1H-indazole-5-carboxamide;
4,6-Difluoro-1-methyl-N-(3-(4-morpholinophenyl)-1H-indazol-5-yl)-1H-indazole-5-carboxamide;
4,6-Difluoro-1-methyl-N-(3-(2-methylthiazol-5-yl)-1H-indazol-5-yl)-1H-indazole-5-carboxamide;
4,6-Difluoro-1-methyl-N-(3-(1-methyl-1H-pyrazol-5-yl)-1H-indazol-5-yl)-1H-indazole-5-carboxamide;
4,6-Difluoro-1-methyl-N-(3-(5-methylthiophen-2-yl)-1H-indazol-5-yl)-1H-indazole-5-carboxamide;
4,6-Difluoro-1-methyl-N-(3-(5-morpholinopyridin-3-yl)-1H-indazol-5-yl)-1H-indazole-5-carboxamide;
4,6-Difluoro-1-methyl-N-(3-propyl-1H-indazol-5-yl)-1H-indazole-5-carboxamide;
4,6-Difluoro-1-methyl-N-(3-(thiophen-3-yl)-1H-indazol-5-yl)-1H-indazole-5-carboxamide;
N-(3-(2,5-Dimethylfuran-3-yl)-1H-indazol-5-yl)-4,6-difluoro-1-methyl-1H-indazole-5-carboxamide;
4,6-Difluoro-N-(3-(4-methoxypyridin-3-yl)-1H-indazol-5-yl)-1-methyl-1H-indazole-5-carboxamide;
4,6-Difluoro-N-(3-(3-methoxypyridin-4-yl)-1H-indazol-5-yl)-1-methyl-1H-indazole-5-carboxamide;
4,6-Difluoro-1-methyl-N-(3-(tetrahydrofuran-3-yl)-1H-indazol-5-yl)-1H-indazole-5-carboxamide;
N-(3-Cyano-1H-indazol-5-yl)-4,6-difluoro-1-methyl-1H-indazole-5-carboxamide;
4,6-Difluoro-N-(3-methoxy-1H-indazol-5-yl)-1-methyl-1H-indazole-5-carboxamide;
1-Methyl-N-(3-phenyl-1H-indazol-5-yl)-1H-indazole-5-carboxamide;
4,6-Difluoro-1-methyl-N-(3-(pyrrolidin-1-yl)-1H-indazol-5-yl)-1H-indazole-5-carboxamide;
4,6-Difluoro-N-(3-(isoindolin-2-yl)-1H-indazol-5-yl)-1-methyl-1H-indazole-5-carboxamide;
N-(3-(Benzylamino)-1H-indazol-5-yl)-4,6-difluoro-1-methyl-1H-indazole-5-carboxamide;
N-(3-Iodo-1H-indazol-5-yl)-2,4-dimethylimidazo[1,5-a]pyrimidine-3-carboxamide;
N-(3-(Furan-3-yl)-1H-indazol-5-yl)-2,4-dimethylimidazo[1,5-a]pyrimidine-3-carboxamide;
N-(3-(Furan-3-yl)-1H-indazol-5-yl)-4-methylimidazo[1,5-a]pyrimidine-3-carboxamide;
N-(3-(Furan-3-yl)-1H-indazol-5-yl)-4-methylimidazo[1,5-a]pyrimidine-3-carboxamide;
4,6-Difluoro-1-methyl-N-(3-(trifluoromethyl)-1H-indazol-5-yl)-1H-indazole-5-carboxamide;
N-(3-(furan-3-yl)-1H-indazol-5-yl)benzo[d]oxazole-6-carboxamide;
N-(3-(Furan-3-yl)-1H-indazol-5-yl)pyrazolo[1,5-a]pyridine-5-carboxamide;
N-(3-(Furan-3-yl)-1H-indazol-5-yl)imidazo[1,2-a]pyridine-7-carboxamide;
N-(3-(Furan-3-yl)-1H-indazol-5-yl)imidazo[1,2-a]pyridine-6-carboxamide;
N-(3-(Furan-3-yl)-1H-indazol-5-yl)benzo[c][1,2,5]thiadiazole-5-carboxamide;
N-(3-(Furan-3-yl)-1H-indazol-5-yl)benzo[d][1,2,3]thiadiazole-5-carboxamide;
N-(3-(Furan-3-yl)-1H-indazol-5-yl)thiazolo[5,4-b]pyridine-5-carboxamide;
N-(3-(Furan-3-yl)-1H-indazol-5-yl)benzo[d]thiazole-6-carboxamide;
N-(3-(Furan-3-yl)-1H-indazol-5-yl)benzo[d]thiazole-5-carboxamide;
N-(3-(Furan-3-yl)-1H-indazol-5-yl)thieno[3,2-b]pyridine-2-carboxamide;
N-(3-(Furan-3-yl)-1H-indazol-5-yl)benzo[d]oxazole-5-carboxamide;
N-(3-(Furan-3-yl)-1H-indazol-5-yl)pyrazolo[1,5-a]pyrimidine-5-carboxamide;
N-(3-(Furan-3-yl)-1H-indazol-5-yl)imidazo[1,2-a]pyrimidine-2-carboxamide;
N-(3-(Furan-3-yl)-1H-indazol-5-yl)imidazo[1,2-a]pyrimidine-6-carboxamide;
N-(3-(Furan-3-yl)-1H-indazol-5-yl)pyrazolo[1,5-a]pyrimidine-6-carboxamide;
N-(3-(Furan-3-yl)-1H-indazol-5-yl)-1-methyl-1H-benzo[d]imidazole-6-carboxamide;
N-(3-(Furan-3-yl)-1H-indazol-5-yl)-1-methyl-1H-benzo[d]imidazole-5-carboxamide;
N-(3-(Furan-3-yl)-1H-indazol-5-yl)-1-methyl-1H-indazole-5-carboxamide;
N-(3-(Furan-3-yl)-1H-indazol-5-yl)-2-methylbenzo[d]oxazole-6-carboxamide;
4,6-Difluoro-N-(3-(isoxazol-3-yl)-1H-indazol-5-yl)-1-methyl-1H-indazole-5-carboxamide;
4,6-Difluoro-1-methyl-N-(3-(oxazol-4-yl)-1H-indazol-5-yl)-1H-indazole-5-carboxamide;
4,6-Difluoro-N-(3-(isoxazol-5-yl)-1H-indazol-5-yl)-1-methyl-1H-indazole-5-carboxamide;
4,6-Difluoro-1-methyl-N-(3-(oxazol-2-yl)-1H-indazol-5-yl)-1H-indazole-5-carboxamide;
N-(3-(Azetidin-1-yl)-1H-indazol-5-yl)-4,6-difluoro-1-methyl-1H-indazole-5-carboxamide;
N-(3-Benzyl-1H-indazol-5-yl)-4,6-difluoro-1-methyl-1H-indazole-5-carboxamide;
N-(3-(1H-Imidazol-1-yl)-1H-indazol-5-yl)-4,6-difluoro-1-methyl-1H-indazole-5-carboxamide;
1-(5-(4,6-difluoro-1-methyl-1H-indazole-5-carboxamido)-1H-indazol-3-yl)azetidine-3-carboxylic acid;
4,6-Difluoro-N-(6-fluoro-3-phenyl-1H-indazol-5-yl)-1-methyl-1H-indazole-5-carboxamide;
4,6-Difluoro-N-(6-fluoro-3-(furan-3-yl)-1H-indazol-5-yl)-1-methyl-1H-indazole-5-carboxamide;
7-Methyl-N-(3-methyl-1H-indazol-5-yl)imidazo[1,5-a]pyridine-6-carboxamide;
N-(3-(Furan-3-yl)-1H-indazol-5-yl)-7-methylimidazo[1,5-a]pyridine-6-carboxamide;
N-(3-(Furan-3-yl)-1H-indazol-5-yl)-1,5,7-trimethylimidazo[1,5-a]pyridine-6-carboxamide;
1,5,7-Trimethyl-N-(3-methyl-1H-indazol-5-yl)imidazo[1,5-a]pyridine-6-carboxamide;
4-Methyl-N-(3-phenyl-1H-indazol-5-yl)-[1,2,3]triazolo[1,5-a]pyridine-5-carboxamide;
N-(3-(Furan-3-yl)-1H-indazol-5-yl)-5,7-dimethyl-[1,2,3]triazolo[1,5-a]pyridine-6-carboxamide;
N-(3-(Furan-3-yl)-1H-indazol-5-yl)-5,7-dimethylimidazo[1,5-a]pyridine-6-carboxamide;
N-(3-(Furan-3-yl)-1H-indazol-5-yl)-6-methylimidazo[1,5-a]pyridine-7-carboxamide;
N-(3-(Furan-3-yl)-1H-indazol-5-yl)-5,7-dimethyl-[1,2,4]triazolo[4,3-a]pyridine-6-carboxamide;
N-(3-(Furan-2-yl)-1H-indazol-5-yl)-6-methylimidazo[1,5-a]pyridine-7-carboxamide;
N-(3-(Furan-2-yl)-1H-indazol-5-yl)-5,7-dimethyl-[1,2,3]triazolo[1,5-a]pyridine-6-carboxamide;
N-(3-(Furan-3-yl)-1H-indazol-5-yl)benzo[d]isothiazole-6-carboxamide;
N-(3-(Furan-3-yl)-1H-indazol-5-yl)-1,4,6-trimethyl-1H-indazole-5-carboxamide;
N-(3-(Furan-3-yl)-1H-indazol-5-yl)-1,6-dimethyl-1H-indazole-5-carboxamide;
2-Methyl-N-(3-(oxazol-5-yl)-1H-indazol-5-yl)-2H-pyrazolo[3,4-c]pyridine-5-carboxamide; and
1,6-Dimethyl-N-(3-(oxazol-5-yl)-1H-indazol-5-yl)-1H-pyrazolo[4,3-b]pyridine-5-carboxamide,

or a pharmaceutically acceptable salt of any of the aforementioned.

In some embodiments, provided herein is a compound selected from:
(R)-N-(3-(Furan-3-yl)-1H-indazol-5-yl)-1-methyl-4,5,6,7-tetrahydro-1H-indazole-5-carboxamide;
(S)-N-(3-(Furan-3-yl)-1H-indazol-5-yl)-1-methyl-4,5,6,7-tetrahydro-1H-indazole-5-carboxamide;
(R)-N-(3-(furan-3-yl)-1H-indazol-5-yl)-2-methyl-4,5,6,7-tetrahydro-2H-indazole-5-carboxamide;
(S)-N-(3-(furan-3-yl)-1H-indazol-5-yl)-2-methyl-4,5,6,7-tetrahydro-2H-indazole-5-carboxamide;
(R)-N-(3-(furan-3-yl)-1H-indazol-5-yl)-5,6,7,8-tetrahydro-[1,2,4]triazolo[4,3-a]pyridine-6-carboxamide;
(S)-N-(3-(furan-3-yl)-1H-indazol-5-yl)-5,6,7,8-tetrahydro-[1,2,4]triazolo[4,3-a]pyridine-6-carboxamide;
N-(3-(Furan-3-yl)-1H-indazol-5-yl)-4,6-dimethylpyrazolo[1,5-a]pyrazine-2-carboxamide;
N-(3-(furan-3-yl)-1H-indazol-5-yl)-2-methylpyrazolo[1,5-a]pyrazine-3-carboxamide;
1-Methyl-N-(3-(oxazol-5-yl)-1H-indazol-5-yl)-1H-pyrazolo[4,3-b]pyridine-5-carboxamide;
2-Methyl-N-(3-(oxazol-5-yl)-1H-indazol-5-yl)-2H-pyrazolo[4,3-b]pyridine-5-carboxamide;
1-Methyl-N-(3-(oxazol-5-yl)-1H-indazol-5-yl)-1H-pyrazolo[3,4-c]pyridine-5-carboxamide;
5-Methyl-N-(3-(oxazol-5-yl)-1H-indazol-5-yl)isothiazolo[5,4-b]pyridine-6-carboxamide;
1,4-Dimethyl-N-(3-(oxazol-5-yl)-1H-indazol-5-yl)-1H-pyrazolo[3,4-c]pyridine-5-carboxamide;
N-(3-(Furan-3-yl)-1H-indazol-5-yl)thiazolo[4,5-c]pyridine-2-carboxamide;
N-(3-(Furan-3-yl)-1H-indazol-5-yl)thiazolo[5,4-c]pyridine-2-carboxamide;
N-(3-(Furan-3-yl)-1H-indazol-5-yl)-8,8-dimethyl-5,6-dihydro-[1,2,4]triazolo[4,3-a]pyrazine-7(8H)-carboxamide;
(S)-N-(3-(Furan-3-yl)-1H-indazol-5-yl)-5,6,7,8-tetrahydroimidazo[1,5-a]pyridine-6-carboxamide; and
(S)-N-(3-(Furan-3-yl)-1H-indazol-5-yl)-5,6,7,8-tetrahydroimidazo[1,5-a]pyridine-6-carboxamide,

or a pharmaceutically acceptable salt of any of the aforementioned.

It is further appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination. Thus, it is contemplated as features described as embodiments of the compounds of Formula I can be combined in any suitable combination.

At various places in the present specification, certain features of the compounds are disclosed in groups or in ranges. It is specifically intended that such a disclosure include each and every individual subcombination of the members of such groups and ranges. For example, the term “C₁₋₆ alkyl” is specifically intended to individually disclose (without limitation) methyl, ethyl, C₃ alkyl, C₄ alkyl, C₅ alkyl and C₆ alkyl.

The term “n-membered,” where n is an integer, typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring and 1,2,3,4-tetrahydro-naphthalene is an example of a 10-membered cycloalkyl group.

At various places in the present specification, variables defining divalent linking groups may be described. It is specifically intended that each linking substituent include both the forward and backward forms of the linking substituent. For example, —NR(CR′R″)ₙ— includes both —NR(CR′R″)ₙ— and —(CR′R″)ₙNR— and is intended to disclose each of the forms individually. Where the structure requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

The term “substituted” means that an atom or group of atoms formally replaces hydrogen as a “substituent” attached to another group. The term “substituted”, unless otherwise indicated, refers to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. It is to be understood that substitution at a given atom results in a chemically stable molecule. The phrase “optionally substituted” means unsubstituted or substituted. The term “substituted” means that a hydrogen atom is removed and replaced by a substituent. A single divalent substituent, e.g., oxo, can replace two hydrogen atoms.

The term “Cn-m” indicates a range which includes the endpoints, wherein n and m are integers and indicate the number of carbons. Examples include C₁₋₄, C₁₋₆ and the like.

The term “alkyl” employed alone or in combination with other terms, refers to a saturated hydrocarbon group that may be straight-chained or branched. The term “Cn-m alkyl”, refers to an alkyl group having n to m carbon atoms. An alkyl group formally corresponds to an alkane with one C—H bond replaced by the point of attachment of the alkyl group to the remainder of the compound. In some embodiments, the alkyl group contains from 1 to 6 carbon atoms, from 1 to 4 carbon atoms, from 1 to 3 carbon atoms, or 1 to 2 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl and the like.

The term “alkenyl” employed alone or in combination with other terms, refers to a straight-chain or branched hydrocarbon group corresponding to an alkyl group having one or more double carbon-carbon bonds. An alkenyl group formally corresponds to an alkene with one C—H bond replaced by the point of attachment of the alkenyl group to the remainder of the compound. The term “Cn-m alkenyl” refers to an alkenyl group having n to m carbons. In some embodiments, the alkenyl moiety contains 2 to 6, 2 to 4, or 2 to 3 carbon atoms. Example alkenyl groups include, but are not limited to, ethenyl, n-propenyl, isopropenyl, n-butenyl, sec-butenyl and the like.

The term “alkynyl” employed alone or in combination with other terms, refers to a straight-chain or branched hydrocarbon group corresponding to an alkyl group having one or more triple carbon-carbon bonds. An alkynyl group formally corresponds to an alkyne with one C—H bond replaced by the point of attachment of the alkyl group to the remainder of the compound. The term “Cn-m alkynyl” refers to an alkynyl group having n to m carbons. Example alkynyl groups include, but are not limited to, ethynyl, propyn-1-yl, propyn-2-yl and the like. In some embodiments, the alkynyl moiety contains 2 to 6, 2 to 4, or 2 to 3 carbon atoms.

The term “alkylene”, employed alone or in combination with other terms, refers to a divalent alkyl linking group. An alkylene group formally corresponds to an alkane with two C—H bond replaced by points of attachment of the alkylene group to the remainder of the compound. The term “Cn-m alkylene” refers to an alkylene group having n to m carbon atoms. Examples of alkylene groups include, but are not limited to, ethan-1,2-diyl, ethan-1,1-diyl, propan-1,3-diyl, propan-1,2-diyl, propan-1,1-diyl, butan-1,4-diyl, butan-1,3-diyl, butan-1,2-diyl, 2-methyl-propan-1,3-diyl and the like.

The term “alkoxy”, employed alone or in combination with other terms, refers to a group of formula —O-alkyl, wherein the alkyl group is as defined above. The term “Cn-m alkoxy” refers to an alkoxy group, the alkyl group of which has n to m carbons. Example alkoxy groups include methoxy, ethoxy, propoxy (e.g., n-propoxy and isopropoxy), t-butoxy and the like. In some embodiments, the alkyl group has 1 to 6, 1 to 4, or 1 to 3 carbon atoms.

The term “Cₙ₋ₘ dialkoxy” refers to a linking group of formula —O—(Cₙ₋ₘ alkyl)-O—, the alkyl group of which has n to m carbons. Example dialkyoxy groups include —OCH₂CH₂O— and OCH₂CH₂CH₂O—. In some embodiments, the two O atoms of a Cₙ₋ₘ dialkoxy group may be attached to the same B atom to form a 5- or 6-membered heterocycloalkyl group.

The term “amino” refers to a group of formula —NH₂.

The term “carbonyl”, employed alone or in combination with other terms, refers to a —C(═O)— group, which also may be written as C(O).

The term “cyano” or “nitrile” refers to a group of formula —C≡N, which also may be written as —CN.

The terms “halo” or “halogen”, used alone or in combination with other terms, refers to fluoro, chloro, bromo and iodo. In some embodiments, “halo” refers to a halogen atom selected from F, Cl, or Br. In some embodiments, halo groups are F.

The term “haloalkyl” as used herein refers to an alkyl group in which one or more of the hydrogen atoms has been replaced by a halogen atom. The term “Cₙ₋ₘ haloalkyl” refers to a Cₙ₋ₘ alkyl group having n to m carbon atoms and from at least one up to {2(n to m)+1}halogen atoms, which may either be the same or different. In some embodiments, the halogen atoms are fluoro atoms. In some embodiments, the haloalkyl group has 1 to 6 or 1 to 4 carbon atoms. Example haloalkyl groups include CF₃, C₂F₅, CHF₂, CH₂F, CCl₃, CHCl₂, C₂Cl₅ and the like. In some embodiments, the haloalkyl group is a fluoroalkyl group.

The term “haloalkoxy”, employed alone or in combination with other terms, refers to a group of formula —O-haloalkyl, wherein the haloalkyl group is as defined above. The term “Cₙ₋ₘ haloalkoxy” refers to a haloalkoxy group, the haloalkyl group of which has n to m carbons. Example haloalkoxy groups include trifluoromethoxy and the like. In some embodiments, the haloalkoxy group has 1 to 6, 1 to 4, or 1 to 3 carbon atoms.

The term “oxo” refers to an oxygen atom as a divalent substituent, forming a carbonyl group when attached to carbon, or attached to a heteroatom forming a sulfoxide or sulfone group, or an N-oxide group. In some embodiments, heterocyclic groups may be optionally substituted by 1 or 2 oxo (═O) substituents.

The term “sulfido” refers to a sulfur atom as a divalent substituent, forming a thiocarbonyl group (C═S) when attached to carbon.

The term “oxidized” in reference to a ring-forming N atom refers to a ring-forming N-oxide.

The term “oxidized” in reference to a ring-forming S atom refers to a ring-forming sulfonyl or ring-forming sulfinyl.

The term “aromatic” refers to a carbocycle or heterocycle having one or more polyunsaturated rings having aromatic character (i.e., having (4n+2) delocalized π (pi) electrons where n is an integer).

The term “aryl,” employed alone or in combination with other terms, refers to an aromatic hydrocarbon group, which may be monocyclic or polycyclic (e.g., having 2 fused rings). The term “Cₙ₋ₘ aryl” refers to an aryl group having from n to m ring carbon atoms.

Aryl groups include, e.g., phenyl, naphthyl, and the like. In some embodiments, aryl groups have from 6 to about 10 carbon atoms. In some embodiments aryl groups have 6 carbon atoms. In some embodiments aryl groups have 10 carbon atoms. In some embodiments, the aryl group is phenyl.

The term “heteroaryl” or “heteroaromatic,” employed alone or in combination with other terms, refers to a monocyclic or polycyclic aromatic heterocycle having at least one heteroatom ring member selected from sulfur, oxygen and nitrogen. In some embodiments, the heteroaryl ring has 1, 2, 3 or 4 heteroatom ring members independently selected from nitrogen, sulfur and oxygen. In some embodiments, any ring-forming N in a heteroaryl moiety can be an N-oxide. In some embodiments, the heteroaryl has 5-14 ring atoms including carbon atoms and 1, 2, 3 or 4 heteroatom ring members independently selected from nitrogen, sulfur and oxygen. In some embodiments, the heteroaryl has 5-10 ring atoms including carbon atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. In other embodiments, the heteroaryl is an eight-membered, nine-membered or ten-membered fused bicyclic heteroaryl ring. Example heteroaryl groups include, but are not limited to, pyridinyl (pyridyl), pyrimidinyl, pyrazinyl, pyridazinyl, and the like.

A five-membered heteroaryl ring is a heteroaryl group having five ring atoms wherein one or more (e.g., 1, 2 or 3) ring atoms are independently selected from N, O and S. Exemplary five-membered ring heteroaryls include thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl and 1,3,4-oxadiazolyl.

A six-membered heteroaryl ring is a heteroaryl group having six ring atoms wherein one or more (e.g., 1, 2 or 3) ring atoms are independently selected from N, O and S. Exemplary six-membered ring heteroaryls are pyridyl, pyrazinyl, pyrimidinyl, triazinyl, isoindolyl, and pyridazinyl.

The term “cycloalkyl,” employed alone or in combination with other terms, refers to a non-aromatic hydrocarbon ring system (monocyclic, bicyclic or polycyclic), including cyclized alkyl and alkenyl groups. The term “Cₙ₋ₘ cycloalkyl” refers to a cycloalkyl that has n to m ring member carbon atoms. Cycloalkyl groups can include mono- or polycyclic (e.g., having 2, 3 or 4 fused rings) groups and spirocycles. Cycloalkyl groups can have 3, 4, 5, 6 or 7 ring-forming carbons (C₃₋₇). In some embodiments, the cycloalkyl group has 3 to 6 ring members, 3 to 5 ring members, or 3 to 4 ring members. In some embodiments, the cycloalkyl group is monocyclic. In some embodiments, the cycloalkyl group is monocyclic or bicyclic. In some embodiments, the cycloalkyl group is a C₃₋₆ monocyclic cycloalkyl group. Ring-forming carbon atoms of a cycloalkyl group can be optionally oxidized to form an oxo or sulfido group. Cycloalkyl groups also include cycloalkylidenes. In some embodiments, cycloalkyl is cyclopropyl, cyclobutyl, cyclopentyl or cyclohexyl. Also included in the definition of cycloalkyl are moieties that have one or more aromatic rings fused (i.e., having a bond in common with) to the cycloalkyl ring, e.g., benzo or thienyl derivatives of cyclopentane, cyclohexane and the like. A cycloalkyl group containing a fused aromatic ring can be attached through any ring-forming atom including a ring-forming atom of the fused aromatic ring. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, bicyclo[1.1.1]pentanyl, bicyclo[2.1.1]hexanyl, and the like. In some embodiments, the cycloalkyl group is cyclopropyl, cyclobutyl, cyclopentyl, or cyclohexyl.

The term “heterocycloalkyl,” employed alone or in combination with other terms, refers to a non-aromatic ring or ring system, which may optionally contain one or more alkenylene groups as part of the ring structure, which has at least one heteroatom ring member independently selected from nitrogen, sulfur, oxygen and phosphorus, and which has 4-10 ring members, 4-7 ring members, or 4-6 ring members. Included within the term “heterocycloalkyl” are monocyclic 4-, 5-, 6- and 7-membered heterocycloalkyl groups. Heterocycloalkyl groups can include mono- or bicyclic (e.g., having two fused or bridged rings) or spirocyclic ring systems. In some embodiments, the heterocycloalkyl group is a monocyclic group having 1, 2 or 3 heteroatoms independently selected from nitrogen, sulfur and oxygen. Ring-forming carbon atoms and heteroatoms of a heterocycloalkyl group can be optionally oxidized to form an oxo or sulfido group or other oxidized linkage (e.g., C(O), S(O), C(S) or S(O)₂, N-oxide etc.) or a nitrogen atom can be quaternized. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the heterocycloalkyl ring, e.g., benzo or thienyl derivatives of piperidine, morpholine, azepine, etc. A heterocycloalkyl group containing a fused aromatic ring can be attached through any ring-forming atom including a ring-forming atom of the fused aromatic ring.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas an azetidin-3-yl ring is attached at the 3-position.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. One method includes fractional recrystallization using a chiral resolving acid which is an optically active, salt-forming organic acid. Suitable resolving agents for fractional recrystallization methods are, e.g., optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane and the like.

Resolution of racemic mixtures can also be carried out by elution on a column packed with an optically active resolving agent (e.g., dinitrobenzoylphenylglycine). Suitable elution solvent composition can be determined by one skilled in the art.

In some embodiments, the compounds of the invention have the (R)-configuration. In other embodiments, the compounds have the (S)-configuration. In compounds with more than one chiral centers, each of the chiral centers in the compound may be independently (R) or (S), unless otherwise indicated.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, e.g., 1H- and 3H-imidazole, 1H-, 2H- and 4H-1,2,4-triazole, 1H- and 2H-isoindole and 1H- and 2H-pyrazole. Tautomeric forms can be in equilibrium or sterically locked into one form by appropriate substitution.

Compounds of the invention can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the invention can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can used in various studies such as NMR spectroscopy, metabolism experiments, and/or assays.

The term, “compound,” as used herein is meant to include all stereoisomers, geometric isomers, tautomers and isotopes of the structures depicted. The term is also meant to refer to compounds of the inventions, regardless of how they are prepared, e.g., synthetically, through biological process (e.g., metabolism or enzyme conversion), or a combination thereof.

All compounds, and pharmaceutically acceptable salts thereof, can be found together with other substances such as water and solvents (e.g., hydrates and solvates) or can be isolated. When in the solid state, the compounds described herein and salts thereof may occur in various forms and may, e.g., take the form of solvates, including hydrates. The compounds may be in any solid state form, such as a polymorph or solvate, so unless clearly indicated otherwise, reference in the specification to compounds and salts thereof should be understood as encompassing any solid state form of the compound.

In some embodiments, the compounds of the invention, or salts thereof, are substantially isolated. By “substantially isolated” is meant that the compound is at least partially or substantially separated from the environment in which it was formed or detected. Partial separation can include, e.g., a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “room temperature,” as used herein, are understood in the art, and refer generally to a temperature, e.g., a reaction temperature, that is about the temperature of the room in which the reaction is carried out, e.g., a temperature from about 20° C. to about 30° C.

The present invention also includes pharmaceutically acceptable salts of the compounds described herein. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, e.g., from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, isopropanol or butanol) or acetonitrile (MeCN) are preferred. Lists of suitable salts are found in Remington's Pharmaceutical Sciences, 17^{th} Ed., (Mack Publishing Company, Easton, 1985), p. 1418, Berge et al., J Pharm. Sci., 1977, 66(1), 1-19 and in Stahl et al., Handbook of Pharmaceutical Salts: Properties, Selection, and Use, (Wiley, 2002). In some embodiments, the compounds described herein include the N-oxide forms.

### Synthesis

Compounds of the invention, including salts thereof, can be prepared using known organic synthesis techniques and can be synthesized according to any of numerous possible synthetic routes, such as those in the Schemes below.

The reactions for preparing compounds of the invention can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., March's Advanced Organic Chemistry: Reactions, Mechanisms, and Structure, 6^{th} Ed. (Wiley, 2007); Peturssion et al., “Protecting Groups in Carbohydrate Chemistry,” J. Chem. Educ., 1997, 74(11), 1297; and Wuts et al., Protective Groups in Organic Synthesis, 4th Ed., (Wiley, 2006).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry or by chromatographic methods such as high performance liquid chromatography (HPLC) or thin layer chromatography (TLC).

The Schemes below provide general guidance in connection with preparing the compounds of the invention. One skilled in the art would understand that the preparations shown in the Schemes can be modified or optimized using general knowledge of organic chemistry to prepare various compounds of the invention.

Compounds of Formula I can be prepared, e.g., using a process as illustrated in the schemes below.

Compounds of Formula I with a variety of substitution such as those described herein can be prepared using a process as illustrated in Scheme 1. In the process depicted in Scheme 1, an appropriately substituted amine (1-1) is coupled with an appropriately substituted carboxylic acid (1-2) using a peptide coupling reagent (e.g., 1-[bis(dimethylamino)methylene]-1H-1,2,3-triazolo[4,5-b]pyridinium 3-oxid hexafluorophosphate (“HATU”) or 1-ethyl-3-(3-dimethylaminopropyl)carbodiimide) in the presence of a base (e.g., triethylamine or Hunig's base) to provide a compound of Formula I.

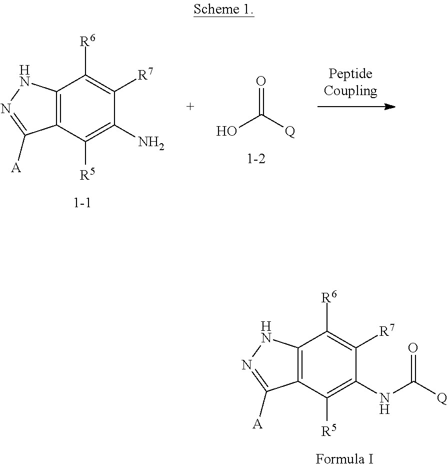

### Methods of Use

Over-activation of LRRK2 kinase activity, e.g., in kinase mutant G2019S, is a mechanism in alpha-synuclein related neurodegeneration, and is implicated in diseases that are characterized by the formation of Lewy bodies. Compounds as described herein, e.g., compounds of Formula I, exhibit inhibitory activity against LRRK2 kinase, including LRRK2 mutant kinase, such as mutant G2019S. Kinase activity can be determined using a kinase assay, which typically employs a kinase substrate and a phosphate group donor, such as ATP (or a derivative thereof). An exemplary kinase assay is described in Example A.

The present disclosure provides methods of modulating (e.g., inhibiting) LRRK2 activity, by contacting LRRK2 with a compound of the invention, or a pharmaceutically acceptable salt thereof. In some embodiments, the contacting can be administering to a patient a compound provided herein, or a pharmaceutically acceptable salt thereof. In certain embodiments, the compounds of the present disclosure, or pharmaceutically acceptable salts thereof, are useful for therapeutic administration to treat neurodegenerative disease. For example, a method of treating a disease or disorder associated with inhibition of LRRK2 interaction can include administering to a patient in need thereof a therapeutically effective amount of a compound provided herein, or a pharmaceutically acceptable salt thereof. The compounds of the present disclosure can be used alone, in combination with other agents or therapies or as an adjuvant or neoadjuvant for the treatment of diseases or disorders, including neurodegenerative diseases. For the uses described herein, any of the compounds of the disclosure, including any of the embodiments thereof, may be used.

Compounds and compositions as described herein, e.g., compounds of Formula I are useful in the treatment and/or prevention of LRRK2 kinase mediated disorders, including LRRK2 kinase mutant mediated diseases. LRRK2 kinase mutant G2019S mediated diseases include, but are not limited to, neurological diseases such as Parkinson's disease and other Lewy body diseases such as Parkinson disease with dementia, Parkinson's disease at risk syndrome, dementia with Lewy bodies (e.g., diffuse Lewy body disease (DLBD), Lewy body dementia, Lewy body disease, cortical Lewy body disease or senile dementia of Lewy type), Lewy body variant of Alzheimer's disease (i.e., diffuse Lewy body type of Alzheimer's disease), combined Parkinson's disease and Alzheimer's disease, as well as diseases associated with glial cortical inclusions, such as syndromes identified as multiple system atrophy, including striatonigral degeneration, olivopontocerebellar atrophy, and Shy-Drager syndrome, or other diseases associated with Parkinsonism, such as Hallervorden-Spatz syndrome (also referred to as Hallervorden-Spatz disease), fronto-temporal dementia, Sandhoff disease, progressive supranuclear palsy, corticobasal degeneration, autonomic dysfunctions (e.g., postural or orthostatic hypotension), cerebellar dysfunctions, ataxia, movement disorders, cognitive deterioration, sleep disorders, hearing disorders, tremors, rigidity (e.g., joint stiffness, increased muscle tone), bradykinesia, akinesia and postural instability (failure of postural reflexes, along other disease related factors such as orthostatic hypotension or cognitive and sensory changes, which lead to impaired balance and falls); cancers, including melanoma, acute myelogenous leukemia, breast carcinoma, lung adenocarincoma, prostate adenocarcinoma, renal cell carcinoma, and papillary thyroid carcinoma; autoimmune diseases such as Inflammatory Bowel Disease (e.g. Crohn's disease and ulcerative colitis); and leprosy.

In some embodiments, a method of treating a disease is provided comprising administering to a patient in need thereof a therapeutically effective amount of a compound of Formula I, or a pharmaceutically acceptable salt thereof, wherein the disease is selected from Parkinson's disease, Parkinson disease with dementia, Parkinson's disease at risk syndrome, dementia with Lewy bodies, Lewy body variant of Alzheimer's disease, combined Parkinson's disease and Alzheimer's disease, multiple system atrophy, striatonigral degeneration, olivopontocerebellar atrophy, Shy-Drager syndrome, Hallervorden-Spatz syndrome, fronto-temporal dementia, Sandhoff disease, progressive supranuclear palsy, corticobasal degeneration, postural hypotension, orthostatic hypotension, cerebellar dysfunctions, ataxia, movement disorders, cognitive deterioration, sleep disorders, hearing disorders, tremors, rigidity, bradykinesia, akinesia, postural instability, melanoma, acute myelogenous leukemia, breast carcinoma, lung adenocarincoma, prostate adenocarcinoma, renal cell carcinoma, papillary thyroid carcinoma, Crohn's disease, ulcerative colitis, and leprosy.

In some embodiments, a method of treating a neurological disease is provided comprising administering to a patient in need thereof a therapeutically effective amount of a compound of Formula I, or a pharmaceutically acceptable salt thereof, wherein the neurological disease is selected from Parkinson's disease, Parkinson disease with dementia, Parkinson's disease at risk syndrome, dementia with Lewy bodies, Lewy body variant of Alzheimer's disease, combined Parkinson's disease and Alzheimer's disease, multiple system atrophy, striatonigral degeneration, olivopontocerebellar atrophy, Shy-Drager syndrome, Hallervorden-Spatz syndrome, fronto-temporal dementia, Sandhoff disease, progressive supranuclear palsy, corticobasal degeneration, postural hypotension, orthostatic hypotension, cerebellar dysfunctions, ataxia, movement disorders, cognitive deterioration, sleep disorders, hearing disorders, tremors, rigidity, bradykinesia, akinesia, and postural instability.

In some embodiments, a method of treating a neurological disease is provided comprising administering to a patient in need thereof a therapeutically effective amount of a compound of Formula I, or a pharmaceutically salt thereof, wherein the neurological disease is selected from Parkinson's disease, Parkinson disease with dementia, Parkinson's disease at risk syndrome, dementia with Lewy bodies, Lewy body variant of Alzheimer's disease, combined Parkinson's disease and Alzheimer's disease, multiple system atrophy, striatonigral degeneration, olivopontocerebellar atrophy, and Shy-Drager syndrome.

In some embodiments, a method of treating Parkinson's disease is provided comprising administering to a patient in need thereof a therapeutically effective amount of a compound of Formula I, or a pharmaceutically acceptable salt thereof.

In some embodiments, a method of treating a cancer is provided comprising administering to a patient in need thereof a therapeutically effective amount of a compound of Formula I, or a pharmaceutically acceptable salt thereof, wherein the cancer is selected from t melanoma, acute myelogenous leukemia, breast carcinoma, lung adenocarincoma, prostate adenocarcinoma, renal cell carcinoma, and papillary thyroid carcinoma.

In some embodiments, a method of treating an autoimmune disease is provided comprising administering to a patient in need thereof a therapeutically effective amount of a compound of Formula I, or a pharmaceutically acceptable salt thereof, wherein the autoimmune disease is selected from Crohn's disease and ulcerative colitis.

In some embodiments, a method of treating leprosy is provided comprising administering to a patient in need thereof a therapeutically effective amount of a compound of Formula I, or a pharmaceutically acceptable salt thereof, or a composition comprising such compound or salt thereof.

In some embodiments, the compounds as described herein, e.g., compounds of Formula I, are inhibitors of LRRK2 kinase activity. In some embodiments, the compounds as described herein, e.g. compounds of Formula I, are inhibitors of LRRK2 mutant kinase activity. In some embodiments, the compounds as described herein, e.g. compounds of Formula I, are inhibitors of LRRK2 mutant G2019S kinase activity.

Compounds as described herein, e.g., compounds of Formula I, exhibit cellular biological activities, including but not limited to reduction in phosphorylation of ser910 or ser935 in HEK-293 cells transfected with either wild-type LRRK2 or LRRK2 G2019S mutant.

In some embodiments, compounds of Formula I are selective LRRK2 G2019S mutant inhibitors as compared to wild-type LRRK2.

As used herein, the term “contacting” refers to the bringing together of the indicated moieties in an in vitro system or an in vivo system such that they are in sufficient physical proximity to interact.

The terms “individual” or “patient,” used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

As used herein, the term “selective” or “selectivity” as it relates to kinase activity, means that a compound as described herein, e.g. a compound of Formula I, is a more potent inhibitor of a particular kinase, such as LRRK2 kinase, when compared to another kinase. While LRRK2 has other enzymatic activities, it is understood that when inhibitory activity or selectivity of LRRK2, or any mutation thereof, is mentioned, it is the LRRK2 kinase activity that is being referred to, unless clearly stated otherwise. As such, selectivity of LRRK2 relative to another kinase indicates a comparison of the IC₅₀ of a compound on the kinase activity of LRRK2 to the IC₅₀ of the compound on the kinase activity of another kinase. For example, a compound that is 10 fold selective for LRRK2 kinase activity relative to another kinase activity will have a ratio of IC₅₀(other kinase)÷IC₅₀(LRRK2)=10 (or a ratio of IC₅₀(LRRK2)÷IC₅₀(other kinase)=0.1).

In some embodiments, a compound as described herein, e.g., a compound of Formula I, is selective for a LRRK2 mutant over wild type LRRK2. Selectivity of LRRK2 mutants relative to wild type LRRK2 indicates a comparison of the IC₅₀ of a compound on the kinase activity of the mutant LRRK2 to the IC₅₀ of the compound on the kinase activity of wild type LRRK2. For example, a compound that is 10 fold selective for LRRK2 mutant kinase activity relative to wild type LRKK2 kinase activity will have a ratio of IC₅₀(wild type LRRK2)÷IC₅₀(mutant LRRK2)=10. In some embodiments, a compound provided herein is greater than 1 fold selective, greater than 2 fold selective, greater than 5 fold selective, greater than 10 fold selective, greater than 25 fold selective, or greater than 50 fold selective for LRRK2 mutant kinase over wild type LRRK2. In some embodiments, the LRRK2 mutant is LRRK2 G2019S.

The term “LRRK2-mediated condition”, “Leucine-rich repeat kinase 2 mediated disorder” or any other variation thereof, as used herein means any disease or other condition in which LRRK2, including any mutations thereof, is known to play a role, or a disease state that is associated with elevated activity or expression of LRRK2, including any mutations thereof. For example, a “LRRK2-mediated condition” may be relieved by inhibiting LRRK2 kinase activity. Such conditions include certain neurodegenerative diseases, such as Lewy body diseases, including, but not limited to, Parkinson's disease, Lewy body variant of Alzheimer's disease, combined Parkinson's disease and Alzheimer's disease, dementia with Lewy bodies, diffuse Lewy body disease, as well as any syndrome identified as multiple system atrophy; certain cancers, such as melanoma, papillary renal cell carcinoma and papillary thyroid carcinoma; certain autoimmune diseases, such as Inflammatory Bowel Disease (e.g. Crohn's disease and ulcerative colitis); and leprosy.

The term “neurodegenerative diseases” includes any disease or condition characterized by problems with movements, such as ataxia, and conditions affecting cognitive abilities (e.g., memory) as well as conditions generally related to all types of dementia. “Neurodegenerative diseases” may be associated with impairment or loss of cognitive abilities, potential loss of cognitive abilities and/or impairment or loss of brain cells. Exemplary “neurodegenerative diseases” include Alzheimer's disease, Parkinson's disease, amyotrophic lateral sclerosis (ALS), Down syndrome, dementia, multi-infarct dementia, mild cognitive impairment (MCI), epilepsy, seizures, Huntington's disease, neurodegeneration induced by viral infection (e.g. AIDS, encephalopathies), traumatic brain injuries, as well as ischemia and stroke.

“Neurodegenerative diseases” also includes any undesirable condition associated with the disease. For instance, a method of treating a neurodegenerative disease includes methods of treating or preventing loss of neuronal function characteristic of neurodegenerative disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

### Combination Therapies

One or more additional pharmaceutical agents or treatment methods can be used in combination with a compound of Formula I for treatment of LRRK2-associated diseases, disorders, or conditions, or diseases or conditions as described herein. The agents can be combined with the present compounds in a single dosage form, or the agents can be administered simultaneously or sequentially as separate dosage forms. In some embodiments, the additional pharmaceutical agent is a dopamine precursor, including, for example, levodopa, melevodopa, and etilevodopa. In some embodiments, the additional pharmaceutical agent is a dopamine agonist, including, for example, pramipexole, ropinorole, apomorphine, rotigotine, bromocriptine, cabergoline, and pergolide. In some embodiments, the additional pharmaceutical agent is a monamine oxidase B (“MAO B”) inhibitor, including, for example, selegiline and rasagiline. In some embodiments, the additional pharmaceutical agent is a catechol O-methyltransferase (“COMT”) inhibitor, including, for example, tolcapone and entacapone. In some embodiments, the additional pharmaceutical agent is an anticholinergic agent including, for example, benztropine, trihexyphenidyl, procyclidine, and biperiden. In some embodiments, the additional pharmaceutical agent is a glutamate (“NMDA”) blocking drug, including, for example, amantadine. In some embodiments, the additional pharmaceutical agent is an adenosine A2a antagonist, including, for example, istradefylline and preladenant. In some embodiments, the additional pharmaceutical agent is a 5-HT1a antagonist, including, for example, piclozotan and pardoprunox. In some embodiments, the additional pharmaceutical agent is an alpha 2 antagonist, including, for example, atipamezole and fipamezole.

### Formulations, Dosage Forms, and Administration

When employed as pharmaceuticals, the compounds of the present disclosure can be administered in the form of pharmaceutical compositions. Thus the present disclosure provides a composition comprising a compound Formula I or any of the formulas as described herein, a compound as recited in any of the claims and described herein, or a pharmaceutically acceptable salt thereof, or any of the embodiments thereof, and at least one pharmaceutically acceptable carrier. These compositions can be prepared in a manner well known in the pharmaceutical arts, and can be administered by a variety of routes, depending upon whether local or systemic treatment is indicated and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, e.g., by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, the compound of the present disclosure or a pharmaceutically acceptable salt thereof, in combination with one or more pharmaceutically acceptable carriers. In some embodiments, the composition is suitable for topical administration. In making the compositions of the invention, the active ingredient is typically mixed with an excipient, diluted by an excipient or enclosed within such a carrier in the form of, e.g., a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, e.g., up to 10% by weight of the active compound, soft and hard gelatin capsules, suppositories, sterile injectable solutions and sterile packaged powders.

In some embodiments, the composition is a sustained release composition comprising at least one compound described herein, or a pharmaceutically acceptable salt thereof, and at least one pharmaceutically acceptable carrier or excipient

The compositions can be formulated in a unit dosage form, each dosage containing from about 5 to about 1,000 mg (1 g). The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound may be effective over a wide dosage range and is generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms and the like.

The therapeutic dosage of a compound of the present invention can vary according to, e.g., the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers.

### EXAMPLES

Experimental procedures for compounds of the invention are provided below. Where the preparation of starting materials is not described, these are commercially available, known in the literature, or readily obtainable by those skilled in the art using standard procedures. Where it is stated that compounds were prepared analogously to earlier examples or intermediates, it will be appreciated by the skilled person that the reaction time, number of equivalents of reagents and temperature can be modified for each specific reaction and that it may be necessary or desirable to employ different work-up or purification techniques. Where reactions are carried out using microwave irradiation, the microwave used is a Biotage Initiator. The actual power supplied varies during the course of the reaction in order to maintain a constant temperature.

All solvents used were commercially available and were used without further purification. Reactions were typically run using anhydrous solvents under an inert atmosphere of nitrogen.

### Liquid Chromatography-Mass Spectrometry Method A

Total ion current (TIC) and DAD UV chromatographic traces together with MS and UV spectra associated with the peaks were taken on a UPLC/MS Acquity™ system equipped with PDA detector and coupled to a Waters single quadrupole mass spectrometer operating in alternated positive and negative electrospray ionization mode. [LC/MS-ES (+/−): analyses performed using an Acquity UPLC® CSH, C18 column (50×2.1 mm, 1.7 μm particle size), column temperature 40° C., mobile phase: A-water+0.10% HCOOH/B-CH₃CN+0.10% HCOOH, flow rate: 1.0 mL/min, runtime=2.0 min, gradient: t=0 min 3% B, t=1.5 min 99.9% B, t=1.9 min 99.9% B, t=2.0 min 3% B, stop time 2.0 min. Positive ES 100-1000, Negative ES 100-1000, UV detection DAD 210-350 nm.

### Liquid Chromatography-Mass Spectrometry Method B

Total ion current (TIC) and DAD UV chromatographic traces together with MS and UV spectra associated with the peaks were taken on a UPLC/MS Acquity™ system equipped with PDA detector and coupled to a Waters single quadrupole mass spectrometer operating in alternated positive and negative electrospray ionization mode. [LC/MS-ES (+/−): analyses performed using an Acquity UPLC™ BEH, C18 column (50×2.1 mm, 1.7 μm particle size), column temperature 40° C., mobile phase: A—0.1% v/v aqueous ammonia solution pH 10/B-CH₃CN, flow rate: 1.0 mL/min, runtime=2.0 min, gradient: t=0 min 3% B, t=1.5 min 99.9% B, t=1.9 min 99.9% B, t=2.0 min 3% B, stop time 2.0 min. Positive ES 100-1000, Negative ES 100-1000, UV detection DAD 210-350 nm.

### Liquid Chromatography-Mass Spectrometry Method A′

Instrument Name: MDAP_Fractionlynx; Method Description: Semi preparative MDAP Method; LC/MS System: Fractionlynx (Waters) with QDa MS detector; LC/MS Conditions: Column: XSelect CSH Prep. C18 5 μm OBD 30×100 mm @ room T; Injection loop: 1 ml; Solvents: A=H₂O+0.10% HCOOH; B=MeCN.

Gradient:

| Time | Flow Rate | | | |
|---|---|---|---|---|
| (min) | (ml/min) | % A | % B | Curve |
| initial | 40.0 | 65.0 | 35.0 | — |
| 10.0 | 40.0 | 40.0 | 60.0 | 6 |
| 10.5 | 40.0 | 0.1 | 99.9 | 6 |
| 14.5 | 40.0 | 0.1 | 99.9 | 6 |
| 15.0 | 40.0 | 55.0 | 45.0 | 6 |

The curve parameter followed Waters definition (6=linear, 11=step); Acquisition stop time: 15 min; UV Conditions: UV detection range: 210 nm to 350 nm; Acquisition rate: 1.0 spectra/s; MS Conditions: Ionisation mode: Positive Electrospray (ES+); Scan Range: ES+100 to 900 AMU; Scan Duration: 0.50 seconds.

### Liquid Chromatography-Mass Spectrometry Method B′

Instrument Name: MDAP_Fractionlynx; Method Description: Semi preparative MDAP Method; LC/MS System: Fractionlynx (Waters) with QDa MS detector; LC/MS Conditions: Column: XSelect CSH Prep. C18 5 μm OBD 30×100 mm @ room T; Injection loop: 1 ml; Solvents: A=H₂O+0.1% HCOOH; B=MeCN.

Gradient:

| Time | Flow Rate | | | |
|---|---|---|---|---|
| (min) | (ml/min) | % A | % B | Curve |
| initial | 40.0 | 65.0 | 35.0 | — |
| 10.0 | 40.0 | 50.0 | 50.0 | 6 |
| 10.5 | 40.0 | 0.1 | 99.9 | 6 |
| 14.5 | 40.0 | 0.1 | 99.9 | 6 |
| 15.0 | 40.0 | 65.0 | 35.0 | 6 |

The curve parameter followed Waters definition (6=linear, 11=step); Acquisition stop time: 15 min; UV Conditions: UV detection range: 210 nm to 350 nm; Acquisition rate: 1.0 spectra/s; MS Conditions: Ionisation mode: Positive Electrospray (ES+); Scan Range: ES+100 to 900 AMU; Scan Duration: 0.50 seconds.

### Liquid Chromatography-Mass Spectrometry Method C

Instrument Name: MDAP_Fractionlynx; Method Description: Semi preparative MDAP Method; LC/MS System: Fractionlynx (Waters) with ZQ MS detector; LC/MS Conditions: Column: XSelect CSH Prep. C18 5 μm OBD 30×100 mm @ room T; Injection loop: 1 ml; Solvents: A=H₂O+0.1% HCOOH; B=MeCN.

Gradient:

| Time | Flow Rate | | | |
|---|---|---|---|---|
| (min) | (ml/min) | % A | % B | Curve |
| initial | 40.0 | 85.0 | 15.0 | — |
| 10.0 | 40.0 | 65.0 | 35.0 | 6 |
| 10.5 | 40.0 | 0.1 | 99.9 | 6 |
| 14.5 | 40.0 | 0.1 | 99.9 | 6 |
| 15.0 | 40.0 | 85.0 | 15.0 | 6 |

The curve parameter followed Waters definition (6=linear, 11=step); Acquisition stop time: 15 min; UV Conditions: UV detection range: 210 nm to 350 nm; Acquisition rate: 1.0 spectra/s; MS Conditions: Ionisation mode: Positive Electrospray (ES+); Scan Range: ES+100 to 900 AMU; Scan Duration: 0.50 seconds.

### Liquid Chromatography-Mass Spectrometry Method D

Instrument Name: MDAP_Fractionlynx; Method Description: Semi preparative MDAP Method; LC/MS System: Fractionlynx (Waters) with ZQ MS detector; LC/MS Conditions: Column: XSelect CSH Prep. C18 5 μm OBD 30×100 mm @ room T; Injection loop: 1 ml; Solvents: A=H₂O+0.1% HCOOH; B=MeCN.

Gradient:

| Time | Flow Rate | | | |
|---|---|---|---|---|
| (min) | (ml/min) | % A | % B | Curve |
| initial | 40.0 | 90.0 | 10.0 | — |
| 6.0 | 40.0 | 75.0 | 25.0 | 6 |
| 6.5 | 40.0 | 0 | 100 | 6 |
| 9.5 | 40.0 | 0 | 100 | 6 |
| 10.0 | 40.0 | 90.0 | 10.0 | 6 |
| 10.5 | 40.0 | 90.0 | 10.0 | 6 |

The curve parameter followed Waters definition (6=linear, 11=step); Acquisition stop time: 10 min; UV Conditions: UV detection range: 210 nm to 350 nm; Acquisition rate: 1.0 spectra/s; MS Conditions: Ionisation mode: Positive Electrospray (ES+); Scan Range: ES+100 to 900 AMU; Scan Duration: 0.50 seconds.

### Liquid Chromatography-Mass Spectrometry Method E

Instrument Name: MDAP_Fractionlynx; Method Description: Semi preparative MDAP Method; LC/MS System: Fractionlynx (Waters) with ZQ MS detector; LC/MS Conditions: Column: XSelect CSH Prep. C18 5 μm OBD 30×100 mm @ room T; Injection loop: 1 ml; Solvents: A=H₂O+0.1% HCOOH; B=MeCN.

Gradient:

| Time | Flow Rate | | | |
|---|---|---|---|---|
| (min) | (ml/min) | % A | % B | Curve |
| initial | 40.0 | 97.0 | 3.0 | — |
| 10.0 | 40.0 | 50.0 | 50.0 | 6 |
| 10.5 | 40.0 | 0.0 | 100.0 | 6 |
| 14.5 | 40.0 | 0.0 | 100.0 | 6 |
| 15.0 | 40.0 | 97.0 | 3.0 | 6 |
| 16.1 | 3.0 | 97.0 | 3.0 | 6 |

The curve parameter followed Waters definition (6=linear, 11=step); Acquisition stop time: 16 min; UV Conditions: UV detection range: 210 nm to 350 nm; Acquisition rate: 1.0 spectra/s; MS Conditions: Ionisation mode: Positive Electrospray (ES+); Scan Range: ES+100 to 900 AMU; Scan Duration: 0.50 seconds.

### Liquid Chromatography-Mass Spectrometry Method F

Instrument Name: MDAP_Fractionlynx; Method Description: Semi preparative MDAP Method; LC/MS System: Fractionlynx (Waters) with ZQ MS detector; LC/MS Conditions: Column: XSelect CSH Prep. C18 5 μm OBD 30×100 mm @ room T; Injection loop: 1 ml; Solvents: A=H₂O+0.10% HCOOH; B=MeCN.

Gradient:

| Time | Flow Rate | | | |
|---|---|---|---|---|
| (min) | (ml/min) | % A | % B | Curve |
| initial | 40.0 | 90.0 | 10.0 | — |
| 10.0 | 40.0 | 70.0 | 30.0 | 6 |
| 10.5 | 40.0 | 0.1 | 99.9 | 6 |
| 14.5 | 40.0 | 0.1 | 99.9 | 6 |
| 15.0 | 40.0 | 90.0 | 10.0 | 6 |

The curve parameter followed Waters definition (6=linear, 11=step); Acquisition stop time: 15 min; UV Conditions: UV detection range: 210 nm to 350 nm; Acquisition rate: 1.0 spectra/s; MS Conditions: Ionisation mode: Positive Electrospray (ES+); Scan Range: ES+100 to 900 AMU; Scan Duration: 0.50 seconds.

### Liquid Chromatography-Mass Spectrometry Method G

Instrument Name: MDAP_Fractionlynx; Method Description: Semi preparative MDAP Method; LC/MS System: Fractionlynx (Waters) with ZQ MS detector; LC/MS Conditions: Column: XSelect CSH Prep. C18 5 μm OBD 30×100 mm @ room T; Injection loop: 1 ml; Solvents: A=H₂O+0.1% HCOOH; B=MeCN.

Gradient:

| Time | Flow Rate | | | |
|---|---|---|---|---|
| (min) | (ml/min) | % A | % B | Curve |
| initial | 40.0 | 90.0 | 10.0 | — |
| 10.0 | 40.0 | 55.0 | 45.0 | 6 |
| 10.5 | 40.0 | 0.1 | 99.9 | 6 |
| 14.5 | 40.0 | 0.1 | 99.9 | 6 |
| 15.0 | 40.0 | 90.0 | 10.0 | 6 |

The curve parameter followed Waters definition (6=linear, 11=step); Acquisition stop time: 15 min; UV Conditions: UV detection range: 210 nm to 350 nm; Acquisition rate: 1.0 spectra/s; MS Conditions: Ionisation mode: Positive Electrospray (ES+); Scan Range: ES+100 to 900 AMU; Scan Duration: 0.50 seconds.

### Liquid Chromatography-Mass Spectrometry Method H

Instrument Name: MDAP_Fractionlynx; Method Description: Semi preparative MDAP Method; LC/MS System: Fractionlynx (Waters) with ZQ MS detector; LC/MS Conditions: Column: XSelect CSH Prep. C18 5 μm OBD 30×100 mm @ room T; Injection loop: 1 ml; Solvents: A=H₂O+0.10% HCOOH; B=MeCN.

Gradient:

| Time | Flow Rate | | | |
|---|---|---|---|---|
| (min) | (ml/min) | % A | % B | Curve |
| initial | 40.0 | 90.0 | 10.0 | — |
| 10.0 | 40.0 | 55.0 | 45.0 | 6 |
| 10.5 | 40.0 | 0.1 | 99.9 | 6 |
| 14.5 | 40.0 | 0.1 | 99.9 | 6 |
| 15.0 | 40.0 | 90.0 | 10.0 | 6 |
| 16.1 | 3 | 90.0 | 10.0 | 6 |

The curve parameter followed Waters definition (6=linear, 11=step); Acquisition stop time: 15 min; UV Conditions: UV detection range: 210 nm to 350 nm; Acquisition rate: 1.0 spectra/s; MS Conditions: Ionisation mode: Positive Electrospray (ES+); Scan Range: ES+100 to 900 AMU; Scan Duration: 0.50 seconds.

### Liquid Chromatography-Mass Spectrometry Method I

Instrument Name: MDAP_Fractionlynx; Method Description: Semi preparative MDAP Method; LC/MS System: Fractionlynx (Waters) with ZQ MS detector; LC/MS Conditions: Column: XSelect CSH Prep. C18 5 μm OBD 30×100 mm @ room T; Injection loop: 1 ml; Solvents: A=H₂O+0.1% HCOOH; B=MeCN.

Gradient:

| Time | Flow Rate | | | |
|---|---|---|---|---|
| (min) | (ml/min) | % A | % B | Curve |
| initial | 40.0 | 80.0 | 20.0 | — |
| 10.0 | 40.0 | 30.0 | 70.0 | 6 |
| 10.5 | 40.0 | 0 | 100 | 6 |
| 14.5 | 40.0 | 0 | 100 | 6 |
| 15.0 | 40.0 | 80.0 | 20.0 | 6 |
| 16.1 | 3 | 80.0 | 20.0 | 6 |

The curve parameter followed Waters definition (6=linear, 11=step); Acquisition stop time: 15 min; UV Conditions: UV detection range: 210 nm to 350 nm; Acquisition rate: 1.0 spectra/s; MS Conditions: Ionisation mode: Positive Electrospray (ES+); Scan Range: ES+100 to 900 AMU; Scan Duration: 0.50 seconds.

### Liquid Chromatography-Mass Spectrometry Method J

Instrument Name: MDAP_Fractionlynx; Method Description: Semi preparative MDAP Method; LC/MS System: Fractionlynx (Waters) with ZQ MS detector; LC/MS Conditions: Column: XSelect CSH Prep. C18 5 μm OBD 30×100 mm @ room T; Injection loop: 1 ml; Solvents: A=H₂O+0.10% HCOOH; B=MeCN.

Gradient:

| Time | Flow Rate | | | |
|---|---|---|---|---|
| (min) | (ml/min) | % A | % B | Curve |
| initial | 40.0 | 93.0 | 7.0 | — |
| 10.0 | 40.0 | 79.0 | 21.0 | 6 |
| 10.5 | 40.0 | 0.1 | 99.9 | 6 |
| 14.5 | 40.0 | 0.1 | 99.9 | 6 |
| 15.0 | 40.0 | 93.0 | 7.0 | 6 |

The curve parameter followed Waters definition (6=linear, 11=step); Acquisition stop time: 15 min; UV Conditions: UV detection range: 210 nm to 350 nm; Acquisition rate: 1.0 spectra/s; MS Conditions: Ionisation mode: Positive Electrospray (ES+); Scan Range: ES+100 to 900 AMU; Scan Duration: 0.50 seconds.

### Liquid Chromatography Method K

Column: Welch Ultimate AQ-C18 150×30 mm×5 um; mobile phase: water (0.1% TFA)-ACN: 25%-55% over 12 minutes.

### Liquid Chromatography Method L

Column: Agela DuraShell C18 250*25 mm*10 um; mobile phase: water (10 mM NH₄HCO₃)—ACN: 0%-22% over 25 minutes.

### Liquid Chromatography Method M

Column: Phenomenex luna C18 250*50 mm*10 um; mobile phase: water (0.1% TFA)—ACN: 15%-45% over 20 minutes.

### Liquid Chromatography Method N

Prep-HPLC (basic condition, column: Waters Xbridge Prep OBD C18 150*40 mm*10 um; mobile phase: [water (0.04% NH₃H₂O+10 mM NH₄HCO₃)-ACN]; B %: 15%-45%, 10 min)

### Liquid Chromatography Method O

SFC conditions: DAICEL CHIRALPAK IC (250 mm×30 mm, 10 um); mobile phase: [0.1% NH₃.H₂O MeOH]; B %: 50%-50%, 15 min

### Liquid Chromatography Method P

SFC conditions: DAICEL CHIRALPAK IC (250 mm×30 mm, 10 um); mobile phase: [0.1% NH₃H₂O MeOH]; B %: 45%-45%, 20 min

### Liquid Chromatography Method Q

Prep-HPLC—basic condition, column: Waters Xbridge Prep OBD C18 150*40 mm*10 um; mobile phase: [water (0.04% NH₄OH+10 mM NH₄HCO₃)-ACN]; B %: 5%-35%, 10 min

### Liquid Chromatography Method R

SFC column: REGIS (s,s) WHELK-O1 (250 mm*30 mm, 5 um); mobile phase: [0.1% NH₃H₂O MEOH]; B %: 50%-50%, 15 min

### Liquid Chromatography Method S

Prep-HPLC—column: Nano-micro Kromasil C18 100*30 mm, 5 um; mobile phase: [water (0.1% TFA)-CAN]; b %: 27%-47%, 10 min.

### Liquid Chromatography Method T

Prep-HPLC—column: Waters Xbridge Prep OBD C18 150*40 mm*10 um; mobile phase: [water (10 mM NH₄HCO₃)-ACN]; B %: 15%-45%, 8 min

### Liquid Chromatography Method U

Prep-HPLC—column: Waters Xbridge Prep OBD C18 150*40 mm*10 um; mobile phase: [water (10 mM NH₄HCO₃)-ACN]; B %: 15%-45%, 8 min

### Liquid Chromatography Method V

Prep-HPLC—column: Waters Xbridge BEH C18 100*30 mm*10 um; mobile phase: [water (10 mM NH₄HCO₃)-ACN]; B %: 25%-45%, 8 min

### Liquid Chromatography Method W

Prep-HPLC—column: Phenomenex Synergi C18 150*25*10 um; mobile phase: [water (0.1% TFA)-ACN]; B %: 15%-45%, 10 min

### Liquid Chromatography Method X

Prep-HPLC (TFA condition) column: Phenomenex Luna C18 150*30 mm*5 um; mobile phase: [water (0.1% TFA)-MeOH]; B %: 30%-60%, 8 min

### Liquid Chromatography Method Y

Instrument Name: MDAP_Fractionlynx; Method Description: Semi preparative MDAP Method; LC/MS System: Fractionlynx (Waters) with QDa MS detector; LC/MS Conditions: Column: XSelect CSH Prep. C18 5 μm OBD 30×100 mm @ room T; Injection loop: 1 ml; Solvents: A=H₂O+0.10% HCOOH; B=MeCN.

Gradient:

| Time | Flow Rate | | | |
|---|---|---|---|---|
| (min) | (ml/min) | % A | % B | Curve |
| initial | 40.0 | 83.0 | 17.0 | — |
| 10.0 | 40.0 | 64.0 | 36.0 | 6 |
| 10.5 | 40.0 | 0.1 | 99.9 | 6 |
| 14.5 | 40.0 | 0.1 | 99.9 | 6 |
| 15.0 | 40.0 | 83.0 | 17.0 | 6 |

The curve parameter followed Waters definition (6=linear, 11=step); Acquisition stop time: 15 min; UV Conditions: UV detection range: 210 nm to 350 nm; Acquisition rate: 1.0 spectra/s; MS Conditions: Ionisation mode: Positive Electrospray (ES+); Scan Range: ES+100 to 900 AMU; Scan Duration: 0.50 seconds.

### Liquid Chromatography Method Z

Prep-HPLC—column: Nano-Micro UniSil 5-100 C18 ULTRA 100*250 mm 5 um; mobile phase: [water (0.1% TFA)-ACN]; B %: 12%-27%, 10 min

### Liquid Chromatography Method AA

Semipreparative chiral HPLC conditions and results:

Column Chiralcel OJ-H (25×2.0 cm), 5μ

Mobile phase n-Hexane/EtOH+0.10% isopropylamine 70/30% v/v

Flow rate (ml/min) 17 ml/min

DAD detection 220 nm Loop 1500 μL Total amount 5 mg

Solubilization 5 mg in 1.5 mL EtOH/MeOH 1/1=3.3 mg/mL

Injection 5 mg/injection

### Other Analytical Methods

¹H Nuclear magnetic resonance (NMR) spectroscopy was carried out using one of the following instruments: a Bruker Avance 400 instrument equipped with probe DUAL 400 MHz 5i, a Bruker Avance 400 instrument equipped with probe 6 SI 400 MHz 5 mm ¹H-¹³C ID, a Bruker Avance III 400 instrument with nanobay equipped with probe Broadband BBFO 5 mm direct, a 400 MHz Agilent Direct Drive instrument with ID AUTO-X PFG probe, all operating at 400 MHz, or an Agilent VNMRS500 Direct Drive instrument equipped with a 5 mm Triple Resonance ¹H{¹³C/¹⁵N} cryoprobe operating at 500 MHz. The spectra were acquired in the stated solvent at around room temperature unless otherwise stated. In all cases, NMR data were consistent with the proposed structures. Characteristic chemical shifts (6) are given in parts-per-million using conventional abbreviations for designation of major peaks: e.g. s, singlet; d, doublet; t, triplet; q, quartet; dd, doublet of doublets; dt, doublet of triplets; br, broad.

Where thin layer chromatography (TLC) has been used it refers to silica gel TLC using silica gel F254 (Merck) plates, Rf is the distance travelled by the compound divided by the distance travelled by the solvent on a TLC plate. Column chromatography was performed using an automatic flash chromatography (Biotage SP1 or Isolera) system over Biotage silica gel cartridges (KP-Sil or KP-NH) or in the case of reverse phase chromatography over Biotage C18 cartridges (KP-C₁₈).

### Intermediate 1. 3-Bromo-1H-indazol-5-amine

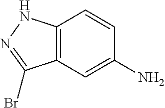 [SMILES: Nc1ccc2[nH]nc(Br)c2c1]

To a solution of 3-bromo-5-nitro-1H-indazole (500 mg, 2.07 mmol) in EtOH (20 mL) was added SnCl₂.2H₂O (2.80 g, 12.40 mmol) portion wise at 0° C. The mixture was stirred at 80° C. for 12 hrs. LCMS showed the reaction was complete. The reaction mixture was concentrated. The residue was poured into water (20 mL) and then adjusted to pH=8 with a saturated aqueous NaHCO₃ solution. The reaction mixture was extracted with EtOAc (50 mL×3). The combined organic layers were washed with brine (50 mL×1), dried over anhydrous Na₂SO₄, filtered and concentrated in vacuum to afford the title compound (350 mg, 1.65 mmol, 79.90% yield) as a brown solid, which was used without further purification.

### Intermediate 2. 6-Methyl-1H-benzo[d][1,2,3]triazole-5-carboxylic acid

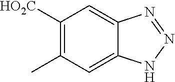

### Step 1. Methyl 4-fluoro-2-methyl-5-nitrobenzoate

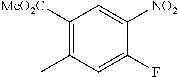

To a solution of methyl 4-fluoro-2-methylbenzoate (10 g, 59.47 mmol) in 98% H₂SO₄ (80 mL) was added a solution of KNO₃ (6.61 g, 65.41 mmol) in 98% H₂SO₄ (8 mL) dropwise at −20° C., the reaction mixture was stirred for 1 hr at −10° C. The reaction mixture was poured onto ice (200 g) and filtered. The solid was dried and purified by column chromatography (SiO₂, petroleum ether/EtOAc=20:1 to 0:1) to afford the title compound (3 g, (14.07 mmol, 23.67% yield) as a white solid.

### Step 2. Methyl 4-amino-2-methyl-5-nitrobenzoate

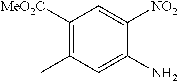

NH₃ (15 psi) was bubbled into a solution of methyl 4-fluoro-2-methyl-5-nitrobenzoate (3.00 g, 14.07 mmol) in THF (30 mL) at 20° C. and the reaction mixture was stirred at for 2 hrs. The reaction mixture was diluted with water (50 mL), then filtered and the solid was dried to afford the title compound (2.70 g, 12.85 mmol, 91.27% yield) as a yellow solid.

### Step 3. Methyl 4,5-diamino-2-methylbenzoate

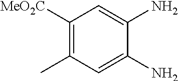

To a solution of methyl 4-amino-2-methyl-5-nitrobenzoate (2.70 g, 12.85 mmol) in MeOH (60 mL) was added Pd/C (0.54 g, 10% purity) under N₂ atmosphere. The suspension was degassed and purged with H₂ for three times. The reaction mixture was stirred at 20° C. for 3 hrs under H₂ (15 psi). The reaction mixture was filtered and the filtrate was concentrated to give a residue. The residue was purified by column chromatography (SiO₂, petroleum ether/EtOAc=20:1 to 0:1) to afford the title compound (2.44 g) as a red solid.

### Step 4. Methyl 6-methyl-1H-benzo[d][1,2,3]triazole-5-carboxylate

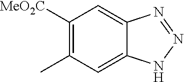

To a solution of methyl 4,5-diamino-2-methylbenzoate (2.44 g, 13.54 mmol) in H₂O (6 mL) was added AcOH (2.03 g, 33.85 mmol, 1.94 mL) at 0° C. followed by dropwise addition of a solution of NaNO₂ (1.12 g, 16.25 mmol) in H₂O (6 mL) at 0° C. After addition, the temperature of the above mixture was slowly raised to 50° C. for 0.25 hr, then the reaction mixture was stirred at 20° C. for 12 hrs. The reaction mixture was poured into water (20 mL) and filtered. The solid was dried to afford the title compound (1.35 g, crude) as a light yellow solid, which was used without further purification.

### Step 5. 6-Methyl-1H-benzo[d][1,2,3]triazole-5-carboxylic acid

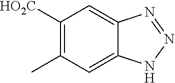

A mixture of methyl 6-methyl-1H-benzo[d][1,2,3]triazole-5-carboxylate (300 mg, 1.57 mmol), NaOH (188.28 mg, 4.71 mmol) in THF (6 mL) and H₂O (6 mL) was degassed and purged with N₂ (3×), and then the mixture was stirred at 20° C. for 12 hr under a N₂ atmosphere. The mixture was concentrated to give a residue. The residue was diluted with H₂O (10 mL), adjusted to pH=3 by addition 1N HCl (1 mL), and then extracted with EtOAc (5 mL×3). The combined organic layers were dried over Na₂SO₄, filtered and concentrated to afford the title compound (190 mg, crude) as a yellow solid which was used without further purification.

### Intermediate 3: 3-(2-Fluorophenyl)-1H-indazol-5-amine

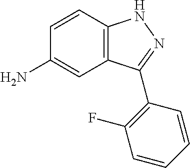 [SMILES: Nc1ccc2[nH]nc(-c3ccccc3F)c2c1]

3-Bromo-1H-indazol-5-amine (Intermediate 1; 150 mg, 0.71 mmol) and (2-fluorophenyl)boronic acid (99 mg, 0.71 mmol) were dissolved in a mixture of DMF (2 mL) and 2M aqueous Na₂CO₃ (1 mL). The mixture was purged with N₂ for 15 minutes and then palladium triphenylphosphine (40.87 mg, 0.04 mmol) was added. The reaction mixture was stirred at 110° C. for 18 hrs. The mixture was then partitioned between water and EtOAc.

The phases were separated; the aqueous layer was extracted with EtOAc and the combined organic layers were washed with brine, dried over anhydrous Na₂SO₄ and the solvent was removed under reduced pressure. The crude material was purified by reverse phase column chromatography on a 30 g C₁₈-silica gel column, eluting with a gradient of ACN in water from 2% to 55% in the presence of 0.1% of ammonia solution to obtain the title compound (40 mg, 0.176 mmol, 24.9% yield). ¹H NMR (400 MHz, DMSO-d₆) δ 12.91 (s, 1H), 7.72 (td, J=7.53, 1.76 Hz, 1H), 7.41-7.48 (m, 1H), 7.27-7.39 (m, 3H), 6.83 (dd, J=8.78, 2.01 Hz, 1H), 6.79 (d, J=2.26 Hz, 1H), 4.86 (s, 2H). MS-ESI (m/z) calc'd for C₁₃H₁₁FN₃ [M+H]⁺: 228.1. Found 228.1.

### Intermediate 4. 5,7-Dimethyl-[1,2,3]triazolo[1,5-a]pyridine-6-carboxylic acid

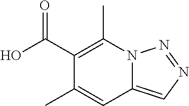

### Step 1. Ethyl 6-chloro-2,4-dimethylnicotinate

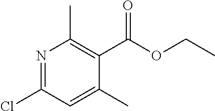

A suspension of 2,4-dimethyl-6-oxo-1,6-dihydro-3-pyridinecarboxylic acid (890 mg, 5.32 mmol) in phosphorus oxychloride (8.9 mL, 95.19 mmol), was stirred at 80° C. for 6 hrs. The reaction mixture was cooled to r.t. and then the solvent was evaporated. The residue was cooled to 0° C. and EtOH (3.5 mL) was added dropwise. The mixture was stirred at r.t. for 30 min, then partitioned between EtOAc and H₂O. The organic phase was concentrated in vacuo and the residue was taken up with phosphorus oxychloride (4 mL) and stirred for an additional 18 hrs at 80° C. The mixture was cooled to r.t. and then the solvent was evaporated. The residue was cooled to 0° C. and EtOH (3 mL) was added dropwise. The mixture was stirred at r.t. for 1.5 hrs and then partitioned between EtOAc and H₂O. The organic phase was concentrated under reduced pressure and the residue was purified by normal phase column chromatography on a 25 g silica gel column using a 0 to 20% gradient of EtOAc in cyclohexane as eluent. The title compound (842 mg, 3.94 mmol, 74% yield) was obtained as a colorless oil. ¹H NMR (400 MHz, DMSO-d₆) δ 7.37 (s, 1H), 4.38 (q, J=7.19 Hz, 2H), 2.43 (s, 3H), 2.30 (s, 3H), 1.33 (t, J=7.15 Hz, 3H). MS-ESI (m/z) calc'd for C₁₀H₁₃ClNO₂ [M+H]⁺: 214.1, 216.1. Found 214.1, 216.1.

### Step 2. Ethyl 2,4-dimethyl-6-vinylnicotinate

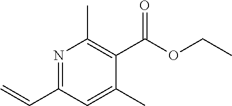

Ethyl 6-chloro-2,4-dimethylnicotinate (840 mg, 3.93 mmol) and toluene (9 mL) were added to a nitrogen-filled sealed vessel. The solution was degassed with nitrogen and then palladium triphenylphosphine (227.15 mg, 0.2 mmol), triphenylphosphine (103.12 mg, 0.39 mmol) and tributyl(ethenyl)stannane (1.49 mL, 5.11 mmol) were added. The mixture was heated at 95° C. for 18 hrs under a nitrogen atmosphere. The reaction mixture was concentrated under reduced pressure and then taken up in DCM. The organic phase was washed with a sat. aqueous solution of NH₄Cl and NH₂CO₃, filtered through a phase separator and then concentrated in vacuum. The crude material was purified by normal phase column chromatography on a 25 g silica gel column using a 0 to 20% gradient of EtOAc in cyclohexane as eluent. The title compound (686 mg, 3.34 mmol, 85% yield) was obtained as a yellow oil. ¹H NMR (400 MHz, CDCl₃) δ 7.27 (s, 1H), 7.04 (s, 1H), 6.76 (dd, J=17.39, 10.78 Hz, 1H), 6.21 (dd, J=17.50, 0.99 Hz, 1H), 5.50 (dd, J=10.78, 1.10 Hz, 1H), 4.42 (d, J=7.04 Hz, 2H), 2.56 (s, 3H), 2.34 (s, 3H), 1.41 (t, J=7.15 Hz, 3H). MS-ESI (m/z) calc'd for C₁₂H₁₆NO₂ [M+H]⁺: 206.1. Found 206.2.

### Step 3. Ethyl 6-formyl-2,4-dimethylnicotinate

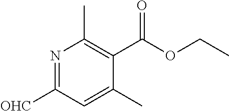

A solution of ethyl 2,4-dimethyl-6-vinylnicotinate (686 mg, 3.34 mmol) in dry DCM (50 mL) was cooled to −78° C. under N₂ atmosphere. A stream of ozone-enriched oxygen was bubbled through the solution until a yellow color persisted. After 20 minutes a stream of dry nitrogen was bubbled through the reaction mixture. A solution of PPh₃ (1.75 g, 6.68 mmol) in DCM (25 mL) was added dropwise and the resulting solution was allowed to warm to room temperature. The reaction mixture was then concentrated in vacuo and the crude residue was purified by normal phase NH-silica gel chromatography using a 0 to 50% gradient of MeOH in EtOAc as eluent. The column purification failed and the desired product was recovered by breaking the column and recovering the silica, which was taken up in 2M HCl and stirred overnight at r.t. The next day the mixture was neutralized with sat. aqueous NaHCO₃ and then extracted with DCM (3×). The combined organic phases were passed through a phase separator and concentrated under reduced pressure to afford the title compound (250 mg, 1.206 mmol, 36.1% yield) as an orange oil. ¹H NMR (400 MHz, DMSO-d₆) δ 9.95 (s, 1H), 7.74 (s, 1H), 4.43 (q, J=7.12 Hz, 2H), 2.55 (s, 3H), 2.38 (s, 3H), 1.35 (t, J=7.15 Hz, 4H). MS-ESI (m/z) calc'd for C₁₁H₁₄NO₃ [M+H]⁺: 208.1. Found 208.2.

### Step 4. Ethyl 2,4-dimethyl-6-((2-tosylhydrazineylidene)methyl)nicotinate

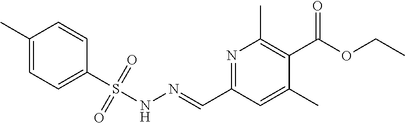 [SMILES: CCOC(=O)c1c(C)cc(/C=N/NS(=O)(=O)c2ccc(C)cc2)nc1C]

To a mixture of 4-methylbenzenesulfonohydrazide (207.6 mg, 1.11 mmol) and EtOH (4 mL), was rapidly added ethyl 6-formyl-2,4-dimethylnicotinate (210 mg, 1.01 mmol). The reaction was stirred at r.t. for 2 hrs. Then the mixture was concentrated in vacuo to afford the title compound as a crude product, which was used without further purification. MS-ESI (m/z) calc'd for C₁₈H₂₂N₃O₄S [M+H]⁺: 376.1. Found 376.3.

### Step 5. Ethyl 5,7-dimethyl-[1,2,3]triazolo[1,5-a]pyridine-6-carboxylate

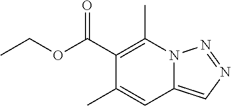 [SMILES: CCOC(=O)c1c(C)cc2cnnn2c1C]

A solution of ethyl 2,4-dimethyl-6-((2-tosylhydrazineylidene)methyl)nicotinate (460 mg, 0.8 mmol) in morpholine (2.5 mL, 83.61 mmol) was stirred at 95° C. for 1 h. The mixture was concentrated in vacuo and the residue was taken up in DCM and washed with water. The aqueous layer was then extracted (3×) with DCM. The combined organic layers were concentrated in vacuo. The product was purified by normal phase silica gel column chromatography using a 0 to 70% gradient of EtOAc in cyclohexane as eluent. The title compound (120 mg, 0.547 mmol, 68.7% yield) was obtained as a pale-yellow oil. ¹H NMR (400 MHz, CDCl₃) δ 8.02 (s, 1H), 7.47 (s, 1H), 4.50 (q, J=7.04 Hz, 2H), 2.96 (s, 3H), 2.44 (s, 3H), 1.46 (t, J=7.15 Hz, 3H). MS-ESI (m/z) calc'd for C₁₁H₁₄N₃O₂ [M+H]⁺: 220.1. Found 220.2.

### Step 6. 5,7-Dimethyl-[1,2,3]triazolo[1,5-a]pyridine-6-carboxylic acid

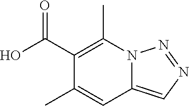

A 2M aqueous solution of NaOH (21.0 mL, 21 mmol) was added to a solution of ethyl 5,7-dimethyltriazolo[1,5-a]pyridine-6-carboxylate (700.0 mg, 3.2 mmol) in MeOH (5 mL) and the reaction was stirred at r.t. overnight. The reaction mixture was then heated at 50° C. for 1 hour. MeOH was remove under reduced pressure and a 2M aqueous HCl solution was added until the pH=1. The precipitate was filtered and washed with water. The white solid was dried under reduced pressure, to afford 5,7-dimethyltriazolo[1,5-a]pyridine-6-carboxylic acid (612 mg, 3.2 mmol, 100% yield) as a white solid. ¹H NMR (400 MHz, DMSO-d₆) δ 13.99 (br. S, 1H), 8.17 (s, 1H), 7.75 (s, 1H), 2.84 (s, 3H), 2.39 (d, J=0.66 Hz, 3H). MS-ESI (m/z) calc'd for C₉H₁₀N₃O₂ [M+H]⁺: 192.1. Found 192.0.

### Intermediate 5. Ethyl 6-methylimidazo[1,5-a]pyridine-7-carboxylate

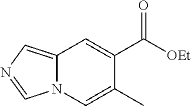

Imidazole-4-carboxaldehyde (0.76 mL, 10.41 mmol) was dissolved in DMF (30 mL). Ethyl (E)-4-bromo-3-methylbut-2-enoate (1.57 mL, 10.41 mmol) was added dropwise and the reaction solution was stirred at r.t. for 5 hrs. EtOAc (50 mL) was added followed by water (50 mL). The organic layer was separated, dried over Na₂SO₄, filtered and concentrated to give a crude product that was purified on Biotage (100 g. silica gel cartridge, eluting with cyclohexane/EtOAc 8:2 to 2:8) to give the title compound (1650 mg, 8.079 mmol, 77.63% yield). ¹H NMR (400 MHz, CDCl₃) δ 8.25 (s, 1H), 8.12 (s, 1H), 7.74 (d, J=1.10 Hz, 1H), 7.61-7.66 (m, 1H), 7.28 (s, 1H), 4.37 (q, J=7.04 Hz, 2H), 2.48 (d, J=1.10 Hz, 3H), 2.06 (s, 2H), 1.37-1.48 (m, 3H). MS-ESI (m/z) calc'd for C₁₁H₁₃N₂O₂ [M+H]⁺: 205.1. Found 205.3.

### Intermediate 6: 4,6-Difluoro-1-methyl-1H-indazole-5-carboxamide

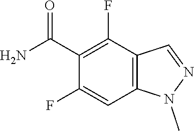

To a solution of 4,6-difluoro-1-methyl-1H-indazole-5-carboxylic acid (400 mg, 1.89 mmol) in THF (4 mL) was added CDI (458.58 mg, 2.83 mmol) and the reaction mixture was stirred at 15° C. for 1.5 hrs. Then NH₃.H₂O (2.11 g, 15.08 mmol, 25%) was added and the reaction mixture was stirred at 15° C. for 0.25 hr. and monitored by TLC (DCM/MeOH=10/1, Rf=0.43). The reaction mixture was concentrated. The residue was poured into EtOAc (20 mL) and extracted. The combined organic phases were washed with 0.1 M HCl (25 mL×1), saturated aqueous NaHCO₃ (25 mL×1) and brine (25 mL×1), dried over anhydrous Na₂SO₄, filtered and concentrated in vacuum to afford the title compound (290 mg, 1.29 mmol, 68.63% yield) as a pale yellow solid.

### Example 1. 5-Methyl-N-(3-phenyl-1H-indazol-5-yl)-1H-benzo[d][1,2,3]triazole-6-carboxamide

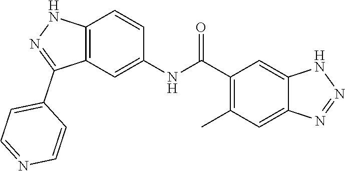 [SMILES: Cc1cc2nn[nH]c2cc1C(=O)Nc1ccc2[nH]nc(-c3ccncc3)c2c1]

### Step 1. 3-(Pyridin-4-yl)-1H-indazol-5-amine

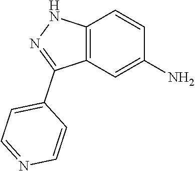 [SMILES: Nc1ccc2[nH]nc(-c3ccncc3)c2c1]

3-Bromo-1H-indazol-5-amine (Intermediate 1; 100 mg, 471.59 umol), pyridin-4-ylboronic acid (86.95 mg, 707.39 umol), RuPhos (22.01 mg, 47.16 umol), K₃PO₄ (200.21 mg, 943.19 umol) and Pd(OAc)₂ (5.29 mg, 23.58 umol) were taken up into a microwave tube in dioxane (2 mL) and H₂O (2 mL) under N₂. The sealed tube was heated at 120° C. for 0.5 hr under microwave. The reaction mixture was concentrated and purified by Prep-TLC (SiO₂, dichloromethane:MeOH=10:1) to afford the title compound (30 mg, 142.70 umol, 30.26% yield) as a yellow solid.

### Step 2. 5-Methyl-N-(3-phenyl-1H-indazol-5-yl)-1H-benzo[d][1,2,3]triazole-6-carboxamide

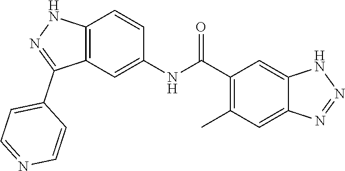 [SMILES: Cc1cc2nn[nH]c2cc1C(=O)Nc1ccc2[nH]nc(-c3ccncc3)c2c1]

To a solution of 3-(pyridin-4-yl)-1H-indazol-5-amine (60 mg, 285.40 umol) in toluene (3 mL) was added methyl 5-methyl-1H-benzo[d][1,2,3]triazole-6-carboxylate (54.56 mg, 285.40 umol) and Al(CH₃)₃ (2 M, 713.49 uL), the reaction mixture was stirred at 90° C. for 12 hrs. The reaction mixture was quenched with water (6 mL) and concentrated. The residue was dissolved in DMF (3 mL) and filtered, the filtrate was concentrated and purified by Prep-HPLC (basic condition) to afford the title compound (25.67 mg, 62.18 umol, 21.79% yield) as a pale yellow solid. ¹H NMR (400 MHz, DMSO-d₆) δ 10.53 (s, 1H), 8.72 (s, 2H), 8.71 (br s, 1H), 8.12 (s, 1H), 7.93 (d, J=6.0 Hz, 2H), 7.80-7.72 (m, 2H), 7.65 (d, J=9.0 Hz, 1H), 2.56 (s, 3H). MS-ESI (m/z) calc'd for C₂₀H₁₇N₇O [M+H]⁺: 370.1. Found 370.1.

### Example 2: 5-Methyl-N-(3-(3-morpholinophenyl)-1H-indazol-5-yl)-1H-benzo[d][1,2,3]triazole-6-carboxamide

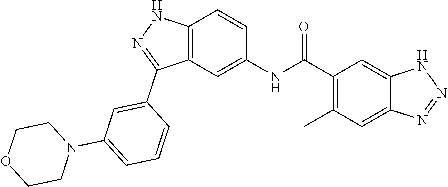 [SMILES: Cc1cc2nn[nH]c2cc1C(=O)Nc1ccc2[nH]nc(-c3cccc(N4CCOCC4)c3)c2c1]

### Step 1. 3-(3-Morpholinophenyl)-1H-indazol-5-amine

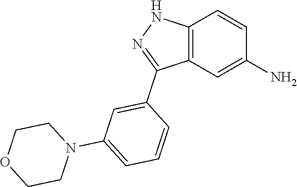 [SMILES: Nc1ccc2[nH]nc(-c3cccc(N4CCOCC4)c3)c2c1]

To 3-bromo-1H-indazol-5-amine (Intermediate 1; 150 mg, 707.39 umol) and (3-morpholinophenyl)boronic acid (219.68 mg, 1.06 mmol) in dioxane (2 mL) was added a solution Na₂CO₃ (374.88 mg, 3.54 mmol) in H₂O (2 mL) and Pd(dppf)Cl₂ (51.76 mg, 70.74 umol). The mixture was taken up into a microwave tube under N₂. The sealed tube was heated at 120° C. for 0.5 hr under microwave. LC-MS showed the reaction was completed. After cooling to 20° C., the reaction mixture was filtered and the filtrate was concentrated. The residue was purified by Prep-TLC (SiO₂, petroleum ether/EtOAc=1:2) to give the title compound (150 mg, 509.60 umol, 72.04% yield) as a yellow solid.

### Step 2. 5-Methyl-N-(3-(3-morpholinophenyl)-1H-indazol-5-yl)-1H-benzo[d][1,2,3]triazole-6-carboxamide

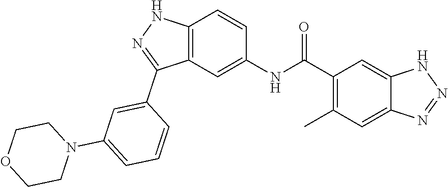 [SMILES: Cc1cc2nn[nH]c2cc1C(=O)Nc1ccc2[nH]nc(-c3cccc(N4CCOCC4)c3)c2c1]

To a solution of 3-(3-morpholinophenyl)-1H-indazol-5-amine (150 mg, 509.60 umol) in toluene (3 mL) was added methyl 5-methyl-1H-benzo[d][1,2,3]triazole-6-carboxylate (97.43 mg, 509.60 umol) and AlMe₃ (2 M, 1.27 mL). The reaction mixture was stirred at 90° C. for 12 hrs. The reaction mixture was poured into water (10 mL) and concentrated. The residue was diluted with DMF (10 mL) and filtered. The filtrate was concentrated and purified by Prep-HPLC (TFA condition) to afford the title compound (21.21 mg, 35.87 umol, 7.04% yield, TFA salt) as a white solid. ¹H NMR (400 MHz, DMSO-d₆) δ 13.18 (br s, 1H), 10.51 (s, 1H), 8.64 (s, 1H), 8.14 (br s, 1H), 7.79 (br s, 1H), 7.72 (dd, J=1.4, 8.9 Hz, 1H), 7.58 (d, J=9.0 Hz, 1H), 7.48 (s, 1H), 7.43-7.35 (m, 2H), 7.05-6.99 (m, 1H), 3.81-3.75 (m, 4H), 3.24-3.19 (m, 4H), 2.55 (s, 3H). MS-ESI (m/z) calc'd for C₂₅H₂₄N₇O₂ [M+H]⁺: 454.2. Found 454.1.

### Example 3: 2,6-Dimethyl-N-(3-methyl-1H-indazol-5-yl)-2H-benzo[d][1,2,3]triazole-5-carboxamide

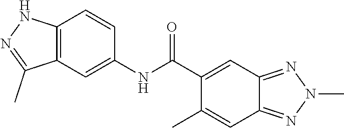 [SMILES: Cc1cc2nn(C)nc2cc1C(=O)Nc1ccc2[nH]nc(C)c2c1]

### Step 1. Methyl 2,6-dimethyl-2H-benzo[d][1,2,3]triazole-5-carboxylate and methyl 1,6-dimethyl-1H-benzo[d][1,2,3]triazole-5-carboxylate and methyl 1,5-dimethyl-1H-benzo[d][1,2,3]triazole-6-carboxylate

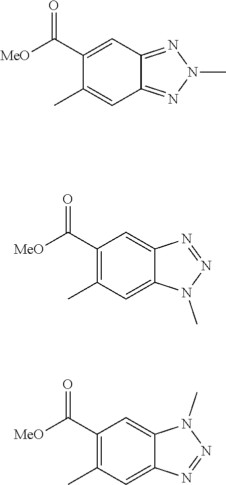

To a solution of methyl 5-methyl-1H-benzo[d][1,2,3]triazole-6-carboxylate (200 mg, 1.05 mmol) in DMF (4 mL) was added NaH (50.21 mg, 1.26 mmol, 60% purity), then the mixture was stirred at 25° C. for 0.5 hr, then Mel (445.45 mg, 3.14 mmol, 195.37 uL) was added and the reaction mixture was stirred for another 12 hrs at 25° C. The reaction mixture was concentrated under vacuum. The residue was purified by Prep-TLC (petroleum ether:EtOAc=3:1) to afford methyl 2,6-dimethyl-2H-benzo[d][1,2,3]triazole-5-carboxylate (80 mg, 29.81% yield) as a yellow solid and a mixture of methyl 1,6-dimethyl-1H-benzo[d][1,2,3]triazole-5-carboxylate and methyl 1,5-dimethyl-1H-benzo[d][1,2,3]triazole-6-carboxylate (155 mg, 28.88% yield) as a brown solid.

### Step 2. 2,6-Dimethyl-2H-benzo[d][1,2,3]triazole-5-carboxylic acid

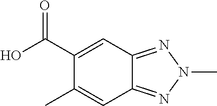

To a solution of methyl 2,6-dimethyl-2H-benzo[d][1,2,3]triazole-5-carboxylate (74 mg, 360.60 umol) in MeOH (1.5 mL) and H₂O (1.5 mL) was added LiOH.H₂O (60.53 mg, 1.44 mmol). The mixture was stirred at 25° C. for 12 hrs. The MeOH was removed under vacuum, the aqueous layer was acidified with 1 N HCl to pH=5, then the aqueous layer was filtered and the filter-cake was dried under vacuum to give the title compound (52 mg, crude) as a white solid, which was used in the next step without further purification.

### Step 3. 2,6-Dimethyl-N-(3-methyl-1H-indazol-5-yl)-2H-benzo[d][1,2,3]triazole-5-carboxamide

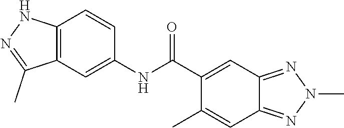 [SMILES: Cc1cc2nn(C)nc2cc1C(=O)Nc1ccc2[nH]nc(C)c2c1]

To a solution of 2,6-dimethyl-2H-benzo[d][1,2,3]triazole-5-carboxylic acid (52 mg, 271.99 umol) and 3-methyl-1H-indazol-5-amine (48.04 mg, 326.38 umol) in DCM (2 mL) was added T₃P/EtOAc (519.25 mg, 815.96 umol, 485.28 uL, 50% purity) and TEA (110.09 mg, 1.09 mmol, 151.43 uL). The mixture was stirred at 25° C. for 12 hrs. LC-MS showed the reaction was completed. The reaction mixture was concentrated and the residue was washed with 2 mL MeOH. The mixture was filtered and the filter-cake was dried under vacuum to afford the title compound (26.92 mg, 84.03 umol, 30.90% yield) as a white solid. ¹H NMR (400 MHz, DMSO-d₆) δ 8.18 (d, J=1.22 Hz, 1H) 8.03 (s, 1H) 7.75 (s, 1H) 7.57 (dd, J=8.93, 1.96 Hz, 1H) 7.44-7.51 (m, 1H) 4.51 (s, 3H) 2.60 (d, J=0.61 Hz, 3H) 2.57 (s, 3H). MS-ESI (m/z) calc'd for C₁₇H₁₇N₆O [M+H]⁺: 321.1. Found 321.1.

### Example 4: 1,6-Dimethyl-N-(3-methyl-1H-indazol-5-yl)-1H-benzo[d][1,2,3]triazole-5-carboxamide and 1,5-dimethyl-N-(3-methyl-1H-indazol-5-yl)-1H-benzo[d][1,2,3]triazole-6-carboxamide

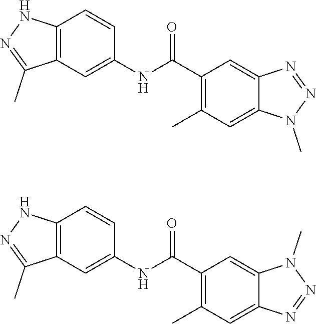 [SMILES: Cc1cc2c(cc1C(=O)Nc1ccc3[nH]nc(C)c3c1)nnn2C ; Cc1cc2nnn(C)c2cc1C(=O)Nc1ccc2[nH]nc(C)c2c1]

### Step 1. 1,6-Dimethyl-1H-benzo[d][1,2,3]triazole-5-carboxylic acid and 1,5-dimethyl-1H-benzo[d][1,2,3]triazole-6-carboxylic acid

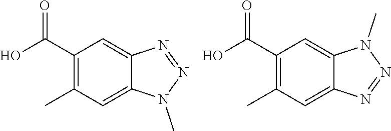

To a solution of methyl 1,6-dimethyl-1H-benzo[d][1,2,3]triazole-5-carboxylate and methyl 1,5-dimethyl-1H-benzo[d][1,2,3]triazole-6-carboxylate (100 mg, 243.65 umol) in MeOH (2 mL) and H₂O (2 mL) was added LiOH.H₂O (40.89 mg, 1.44 mmol). The mixture was stirred at 25° C. for 12 hrs. TLC and LCMS showed the reaction was completed. The MeOH was removed under vacuum and the aqueous layer was acidified with 1 N HCl to pH=5. The aqueous layer was then filtered and the filter-cake was dried under vacuum to give the title compounds (56 mg, crude) as a light pink solid mixture and the mixture was used in the next step without further purification.

### Step 2. 1,6-Dimethyl-N-(3-methyl-1H-indazol-5-yl)-1H-benzo[d][1,2,3]triazole-5-carboxamide and 1,5-dimethyl-N-(3-methyl-1H-indazol-5-yl)-1H-benzo[d][1,2,3]triazole-6-carboxamide

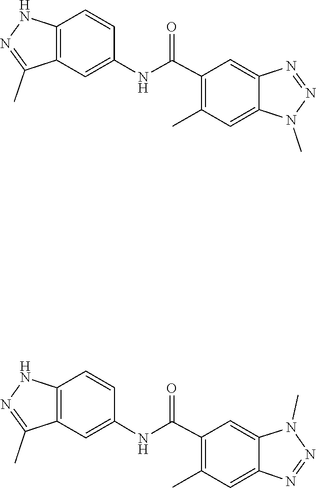

To a solution of 1,6-dimethyl-1H-benzo[d][1,2,3]triazole-5-carboxylic acid and 1,5-dimethyl-1H-benzo[d][1,2,3]triazole-6-carboxylic acid (56 mg, 146.45 umol) and 3-methyl-1H-indazol-5-amine (25.87 mg, 175.74 umol) in DCM (2 mL) was added T₃P/EtOAc (279.59 mg, 439.36 umol, 361.30 uL, 50% purity) and TEA (59.28 mg, 585.82 umol, 81.54 uL). The mixture was stirred at 25° C. for 12 hrs. LC-MS showed the starting material: desired product=1:1. The reaction was concentrated under vacuum. The residue was purified by prep-HPLC (TFA condition), then the residue was separated by SFC (Instrument: Thar SFC80 preparative SFC; Column: Chiralpak AD-H 250*30 mm i.d. 5 u; Mobile phase: A for C₀₂ and B for EtOH (0.1% NH₃.H₂O); Gradient: B %=41%; Flow rate: 60 g/min; Wavelength: 220 nm; Column temperature: 40° C.; System back pressure: 100 bar) to afford the title compounds. 1,6-dimethyl-N-(3-methyl-1H-indazol-5-yl)-1H-benzo[d][1,2,3]triazole-5-carboxamide (5.62 mg, 17.45 umol, 11.91% yield) (Rt=2.11 min) was isolated as a white solid. ¹H NMR (400 MHz, DMSO-d₆) δ 8.17 (s, 1H) 8.14 (s, 1H) 7.69 (s, 1H) 7.57 (dd, J=8.77, 1.75 Hz, 1H) 7.44-7.50 (m, 1H) 4.34 (s, 3H) 2.67 (s, 3H) 2.56 (s, 3H). MS-ESI (m/z) calc'd for C₁₇H₁₇N₆O [M+H]⁺: 321.1. Found 321.1. 1,5-Dimethyl-N-(3-methyl-1H-indazol-5-yl)-1H-benzo[d][1,2,3]triazole-6-carboxamide (5.86 mg, 18.03 umol, 12.31% yield) (Rt=1.74 min) was isolated as a pale yellow solid. ¹H NMR (400 MHz, DMSO-d₆) δ 8.19 (s, 1H) 7.97 (s, 1H) 7.89 (s, 1H) 7.55-7.60 (m, 1H) 7.46-7.51 (m, 1H) 4.37 (s, 3H) 2.63 (s, 3H) 2.57 (s, 3H). MS-ESI (m/z) calc'd for C₁₇H₁₇N₆O [M+H]⁺: 321.1. Found 321.1.

### Example 5. N-(3-Methyl-1H-indazol-5-yl)-[1,2,4]triazolo[4,3-a]pyridine-7-carboxamide

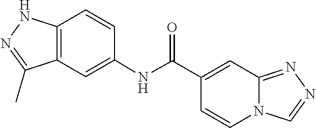 [SMILES: Cc1n[nH]c2ccc(NC(=O)c3ccn4cnnc4c3)cc12]

To a solution of [1,2,4]triazolo[4,3-a]pyridine-7-carboxylic acid (100 mg, 612.99 umol) in pyridine (2 mL) was added 3-methyl-1H-indazol-5-amine (99.24 mg, 674.29 umol) and EDCI (176.27 mg, 919.49 umol). The mixture was stirred at 25° C. for 12 hrs. The reaction mixture was concentrated under reduced pressure to give a residue. The residue was purified by Prep-HPLC (TFA condition) to afford the title compound (153.08 mg, 374.71 umol, 61.13% yield, TFA salt) as a yellow solid. ¹H NMR (400 MHz, DMSO-d₆) δ 10.55 (s, 1H) 9.41 (s, 1H) 8.70 (d, J=7.06 Hz, 1H) 8.51 (s, 1H) 8.21 (s, 1H) 7.63 (dd, J=8.93, 1.87 Hz, 1H) 7.45-7.49 (m, 2H) 2.48 (s, 3H). MS-ESI (m/z) calc'd for C₁₅H₁₃N₆O [M+H]⁺: 293.1. Found 293.0.

### Example 6. 3-Methyl-N-(3-methyl-1H-indazol-5-yl)-[1,2,4]triazolo[4,3-a]pyridine-7-carboxamide

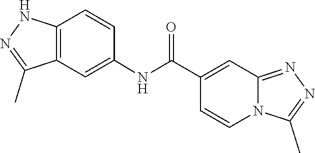 [SMILES: Cc1n[nH]c2ccc(NC(=O)c3ccn4c(C)nnc4c3)cc12]

### Step 1. Methyl 3-methyl-[1,2,4]triazolo[4,3-a]pyridine-7-carboxylate

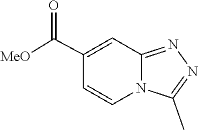

To a solution of 7-bromo-3-methyl-[1,2,4]triazolo[4,3-a]pyridine (300 mg, 1.41 mmol) in MeOH (4 mL) was added TEA (1.43 g, 14.15 mmol) and Pd(dppf)Cl₂ (103.52 mg, 141.48 umol) under N₂. The suspension was degassed under vacuum and purged with CO several times. The mixture was stirred at 70° C. for 12 hrs under CO atmosphere (50 Psi). The reaction mixture was concentrated under reduced pressure to remove the solvent. The residue was diluted with EtOAc (15 mL), filtered and the solid was dried under vacuum to afford the title compound (260 mg, crude) as a red solid.

### Step 2. 3-Methyl-N-(3-methyl-1H-indazol-5-yl)-[1,2,4]triazolo[4,3-a]pyridine-7-carboxamide

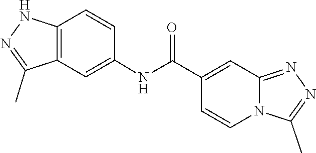 [SMILES: Cc1n[nH]c2ccc(NC(=O)c3ccn4c(C)nnc4c3)cc12]

To a solution of methyl 3-methyl-[1,2,4]triazolo[4,3-a]pyridine-7-carboxylate (150 mg, 784.58 umol) and 3-methyl-1H-indazol-5-amine (115.47 mg, 784.58 umol) in toluene (5 mL) was added AlMe₃ (2 M, 1.57 mL). The mixture was stirred at 20° C. for 12 hrs. The reaction mixture was quenched with H₂O (10 mL) and concentrated under reduced pressure to remove the solvent. The residue was diluted with DMF (5 mL), filtered and the filtrate was concentrated and purified by Prep-HPLC (TFA condition) to afford the title compound (80.91 mg, 192.49 umol, 24.53% yield, TFA salt) as a yellow solid. ¹H NMR (400 MHz, DMSO-d₆) δ 10.57 (s, 1H) 8.58 (d, J=7.3 Hz, 1H) 8.47 (s, 1H) 8.20 (d, J=1.3 Hz, 1H) 7.64 (dd, J=1.8, 8.8 Hz, 1H) 7.53 (d, J=7.3 Hz, 1H) 7.48 (d, J=8.8 Hz, 1H) 2.77 (s, 3H) 2.49 (s, 3H). MS-ESI (m/z) calc'd for C₁₆H₁₅N₆O [M+H]⁺: 307.1. Found 307.1.

### Example 7. N-(3-Methyl-1H-indazol-5-yl)-[1,2,4]triazolo[4,3-a]pyridine-6-carboxamide

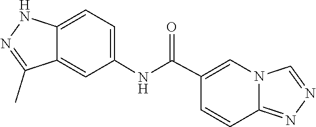 [SMILES: Cc1n[nH]c2ccc(NC(=O)c3ccc4nncn4c3)cc12]

To a solution of [1,2,4]triazolo[4,3-a]pyridine-6-carboxylic acid (100 mg, 613.00 umol) and 3-methyl-1H-indazol-5-amine (90.22 mg, 613.00 umol) in DCM (2 mL) was added T₃P/EtOAc (585.13 mg, 919.50 umol, 546.85 uL, 50% purity) and the reaction mixture was stirred at 25° C. for 0.5 hr. TEA (186.09 mg, 1.84 mmol, 255.97 uL) was then added and the reaction mixture was stirred at 25° C. for 3 hrs. The mixture was concentrated to give a residue. The residue was washed by MeOH (2 mL) and filtered and the solid was concentrated in vacuum to give the title compound (21.7 mg, 66.82 umol, 10.90% yield) as a white solid. ¹H NMR (400 MHz, DMSO-d₆) δ 12.64 (br s, 1H) 10.48 (s, 1H) 9.45 (s, 1H) 9.28 (s, 1H) 8.16 (s, 1H) 7.91 (s, 2H) 7.60 (br d, J=7.82 Hz, 1H) 7.49 (d, J=8.80 Hz, 1H) 2.50 (br s, 3H). MS-ESI (m/z) calc'd for C₁₅H₁₃N₆O [M+H]⁺: 293.1. Found 293.0.

### Example 8. 3-Methyl-N-(3-methyl-1H-indazol-5-yl)-[1,2,4]triazolo[4,3-a]pyridine-6-carboxamide

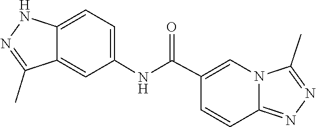 [SMILES: Cc1n[nH]c2ccc(NC(=O)c3ccc4nnc(C)n4c3)cc12]

### Step 1. 3-Methyl-[1,2,4]triazolo[4,3-a]pyridine-6-carboxylic acid

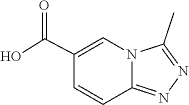

A solution of 6-hydrazinonicotinic acid (400 mg, 2.61 mmol) and AcOH (2.10 g, 34.97 mmol) was stirred at 120° C. for 14 hrs. The reaction mixture was concentrated to afford the title compound (460 mg, crude) as a white solid which was used without further purification.

### Step 2. 3-Methyl-N-(3-methyl-1H-indazol-5-yl)-[1,2,4]triazolo[4,3-a]pyridine-6-carboxamide

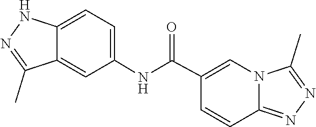 [SMILES: Cc1n[nH]c2ccc(NC(=O)c3ccc4nnc(C)n4c3)cc12]

To a solution of 3-methyl-[1,2,4]triazolo[4,3-a]pyridine-6-carboxylic acid (70 mg, 395.12 umol) in DCM (2 mL) was added 3-methyl-1H-indazol-5-amine (69.78 mg, 474.15 umol), T₃P/EtOAc (326.87 mg, 513.66 umol, 50% purity) and TEA (119.95 mg, 1.19 mmol). The mixture was stirred at 25° C. for 12 hrs. LC-MS showed incomplete reaction. The reaction mixture was then warmed to 40° C. and stirred for another 6 hr. The reaction mixture was concentrated to remove the solvent and purified by Prep-HPLC (neutral condition) to afford the title compound (14.96 mg, 48.84 umol, 12.36% yield) as a white solid. ¹H NMR (400 MHz, DMSO-d₆) δ 12.64 (br s, 1H) 10.41 (s, 1H) 9.05 (s, 1H) 8.12 (d, J=1.34 Hz, 1H) 7.82 (d, J=0.86 Hz, 2H) 7.60 (dd, J=8.86, 1.77 Hz, 1H) 7.48 (d, J=8.80 Hz, 1H) 2.78 (s, 3H) 2.48 (s, 3H). MS-ESI (m/z) calc'd for C₁₆H₁₅N₆O [M+H]⁺: 307.1. Found 307.0.

### Example 9. N-(3-Methyl-1H-indazol-5-yl)-1H-benzo[d][1,2,3]triazole-6-carboxamide

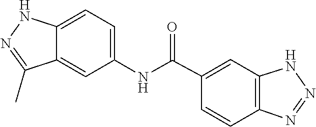

### Step 1. Ethyl 1H-benzo[d][1,2,3]triazole-6-carboxylate

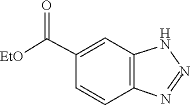

To a solution of ethyl 3,4-diaminobenzoate (1 g, 5.55 mmol) in H₂O (9 mL) was added AcOH (833.11 mg, 13.87 mmol) at 5° C. followed by a solution of NaNO₂ (459.45 mg, 6.66 mmol) in H₂O (1 mL) added drop-wise at 5° C. After addition, the temperature of the above mixture was slowly raised to 50° C. for 0.25 hr. Then the reaction mixture was cooled to 20° C. and stirred at for 12 hrs. The reaction mixture was quenched with water (25 mL), filtered and the solid was washed with water (10 mL) and dried under vacuum to afford the title compound (1.05 g, crude) as a reddish-brown solid which was used without further purification.

### Step 2. 1H-Benzo[d][1,2,3]triazole-6-carboxylic acid

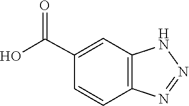 [SMILES: O=C(O)c1ccc2nn[nH]c2c1]

To a solution of ethyl 1H-benzo[d][1,2,3]triazole-6-carboxylate (0.3 g, 1.57 mmol) in THF (3 mL) and MeOH (3 mL) was added LiOH.H₂O (2 M, 2.35 mL). The mixture was stirred at 25° C. for 13 hrs. The reaction mixture was concentrated under reduced pressure to remove the solvent. The residue was diluted with H₂O 8 mL and adjusted to pH-1-2 with 1N HCl. The resulting precipitate was collected by filtration and dried under vacuum to afford the title compound (210 mg, crude) as a brown solid which was used without further purification.

### Step 3. N-(3-Methyl-1H-indazol-5-yl)-1H-benzo[d][1,2,3]triazole-6-carboxamide

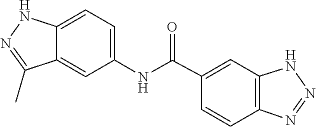

To a solution of 1H-benzo[d][1,2,3]triazole-6-carboxylic acid (100 mg, 613.00 umol) and 3-methyl-1H-indazol-5-amine (90.22 mg, 613.00 umol) in DCM (4 mL) was added T₃P/EtOAc (507.11 mg, 796.89 umol) and TEA (186.09 mg, 1.84 mmol). The mixture was stirred at 25° C. for 3 hrs. The reaction mixture was concentrated under reduced pressure to give a residue. The residue was purified by Prep-HPLC (TFA condition) to afford the title compound (41.92 mg, 103.05 umol, 16.81% yield, TFA salt) as a white solid. ¹H NMR (400 MHz, DMSO-d₆) δ 12.60 (br s, 1H), 10.43 (s, 1H), 8.65 (br s, 1H), 8.21 (d, J=1.1 Hz, 1H), 8.11-7.94 (m, 2H), 7.65 (dd, J=1.7, 8.9 Hz, 1H), 7.46 (d, J=8.8 Hz, 1H), 2.49 (s, 3H). MS-ESI (m/z) calc'd for C₁₅H₁₃N₆O [M+H]⁺: 293.1. Found 293.1.

### Example 10. N-(3-Methyl-1H-indazol-5-yl)-6-(trifluoromethyl)-1H-benzo[d][1,2,3]triazole-5-carboxamide

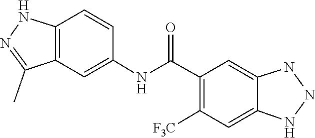

### Step 1. 4-Bromo-5-(trifluoromethyl)benzene-1,2-diamine

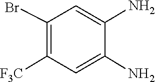 [SMILES: Nc1cc(Br)c(C(F)(F)F)cc1N]

To a solution of 4-bromo-2-nitro-5-(trifluoromethyl)aniline (500 mg, 1.75 mmol) in EtOH (2 mL) and EtOAc (8 mL) was added SnCl₂.2H₂O (1.98 g, 8.77 mmol). The mixture was stirred at 80° C. for 4 hr. The reaction mixture was combined with a second 100 mg batch. The reaction mixture was basified to pH=8 with saturated aqueous NaHCO₃. The reaction mixture was filtered and the filtrate was extracted with EtOAc (30 mL×3). The combined organic layers were washed with brine (20 mL×3), dried over anhydrous Na₂SO₄ and concentrated under reduced pressure to afford the title compound (550 mg, crude) as a yellow oil.

### Step 2. 5-Bromo-6-(trifluoromethyl)-1H-benzo[d][1,2,3]triazole

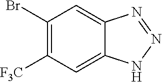 [SMILES: FC(F)(F)c1cc2[nH]nnc2cc1Br]

To a solution of 4-bromo-5-(trifluoromethyl)benzene-1,2-diamine (550 mg, 2.16 mmol) in H₂O (6 mL) was added AcOH (323.77 mg, 5.39 mmol) at 5° C. NaNO₂ (178.55 mg, 2.59 mmol) in H₂O (2 mL) was then added to the mixture at 5° C. The mixture was stirred at 25° C. for 12 hrs. The reaction mixture was filtered and the solid was washed with H₂O (50 mL) and dried under vacuum to afford the title compound (400 mg, crude) as a yellow solid.

### Step 3. Methyl 6-(trifluoromethyl)-1H-benzo[d][1,2,3]triazole-5-carboxylate

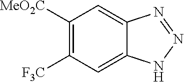 [SMILES: COC(=O)c1cc2nn[nH]c2cc1C(F)(F)F]

A mixture of 5-bromo-6-(trifluoromethyl)-1H-benzo[d][1,2,3]triazole (200 mg, 751.83 umol), TEA (2.28 g, 22.55 mmol, 3.14 mL) and Pd(dppf)Cl₂ (27.51 mg, 37.59 umol) in MeOH (6 mL) was degassed and purged with CO thrice and then the reaction mixture was stirred at 70° C. for 48 hrs under CO atmosphere (50 Psi). The reaction mixture was concentrated and purified by column chromatography (SiO₂, petroleum ether/EtOAc=1:0 to 1:1) to afford the title compound (120 mg, 293.69 umol, 39% yield) as a yellow oil.

### Step 4. 6-(Trifluoromethyl)-1H-benzo[d][1,2,3]triazole-5-carboxylic acid

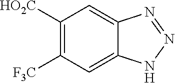 [SMILES: O=C(O)c1cc2nn[nH]c2cc1C(F)(F)F]

To a solution of methyl 6-(trifluoromethyl)-1H-benzo[d][1,2,3]triazole-5-carboxylate (120 mg, 489.48 umol) in THF (4 mL) and H₂O (2 mL) was added an aqueous NaOH solution (2 M, 2.45 mL). The mixture was stirred at 50° C. for 12 hrs. The reaction mixture was acidified with 1 N HCl to pH=3 and extracted with EtOAc (5 mL×3). The combined organic layers were dried over anhydrous Na₂SO₄, filtered and the filtrate was concentrated to afford the title compound (35 mg, crude) as a yellow solid.

### Step 5. N-(3-Methyl-1H-indazol-5-yl)-6-(trifluoromethyl)-1H-benzo[d][1,2,3]triazole-5-carboxamide

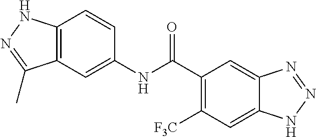 [SMILES: Cc1n[nH]c2ccc(NC(=O)c3cc4nn[nH]c4cc3C(F)(F)F)cc12]

To a solution of 6-(trifluoromethyl)-1H-benzo[d][1,2,3]triazole-5-carboxylic acid (30 mg, 129.80 umol) in DMF (2 mL) was added DIEA (50.33 mg, 389.39 umol, 67.82 uL), 3-methyl-1H-indazol-5-amine (19.10 mg, 129.80 umol) and HATU (74.03 mg, 194.69 umol). The mixture was stirred at 25° C. for 12 hr. The reaction mixture was concentrated and purified by Prep-HPLC (TFA condition) to afford the title compound (20.27 mg, 42.46 umol, 32.71% yield, TFA salt) as a red solid. ¹H NMR (400 MHz, DMSO-d₆) δ 12.60 (br s, 1H) 10.59 (s, 1H) 8.56 (br s, 1H) 8.31 (br s, 1H) 8.18 (s, 1H) 7.55-7.35 (m, 3H) 2.48 (br s, 3H). MS-ESI (m/z) calc'd for C₁₆H₁₂F₃N₆O [M+H]⁺: 361.1. Found 361.0.

### Example 11. 4,6-Dimethyl-N-(3-methyl-1H-indazol-5-yl)-1H-benzo[d][1,2,3]triazole-5-carboxamide

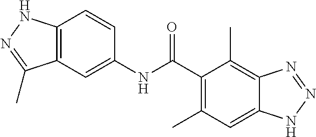 [SMILES: Cc1cc2[nH]nnc2c(C)c1C(=O)Nc1ccc2[nH]nc(C)c2c1]

### Step 1. Methyl 4-amino-2,6-dimethylbenzoate

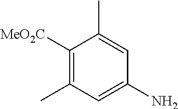

A mixture of 4-bromo-3,5-dimethylaniline (2 g, 10.00 mmol), TEA (2.02 g, 19.99 mmol), Pd(dppf)Cl₂ (365.71 mg, 499.81 umol) in MeOH (158.36 g, 4.94 mol, 200.00 mL) was degassed and purged with CO (3×), and then the reaction mixture was stirred at 80° C. for 12 hrs under CO (50 psi). The mixture was concentrated and purified by column chromatography (SiO₂, petroleum ether/EtOAc=100:0 to 70:30) to afford the title compound (1 g, 5.58 mmol, 55.82% yield) as a white solid.

### Step 2. Methyl 4-amino-2,6-dimethyl-3-nitrobenzoate

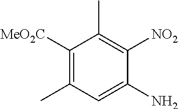 [SMILES: COC(=O)c1c(C)cc(N)c([N+](=O)[O-])c1C]

To a solution of methyl 4-amino-2,6-dimethylbenzoate (0.5 g, 2.79 mmol) in (purity: 98%) H₂SO₄ (10 mL) was added (purity: 65%) HNO₃ (297.51 mg, 3.07 mmol, 212.51 uL) slowly at 0-15° C. and the reaction mixture was stirred at 25° C. for 3 hrs. The mixture was quenched with NH₃.H₂O and the pH was adjusted to 10 at 0° C. The aqueous phase was extracted with EtOAc (30 mL×3). The combined organic layers were washed with brine (30 mL×1), dried with anhydrous Na₂SO₄, filtered and concentrated under vacuum. The residue was purified by column chromatography (SiO₂, petroleum ether/EtOAc=100:0 to 93:7) to afford the title compound (370 mg, 1.65 mmol, 59.15% yield) as a yellow solid.

### Step 3. Methyl 3,4-diamino-2,6-dimethylbenzoate

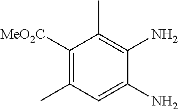

A mixture of methyl 4-amino-2,6-dimethyl-3-nitrobenzoate (800 mg, 3.57 mmol), Pd/C (0.4 g, 10% purity) in MeOH (2 mL) was degassed and purged with H₂ (3×) and then the mixture was stirred at 25° C. for 0.5 hr under H₂ (15 psi). The reaction mixture was filtered and the filtrate was concentrated to afford the title compound (600 mg, 3.09 mmol, 86.58% yield) as a red oil, which was used without further purification.

### Step 4. Methyl 4,6-dimethyl-1H-benzo[d][1,2,3]triazole-5-carboxylate

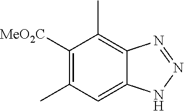

To a solution of methyl 3,4-diamino-2,6-dimethylbenzoate (200.00 mg, 1.03 mmol) in H₂O (2 mL) was added AcOH (154.59 mg, 2.57 mmol, 147.23 uL) at 5° C., then a solution of NaNO₂ (85.25 mg, 1.24 mmol) in H₂O (1 mL) was added dropwise at 5° C. After addition, the temperature of the mixture was slowly raised to 50° C. for 0.25 hr. The reaction mixture was then cooled to 20° C. and stirred at 20° C. for 12 hrs. The reaction mixture was filtered and the solid was dried under vacuum to afford the title compound (150 mg, crude) as a red solid, which was used without further purification.

### Step 5. 4,6-Dimethyl-N-(3-methyl-1H-indazol-5-yl)-1H-benzo[d][1,2,3]triazole-5-carboxamide

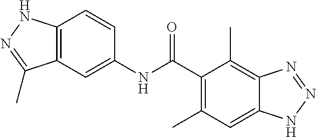 [SMILES: Cc1cc2[nH]nnc2c(C)c1C(=O)Nc1ccc2[nH]nc(C)c2c1]

To a solution of methyl 4,6-dimethyl-1H-benzo[d][1,2,3]triazole-5-carboxylate (150.00 mg, 730.95 umol) and 3-methyl-1H-indazol-5-amine (107.58 mg, 730.95 umol) in toluene (2 mL) was added AlMe₃ (2 M, 1.10 mL) at 0° C. The reaction mixture was then heated to 120° C. and stirred for 5 hrs. The mixture was diluted with DMF (1 mL), quenched with H₂O (1 mL) and filtered. The filtrate was concentrated and purified by Prep-HPLC (neutral condition) to afford the title compound (21.78 mg, 66.27 umol, 9.07% yield) as a yellow oil. ¹H NMR (400 MHz, MeOD) δ 8.20 (d, J=1.22 Hz, 1H) 7.54-7.63 (m, 2H) 7.46-7.53 (m, 1H) 2.79 (br s, 3H) 2.59 (s, 6H). MS-ESI (m/z) calc'd for C₁₇H₁₇N₆O [M+H]⁺: 321.1. Found 321.1.

### Example 12. 4-Methyl-N-(3-(pyridin-4-yl)-1H-indazol-5-yl)-1H-benzo[d][1,2,3]triazole-5-carboxamide

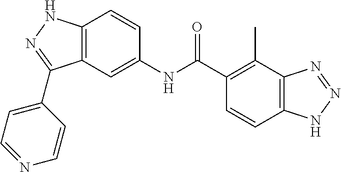 [SMILES: Cc1c(C(=O)Nc2ccc3[nH]nc(-c4ccncc4)c3c2)ccc2[nH]nnc12]

### Step 1. 5-Bromo-4-methyl-1H-benzo[d][1,2,3]triazole

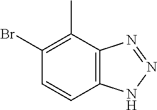

To a solution of 4-bromo-3-methylbenzene-1,2-diamine (400 mg, 1.99 mmol) in H₂O (4 mL) was added AcOH (298.67 mg, 4.97 mmol) at 5° C. followed by a solution of NaNO₂ (164.71 mg, 2.39 mmol) in H₂O (1 mL). The mixture was warmed to 25° C. and stirred for 12 hrs. The reaction mixture was filtered, the solid was washed with H₂O (50 mL) and dried under vacuum to afford the title compound (500 mg, crude) as a pale yellow solid.

### Step 2. Methyl 4-methyl-1H-benzo[d][1,2,3]triazole-5-carboxylate

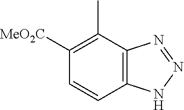

A mixture of 5-bromo-4-methyl-1H-benzo[d][1,2,3]triazole (500 mg, 2.36 mmol), TEA (1.43 g, 14.15 mmol, 1.97 mL) and Pd(dppf)Cl₂ (86.27 mg, 117.90 umol) in MeOH (15 mL) was degassed and purged with CO (3×), and then the mixture was stirred at 70° C. for 36 hrs under a CO atmosphere (50 Psi). The reaction mixture was concentrated under reduced pressure to remove the solvent and purified by column chromatography (SiO₂, petroleum ether/EtOAc=1/0 to 1/1) to give the title compound (400 mg, 2.09 mmol, 88.73% yield) as a yellow solid.

### Step 3. 4-Methyl-1H-benzo[d][1,2,3]triazole-5-carboxylic acid

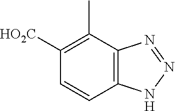

To a solution of methyl 4-methyl-1H-benzo[d][1,2,3]triazole-5-carboxylate (200 mg, 1.05 mmol) in THF (3 mL) and H₂O (3 mL) was added LiOH (150.31 mg, 6.28 mmol). The mixture was stirred at 25° C. for 12 hrs. The mixture was acidified with 1N HCl to pH=3 and extracted with EtOAc (5 mL×3). The combined organic layers were dried over anhydrous Na₂SO₄, filtered and the filtrate was concentrated to give the title compound (200 mg, crude) as a yellow solid which was used without further purification.

### Step 4. 4-Methyl-N-(3-(pyridin-4-yl)-1H-indazol-5-yl)-1H-benzo[d][1,2,3]triazole-5-carboxamide

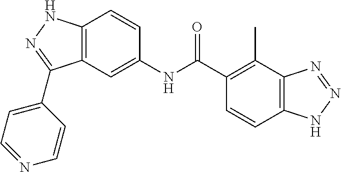 [SMILES: Cc1c(C(=O)Nc2ccc3[nH]nc(-c4ccncc4)c3c2)ccc2[nH]nnc12]

To a solution of 4-methyl-1H-benzo[d][1,2,3]triazole-5-carboxylic acid (58.99 mg, 332.96 umol) in DCM (4 mL) was added T₃P/EtOAc (317.83 mg, 499.44 umol, 50% purity), TEA (101.08 mg, 998.88 umol, 139.03 uL,) and 3-(pyridin-4-yl)-1H-indazol-5-amine (70 mg, 332.96 umol). The mixture was stirred at 25° C. for 12 hrs. The reaction mixture was concentrated under reduced pressure to remove the solvent. The residue was purified by Prep-HPLC (TFA condition) to afford the title compound (24.5 mg, 49.77 umol, 14.95% yield, TFA salt) as a yellow solid. ¹H NMR (400 MHz, DMSO-d₆) δ 13.90 (br s, 1H), 10.56 (s, 1H), 8.88-8.77 (m, 4H), 8.21 (br d, J=5.5 Hz, 2H), 7.76-7.58 (m, 4H), 2.79 (br s, 3H). MS-ESI (m/z) calc'd for C₂₀H₁₆N₇O [M+H]⁺: 370.1. Found 370.1.

### Example 13. 7-Methyl-N-(3-(pyridin-4-yl)-1H-indazol-5-yl)-1H-benzo[d][1,2,3]triazole-5-carboxamide

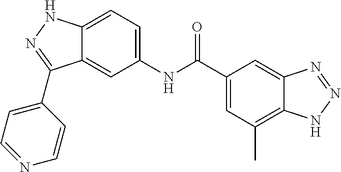 [SMILES: Cc1cc(C(=O)Nc2ccc3[nH]nc(-c4ccncc4)c3c2)cc2nn[nH]c12]

### Step 1. 5-Bromo-7-methyl-1H-benzo[d][1,2,3]triazole

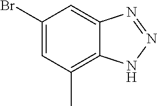

To a solution of 5-bromo-3-methylbenzene-1,2-diamine (1 g, 4.97 mmol) in H₂O (11 mL) was added AcOH (746.68 mg, 12.43 mmol) at 5° C. followed by a solution of NaNO₂ (411.78 mg, 5.97 mmol) in H₂O (1 mL). The mixture was stirred at 50° C. for 0.25 hr and then stirred at 25° C. for another 12 hrs. The reaction mixture was filtered and the solid was dried under vacuum to afford the title compound (1 g, crude) as a yellow solid which was used without further purification.

### Step 2. Methyl 7-methyl-1H-benzo[d][1,2,3]triazole-5-carboxylate

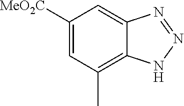

A mixture of 5-bromo-7-methyl-1H-benzo[d][1,2,3]triazole (300 mg, 1.41 mmol), Pd(dppf)Cl₂ (51.76 mg, 70.74 umol), TEA (858.97 mg, 8.49 mmol) in MeOH (10 mL) was degassed and purged with CO (3×), and then the mixture was stirred at 70° C. for 12 hr under a CO atmosphere (50 Psi). The reaction mixture was concentrated to give a residue. The residue was purified by column chromatography (SiO₂, petroleum ether/EtOAc=1:0 to 1:1) to afford the title compound (150 mg, 784.58 umol, 55.46% yield) as a yellow solid.

### Step 3. 7-Methyl-1H-benzo[d][1,2,3]triazole-5-carboxylic acid

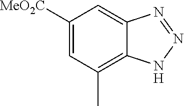

To a solution of methyl 7-methyl-1H-benzo[d][1,2,3]triazole-5-carboxylate (90 mg, 470.75 umol) in THF (2 mL) and H₂O (2 mL) was added LiOH (56.37 mg, 2.35 mmol). The mixture was stirred at 25° C. for 12 hrs. The reaction mixture was acidified with 1N HCl to pH=3, some solid was formed. The mixture was filtered and the solid was washed with H₂O (3 mL×2) and dried under vacuum to afford the title compound (80 mg, crude) as an orange solid which was used without further purification.

### Step 4. 7-Methyl-N-(3-(pyridin-4-yl)-1H-indazol-5-yl)-1H-benzo[d][1,2,3]triazole-5-carboxamide

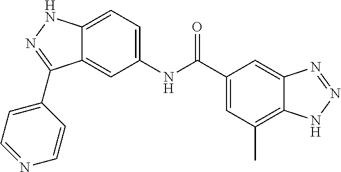 [SMILES: Cc1cc(C(=O)Nc2ccc3[nH]nc(-c4ccncc4)c3c2)cc2nn[nH]c12]

To a solution of 7-methyl-1H-benzo[d][1,2,3]triazole-5-carboxylic acid (80 mg, 451.57 umol) and 3-(pyridin-4-yl)-1H-indazol-5-amine (94.94 mg, 451.57 umol) in DCM (4 mL) was added T₃P/EtOAc (431.04 mg, 677.35 umol, 50% purity) and TEA (137.08 mg, 1.35 mmol). The mixture was stirred at 20° C. for 4 hrs. The reaction mixture was concentrated and purified by Prep-HPLC (TFA condition) to afford the title compound (53.45 mg, 101.09 umol, 22.39% yield, TFA salt) as a yellow solid. ¹H NMR (400 MHz, DMSO-d₆) δ 13.97 (br s, 1H) 10.56 (s, 1H) 8.86 (d, J=6 Hz, 2H) 8.78 (s, 1H) 8.48 (br s, 1H) 8.27 (d, J=6 Hz, 2H) 7.85 (br d, J=7 Hz, 2H) 7.72 (d, J=9 Hz, 1H) 2.70 (br s, 3H). MS-ESI (m/z) calc'd for C₂₀H₁₆N₇O [M+H]⁺: 370.1. Found 370.0.

### Example 14. 4-Cyano-N-(3-methyl-1H-indazol-5-yl)-1H-benzo[d][1,2,3]triazole-5-carboxamide

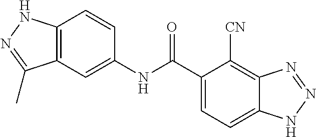 [SMILES: Cc1n[nH]c2ccc(NC(=O)c3ccc4[nH]nnc4c3C#N)cc12]

### Step 1. 3-Amino-6-bromo-2-nitrobenzonitrile

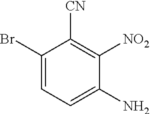 [SMILES: N#Cc1c(Br)ccc(N)c1[N+](=O)[O-]]

To a solution of 4-bromo-3-fluoro-2-nitroaniline (1 g, 4.26 mmol) in DMF (15 mL) was added NaCN (229.39 mg, 4.68 mmol). The mixture was stirred at 25° C. for 18 hr. The reaction mixture was diluted with H₂O (30 mL) and extracted with EtOAc (15 mL×3). The combined organic layers were washed with brine (20 mL×3), dried over Na₂SO₄, filtered and concentrated to give a residue. The residue was purified by column chromatography (SiO₂, petroleum ether/EtOAc=1:0 to 1:1) to afford the title compound (720 mg, 2.97 mmol, 69.91% yield) as a yellow solid.

### Step 2. Methyl 3,4-diamino-2-cyanobenzoate

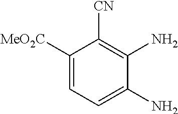 [SMILES: COC(=O)c1ccc(N)c(N)c1C#N]

A mixture of 3-amino-6-bromo-2-nitrobenzonitrile (500 mg, 2.07 mmol), Pd(dppf)Cl₂ (151.16 mg, 206.59 umol) and AcOK (4.05 g, 41.32 mmol) in MeOH (30 mL) was degassed and purged with CO (3×). The mixture was then stirred at 100° C. for 12 hrs under CO atmosphere (2.5 MPa). The reaction mixture was concentrated to give a residue. The residue was diluted with H₂O (50 mL) and extracted with EtOAc (20 mL×3), the combined organic layers were dried over Na₂SO₄, filtered and concentrated to give a residue. The residue was purified by column chromatography (SiO₂, petroleum ether/EtOAc=1:0 to 2:1) to afford the title compound (260 mg, 1.36 mmol, 65.83% yield) as a yellow solid.

### Step 3. Methyl 4-cyano-1H-benzo[d][1,2,3]triazole-5-carboxylate

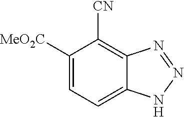 [SMILES: COC(=O)c1ccc2[nH]nnc2c1C#N]

To a solution of methyl 3,4-diamino-2-cyanobenzoate (260 mg, 1.36 mmol) in H₂O (4 mL) was added AcOH (204.16 mg, 3.40 mmol) at 5° C., then NaNO₂ (112.60 mg, 1.63 mmol) in H₂O (1 mL) was added at 5° C. The mixture was stirred at 25° C. for 12 hrs. The reaction mixture was filtered and the solid was collected and washed with H₂O (5 mL×3) and dried under vacuum to afford the title compound (225 mg, crude) as a yellow solid.

### Step 4. 4-Cyano-1H-benzo[d][1,2,3]triazole-5-carboxylic acid

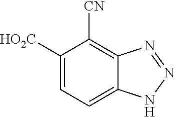 [SMILES: N#Cc1c(C(=O)O)ccc2[nH]nnc12]

To a solution of methyl 4-cyano-1H-benzo[d][1,2,3]triazole-5-carboxylate (200 mg, 989.27 umol) in THF (5 mL) was added an aqueous NaOH solution (2M, 2.47 mL). The mixture was stirred at 25° C. for 1.5 hr. The reaction mixture was acidified with 2N HCl to pH=3 and then it was extracted with EtOAc (10 mL×3). The combined organic layers were dried over Na₂SO₄, filtered and concentrated to afford the title compound (100 mg, crude) as a brown solid which was used without further purification.

### Step 5. 4-Cyano-N-(3-methyl-1H-indazol-5-yl)-1H-benzo[d][1,2,3]triazole-5-carboxamide

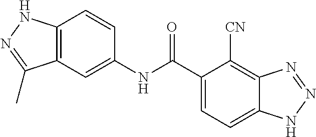 [SMILES: Cc1n[nH]c2ccc(NC(=O)c3ccc4[nH]nnc4c3C#N)cc12]

To a solution of 4-cyano-1H-benzo[d][1,2,3]triazole-5-carboxylic acid (95 mg, 504.94 umol) and 3-methyl-1H-indazol-5-amine (81.75 mg, 555.43 umol) in DMF (5 mL) was added DIEA (195.78 mg, 1.51 mmol) and HATU (287.99 mg, 757.40 umol) at 0° C. The mixture was stirred at 25° C. for 12 hr. The reaction mixture was concentrated to give a residue. The residue was first purified by Prep-HPLC (basic condition) and further purified by Prep-HPLC (neutral condition) to afford the title compound (34.23 mg, 98.96 umol, 19.60% yield) as a yellow solid. ¹H NMR (400 MHz, DMSO-d₆) δ 12.85 (br s, 1H) 9.74 (br s, 1H) 8.28 (br d, J=8 Hz, 1H) 7.80 (d, J=1 Hz, 1H) 7.73 (br d, J=8 Hz, 1H) 7.59 (d, J=9 Hz, 1H) 7.38 (dd, J=9, 2 Hz, 1H) 2.52-2.52 (m, 3H). MS-ESI (m/z) calc'd for C₁₆H₁₂N₇O [M+H]⁺: 318.1. Found 318.1.

### Example 15. N-(3-Bromo-1H-indazol-5-yl)-5-methyl-1H-benzo[d][1,2,3]triazole-6-carboxamide

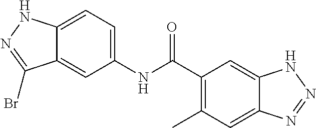 [SMILES: Cc1cc2nn[nH]c2cc1C(=O)Nc1ccc2[nH]nc(Br)c2c1]

To a solution of methyl 6-methyl-1H-benzo[d][1,2,3]triazole-5-carboxylate (100 mg, 523.05 umol) and 3-bromo-1H-indazol-5-amine (Intermediate 1; 110.91 mg, 523.05 umol) in toluene (4 mL) was added AlMe₃ (2 M, 1.31 mL). The reaction mixture was stirred at 25° C. for 12 hrs and then at 100° C. for another 2 hrs. The reaction mixture was quenched by addition of H₂O (4 mL) at 0° C. and then extracted with EtOAc (2 mL×3). The combined organic layers were washed with H₂O (6 mL×1), dried over anhydrous Na₂SO₄, filtered and concentrated in vacuum. The residue was purified by Prep-HPLC (TFA condition) to afford the title compound (41.35 mg, 85.22 umol, 16.29% yield, TFA salt) as a gray solid. ¹H NMR (400 MHz, DMSO-d₆) δ 15.51 (br s, 1H), 13.28-13.10 (m, 1H), 10.34 (br s, 1H), 8.16 (s, 2H), 7.87-7.64 (m, 2H), 7.56 (d, J=9.0 Hz, 1H), 2.58 (s, 3H). MS-ESI (m/z) calc'd for C₁₅H₁₂BrN₆O [M+H]⁺: 371.0, 373.0. Found 371.0, 373.0.

### Example 16. 5-Methyl-N-(3-methyl-1H-indazol-5-yl)-1H-benzo[d][1,2,3]triazole-6-carboxamide

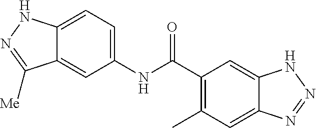 [SMILES: Cc1cc2nn[nH]c2cc1C(=O)Nc1ccc2[nH]nc(C)c2c1]

Prepared as described for N-(3-bromo-1H-indazol-5-yl)-5-methyl-1H-benzo[d][1,2,3]triazole-6-carboxamide (Example 15) using 3-methyl-1H-indazol-5-amine. ¹H NMR (400 MHz, DMSO-d₆) δ 12.59 (s, 1H), 10.42 (s, 1H), 8.26 (s, 1H), 8.09 (s, 1H), 7.78 (s, 1H), 7.52 (m, 1H), 7.49-7.39 (m, 1H), 2.58-2.53 (s, 3H), 2.49-2.46 (s, 3H). MS-ESI (m/z) calc'd for C₁₆H₁₅N₆O [M+H]⁺: 307.1. Found 307.1.

### Example 17. 6-Methyl-N-(3-phenyl-1H-indazol-5-yl)-1H-benzo[d][1,2,3]triazole-5-carboxamide

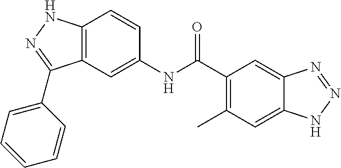 [SMILES: Cc1cc2[nH]nnc2cc1C(=O)Nc1ccc2[nH]nc(-c3ccccc3)c2c1]

A mixture of 3-phenyl-1H-indazol-5-amine (60 mg, 286.74 umol), 6-methyl-1H-benzo[d][1,2,3]triazole-5-carboxylic acid (Intermediate 2; 60.96 mg, 344.09 umol), T₃P/EtOAc (273.71 mg, 430.12 umol, 50% purity) and TEA (87.05 mg, 860.23 umol) in DCM (1 mL) was degassed and purged with N₂ (3×). The mixture was then stirred at 20° C. for 12 hrs under a N₂ atmosphere. The reaction mixture was concentrated and purified by Prep-HPLC (TFA condition) to afford the title compound (34.64 mg, 91.04 umol, 31.75% yield, TFA salt) as a white solid. ¹H NMR (400 MHz, DMSO-d₆) δ 13.24 (br s, 1H), 10.52 (s, 1H), 8.64 (s, 1H), 8.30-8.08 (m, 1H), 7.95 (d, J=7.3 Hz, 2H), 7.72 (br d, J=9.0 Hz, 2H), 7.62-7.51 (m, 3H), 7.44-7.38 (m, 1H), 2.56 (s, 3H). MS-ESI (m/z) calc'd for C₂₁H₁₇N₆O [M+H]⁺: 369.1. Found 369.1.

### Example 18. N-(3-Bromo-1H-indazol-5-yl)-6-methyl-1H-benzo[d]imidazole-5-carboxamide

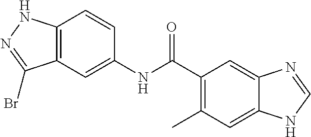 [SMILES: Cc1cc2[nH]cnc2cc1C(=O)Nc1ccc2[nH]nc(Br)c2c1]

To a solution of 3-bromo-1H-indazol-5-amine (Intermediate 1; 80 mg, 377.28 umol) and 6-methyl-1H-benzo[d]imidazole-5-carboxylic acid (73.11 mg, 415.01 umol, HCl) in DMF (2 mL) was added DIEA (146.28 mg, 1.13 mmol, 197.14 uL) and HATU (215.18 mg, 565.91 umol) at 0° C. The mixture was then stirred at 25° C. for 12 hrs. The reaction mixture was concentrated under reduced pressure to remove the solvent and then purified by Prep-HPLC (TFA condition) to afford the title compound (31.32 mg, 78.91 umol, 22.42% yield, TFA salt) as a pale purple solid. ¹H NMR (400 MHz, DMSO-d₆) δ 13.40 (br s, 1H) 10.54 (br d, J=4.40 Hz, 1H) 9.10-9.24 (m, 1H) 8.24 (s, 1H) 7.93 (br d, J=3.79 Hz, 1H) 7.56-7.71 (m, 3H) 2.56 (s, 3H). MS-ESI (m/z) calc'd for C₁₆H₁₃BrN₅O [M+H]⁺: 370.0, 372.0. Found 370.0, 372.0.

### Example 19. 6-Methyl-N-(3-phenyl-1H-indazol-5-yl)-1H-benzo[d]imidazole-5-carboxamide

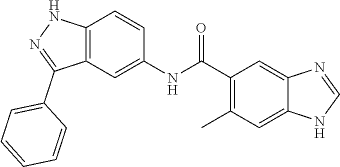 [SMILES: Cc1cc2[nH]cnc2cc1C(=O)Nc1ccc2[nH]nc(-c3ccccc3)c2c1]

To a solution of 3-phenyl-1H-indazol-5-amine (170 mg, 812.44 umol) in DCM (5 mL) was added 6-methyl-1H-benzo[d]imidazole-5-carboxylic acid (172.75 mg, 812.44 umol, HCl), T₃P/EtOAc (517.00 mg, 812.44 umol, 483.18 uL, 50% purity) and TEA (246.63 mg, 2.44 mmol, 339.25 uL). The mixture was stirred at 25° C. for 12 hrs. The reaction mixture was concentrated under reduced pressure to give a residue. The residue was purified by Prep-HPLC (TFA condition) to afford the title compound (37.07 mg, 75.44 umol, 9.29% yield, TFA salt) as a pale pink solid. ¹H NMR (400 MHz, DMSO-d₆) δ 13.25 (br s, 2H) 10.50 (s, 1H) 9.21 (br s, 1H) 8.63 (s, 1H) 7.95 (br d, J=5.95 Hz, 3H) 7.69-7.74 (m, 2H) 7.53-7.61 (m, 3H) 7.40-7.44 (m, 1H) 2.57 (s, 3H). MS-ESI (m/z) calc'd for C₂₂H₁₈N₅O [M+H]⁺: 368.1. Found 368.1.

### Example 20. 6-Methyl-N-(3-phenyl-1H-indazol-5-yl)-1H-indole-5-carboxamide

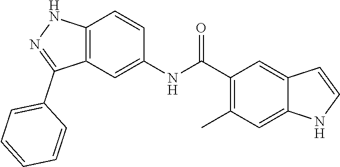 [SMILES: Cc1cc2[nH]ccc2cc1C(=O)Nc1ccc2[nH]nc(-c3ccccc3)c2c1]

To a solution of 6-methyl-1H-indole-5-carboxylic acid (100 mg, 570.83 umol) and 3-phenyl-1H-indazol-5-amine (119.44 mg, 570.83 umol) in DCM (4 mL) was added T₃P/EtOAc (435.90 mg, 685.00 umol, 50% purity), and TEA (231.05 mg, 2.28 mmol). The mixture was stirred at 25° C. for 12 hrs. The reaction mixture was concentrated under reduced pressure to remove the solvent. The residue was first purified by Prep-HPLC (neutral condition) and further purified by Prep-HPLC (TFA condition) to afford the title compound (24.13 mg, 49.22 umol, 8.62% yield, TFA salt) as a pale-yellow gum. ¹H NMR (400 MHz, MeOD) δ 8.54 (s, 1H), 7.96 (br d, J=7.7 Hz, 2H), 7.81 (s, 1H), 7.70-7.62 (m, 1H), 7.61-7.48 (m, 3H), 7.47-7.37 (m, 1H), 7.27 (br d, J=17.5 Hz, 2H), 6.50 (d, J=3.1 Hz, 1H), 2.59 (s, 3H). MS-ESI (m/z) calc'd for C₂₃H₁₉N₄O [M+H]⁺: 367.2. Found 367.1.

### Example 21. 6-Methyl-N-(3-phenyl-1H-indazol-5-yl)-1H-indazole-5-carboxamide

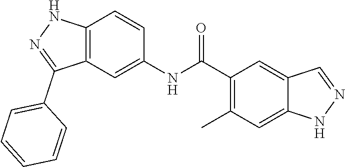 [SMILES: Cc1cc2[nH]ncc2cc1C(=O)Nc1ccc2[nH]nc(-c3ccccc3)c2c1]

### Step 1. Methyl 6-methyl-1H-indazole-5-carboxylate

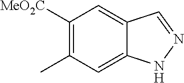

To a solution of 5-bromo-6-methyl-1H-indazole (334.5 mg, 1.58 mmol) in MeOH (5.5 mL) was added Pd(dppf)Cl₂ (115.97 mg, 158.49 umol) and TEA (1.60 g, 15.85 mmol) under N₂. The suspension was degassed under vacuum and purged with CO several times. The mixture was stirred at 70° C. for 24 hrs under CO (50 Psi). The reaction mixture was concentrated under reduced pressure to give a residue. The residue was purified by column chromatography (SiO₂, petroleum ether/EtOAc=1:0 to 1:1) to afford the title compound (240 mg, 1.17 mmol, 73.93% yield) as a yellow solid.

### Step 2. 6-Methyl-1H-indazole-5-carboxylic acid

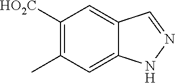

A mixture of methyl 6-methyl-1H-indazole-5-carboxylate (80 mg, 420.61 umol) and NaOH (50.47 mg, 1.26 mmol) in MeOH (1 mL), H₂O (1 mL) and THF (1 mL) was degassed, and stirred at 60° C. for 12 hr. The reaction mixture was concentrated under reduced pressure to remove the solvent. The residue was diluted with H₂O (5 mL) and adjusted to pH=8 by the careful addition of saturated aqueous Na₂CO₃ and extracted with EtOAc (3 mL×4). The combined organic layers were dried over Na₂SO₄, filtered and concentrated under reduced pressure to afford the title compound (79.3 mg, crude) as a light red solid.

### Step 3. 6-Methyl-N-(3-phenyl-1H-indazol-5-yl)-1H-indazole-5-carboxamide

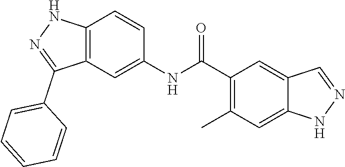 [SMILES: Cc1cc2[nH]ncc2cc1C(=O)Nc1ccc2[nH]nc(-c3ccccc3)c2c1]

To a solution of 6-methyl-1H-indazole-5-carboxylic acid (79.30 mg, 450.13 umol), 3-phenyl-1H-indazol-5-amine (94.19 mg, 450.13 umol) in DCM (4 mL) was added T₃P/EtOAc (343.73 mg, 540.16 umol, 50% purity) and TEA (182.19 mg, 1.80 mmol). The mixture was stirred at 25° C. for 12 hrs. The reaction mixture was concentrated and purified by Prep-HPLC (TFA condition) to afford the title compound (38.19 mg, 78.17 umol, 17.37% yield, TFA salt) as a pink solid. ¹H NMR (400 MHz, DMSO-d₆) δ 13.18 (br s, 2H), 10.39 (s, 1H), 8.63 (s, 1H), 8.13 (s, 1H), 8.01-7.92 (m, 3H), 7.73 (dd, J=1.5, 9.0 Hz, 1H), 7.61-7.51 (m, 3H), 7.46-7.38 (m, 2H), 2.53 (s, 3H). MS-ESI (m/z) calc'd for C₂₂H₁₈N₅O [M+H]⁺: 368.1. Found 368.1.

### Example 22. 5-Methyl-N-(3-phenyl-1H-indazol-5-yl)-1H-indole-6-carboxamide

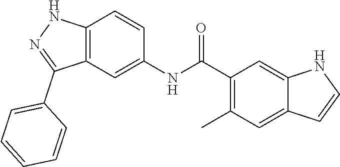 [SMILES: Cc1cc2cc[nH]c2cc1C(=O)Nc1ccc2[nH]nc(-c3ccccc3)c2c1]

### Step 1. Methyl 5-methyl-1H-indole-6-carboxylate

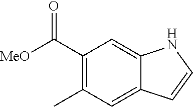

To a solution of 6-bromo-5-methyl-1H-indole (500 mg, 2.38 mmol) in MeOH (5 mL) was added TEA (9.63 g, 95.21 mmol, 13.25 mL) and Pd(dppf)Cl₂ (348.31 mg, 476.03 umol) under a N₂ atmosphere. The suspension was degassed and purged with CO (3×). The mixture was stirred at 70° C. for 12 hr under CO (50 psi), then it was warmed to 100° C. and stirred for another 4 hr under CO atmosphere (3 Mpa). The reaction mixture was concentrated and purified by flash silica gel chromatography (ISCO®; 12 g SepaFlash® Silica Flash Column, eluent of 0˜8% EtoAc/petroleum ether gradient at 80 mL/min) to afford the title compound (40 mg, 211.41 umol, 8.88% yield) as a yellow solid.

### Step 2. 5-Methyl-N-(3-phenyl-1H-indazol-5-yl)-1H-indole-6-carboxamide

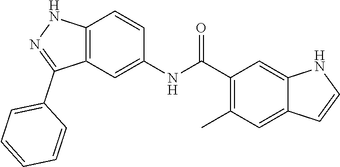 [SMILES: Cc1cc2cc[nH]c2cc1C(=O)Nc1ccc2[nH]nc(-c3ccccc3)c2c1]

To a solution of methyl 5-methyl-1H-indole-6-carboxylate (15 mg, 79.28 umol) and 3-phenyl-1H-indazol-5-amine (16.59 mg, 79.28 umol) in toluene (2 mL) was added AlMe₃ (2 M, 158.55 uL). The mixture was stirred at 25° C. for 12 hrs then warmed to 60° C. and stirred for an additional 12 hrs. The reaction mixture was concentrated and purified by Prep-HPLC (TFA condition) twice to afford the title compound (2.14 mg, 4.41 umol, 2.78% yield, TFA salt) as a pale yellow solid. ¹H NMR (400 MHz, MeOD) δ 8.55 (s, 1H) 7.98 (d, J=6.72 Hz, 2H) 7.51-7.70 (m, 5H) 7.41-7.50 (m, 2H) 7.34 (s, 1H) 6.45 (s, 1H) 2.58 (s, 3H). MS-ESI (m/z) calc'd for C₂₃H₁₈N₄O [M+H]⁺: 367.2. Found 367.1.

### Example 23. 5-Methyl-N-(3-phenyl-1H-indazol-5-yl)-1H-indazole-6-carboxamide

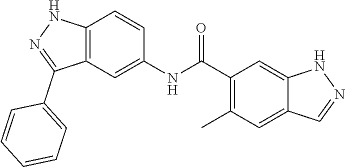 [SMILES: Cc1cc2cn[nH]c2cc1C(=O)Nc1ccc2[nH]nc(-c3ccccc3)c2c1]

### Step 1. Methyl 5-methyl-1H-indazole-6-carboxylate

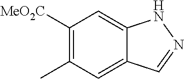

A mixture of 6-bromo-5-methyl-1H-indazole (300 mg, 1.42 mmol), Pd(dppf)Cl₂ (104.00 mg, 142.14 umol) and TEA (862.99 mg, 8.53 mmol) in MeOH (6 mL) was degassed and purged with CO (3×), and then the mixture was stirred at 70° C. for 12 hrs under a CO atmosphere (50 Psi). The reaction mixture was concentrated and purified by column chromatography (SiO₂, petroleum ether/EtOAc=1:0 to 1:1) to afford the title compound (220 mg, 1.16 mmol, 81.38% yield) as an orange solid.

### Step 2. 5-Methyl-1H-indazole-6-carboxylic acid

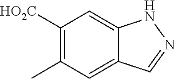

To a solution of methyl 5-methyl-1H-indazole-6-carboxylate (220 mg, 1.16 mmol) in THF (4 mL) was added an aqueous solution of NaOH (2 M, 3.47 mL). The mixture was stirred at 25° C. for 12 hrs. The reaction mixture was then acidified with 1N HCl to pH=2 at which point a solid precipitated. The mixture was filtered, and the solid was dried under vacuum to give the title compound (137 mg, crude) as a yellow solid.

### Step 3. 5-Methyl-N-(3-phenyl-1H-indazol-5-yl)-1H-indazole-6-carboxamide

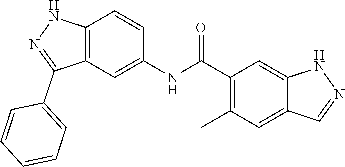 [SMILES: Cc1cc2cn[nH]c2cc1C(=O)Nc1ccc2[nH]nc(-c3ccccc3)c2c1]

To a solution of 5-methyl-1H-indazole-6-carboxylic acid (70 mg, 397.34 umol) in DCM (3 mL) was added 3-phenyl-1H-indazol-5-amine (141.34 mg, 675.48 umol), T₃P/EtOAc (328.71 mg, 516.54 umol, 50% purity) and TEA (120.62 mg, 1.19 mmol). The mixture was stirred at 25° C. for 12 hrs. The reaction mixture was concentrated and purified by Prep-HPLC (TFA condition) to afford the title compound (11.56 mg, 23.68 umol, 5.96% yield, TFA salt) as a white solid. ¹H NMR (400 MHz, DMSO-d₆) δ 12.99-13.24 (m, 2H) 10.30-10.45 (m, 1H) 8.64 (s, 1H) 8.04 (s, 1H) 7.93 (d, J=7.50 Hz, 2H) 7.60-7.71 (m, 3H) 7.44-7.60 (m, 3H) 7.35-7.43 (m, 1H) 2.45-2.46 (m, 3H). MS-ESI (m/z) calc'd for C₂₂H₁₇N₅O [M+H]⁺: 368.1. Found 368.1.

### Example 24. 5-Bromo-N-(3-phenyl-1H-indazol-5-yl)-1H-indazole-6-carboxamide

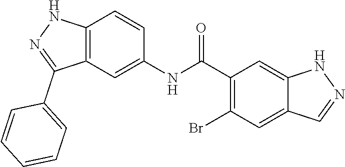 [SMILES: O=C(Nc1ccc2[nH]nc(-c3ccccc3)c2c1)c1cc2[nH]ncc2cc1Br]

To a solution of 5-bromo-1H-indazole-6-carboxylic acid (130 mg, 539.33 umol) and 3-phenyl-1H-indazol-5-amine (112.85 mg, 539.33 umol) in DCM (6 mL) was added T₃P/EtOAc (446.17 mg, 701.12 umol, 50% purity) and TEA (163.72 mg, 1.62 mmol). The mixture was stirred at 20° C. for 13 hrs. The reaction mixture was concentrated under reduced pressure to give a residue. The residue was purified by Prep-HPLC (TFA condition) to afford the title compound (53.6 mg, 96.16 umol, 17.83% yield, TFA salt) as a pale purple solid. 1H NMR (400 MHz, MeOD) δ 8.54 (d, J=0.9 Hz, 1H), 8.13 (d, J=12.0 Hz, 2H), 7.95 (br d, J=7.2 Hz, 2H), 7.79 (s, 1H), 7.69-7.62 (m, 1H), 7.61-7.57 (m, 1H), 7.53 (br t, J=7.5 Hz, 2H), 7.46-7.38 (m, 1H). MS-ESI (m/z) calc'd for C₂₁H₁₄BrN₅O [M+H]⁺: 432.04, 434.04. Found 432.0, 434.0.

### Example 25. 4,6-Difluoro-N-(3-(furan-2-yl)-1H-indazol-5-yl)-1-methyl-1H-indazole-5-carboxamide

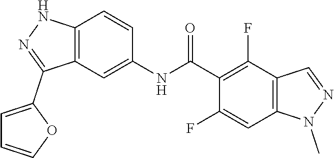 [SMILES: Cn1ncc2c(F)c(C(=O)Nc3ccc4[nH]nc(-c5ccco5)c4c3)c(F)cc21]

### Step 1. N-(3-Bromo-1H-indazol-5-yl)-4,6-difluoro-1-methyl-1H-indazole-5-carboxamide

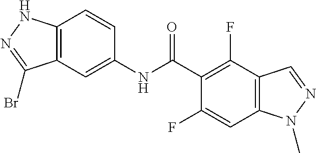 [SMILES: Cn1ncc2c(F)c(C(=O)Nc3ccc4[nH]nc(Br)c4c3)c(F)cc21]

To a solution of 4,6-difluoro-1-methyl-1H-indazole-5-carboxylic acid (50 mg, 235.68 umol) and 3-bromo-1H-indazol-5-amine (Intermediate 1; 54.97 mg, 259.25 umol) in pyridine (1 mL) was added EDCI (90.36 mg, 471.36 umol). The mixture was stirred at 25° C. for 12 hrs. The reaction mixture was concentrated under reduced pressure to remove the solvent. The residue was diluted with H₂O (10 mL) and extracted with EtOAc (3 mL×3). The combined organic layers were washed with brine (10 mL×1), dried over Na₂SO₄, filtered and concentrated under reduced pressure to give a residue. The residue was purified by Prep-TLC (SiO₂, petroleum ether/EtOAc=0/1) to afford the title compound (90 mg, 221.57 umol, 94.02% yield) as a red solid.

### Step 2. 4,6-Difluoro-N-(3-(furan-2-yl)-1H-indazol-5-yl)-1-methyl-1H-indazole-5-carboxamide

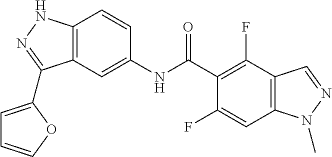 [SMILES: Cn1ncc2c(F)c(C(=O)Nc3ccc4[nH]nc(-c5ccco5)c4c3)c(F)cc21]

To a solution of N-(3-bromo-1H-indazol-5-yl)-4,6-difluoro-1-methyl-1H-indazole-5-carboxamide (50 mg, 123.10 umol) and furan-2-ylboronic acid (17.91 mg, 160.03 umol) in EtOH (2 mL) and H₂O (0.5 mL) was added Pd(Amphos)Cl₂ (8.72 mg, 12.31 umol, 8.72 uL) and KOAc (36.24 mg, 369.29 umol). The mixture was stirred at 100° C. for 12 hrs under N₂. The reaction mixture was concentrated under reduced pressure to remove the solvent. The residue was purified by Prep-HPLC (neutral condition) to afford the title compound (5.37 mg, 13.38 umol, 10.87% yield) as a white solid. ¹H NMR (400 MHz, DMSO-d₆) δ 10.84 (s, 1H) 8.60 (s, 1H) 8.34 (s, 1H) 7.87 (d, J=1.22 Hz, 1H) 7.51-7.73 (m, 3H) 6.89 (d, J=3.18 Hz, 1H) 6.69 (dd, J=3.24, 1.77 Hz, 1H) 4.09 (s, 3H). MS-ESI (m/z) calc'd for C₂₀H₁₃F₂N₅O₂ [M+H]⁺: 394.1. Found 394.1.

### Example 26. 4,6-Difluoro-1-methyl-N-(3-(oxazol-5-yl)-1H-indazol-5-yl)-1H-indazole-5-carboxamide

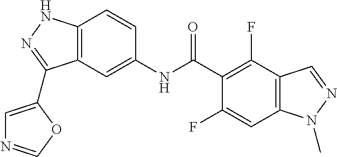 [SMILES: Cn1ncc2c(F)c(C(=O)Nc3ccc4[nH]nc(-c5cnco5)c4c3)c(F)cc21]

Prepared as described for 4,6-difluoro-N-(3-(furan-2-yl)-1H-indazol-5-yl)-1-methyl-1H-indazole-5-carboxamide (Example 25) using oxazol-5-ylboronic acid. ¹H NMR (400 MHz, DMSO-d₆) δ 10.89 (s, 1H), 8.58 (s, 1H), 8.56 (s, 1H), 8.34 (s, 1H), 7.69-7.60 (m, 4H), 4.08 (s, 3H). MS-ESI (m/z) calc'd for C₁₉H₁₃F₂N₆O₂ [M+H]⁺: 395.1. Found 395.0.

### Example 27. 4,6-Difluoro-N-(3-(isoxazol-4-yl)-1H-indazol-5-yl)-1-methyl-1H-indazole-5-carboxamide

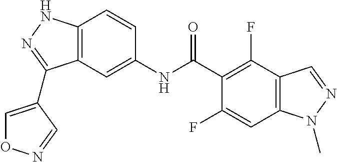 [SMILES: Cn1ncc2c(F)c(C(=O)Nc3ccc4[nH]nc(-c5cnoc5)c4c3)c(F)cc21]

Prepared as described for 4,6-difluoro-N-(3-(furan-2-yl)-1H-indazol-5-yl)-1-methyl-1H-indazole-5-carboxamide (Example 25) using isoxazol-4-ylboronic acid. ¹H NMR (400 MHz, DMSO-d₆) δ 13.34 (s, 1H) 10.81 (s, 1H) 9.52 (s, 1H) 9.12 (s, 1H) 8.34 (d, J=17.20 Hz, 2H) 7.65 (d, J=9.70 Hz, 1H) 7.60 (s, 2H) 4.06 (s, 3H). MS-ESI (m/z) calc'd for C₁₉H₁₃F₂N₆O₂ [M+H]⁺: 395.1. Found 395.1.

### Example 28. 4,6-Difluoro-N-(3-(furan-3-yl)-1H-indazol-5-yl)-1-methyl-1H-indazole-5-carboxamide

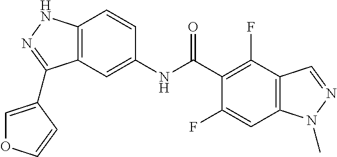 [SMILES: Cn1ncc2c(F)c(C(=O)Nc3ccc4[nH]nc(-c5ccoc5)c4c3)c(F)cc21]

Prepared as described for 4,6-difluoro-N-(3-(furan-2-yl)-1H-indazol-5-yl)-1-methyl-1H-indazole-5-carboxamide (Example 25) using furan-3-ylboronic acid. ¹H NMR (400 MHz, DMSO-d₆) δ 13.13 (br s, 1H) 10.80 (s, 1H) 8.40 (s, 1H) 8.34 (s, 1H) 8.21 (s, 1H) 7.85 (t, J=1.54 Hz, 1H) 7.54-7.70 (m, 3H) 6.99 (d, J=1.10 Hz, 1H) 4.08 (s, 3H). MS-ESI (m/z) calc'd for C₂₀H₁₄F₂N₅O₂ [M+H]⁺: 394.1. Found 394.0.

### Example 29. 4,6-Difluoro-1-methyl-N-(3-(5-methylisoxazol-4-yl)-1H-indazol-5-yl)-1H-indazole-5-carboxamide

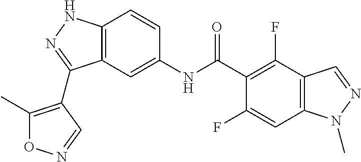 [SMILES: Cc1oncc1-c1n[nH]c2ccc(NC(=O)c3c(F)cc4c(cnn4C)c3F)cc12]

Prepared as described for 4,6-difluoro-N-(3-(furan-2-yl)-1H-indazol-5-yl)-1-methyl-1H-indazole-5-carboxamide (Example 25) using (5-methylisoxazol-4-yl)boronic acid. ¹H NMR (400 MHz, CDCl₃) δ 13.34 (br s, 1H), 10.82 (s, 1H), 8.96 (s, 1H), 8.34 (d, J=2.2 Hz, 2H), 7.68-7.59 (m, 3H), 4.08 (s, 3H), 2.69 (s, 3H). MS-ESI (m/z) calc'd for C₂₀H₁₅F₂N₆O₂ [M+H]⁺: 409.1. Found 409.1.

### Example 30. 4,6-Difluoro-1-methyl-N-(3-(3-methylisoxazol-4-yl)-1H-indazol-5-yl)-1H-indazole-5-carboxamide

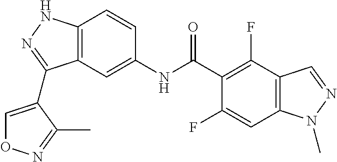 [SMILES: Cc1nocc1-c1n[nH]c2ccc(NC(=O)c3c(F)cc4c(cnn4C)c3F)cc12]

Prepared as described for 4,6-difluoro-N-(3-(furan-2-yl)-1H-indazol-5-yl)-1-methyl-1H-indazole-5-carboxamide (Example 25) using (3-methylisoxazol-4-yl)boronic acid. ¹H NMR (400 MHz, DMSO-d₆) δ 13.35 (br s, 1H), 10.83 (s, 1H), 9.38 (s, 1H), 8.34 (s, 2H), 7.69-7.59 (m, 3H), 4.08 (s, 3H), 2.54-2.52 (m, 3H). MS-ESI (m/z) calc'd for C₂₀H₁₅F₂N₆O₂ [M+H]⁺: 409.1. Found 409.0.

### Example 31. N-(3-(2,3-Dimethylphenyl)-1H-indazol-5-yl)-4,6-difluoro-1-methyl-1H-indazole-5-carboxamide

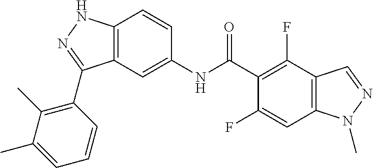 [SMILES: Cc1cccc(-c2n[nH]c3ccc(NC(=O)c4c(F)cc5c(cnn5C)c4F)cc23)c1C]

Prepared as described for 4,6-difluoro-N-(3-(furan-2-yl)-1H-indazol-5-yl)-1-methyl-1H-indazole-5-carboxamide (Example 25) using (2,3-dimethylphenyl)boronic acid. ¹H NMR (400 MHz, MeOD) δ 8.16 (d, J=0.66 Hz, 1H) 8.02 (t, J=1.32 Hz, 1H) 7.60 (t, J=1.43 Hz, 2H) 7.22-7.36 (m, 4H) 4.07 (s, 3H) 2.39 (s, 3H) 2.23 (s, 3H). MS-ESI (m/z) calc'd for C₂₄H₁₉F₂N₅O [M+H]⁺: 432.2. Found 432.2.

### Example 32. 4,6-Difluoro-1-methyl-N-(3-(pyridin-3-yl)-1H-indazol-5-yl)-1H-indazole-5-carboxamide

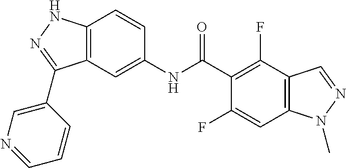 [SMILES: Cn1ncc2c(F)c(C(=O)Nc3ccc4[nH]nc(-c5cccnc5)c4c3)c(F)cc21]

Prepared as described for 4,6-difluoro-N-(3-(furan-2-yl)-1H-indazol-5-yl)-1-methyl-1H-indazole-5-carboxamide (Example 25) using pyridin-3-ylboronic acid. ¹H NMR (400 MHz, MeOD) δ 13.44-13.70 (m, 1H) 10.89 (s, 1H) 9.14-9.24 (m, 1H) 8.66-8.72 (m, 1H) 8.63 (s, 1H) 8.37-8.49 (m, 1H) 8.35 (s, 1H) 7.61-7.77 (m, 4H) 4.09 (s, 3H). MS-ESI (m/z) calc'd for C₂₁H₁₅F₂N₆O [M+H]⁺: 404.1. Found 405.0.

### Example 33. 4,6-Difluoro-1-methyl-N-(3-(5-methylfuran-2-yl)-1H-indazol-5-yl)-1H-indazole-5-carboxamide

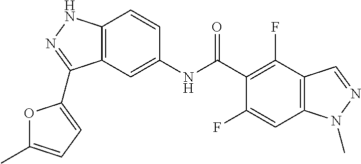 [SMILES: Cc1ccc(-c2n[nH]c3ccc(NC(=O)c4c(F)cc5c(cnn5C)c4F)cc23)o1]

Prepared as described for 4,6-difluoro-N-(3-(furan-2-yl)-1H-indazol-5-yl)-1-methyl-1H-indazole-5-carboxamide (Example 25) using (5-methylfuran-2-yl)boronic acid. ¹H NMR (400 MHz, DMSO-d₆) δ 13.16 (br s, 1H), 10.83 (s, 1H), 8.53 (s, 1H), 8.34 (s, 1H), 7.67 (br d, J=9.3 Hz, 2H), 7.60-7.52 (m, 1H), 6.75 (d, J=3.1 Hz, 1H), 6.28 (d, J=2.4 Hz, 1H), 4.09 (s, 3H), 2.40 (s, 3H). MS-ESI (m/z) calc'd for C₂₁H₁₆F₂N₅O₂ [M+H]⁺: 408.1. Found 408.0.

### Example 34. 4,6-Difluoro-1-methyl-N-(3-(pyrimidin-5-yl)-1H-indazol-5-yl)-1H-indazole-5-carboxamide

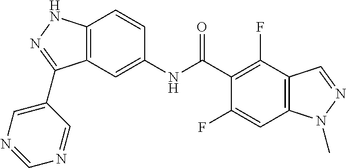 [SMILES: Cn1ncc2c(F)c(C(=O)Nc3ccc4[nH]nc(-c5cncnc5)c4c3)c(F)cc21]

Prepared as described for 4,6-difluoro-N-(3-(furan-2-yl)-1H-indazol-5-yl)-1-methyl-1H-indazole-5-carboxamide (Example 25) using pyrimidin-5-ylboronic acid. ¹H NMR (400 MHz, MeOD) δ 9.40 (s, 2H), 9.18 (s, 1H), 8.57 (d, J=0.9 Hz, 1H), 8.19 (d, J=0.7 Hz, 1H), 7.74-7.62 (m, 2H), 7.36 (d, J=9.5 Hz, 1H), 4.08 (s, 3H). MS-ESI (m/z) calc'd for C₂₀H₁₄F₂N₇O [M+H]⁺: 406.1. Found 406.1.

### Example 35. 4,6-Difluoro-1-methyl-N-(3-(1-methyl-1H-pyrazol-4-yl)-1H-indazol-5-yl)-1H-indazole-5-carboxamide

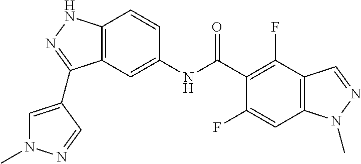 [SMILES: Cn1cc(-c2n[nH]c3ccc(NC(=O)c4c(F)cc5c(cnn5C)c4F)cc23)cn1]

Prepared as described for 4,6-difluoro-N-(3-(furan-2-yl)-1H-indazol-5-yl)-1-methyl-1H-indazole-5-carboxamide (Example 25) using (1-methyl-1H-pyrazol-4-yl)boronic acid. 1H NMR (400 MHz, MeOD) δ 8.45 (d, J=0.9 Hz, 1H), 8.18 (s, 2H), 8.02 (d, J=0.6 Hz, 1H), 7.63-7.50 (m, 2H), 7.35 (d, J=9.4 Hz, 1H), 4.08 (s, 3H), 4.01 (s, 3H). MS-ESI (m/z) calc'd for C₂₀H₁₅F₂N₇O [M+H]⁺: 408.1. Found 408.1.

### Example 36. N-(3-(3,5-Dimethylisoxazol-4-yl)-1H-indazol-5-yl)-4,6-difluoro-1-methyl-1H-indazole-5-carboxamide

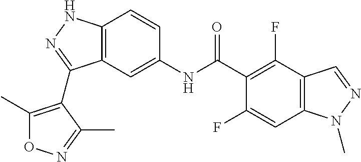 [SMILES: Cc1noc(C)c1-c1n[nH]c2ccc(NC(=O)c3c(F)cc4c(cnn4C)c3F)cc12]

Prepared as described for 4,6-difluoro-N-(3-(furan-2-yl)-1H-indazol-5-yl)-1-methyl-1H-indazole-5-carboxamide (Example 25) using (3,5-dimethylisoxazol-4-yl)boronic acid. ¹H NMR (400 MHz, DMSO-d₆) δ 10.85 (br s, 1H) 8.34 (d, J=0.73 Hz, 1H) 8.20 (s, 1H) 7.67 (d, J=9.78 Hz, 1H) 7.55-7.65 (m, 2H) 6.06 (br s, 1H) 4.09 (s, 3H) 2.48 (s, 3H) 2.29 (s, 3H). MS-ESI (m/z) calc'd for C₂₁H₁₇F₂N₆O₂ [M+H]⁺: 423.1. Found 423.1.

### Example 37. 4,6-Difluoro-1-methyl-N-(3-(2-methylpyridin-4-yl)-1H-indazol-5-yl)-1H-indazole-5-carboxamide

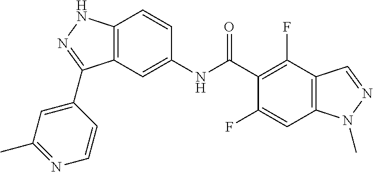 [SMILES: Cc1cc(-c2n[nH]c3ccc(NC(=O)c4c(F)cc5c(cnn5C)c4F)cc23)ccn1]

Prepared as described for 4,6-difluoro-N-(3-(furan-2-yl)-1H-indazol-5-yl)-1-methyl-1H-indazole-5-carboxamide (Example 25) using (2-methylpyridin-4-yl)boronic acid. 1H NMR (400 MHz, DMSO-d₆) δ 13.59 (br s, 1H), 10.90 (s, 1H), 8.62 (d, J=1.0 Hz, 1H), 8.58 (d, J=5.1 Hz, 1H), 8.35 (d, J=0.7 Hz, 1H), 7.79 (s, 1H), 7.74-7.70 (m, 2H), 7.69-7.64 (m, 2H), 4.09 (s, 3H), 2.57 (s, 3H). MS-ESI (m/z) calc'd for C₂₂H₁₇F₂N₆O [M+H]⁺: 419.1. Found 419.1.

### Example 38. 4,6-Difluoro-1-methyl-N-(3-(4-methylpyridin-3-yl)-1H-indazol-5-yl)-1H-indazole-5-carboxamide

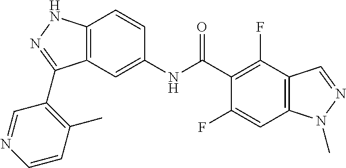 [SMILES: Cc1ccncc1-c1n[nH]c2ccc(NC(=O)c3c(F)cc4c(cnn4C)c3F)cc12]

Prepared as described for 4,6-difluoro-N-(3-(furan-2-yl)-1H-indazol-5-yl)-1-methyl-1H-indazole-5-carboxamide (Example 25) using (4-methylpyridin-3-yl)boronic acid. ¹H NMR (400 MHz, DMSO-d₆) δ 13.40 (br s, 1H) 10.80 (s, 1H) 8.65 (s, 1H) 8.49 (d, J=4.85 Hz, 1H) 8.30 (s, 1H) 8.21 (s, 1H) 7.55-7.73 (m, 3H) 7.43 (d, J=4.85 Hz, 1H) 4.05 (s, 3H) 2.39 (s, 3H). MS-ESI (m/z) calc'd for C₂₂H₁₇F₂N₆O [M+H]⁺: 419.1. Found 419.2.

### Example 39. 4,6-Difluoro-1-methyl-N-(3-(2-methylpyridin-3-yl)-1H-indazol-5-yl)-1H-indazole-5-carboxamide

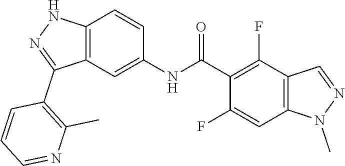 [SMILES: Cc1ncccc1-c1n[nH]c2ccc(NC(=O)c3c(F)cc4c(cnn4C)c3F)cc12]

Prepared as described for 4,6-difluoro-N-(3-(furan-2-yl)-1H-indazol-5-yl)-1-methyl-1H-indazole-5-carboxamide (Example 25) using (2-methylpyridin-3-yl)boronic acid. ¹H NMR (400 MHz, DMSO-d₆) δ 13.37 (br s, 1H), 10.81 (s, 1H), 8.56-8.53 (m, 1H), 8.32 (d, J=0.6 Hz, 1H), 8.19 (s, 1H), 7.91-7.87 (m, 1H), 7.67-7.63 (m, 1H), 7.63-7.62 (m, 1H), 7.62-7.58 (m, 1H), 7.42-7.37 (m, 1H), 4.07 (s, 3H), 2.56 (s, 3H). MS-ESI (m/z) calc'd for C₂₂H₁₇F₂N₆O [M+H]⁺: 419.1. Found 419.2.

### Example 40. 4,6-Difluoro-1-methyl-N-(3-(3-methylpyridin-4-yl)-1H-indazol-5-yl)-1H-indazole-5-carboxamide

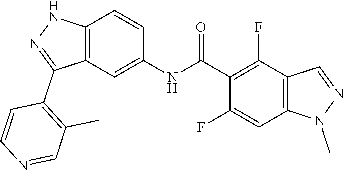 [SMILES: Cc1cnccc1-c1n[nH]c2ccc(NC(=O)c3c(F)cc4c(cnn4C)c3F)cc12]

A solution of N-(3-bromo-1H-indazol-5-yl)-4,6-difluoro-1-methyl-1H-indazole-5-carboxamide (50 mg, 123.10 umol), (3-methylpyridin-4-yl)boronic acid (20.23 mg, 147.72 umol), Pd(dppf)Cl₂ (9.01 mg, 12.31 umol) and K₂CO₃ (51.04 mg, 369.29 umol) in H₂O (0.5 mL) and dioxane (2 mL) was degassed and then heated to 100° C. for 12 hrs under N₂. After cooling to 20° C., the reaction mixture was filtered and the filtrate was concentrated. The residue was purified by Prep-HPLC (neutral condition) to afford the title compound (5.48 mg, 12.49 umol, 10.15% yield) as a red solid. ¹H NMR (400 MHz, DMSO-d₆) δ 13.52 (br s, 1H), 10.84 (s, 1H), 8.61 (s, 1H), 8.55 (d, J=5.0 Hz, 1H), 8.33 (d, J=0.7 Hz, 1H), 8.30-8.28 (m, 1H), 7.72-7.66 (m, 1H), 7.66-7.63 (m, 2H), 7.58-7.55 (m, 1H), 4.07 (s, 3H), 2.43 (s, 3H). MS-ESI (m/z) calc'd for C₂₂H₁₇F₂N₆O [M+H]⁺: 419.1. Found 419.1.

### Example 41. 4,6-Difluoro-1-methyl-N-(3-(pyridin-2-yl)-1H-indazol-5-yl)-1H-indazole-5-carboxamide

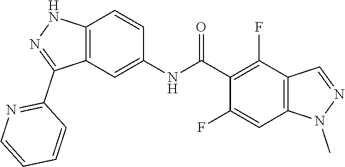 [SMILES: Cn1ncc2c(F)c(C(=O)Nc3ccc4[nH]nc(-c5ccccn5)c4c3)c(F)cc21]

To a solution of N-(3-bromo-1H-indazol-5-yl)-4,6-difluoro-1-methyl-1H-indazole-5-carboxamide (50 mg, 123.10 umol) and 2-(tributylstannyl)pyridine (72.51 mg, 196.96 umol) in dioxane (2 mL) was added Pd(PPh₃)₂Cl₂ (8.64 mg, 12.31 umol) and the reaction mixture was stirred at 120° C. for 12 hrs under N₂. The reaction mixture was filtered and the filtrate was concentrated. The residue purified by Prep-HPLC (TFA condition) to afford the title compound (17.51 mg, 33.78 umol, 27.44% yield, TFA salt) as a pale yellow solid. ¹H NMR (400 MHz, DMSO-d₆) δ 13.47 (br s, 1H) 10.85 (s, 1H) 8.93 (d, J=1.32 Hz, 1H) 8.72 (d, J=4.41 Hz, 1H) 8.34 (s, 1H) 8.20 (d, J=8.16 Hz, 1H) 7.92-8.01 (m, 1H) 7.75 (dd, J=8.93, 1.87 Hz, 1H) 7.64 (dd, J=12.68, 9.37 Hz, 2H) 7.41 (dd, J=6.84, 5.51 Hz, 1H) 4.09 (s, 3H). MS-ESI (m/z) calc'd for C₂₁H₁₅F₂N₆O [M+H]⁺: 405.1. Found 405.1.

### Example 42. 4,6-Difluoro-1-methyl-N-(3-(thiazol-5-yl)-1H-indazol-5-yl)-1H-indazole-5-carboxamide

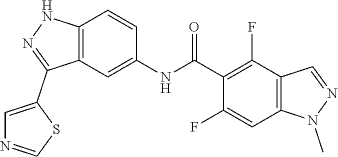 [SMILES: Cn1ncc2c(F)c(C(=O)Nc3ccc4[nH]nc(-c5cncs5)c4c3)c(F)cc21]

A solultion of N-(3-bromo-1H-indazol-5-yl)-4,6-difluoro-1-methyl-1H-indazole-5-carboxamide (30 mg, 73.86 umol), 5-(tributylstannyl)thiazole (55.27 mg, 147.72 umol) and Pd(PPh₃)₂Cl₂ (5.18 mg, 7.39 umol) in dioxane (2 mL) was degassed and then heated to 80° C. for 12 hrs under N₂. After cooling to 20° C., the reaction mixture was filtered and the filtrate was concentrated. The residue was purified by Prep-HPLC (neutral condition) to afford the title compound (4.6 mg, 10.94 umol, 14.82% yield) as a white solid. ¹H NMR (400 MHz, DMSO-d₆) δ 10.89 (s, 1H), 9.16 (d, J=0.4 Hz, 1H), 8.59 (d, J=0.6 Hz, 1H), 8.38 (d, J=0.4 Hz, 1H), 8.35 (d, J=0.9 Hz, 1H), 7.70-7.62 (m, 3H), 4.09 (s, 3H). MS-ESI (m/z) calc'd for C₁₉H₁₃F₂N₆OS [M+H]⁺: 411.1. Found 411.0.

### Example 43. 5-Methyl-N-(3-phenyl-1H-indazol-5-yl)-[1,2,3]triazolo[1,5-a]pyridine-6-carboxamide

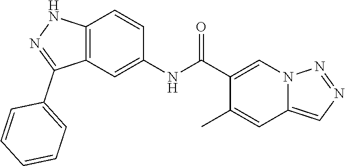 [SMILES: Cc1cc2cnnn2cc1C(=O)Nc1ccc2[nH]nc(-c3ccccc3)c2c1]

### Step 1. Methyl 6-chloro-4-methylnicotinate

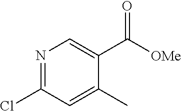

A suspension of 6-chloro-4-methylpyridine-3-carboxylic acid (1.0 g, 5.83 mmol) in phosphorus (V) oxychloride (10 mL, 107 mmol) was heated at 100° C. for 15 hours. The excess POCl₃ was evaporated and the residue was carefully quenched with MeOH (10 mL, 247 mmol) at 0° C. The solvent was evaporated and the residue was taken up in sat. aqueous NaHCO₃ and stirred for 15 minutes. A solid formed which was filtered under vacuum to obtain the title compound (650 mg, 3.502 mmol, 60% yield) as an off white solid. ¹H NMR (400 MHz, DMSO-d₆) δ 8.76 (s, 1H), 7.60 (s, 1H), 3.86 (s, 3H), 2.55 (s, 3H). MS-ESI (m/z) calculated for C₈H₉ClNO₂ [M+H]⁺: 186.0, 188.0. Found 186.0, 188.0.

### Step 2. Methyl 4-methyl-6-vinylnicotinate

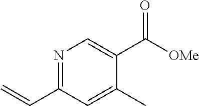

A solution of methyl 6-chloro-4-methylnicotinate (650 mg, 3.5 mmol) and tetrakis(triphenylphosphine)palladium(0) (202 mg, 0.180 mmol) in toluene (17 mL) was sparged with N₂ for 15 minutes. tributyl(ethenyl)stannane (1.23 mL, 4.2 mmol) was added and the mixture was stirred at 100° C. for 15 hours. The solvent was evaporated and the residue was purified by chromatography (SiO₂, 50 g, EtOAc in cyclohexane [0%, 3CV; 50%, 50%, 10 CV]) to obtain the title compound (300 mg, 1.693 mmol, 48.34% yield) as a yellow oil. ¹H NMR (400 MHz, DMSO-d₆) δ 8.89 (s, 1H), 7.49 (s, 1H), 6.83 (dd, J=17.4, 10.7 Hz, 1H), 6.36 (dd, J=17.5, 1.6 Hz, 1H), 5.60 (dd, J=10.7, 1.6 Hz, 1H), 3.86 (s, 3H), 2.55 (d, J=0.6 Hz, 3H). MS-ESI (m/z) calculated for C₁₀H₁₂NO₂ [M+H]⁺: 178.1. Found 178.0.

### Step 3. Methyl 6-formyl-4-methylnicotinate

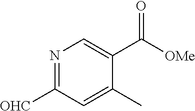

To a solution of methyl 4-methyl-6-vinylnicotinate (300 mg, 1.69 mmol) in 1,4-dioxane (5 mL) was added 4% osmium tetroxide (0.54 mL, 0.080 mmol) and a solution of sodium periodate (724 mg, 3.39 mmol) in water (5 mL). The mixture was stirred at room temperature for 15 hours. The mixture was diluted with water and extracted with DCM (3×). The combined organic layers were passed through a phase separator and evaporated to obtain the title compound (300 mg, 1.674 mmol, 99% yield) as a dark oil. ¹H NMR (400 MHz, CDCl₃) δ 10.11 (s, 1H), 9.19 (s, 1H), 7.83 (t, J=0.8 Hz, 1H), 3.98 (s, 3H), 2.70 (d, J=0.7 Hz, 3H). MS-ESI (m/z) calculated for C₉H₁₀NO₃ [M+H]⁺: 180.1. Found 180.0.

### Step 4. Methyl (E)-4-methyl-6-((2-tosylhydrazineylidene)methyl)nicotinate

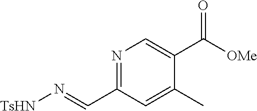

To a solution of methyl 6-formyl-4-methylnicotinate (300 mg, 1.67 mmol) in EtOH (6.7 mL) was added 4-methylbenzenesulfonohydrazide (312 mg, 1.67 mmol) and the mixture was stirred at room temperature for 3 hours. The solvent was evaporated to obtain the title compound (581 mg, 1.672 mmol, 100% yield) as a dark oil which was used without further purification. MS-ESI (m/z) calculated for C₁₆H₁₈N₃O₄S [M+H]⁺: 348.2. Found 348.2.

### Step 5. Methyl 5-methyl-[1,2,3]triazolo[1,5-a]pyridine-6-carboxylate

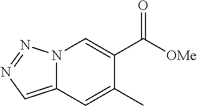

A mixture of methyl (E)-4-methyl-6-((2-tosylhydrazineylidene)methyl)nicotinate (581 mg, 0.840 mmol) in 4-methylmorpholine (2.5 mL, 22.74 mmol) was heated at 100° C. for 1 hour. The solvent was evaporated and the residue was taken up in water and extracted with EtOAc (3×). The combined organic layers were passed through a phase separator and evaporated to obtain a residue which was purified by column chromatography (SiO₂, 25 g, EtOAc in cyclohexane [0%, 100%, 10 CV]) to obtain the title compound (71 mg, 0.371 mmol, 44.41% yield) as a yellow solid. ¹H NMR (400 MHz, DMSO-d₆) δ 9.49 (s, 1H), 8.16 (d, J=1.0 Hz, 1H), 7.85 (q, J=1.1 Hz, 1H), 3.90 (s, 3H), 2.56 (d, J=1.2 Hz, 3H). MS-ESI (m/z) calculated for C₉H₁₀N₃O₂ [M+H]⁺: 192.1. Found 192.0.

### Step 6. 5-Methyl-[1,2,3]triazolo[1,5-a]pyridine-6-carboxylic acid

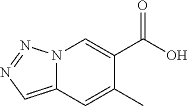

To a solution of methyl 5-methyltriazolo[1,5-a]pyridine-6-carboxylate (71 mg, 0.370 mmol) in THF (5 mL) was added lithium hydroxide hydrate (47 mg, 1.11 mmol) in water (1 mL) and the mixture was stirred at room temperature for 15 hrs. The organic solvent was evaporated and the residue was taken up in HCl and extracted with EtOAc (3×). The combined organic layers were passed through a phase separator and evaporated to obtain the title compound (50 mg, 0.282 mmol, 76% yield) as a yellow solid. ¹H NMR (400 MHz, DMSO-d₆) δ 13.49 (s, 1H), 9.43 (s, 1H), 8.14 (d, J=1.0 Hz, 1H), 7.82 (t, J=1.2 Hz, 1H), 2.58 (d, J=1.2 Hz, 3H). MS-ESI (m/z) calculated for C₈H₈N₃O₂ [M+H]⁺: 178.1. Found 178.0.

### Step 7. 5-Methyl-N-(3-phenyl-1H-indazol-5-yl)-[1,2,3]triazolo[1,5-a]pyridine-6-carboxamide

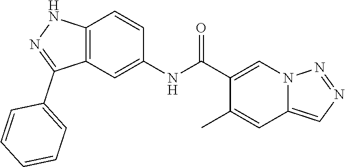 [SMILES: Cc1cc2cnnn2cc1C(=O)Nc1ccc2[nH]nc(-c3ccccc3)c2c1]

To a suspension of 5-methyltriazolo[1,5-a]pyridine-6-carboxylic acid (50 mg, 0.280 mmol) in acetonitrile (3 mL) was added triethylamine (39 uL, 0.280 mmol). The suspension became a solution. HATU (107 mg, 0.280 mmol) was then added and the mixture was stirred at room temperature for 10 minutes 3-Phenyl-1H-indazol-5-amine (63 mg, 0.280 mmol) was added and the mixture was stirred at room temperature for 2 hrs. The solvent was evaporated; the residue was taken up in water and extracted with EtOAc (3×). The combined organic layers were passed through a phase separator and evaporated to obtain the crude product (100 mg, 0.271 mmol, 96% yield) as a brown oil which was further purified by preparative HPLC, acid method, to obtain the title compound (13 mg, 0.035 mmol, 12.5% yield) as a yellow solid. ¹H NMR (400 MHz, DMSO-d₆) δ 13.26 (s, 1H), 10.70 (s, 1H), 9.42 (s, 1H), 8.60 (d, J=1.8 Hz, 1H), 8.16 (d, J=0.9 Hz, 1H), 7.98-7.93 (m, 2H), 7.86 (t, J=1.2 Hz, 1H), 7.70 (dd, J=8.9, 1.8 Hz, 1H), 7.62 (d, J=8.9 Hz, 1H), 7.56 (t, J=7.7 Hz, 2H), 7.46-7.38 (m, 1H), 2.49 (s, 3H). MS-ESI (m/z) calc'd for C₂₁H₁₇N₆O [M+H]⁺: 369.1. Found 369.3.

### Example 44. N-(3-(2-Fluorophenyl)-1H-indazol-5-yl)-5,7-dimethyl-[1,2,3]triazolo[1,5-a]pyridine-6-carboxamide

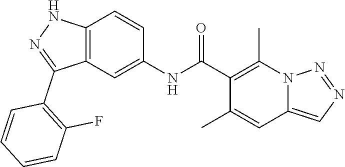 [SMILES: Cc1cc2cnnn2c(C)c1C(=O)Nc1ccc2[nH]nc(-c3ccccc3F)c2c1]

Triethylamine (0.04 mL, 0.260 mmol) and HATU (66.92 mg, 0.180 mmol) were sequentially added to a cooled solution (0° C.) of 3-(2-fluorophenyl)-1H-indazol-5-amine (Intermediate 3; 40 mg, 0.18 mmol) and 5,7-dimethyltriazolo[1,5-a]pyridine-6-carboxylic acid (33.7 mg, 0.18 mmol) in dry DMF (1 mL). The mixture was stirred at 0° C. for 10 minutes and then heated to 40° C. and stirred for an additional 18 hrs. The mixture was cooled to room temperature, diluted with water (50 mL) and extracted with EtOAc (50 mL). The organic phase was separated and concentrated under reduced pressure. The product was purified by reverse phase column chromatography, eluting with a gradient of ACN in water from 2% to 60% in the presence of 0.1% formic acid. The title compound (34 mg, 0.085 mmol, 48.2% yield) was obtained as a white solid. ¹H NMR (400 MHz, DMSO-d₆) δ 13.44 (br. s., 1H), 10.72 (s, 1H), 8.30-8.35 (m, 1H), 8.21 (s, 1H), 7.77-7.83 (m, 2H), 7.63 (d, J=0.75 Hz, 2H), 7.49-7.56 (m, 1H), 7.34-7.47 (m, 2H), 2.84 (s, 3H), 2.40 (d, J=0.75 Hz, 3H). MS-ESI (m/z) calc'd for C₂₂H₁₈FN₆O [M+H]⁺: 401.1. Found 401.2.

### Example 45. N-(3-Bromo-1H-indazol-5-yl)-5,7-dimethyl-[1,2,3]triazolo[1,5-a]pyridine-6-carboxamide

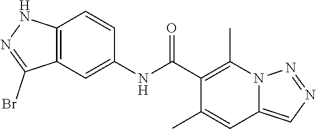 [SMILES: Cc1cc2cnnn2c(C)c1C(=O)Nc1ccc2[nH]nc(Br)c2c1]

5,7-Dimethyl-[1,2,3]triazolo[1,5-a]pyridine-6-carboxylic acid (Intermediate 4; 610.0 mg, 3.13 mmol) and 3-bromo-1H-indazol-5-amine (Intermediate 1; 1.3 g, 6.25 mmol) were dissolved in dry DMF (37 mL). The solution was cooled to 0° C. with an ice-water bath and triethylamine (0.65 mL, 4.69 mmol) and HATU (1.43 g, 3.75 mmol) were added. The mixture was stirred at 0° C. for 5 minutes and then at room temperature overnight. The reaction mixture was partitioned between water and EtOAc. The phases were separated and the aqueous layer was extracted with EtOAc (2×). The combined organic phases were washed with water (1×), dried over anhydrous Na₂SO₄ and evaporated to dryness. The crude material was purified by flash chromatography on an NH silica gel column, using a 0 to 5% gradient of MeOH in EtOAc as eluent. The product-containing fractions were combined and purified again by reverse phase column chromatography on a 12 g C18 column, using a 5 to 20% gradient of CH₃CN in water (0.1% formic acid) as eluent. The title compound (2.65 mg, 0.007 mmol, 0.220% yield) was obtained as a white solid. ¹H NMR (400 MHz, DMSO-d₆) δ 13.43 (br. S, 1H), 10.80 (s, 1H), 8.20-8.25 (m, 2H), 7.82 (s, 1H), 7.52-7.64 (m, 2H), 2.85 (s, 3H), 2.42 (d, J=0.88 Hz, 3H). MS-ESI (m/z) calc'd for C₁₆H₁₄BrN₆O [M+H]⁺: 385.0, 386.03. Found 385.1, 387.1.

### Example 46. N-(3-(2-Methoxyphenyl)-1H-indazol-5-yl)-5,7-dimethyl-[1,2,3]triazolo[1,5-a]pyridine-6-carboxamide

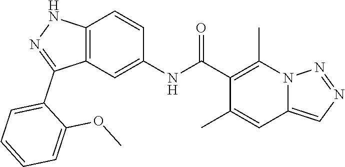 [SMILES: COc1ccccc1-c1n[nH]c2ccc(NC(=O)c3c(C)cc4cnnn4c3C)cc12]

N-(3-bromo-1H-indazol-5-yl)-5,7-dimethyl-[1,2,3]triazolo[1,5-a]pyridine-6-carboxamide (25.0 mg, 0.065 mmol, 1 eq) and 2-(2-methoxyphenyl)-4,4,5,5-tetramethyl-1,3,2-dioxaborolane (30.39 mg, 0.130 mmol, 2 eq) were suspended in DMF (1 mL) and a 2M aqueous solution of Na₂CO₃ (0.1 mL, 0.195 mmol, 3 eq) was added. The mixture was purged with N₂ for 5 min, then tetrakis(triphenylphosphine)palladium(0) (3.75 mg, 0.003 mmol, 0.05 eq) was added and the mixture was stirred at 120° C. for 3 hrs. The mixture was partitioned between water and EtOAc. The phases were separated. The aqueous layer was extracted with EtOAc (2×) and the combined organic layers were washed with water (1×), dried over anhydrous Na₂SO₄ and the solvent was removed under reduced pressure. The crude material was purified by normal phase chromatography on an 11 g NH-silica gel column, eluting with a 0 to 100% gradient of EtOAc in cyclohexane. The purest fractions were combined, evaporated to dryness and purified again by reverse phase chromatography on a 12 g C18 column, eluting with a 5 to 35% (0.1% formic acid) gradient of CH₃CN in H₂O. The title compound (9 mg, 0.022 mmol, 33.62% yield) was obtained as a white solid. ¹H NMR (400 MHz, DMSO-d₆) δ 13.14 (s, 1H), 10.63 (s, 1H), 8.16-8.23 (m, 2H), 7.80 (s, 1H), 7.52-7.60 (m, 3H), 7.41-7.48 (m, 1H), 7.21 (d, J=8.14 Hz, 1H), 7.09 (t, J=7.15 Hz, 1H), 3.85 (s, 3H), 2.84 (s, 3H), 2.41 (s, 3H). MS-ESI (m/z) calc'd for C₂₃H₂₁N₆O₂ [M+H]⁺: 413.2. Found 413.2.

### Example 47. N-(3-(2,3-Dimethylphenyl)-1H-indazol-5-yl)-5,7-dimethyl-[1,2,3]triazolo[1,5-a]pyridine-6-carboxamide

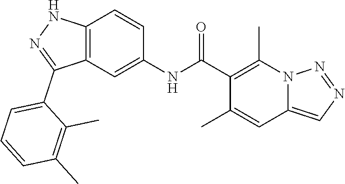 [SMILES: Cc1cccc(-c2n[nH]c3ccc(NC(=O)c4c(C)cc5cnnn5c4C)cc23)c1C]

In a microwave vial, N-(3-bromo-1H-indazol-5-yl)-5,7-dimethyl-[1,2,3]triazolo[1,5-a]pyridine-6-carboxamide (20.0 mg, 0.050 mmol), (2,3-dimethylphenyl)boronic acid (15.57 mg, 0.100 mmol) and Na₂CO₃ (16.51 mg, 0.156 mmol) were suspended in DMF (1.143 mL) and water (0.286 mL). The mixture was purged with N₂ for 5 minutes and then tetrakis(triphenylphosphine)palladium(0) (3.0 mg, 0 mmol) was added. The vial was capped and the reaction mixture was irradiated in a microwave at 120° C. (2×1 h). The mixture was partitioned between water and EtOAc and the phases were separated. The aqueous layer was extracted with EtOAc (2×) and the combined organic layers were washed with water, dried over Na₂SO₄ and the solvent was removed under reduced pressure. The crude material was separated using chiral chromatography to give N-[3-(2,3-dimethylphenyl)-1H-indazol-5-yl]-5,7-dimethyltriazolo[1,5-a]pyridine-6-carboxamide (1.8 mg, 0.004 mmol, 8.446% yield) Method: Analytical chiral HPLC conditions and results: Column Chiralpak IC (25×0.46 cm), 5μ Mobile phase n-hexane/EtOH 75/25% v/v Flow rate (mL/min) 1.0 DAD 220 nm Loop 20 μL Target 32.6% a/a by UV (11.8 min) semi-preparative chiral HPLC conditions and results: Column Chiralpak IC (25×2.0 cm), 5 p Mobile phase n-hexane/EtOH 75/25% v/v Flow rate (mL/min) 17 mL/minutes DAD detection 220 nm Loop 900 μL Total amount 40 mg Solubilisation 40 mg in 3.2 mL DCM/MeOH 1/1=12.5 mg/mL Injection 11.2 mg/injection. ¹H NMR (400 MHz, DMSO-d₆) δ 13.18 (s, 1H), 10.66 (s, 1H), 8.16-8.23 (m, 1H), 8.09 (s, 1H), 7.79 (s, 1H), 7.56-7.64 (m, 2H), 7.22-7.31 (m, 3H), 2.82 (s, 3H), 2.34-2.39 (m, 6H), 2.22 (s, 3H). MS-ESI (m/z) calc'd for C₂₄H₂₃N₆O [M+H]⁺: 411.2. Found 411.3.

### Example 48. 5,7-Dimethyl-N-(3-phenyl-1H-indazol-5-yl)-[1,2,3]triazolo[1,5-a]pyridine-6-carboxamide

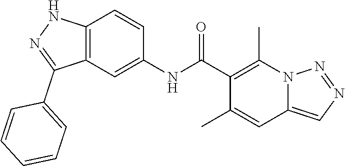 [SMILES: Cc1cc2cnnn2c(C)c1C(=O)Nc1ccc2[nH]nc(-c3ccccc3)c2c1]

5,7-Dimethyl-[1,2,3]triazolo[1,5-a]pyridine-6-carboxylic acid (Intermediate 4; 40.0 mg, 0.210 mmol, 1 eq) and 3-phenyl-1H-indazol-5-amine (65.67 mg, 0.310 mmol, 1.5 eq) were dissolved in dry DMF (1.5 mL). Then the solution was cooled to 0° C. with an ice-water bath and triethylamine (43.74 uL, 0.310 mmol, 1.5 eq) and HATU (95.46 mg, 0.250 mmol, 1.2 eq) were added. The mixture was stirred at 0° C. for 5 minutes and then at r.t. for 3 days. The crude material was loaded directly onto a 12 g C18 column and purified by reverse phase chromatography using a 5 to 40% gradient of CH₃CN in H₂O (0.1% formic acid) as eluent. The purest fractions were combined, evaporated to dryness and purified again by normal phase column chromatography on a 11 g NH-silica gel column using a 50 to 100% gradient of EtOAc in cyclohexane as eluent. The title compound (14 mg, 0.037 mmol, 17.5% yield) was obtained pure as a white solid. ¹H NMR (400 MHz, DMSO-d₆) δ 13.28 (br. s., 1H), 10.74 (s, 1H), 8.61 (s, 1H), 8.22 (s, 1H), 8.00-7.91 (m, 2H), 7.82 (s, 1H), 7.70-7.60 (m, 2H), 7.59-7.53 (m, 2H), 7.47-7.37 (m, 1H), 2.86 (s, 3H), 2.42 (d, J=0.9 Hz, 3H). MS-ESI (m/z) calc'd for C₂₂H₁₉N₆O [M+H]⁺: 383.2. Found 383.4.

### Example 49. 5,7-Dimethyl-N-(3-methyl-1H-indazol-5-yl)-[1,2,3]triazolo[1,5-a]pyridine-6-carboxamide

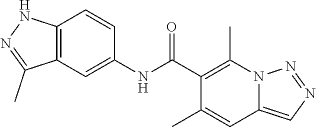 [SMILES: Cc1cc2cnnn2c(C)c1C(=O)Nc1ccc2[nH]nc(C)c2c1]

5,7-Dimethyltriazolo[1,5-a]pyridine-6-carboxylic acid (62 mg, 0.32 mmol) and 3-methyl-1H-indazol-5-amine (95.46 mg, 0.65 mmol) were dissolved in dry DMF (2 mL) and the solution was cooled to 0° C. with an ice-water bath. Then TEA (68 uL, 0.49 mmol) and HATU (147.96 mg, 0.390 mmol) were sequentially added. The mixture was allowed to reach r.t. and left stirring for 18 hrs. The mixture was diluted with water (20 mL) and extracted with EtOAc (2×20 mL). The organic phases were combined, dried over Na₂SO₄, filtered and then concentrated under vacuum. The crude material was purified by reverse phase column chromatography on a C18 cartridge, eluting with a 5% to 50% gradient of acetonitrile in water (containing 0.1% formic acid). Pure fractions were collected and concentrated to give the title compound (21 mg, 0.066 mmol, 20.2% yield). Impure fractions were concentrated and further purified by a second column chromatography eluting with a 0 to 100% gradient of EtOAc in cyclohexane to give additional product (22 mg, 0.069 mmol, 21.2% yield). ¹H NMR (400 MHz, DMSO-d₆) δ 12.63 (s, 1H), 10.64 (s, 1H), 8.23 (s, 1H), 8.21 (s, 1H), 7.81 (s, 1H), 7.47 (d, J=1.10 Hz, 2H), 2.85 (s, 3H), 2.50 (s., 3H), 2.42 (s, 3H). MS-ESI (m/z) calc'd for C₁₇H₁₇N₆O [M+H]⁺: 321.1. Found 321.2.

### Example 50. 4,6-Difluoro-1-methyl-N-(3-phenyl-1H-indazol-5-yl)-1H-indazole-5-carboxamide

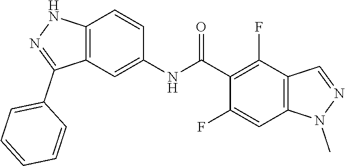 [SMILES: Cn1ncc2c(F)c(C(=O)Nc3ccc4[nH]nc(-c5ccccc5)c4c3)c(F)cc21]

4,6-difluoro-1-methylindazole-5-carboxylic acid (50.0 mg, 0.240 mmol, 1 eq) and 3-phenyl-1H-indazol-5-amine (98.63 mg, 0.470 mmol, 2 eq) were dissolved in dry DMF (1.5 mL). Then the solution was cooled to 0° C. with an ice-water bath and triethylamine (49.27 uL, 0.350 mmol, 1.5 eq) and HATU (107.54 mg, 0.280 mmol, 1.2 eq) were added. The mixture was stirred at 0° C. for 5 minutes and then at room temperature for 2 hrs. The reaction mixture was partitioned between water and EtOAc and the phases were separated. The aqueous layer was extracted with EtOAc (2×) and the combined organic phases were washed with water (1×), dried over anhydrous Na₂SO₄ and evaporated to dryness. The crude material was purified by normal phase chromatography, first on a 28 g NH-silica gel column, using a 0 to 100% gradient of EtOAc in cyclohexane as eluent, and then on a 25 g silica gel column using a 20 to 100% gradient of EtOAc in cyclohexane as eluent. The title compound (21 mg, 0.052 mmol, 22.09% yield) was obtained as a white solid. ¹H NMR (400 MHz, DMSO-d₆) δ 13.27 (br. s., 1H), 10.83 (s, 1H), 8.59 (s, 1H), 8.34 (d, J=0.88 Hz, 1H), 7.91-7.99 (m, 2H), 7.60-7.70 (m, 3H), 7.56 (t, J=7.70 Hz, 2H), 7.39-7.46 (m, 1H), 4.09 (s, 3H). MS-ESI (m/z) calc'd for C₂₂H₁₆F₂N₅O [M+H]⁺: 404.1. Found 404.2.

### Example 51. 4,6-Difluoro-1-methyl-N-(3-methyl-1H-indazol-5-yl)-1H-indazole-5-carboxamide

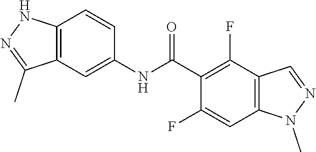 [SMILES: Cc1n[nH]c2ccc(NC(=O)c3c(F)cc4c(cnn4C)c3F)cc12]

4,6-difluoro-1-methylindazole-5-carboxylic acid (50.0 mg, 0.240 mmol, 1 eq) and 3-methyl-1H-indazol-5-amine (69.38 mg, 0.470 mmol, 2 eq) were dissolved in dry DMF (1.5 mL). Then the solution was cooled to 0° C. with an ice-water bath and triethylamine (49.27 uL, 0.350 mmol, 1.5 eq) and HATU (107.54 mg, 0.280 mmol, 1.2 eq) were added. The mixture was stirred at 0° C. for 5 minutes and then at room temperature for 2 hrs. The reaction mixture was partitioned between water and EtOAc, the phases were separated, the aqueous layer was extracted with EtOAc (2×) and the combined organic phases washed with water (1×), dried over anhydrous Na₂SO₄ and evaporated to dryness. The crude material was purified by normal phase chromatography on a 28 g NH-silica gel column using a 0 to 100% gradient of EtOAc in cyclohexane as eluent. The title compound (54 mg, 0.158 mmol, 67.13% yield) was obtained pure as a white solid. ¹H NMR (400 MHz, DMSO-d₆) δ 12.62 (s, 1H), 10.72 (s, 1H), 8.33 (d, J=0.88 Hz, 1H), 8.21 (s, 1H), 7.65 (d, J=9.68 Hz, 1H), 7.46 (s, 2H), 4.09 (s, 3H), 2.49 (s, 3H). MS-ESI (m/z) calc'd for C₁₇H₁₄F₂N₅O [M+H]⁺: 342.1. Found 342.4.

### Example 52. 6-Methyl-N-(3-phenyl-1H-indazol-5-yl)imidazo[1,5-a]pyridine-7-carboxamide

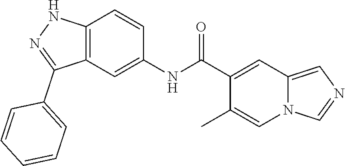 [SMILES: Cc1cn2cncc2cc1C(=O)Nc1ccc2[nH]nc(-c3ccccc3)c2c1]

Ethyl 6-methylimidazo[1,5-a]pyridine-7-carboxylate (Intermediate 5; 50.0 mg, 0.120 mmol) was dissolved in toluene (10 mL) and 3-phenyl-1H-indazol-5-amine (33.3 mg, 0.160 mmol) was added followed by a 2M solution of trimethylaluminum (17.65 mg, 0.240 mmol) in toluene. The reaction mixture was stirred at 90° C. for 2 hrs. An additional portion of trimethylaluminum (17.65 mg, 0.240 mmol) was then added and the reaction was stirred for another 2 hrs at 90° C. The reaction was cooled to r.t. and diluted with water (20 mL) and EtOAc (30 mL). The organic layer was separated, dried over sodium sulphate, filtered and concentrated to give a crude product that was purified via preparative HPLC to give the title compound (6.1 mg, 0.017 mmol, 13.56% yield) as a grey solid. ¹H NMR (400 MHz, acetone-d₆) δ 12.34 (br. s., 1H), 9.66 (br. s., 1H), 8.73 (br. s., 1H), 8.27 (s, 1H), 8.16 (d, J=0.88 Hz, 1H), 8.07 (d, J=7.04 Hz, 2H), 7.90 (s, 1H), 7.83 (d, J=8.80 Hz, 1H), 7.65 (d, J=8.80 Hz, 1H), 7.55 (t, J=7.70 Hz, 2H), 7.49 (s, 1H), 7.40-7.46 (m, 1H), 7.40-7.46 (m, 1H), 2.39 (d, J=1.10 Hz, 3H). MS-ESI (m/z) calc'd for C₁₇H₁₄F₂N₅O [M+H]⁺: 368.1. Found 368.2.

### Example 53. 1-Methyl-N-(3-methyl-1H-indazol-5-yl)-1H-indazole-5-carboxamide

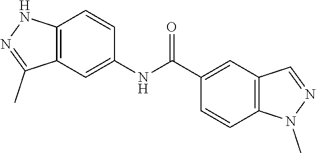 [SMILES: Cc1n[nH]c2ccc(NC(=O)c3ccc4c(cnn4C)c3)cc12]

1-Methyl-1H-indazole-5-carboxylic acid (40.0 mg, 0.230 mmol, 1 eq) and 3-methyl-1H-indazol-5-amine (66.84 mg, 0.450 mmol, 2 eq) were dissolved in dry DMF (1.5 mL). Then the solution was cooled to 0° C. using an ice-water bath. Triethylamine (47.47 uL, 0.340 mmol, 1.5 eq) and HATU (103.6 mg, 0.270 mmol, 1.2 eq) were then added. The mixture was stirred at 0° C. for 5 minutes and then at r.t. overnight. The reaction mixture was partitioned between water and EtOAc and the phases were separated. The aqueous layer was extracted with EtOAc (2×) and the combined organic phases were washed with water (1×), dried over anhydrous Na₂SO₄ and evaporated to dryness. The crude material was purified by flash chromatography on an 11 g NH-silica gel column, using a 0 to 10% gradient of MeOH in EtOAc as eluent. The purest fractions were combined, evaporated to dryness and then re-dissolved in DMSO and loaded on a 12 g C18 column and further purified by reverse phase chromatography using a 5 to 35% gradient of CH₃CN in H₂O (containing 0.1% formic acid) as eluent. The title compound (22 mg, 0.072 mmol, 31.73% yield) was obtained as a white solid. ¹H NMR (400 MHz, DMSO-d₆) δ 12.57 (s, 1H), 10.27 (s, 1H), 8.50 (d, J=0.88 Hz, 1H), 8.25 (d, J=0.88 Hz, 1H), 8.19 (d, J=1.32 Hz, 1H), 8.04 (dd, J=8.91, 1.65 Hz, 1H), 7.77 (d, J=8.80 Hz, 1H), 7.65 (dd, J=8.80, 1.76 Hz, 1H), 7.45 (d, J=8.80 Hz, 1H), 4.11 (s, 3H), 2.49 (s, 3H). MS-ESI (m/z) calc'd for C₁₇H₁₆N₅O [M+H]⁺: 306.1. Found 306.3.

### Example 54. (racemic)-7,7-Dimethyl-N-(3-phenyl-1H-indazol-5-yl)-4,5,6,7-tetrahydro-[1,2,3]triazolo[1,5-a]pyridine-6-carboxamide

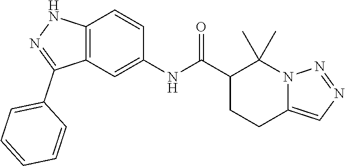 [SMILES: CC1(C)C(C(=O)Nc2ccc3[nH]nc(-c4ccccc4)c3c2)CCc2cnnn21]

### Step 1. Ethyl hex-5-ynoate

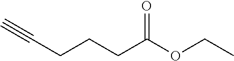

A 100 mL round flask was charged with 5-hexynoic acid (10 g, 45.3 mmol), EtOH (50 mL), and H₂SO₄ (0.95 mL, 17.83 mmol). The solution was refluxed for 3 hrs, then saturated aqueous Na₂CO₃ (200 mL) was added and the mixture was extracted with Et₂O (3×100 mL). The combined organic layers were dried (Na₂SO₄) and evaporated to afford the title compound (8.70 g, 62.06 mmol, 70%) as a colorless oil. ¹H NMR (400 MHz, DMSO-d₆) δ 4.15 (q, J=7.19 Hz, 2H) 2.45 (t, J=7.37 Hz, 2H) 2.28 (td, J=6.93, 2.64 Hz, 2H) 1.98 (t, J=2.64 Hz, 1H) 1.86 (quin, J=7.21 Hz, 2H), 1.27 (t, J=7.04 Hz, 3H).

### Step 2. Ethyl 6-(tert-butyldimethylsilyl)hex-5-ynoate

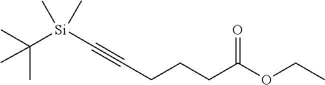 [SMILES: CCOC(=O)CCCC#C[Si](C)(C)C(C)(C)C]

A 2-necked 100 mL round flask was charged with ethyl hex-5-ynoate (4.63 g, 33.03 mmol), and THF (100 mL). The solution was stirred at −78° C. while lithium diisopropylamide (18.17 mL, 36.33 mmol) was added. After 10 min, tert-butyldimethylsilyl chloride (5.48 g, 36.33 mmol) was added. The solution was allowed to reach room temperature overnight. After 16 hrs the volatiles were removed under reduced pressure and the product was purified by normal phase column chromatography on a 50 g silica gel column, using a 0 to 10% gradient of EtOAc in cyclohexane as eluent. The title compound (2.36 g, 9.275 mmol, 28% yield) was obtained as a pale-yellow oil. ¹H NMR (400 MHz, DMSO-d₆) δ 4.06 (d, J=7.04 Hz, 2H), 2.40 (s, 2H), 2.28 (t, J=6.93 Hz, 2H), 1.69 (quin, J=7.15 Hz, 2H), 1.18 (s, 3H), 0.87-0.95 (m, 9H), 0.06 (s, 6H).

### Step 3. Ethyl 6-(tert-butyldimethylsilyl)-2-(2-hydroxypropan-2-yl)hex-5-ynoate

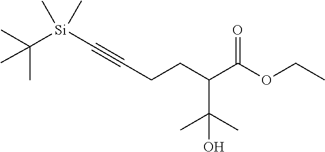 [SMILES: CCOC(=O)C(CCC#C[Si](C)(C)C(C)(C)C)C(C)(C)O]

A flame-dried 2-necked 10 mL flask was charged with ethyl 6-(tert-butyldimethylsilyl)hex-5-ynoate (2.36 g, 9.28 mmol) and dry THF (100 mL). The solution was stirred at −78° C. under a N₂ atmosphere while a lithium diisopropylamide solution (2M in THF/hexane, 9.74 mL, 19.48 mmol) was added. The solution was stirred at −78° C. for 15 min, then acetone (1.44 mL, 19.48 mmol) (previously dried over Na₂SO₄) was added. The final mixture was allowed to slowly reach room temperature and after 4 hrs the mixture was quenched with ice and water and extracted with EtOAc (3×). The organic phases were collected and concentrated under reduced pressure. The product was purified by normal phase column chromatography on a 50 g silica gel column, using a 0 to 10% gradient of EtOAc in cyclohexane as eluent. The title compound (1.71 g, 5.47 mmol, 59% yield) was obtained as a pale-yellow oil. ¹H NMR (400 MHz, DMSO-d₆) δ 4.57 (s, 1H), 4.02-4.12 (m, 2H), 2.14-2.25 (m, 1H), 2.01-2.13 (m, 1H), 1.63-1.84 (m, 2H), 1.18 (t, J=7.15 Hz, 3H), 1.12 (d, J=5.72 Hz, 6H), 0.91 (s, 9H), 0.06 (s, 6H).

### Step 4. Ethyl 2-(2-hydroxypropan-2-yl)hex-5-ynoate

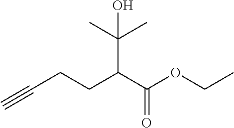

In a 250 mL round flask ethyl 6-(tert-butyldimethylsilyl)-2-(2-hydroxypropan-2-yl)hex-5-ynoate (1.71 g, 5.47 mmol) was dissolved in THF (80 mL) and stirred at 0° C. while a 1 M TBAF solution in THF (6.57 mL, 6.57 mmol) was added. The solution was stirred at room temperature for 4 hrs, then diluted with water (30 mL) and extracted with EtOAc (2×100 mL). The combined organic layers were dried (Na₂SO₄) and evaporated under reduced pressure to give a crude product (1.3 g). This was dissolved in THF (80 mL) and stirred at 0° C. while another 1M TBAF solution in THF (3.28 mL, 3.28 mmol) was added. The solution was stirred at room temperature for 4 hrs, then diluted with water (30 mL) and extracted with EtOAc (2×100 mL). The combined organic layers were dried (Na₂SO₄), filtered and evaporated under reduced pressure. The product was purified by normal phase column chromatography on a 25 g silica gel column using a 0 to 20% gradient of EtOAc in cyclohexane as eluent. The title compound (605 mg, 3.05 mmol, 55.8% yield) was obtained as a pale yellow oil. ¹H NMR (400 MHz, CDCl₃) δ 4.24 (q, J=7.04 Hz, 2H), 2.77 (s, 1H), 2.57 (dd, J=11.22, 3.52 Hz, 1H), 2.25-2.37 (m, 1H), 2.09-2.21 (m, 1H), 1.96-2.08 (m, 2H), 1.84-1.95 (m, 1H), 1.57 (s, 2H), 1.33 (t, J=7.04 Hz, 3H), 1.27 (d, J=9.02 Hz, 6H).

### Step 5. Ethyl 2-(2-(1H-1,2,3-triazol-5-yl)ethyl)-3-hydroxy-3-methylbutanoate

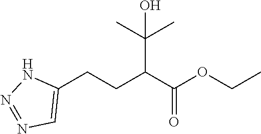 [SMILES: CCOC(=O)C(CCc1cnn[nH]1)C(C)(C)O]

A 2-necked 25 mL round flask was charged with ethyl 2-(2-hydroxypropan-2-yl)hex-5-ynoate (605 mg, 3.05 mmol), crystalline (+)-sodium L-ascorbate (605 mg, 3.05 mmol), azidomethyl 2,2-dimethylpropanoate (623.5 mg, 3.97 mmol), tert-butanol (33 mL) and water (11 mL). The mixture was degassed by bubbling N₂ through the solution for 10 minutes after which copper sulfate pentahydrate (154 mg, 0.61 mmol) was added. The mixture was stirred at r.t. for 16 hrs, then diluted with water (300 mL) and extracted with DCM (3×100 mL). The combined organic layers were dried (Na₂SO₄), filtered and evaporated under reduced pressure to give a pale yellow oil (1.17 g). This crude material was re-dissolved in MeOH (2 mL) and a 1 M aqueous NaOH solution (2 mL) was added. The solution was stirred at r.t. for 30 minutes and then was neutralized by the addition of 1 M HCl aq. solution. The final solution was extracted with DCM (4×20 mL), the organic layers were dried (Na₂SO₄), filtered and evaporated under reduced pressure. The product was purified by normal phase column chromatography on a 50 g silica gel column using a 20 to 100% gradient of EtOAc in cyclohexane as eluent, to afford a mixture containing the title compound (710 mg, 2.94 mmol, 96% yield) as a colorless oil. MS-ESI (m/z) calc'd for C₁₁H₂₀N₃O₃ [M+H]⁺: 242.1. Found 242.3.

### Step 6. Ethyl 7,7-dimethyl-4,5,6,7-tetrahydro-[1,2,3]triazolo[1,5-a]pyridine-6-carboxylate

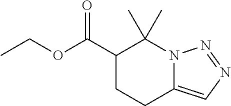 [SMILES: CCOC(=O)C1CCc2cnnn2C1(C)C]

A 5 mL microwave vial was charged with ethyl 2-(2-(1H-hydroxy-3-methylbutanoate (650 mg, 2.69 mmol), trifluoroacetic acid (10.73 mL, 140.08 mmol), and DCE (10 mL). The vial was sealed and the solution was heated in a microwave reactor for 1.5 hrs at 130° C. The reaction was concentrated under reduced pressure to give yellow oil. This crude was collected with the crude obtained from a test reaction on 60 mg of ethyl 2-(2-(1H-1,2,3-triazol-5-yl)ethyl)-3-hydroxy-3-methylbutanoate by using the same reaction conditions. The desired product was purified by reverse phase column chromatography on a 28 g C18 column, using a 3 to 50% gradient of CH₃CN in H₂O (0.1% formic acid) as eluent. The title compound (280 mg, 1.25 mmol, 46.6% yield) was obtained as a brown oil. ¹H NMR (400 MHz, Methanol-d) δ 7.45 (s, 1H), 4.29-4.18 (m, 2H), 3.16-2.98 (m, 2H), 2.91-2.76 (m, 1H), 2.27-2.07 (m, 2H), 1.85 (s, 3H), 1.64 (s, 3H), 1.34-1.28 (m, 3H). MS-ESI (m/z) calc'd for C₁₁H₁₈N₃O₂ [M+H]⁺: 224.1. Found 224.3.

### Step 7. 7,7-Dimethyl-4,5,6,7-tetrahydro-[1,2,3]triazolo[1,5-a]pyridine-6-carboxylic acid

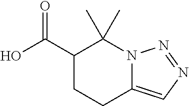

To a solution of ethyl 7,7-dimethyl-4,5,6,7-tetrahydro-[1,2,3]triazolo[1,5-a]pyridine-6-carboxylate (100 mg, 0.45 mmol) in MeOH (1 mL), was added a solution of lithium hydroxide hydrate (57.74 mg, 1.34 mmol) in water (3 mL). The mixture was stirred for 18 hrs at 50° C. MeOH was removed under reduced pressure and then HCl (1M aq. solution) was added dropwise until pH 6. The solution was concentrated under reduced pressure to obtain the desired product as a crude material (311 mg), which was directly used in the next step without further purification. MS-ESI (m/z) calc'd for C₉H₁₄N₃O₂ [M+H]⁺: 196.1. Found 196.3.

### Step 8. 7,7-Dimethyl-N-(3-phenyl-1H-indazol-5-yl)-4,5,6,7-tetrahydro-[1,2,3]triazolo[1,5-a]pyridine-6-carboxamide

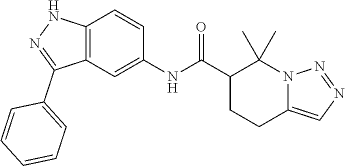 [SMILES: CC1(C)C(C(=O)Nc2ccc3[nH]nc(-c4ccccc4)c3c2)CCc2cnnn21]

7,7-Dimethyl-4,5,6,7-tetrahydro-[1,2,3]triazolo[1,5-a]pyridine-6-carboxylic acid (87.46 mg, 0.45 mmol) and 3-phenyl-1H-indazol-5-amine (96.6 mg, 0.45 mmol) were dissolved in dry DMF (2 mL) and then triethylamine (0.09 mL, 0.67 mmol) and HATU (170.3 mg, 0.45 mmol) were sequentially added. The mixture was stirred at r.t. for 18 hrs. Additional HATU (170.3 mg, 0.45 mmol) was added and stirring was continued for 1 hr. The reaction was diluted with water and EtOAc. The organic phase was dried over Na₂SO₄, filtered and concentrated under reduced pressure. The crude material obtained (194 mg) was purified by preparative HPLC (acid conditions) to afford the title compound (12.4 mg, 0.032 mmol, 7% yield) as a racemate. MS-ESI (m/z) calc'd for C₂₂H₂₃N₆O [M+H]⁺: 387.2. Found 387.3.

### Separation of Enantiomers of 7,7-Dimethyl-N-(3-phenyl-1H-indazol-5-yl)-4,5,6,7-tetrahydro-[1,2,3]triazolo[1,5-a]pyridine-6-carboxamide

Racemic 7,7-dimethyl-N-(3-phenyl-1H-indazol-5-yl)-4,5,6,7-tetrahydro-[1,2,3]triazolo[1,5-a]pyridine-6-carboxamide (Intermediate 8) was subjected to semi-preparative chiral HPLC: (Column: Chiralpak IC (25×2.0 cm), 5μ; mobile phase: n-hexane/EtOH 75/25% v/v; Flow rate (mL/min): 17 mL/min. DAD detection: 220 nm; loop: 1000 μL. total amount: 12.4 mg; Solubilsation: 12.4 mg in 2.5 mL DCM/MeOH 1/1=4.96 mg/mL; injection: 4.96 mg/injection). Analytic chiral HPLC: (column: Chiralpak IC (25×0.46 cm), 5μ; mobile phase: n-hexane/EtOH 75/25% v/v; flow rate (mL/min): 1.0 mL/min. DAD detection: 220 nm; loop: 20 μL).

### First Eluting Enantiomer (Example 54a)

(R or S)-7,7-Dimethyl-N-(3-phenyl-1H-indazol-5-yl)-4,5,6,7-tetrahydro-[1,2,3]triazolo[1,5-a]pyridine-6-carboxamide (2.3 mg, 0.006 mmol) as a white solid. 96% pure, e.e.=100%. Analytic chiral HPLC: 11.48 min. Semi-preparative chiral HPLC: 12.34 min. ¹H NMR (400 MHz, MeOD) δ 8.40 (dd, J=1.63, 0.88 Hz, 1H), 7.91-7.99 (m, 2H), 7.50-7.63 (m, 4H), 7.49 (s, 1H), 7.39-7.46 (m, 1H), 3.12-3.25 (m, 1H), 3.05 (dd, J=8.78, 3.76 Hz, 1H), 2.87-3.00 (m, 1H), 2.20-2.42 (m, 2H), 1.87 (s, 3H), 1.78 (s, 3H). MS-ESI (m/z) calc'd for C₂₂H₂₃N₆O [M+H]⁺: 387.2. Found 387.3.

### Second Eluting Enantiomer (Example 54b)

(S or R)-7,7-Dimethyl-N-(3-phenyl-1H-indazol-5-yl)-4,5,6,7-tetrahydro-[1,2,3]triazolo[1,5-a]pyridine-6-carboxamide (2.7 mg, 0.007 mmol) as a white solid. 95% pure, e.e.=94%. Analytic chiral HPLC: 13.2 min. Semi-preparative chiral HPLC: 14.14 min. ¹H NMR (400 MHz, CD₃OD) δ 8.40 (dd, J=1.76, 1.00 Hz, 1H), 7.94 (dd, J=8.28, 1.25 Hz, 2H), 7.50-7.62 (m, 4H), 7.49 (s, 1H), 7.40-7.46 (m, 1H), 3.12-3.24 (m, 1H), 3.05 (dd, J=8.78, 3.51 Hz, 1H), 2.83-2.99 (m, 1H), 2.19-2.45 (m, 2H), 1.87 (s, 3H), 1.78 (s, 3H). MS-ESI (m/z) calc'd for C₂₂H₂₃N₆O [M+H]⁺: 387.2. Found 387.3.

### Example 55. 5,7-Dimethyl-N-(3-methyl-1H-indazol-5-yl)imidazo[1,5-a]pyridine-6-carboxamide

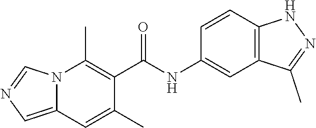 [SMILES: Cc1cc2cncn2c(C)c1C(=O)Nc1ccc2[nH]nc(C)c2c1]

5,7-dimethylimidazo[1,5-a]pyridine-6-carboxylic acid (35.0 mg, 0.180 mmol, 1 eq) and 3-methyl-1H-indazol-5-amine (54.17 mg, 0.370 mmol, 2 eq) were dissolved in dry DMF (2 mL). Then the solution was cooled to 0° C. with an ice-water bath and triethylamine (27.93 mg, 0.280 mmol, 1.5 eq) and HATU (83.96 mg, 0.220 mmol, 1.2 eq) were added. The mixture was stirred at 0° C. for 5 minutes and then at room temperature overnight. The reaction mixture was partitioned between water and EtOAc, the phases were separated, the aqueous layer was extracted with EtOAc (2×) and the combined organic phases washed with water (1×), dried over anhydrous Na₂SO₄ and evaporated to dryness. The crude was purified by normal phase chromatography on a 28 g NH-silica gel column, using as eluent a gradient of MeOH in EtOAc from 0 to 5%. The purest fractions were combined and purified again by reverse phase chromatography on a 12 g C18 column, using as eluent a gradient of CH₃CN in H₂O from 5 to 20% in presence of 0.1% formic acid. The target compound (18 mg, 0.056 mmol, 30.63% yield) was obtained as a white solid. ¹H NMR (400 MHz, DMSO-d₆) δ 12.60 (s, 1H), 10.49 (s, 1H), 8.36 (s, 1H), 8.23 (s, 1H), 7.51-7.34 (m, 4H), 2.60 (s, 3H), 2.49 (s, 3H), 2.27 (d, J=0.7 Hz, 3H). MS-ESI (m/z) calc'd for C₁₈H₁₈N₅O [M+H]⁺: 320.1. Found 320.2.

### Example 56. 6,8-Dichloro-N-(3-methyl-1H-indazol-5-yl)-[1,2,4]triazolo[4,3-a]pyridine-7-carboxamide

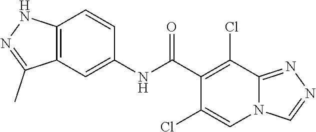 [SMILES: Cc1n[nH]c2ccc(NC(=O)c3c(Cl)cn4cnnc4c3Cl)cc12]

### Step 1. 3,5-Dichloro-2-(hydrazin-1-ium-2-yl)isonicotinate

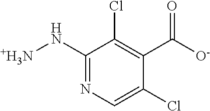 [SMILES: [NH3+]Nc1ncc(Cl)c(C(=O)[O-])c1Cl]

A mixture 2,3,5-trichloropyridine-4-carboxylic acid (800.0 mg, 3.53 mmol), hydrazine hydrate (4.0 mL, 200 mmol), and 1-propanol (4 mL) was boiled with stirring for 6 hrs. The mixture was allowed to cool to room temperature and evaporated to dryness. The residue was washed with EtOH and diethyl ether to give the title compound (784 mg, 3.53 mmol, 100% yield). ¹H NMR (400 MHz, DMSO-d₆) δ 7.90 (s, OH), 7.41 (s, 1H), 7.09 (s, 3H). MS-ESI (m/z) calculated for C₆H₆Cl₂N₃O₂ [M+H]⁺: 222.0, 224.0. Found 222.1, 224.1.

### Step 2. 6,8-Dichloro-[1,2,4]triazolo[4,3-a]pyridine-7-carboxylic acid

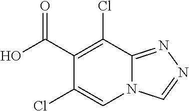 [SMILES: O=C(O)c1c(Cl)cn2cnnc2c1Cl]

3,5-Dichloro-2-(hydrazin-1-ium-2-yl)isonicotinate (300.0 mg, 1.35 mmol) was suspended in trimethyl orthoformate (3.25 mL, 29.73 mmol) and the mixture was heated to 60° C. After 1 hr, the mixture was cooled to ambient temperature and concentrated to afford the title compound (286 mg, 1.233 mmol, 91% yield), that was used in next step without further purification. ¹H NMR (400 MHz, DMSO-d₆) δ 9.34 (s, 1H), 8.98 (s, 1H). MS-ESI (m/z) calculated for C₇H₄Cl₂N₃O₂ [M+H]⁺: 232.0, 234.0. Found 232.1, 234.1.

### Step 3. 6,8-Dichloro-N-(3-methyl-1H-indazol-5-yl)-[1,2,4]triazolo[4,3-a]pyridine-7-carboxamide

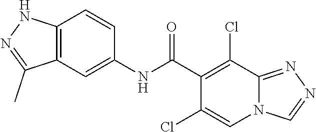 [SMILES: Cc1n[nH]c2ccc(NC(=O)c3c(Cl)cn4cnnc4c3Cl)cc12]

6,8-Dichloro-[1,2,4]triazolo[4,3-a]pyridine-7-carboxylic acid (50.0 mg, 0.140 mmol) and 3-methyl-1H-indazol-5-amine (41 mg, 0.280 mmol) were dissolved in dry DMF (0.700 mL). The solution was cooled to 0° C. with an ice-water bath and TEA (29 uL, 0.210 mmol) and HATU (63 mg, 0.170 mmol) were added. The mixture was stirred at 0° C. for 5 minutes and then at room temperature overnight. The crude was purified by preparative HPLC and then by chiral preparative HPLC to afford the title compound (4.2 mg, 0.012 mmol, 8% yield) as a yellow solid. ¹H NMR (400 MHz, DMSO-d₆) δ 12.66 (s, 1H), 10.89 (s, 1H), 9.39 (s, 1H), 9.04 (s, 1H), 8.17 (d, J=1.8 Hz, 1H), 7.48 (d, J=8.8 Hz, 1H), 7.42 (dd, J=8.9, 1.9 Hz, 1H), 2.50 (s, 3H). MS-ESI (m/z) calculated for C₁₅H₁₁Cl₂N₆O [M+H]⁺: 361.0, 363.0. Found 361.2, 363.3.

### Example 57. 6,8-Dimethyl-N-(3-phenyl-1H-indazol-5-yl)-[1,2,4]triazolo[4,3-a]pyridine-7-carboxamide

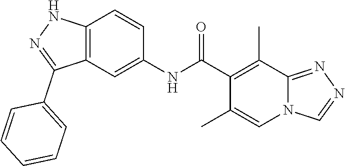 [SMILES: Cc1cn2cnnc2c(C)c1C(=O)Nc1ccc2[nH]nc(-c3ccccc3)c2c1]

### Step 1. Methyl 6,8-dichloro-[1,2,4]triazolo[4,3-a]pyridine-7-carboxylate

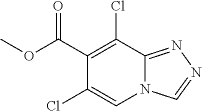

To a solution of 6,8-dichloro-[1,2,4]triazolo[4,3-a]pyridine-7-carboxylic acid (721 mg, 3.11 mmol) in MeOH (12 mL) was added an ether solution of (trimethylsilyl)diazomethane (2M, 1.86 mL, 3.73 mmol) dropwise until the yellow color persists over 30 minutes at ambient temperature. A few drops of TFA to quench the reaction were added and the mixture was evaporated to dryness. The resulting residue was purified by NH-chromatography eluting with EtOAc/MeOH mixture (0% to 20%). Related fractions were pooled and evaporated to afford the title compound (172 mg, 0.699 mmol, 22.5% yield). ¹H NMR (400 MHz, DMSO-d₆) δ 9.37 (s, 1H), 9.03 (s, 1H), 4.00 (s, 3H). MS-ESI (m/z) calculated for C₈H₆Cl₂N₃O₂ [M+H]⁺: 246.0. Found 246.2.

### Step 2. Methyl 6,8-dimethyl-[1,2,4]triazolo[4,3-a]pyridine-7-carboxylate

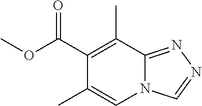

Methyl 6,8-dichloro-[1,2,4]triazolo[4,3-a]pyridine-7-carboxylate (142 mg, 0.58 mmol), 2,4,6-trimethyl-1,3,5,2,4,6-trioxatriborinane (145 mg, 1.15 mmol), potassium carbonate (478 mg, 3.46 mmol) were suspended in 1,4-dioxane (2 mL)/Water (0.2 mL) in microwave vial. The mixture was purged with N₂ for 5 minutes, and then was added palladium tetrakis (133 mg, 0.12 mmol). The reaction mixture was stirred at 110° C. overnight. After cooling to room temperature, the mixture was filtered through a pad of Celite, washed with THF and concentrated under reduced pressure. The residue was purified by NH-chromatography (from 100% AcOEt to 90/10 AcOEt/MeOH) to afford the title compound (133 mg, 0.648 mmol) which was used as such for the next step. ¹H NMR (400 MHz, DMSO-d₆) δ 9.23 (s, 1H), 8.39-8.30 (m, 1H), 3.93 (s, 3H), 2.52 (d, J=0.7 Hz, 3H), 2.19 (d, J=1.3 Hz, 3H). MS-ESI (m/z) calculated for C₁₀H₁₂N₃O₂ [M+H]⁺: 206.1. Found 206.2.

### Step 3. 6,8-Dimethyl-[1,2,4]triazolo[4,3-a]pyridine-7-carboxylic acid

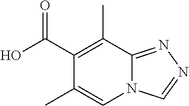

A 2M aq solution of sodium hydroxide (4.43 mL, 8.87 mmol) was added to a solution of methyl 6,8-dimethyl-[1,2,4]triazolo[4,3-a]pyridine-7-carboxylate (124 mg, 0.61 mmol) in MeOH (2 mL) and the reaction was stirred at room temperature for 18 hrs and then heated at 50° for 1 hr. The mixture was then cooled to room temperature and MeOH was evaporated under reduced pressure. 2M HCl was added until pH 1. The aqueous phase was concentrated and then the solid formed was triturated with EtOH. The salts precipitated were filtered off and the filtrate was evaporated to afford the title compound (82 mg, 0.429 mmol, 71% yield). ¹H NMR (400 MHz, DMSO-d₆) δ 14.06 (bs, 1H), 9.37 (s, 1H), 8.45 (s, 1H), 2.55 (s, 3H), 2.27 (d, J=1.2 Hz, 3H). MS-ESI (m/z) calculated for C₉H₁₀N₃O₂ [M+H]⁺: 192.1. Found 192.2.

### Step 4. 6,8-Dimethyl-N-(3-phenyl-1H-indazol-5-yl)-[1,2,4]triazolo[4,3-a]pyridine-7-carboxamide

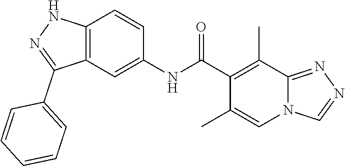 [SMILES: Cc1cn2cnnc2c(C)c1C(=O)Nc1ccc2[nH]nc(-c3ccccc3)c2c1]

6,8-Dimethyl-[1,2,4]triazolo[4,3-a]pyridine-7-carboxylic acid (41 mg, 0.210 mmol) and 3-phenyl-1H-indazol-5-amine (67 mg, 0.320 mmol) were dissolved in dry DMF (0.700 mL). Then the solution was cooled to 0° C. with an ice-water bath and triethylamine (0.06 mL, 0.430 mmol) and HATU (98 mg, 0.260 mmol) were added. The mixture was stirred at 0° C. for 5 minutes and then at room temperature for 18 hrs. The mixture was concentrated to give a crude which was purified by C-18 chromatography (from 100% water+0.10% formic acid to 60/40 water+0.10% formic acid/CAN+0.10% formic acid in 12CV) then the crude material was re-purified by NH-chromatography (from 100% EtOAc to 90/10 EtOAc/MeOH) to afford the title compound (4.2 mg, 0.011 mmol, 5% yield). ¹H NMR (400 MHz, DMSO-d₆) δ 13.28 (s, 1H), 10.67 (s, 1H), 9.24 (s, 1H), 8.59 (t, J=1.3 Hz, 1H), 8.38 (d, J=1.5 Hz, 1H), 8.00-7.87 (m, 2H), 7.64 (dd, J=8.9, 1.7 Hz, 1H), 7.62-7.59 (m, 1H), 7.55 (t, J=7.7 Hz, 2H), 7.45-7.39 (m, 1H), 2.56 (s, 3H), 2.26 (d, J=1.3 Hz, 3H). MS-ESI (m/z) calculated for C₂₂H₁₉N₆O [M+H]⁺: 383.2. Found 383.4.

### Example 58. 6,8-Dimethyl-N-(3-methyl-1H-indazol-5-yl)-[1,2,4]triazolo[4,3-a]pyridine-7-carboxamide

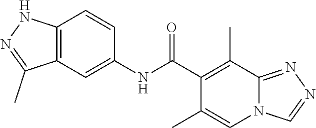 [SMILES: Cc1cn2cnnc2c(C)c1C(=O)Nc1ccc2[nH]nc(C)c2c1]

6,8-Dimethyl-[1,2,4]triazolo[4,3-a]pyridine-7-carboxylic acid (41.0 mg, 0.210 mmol) and 3-phenyl-1H-indazol-5-amine (63 mg, 0.43 mmol) were dissolved in dry DMF (0.700 mL). Then the solution was cooled to 0° C. with an ice-water bath and TEA (45 μL, 0.32 mmol) and HATU (98 mg, 0.26 mmol) were added. The mixture was stirred at 0° C. for 5 minutes and then at room temperature overnight. The mixture was concentrated to give a crude which was purified by C-18 chromatography (from 100% water+0.1% formic acid to 60/40 water+0.1% formic acid/CAN+0.1% formic acid in 12 CV) to give crude product which was re-purified by NH-chromatography (from 100% EtOAc to 90/10 EtOAc/MeOH) to afford the title compound (13 mg, 0.041 mmol, 19% yield). ¹H NMR (400 MHz, DMSO-d₆) δ 12.62 (s, 1H), 10.56 (s, 1H), 9.24 (s, 1H), 8.37 (d, J=1.6 Hz, 1H), 8.20 (t, J=1.3 Hz, 1H), 7.49-7.43 (m, 2H), 2.55 (s, 3H), 2.48 (s, 3H), 2.26 (d, J=1.3 Hz, 3H). MS-ESI (m/z) calculated for C₁₇H₁₇N₆O [M+H]⁺: 321.1. Found 321.4.

### Example 59. 6-Chloro-8-methyl-N-(3-phenyl-1H-indazol-5-yl)-[1,2,4]triazolo[4,3-a]pyridine-7-carboxamide

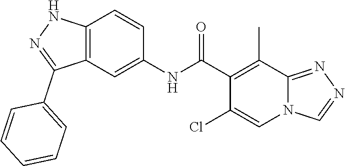 [SMILES: Cc1c(C(=O)Nc2ccc3[nH]nc(-c4ccccc4)c3c2)c(Cl)cn2cnnc12]

### Step 1. Methyl 6-chloro-8-methyl-[1,2,4]triazolo[4,3-a]pyridine-7-carboxylate

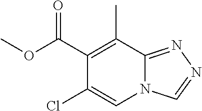

Palladium tetrakis (64 mg, 0.060 mmol), potassium carbonate (153 mg, 1.11 mmol) and methyl 6,8-dichloro-[1,2,4]triazolo[4,3-a]pyridine-7-carboxylate (300 mg, 1.11 mmol) were suspended in 1,4-dioxane (4 mL)/water (0.400 uL) in microwave vial. The mixture was purged with N₂ for 5 minutes, and then 2,4,6-trimethyl-1,3,5,2,4,6-trioxatriborinane (139 mg, 1.11 mmol) was added. The reaction mixture was stirred at 110° C. for 15 hrs and after cooling to room temperature, it was filtered and concentrated under reduced pressure to obtain a residue which was purified by prep-HPLC to afford the title compound (25 mg, 0.112 mmol, 10% yield). ¹H NMR (500 MHz, DMSO-d₆) δ 2.55 (s, 3H), 3.95 (s, 3H), 8.85 (d, J=0.82 Hz, 1H), 9.26 (s, 1H) MS-ESI (m/z) calculated for C₉H₉ClN₃O₂ [M+H]⁺: 226.0, 228.0. Found 226.2, 228.2.

### Step 2. 6-Chloro-8-methyl-[1,2,4]triazolo[4,3-a]pyridine-7-carboxylic acid

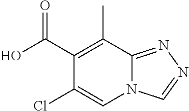

A 2M aq solution of sodium hydroxide (0.78 mL, 1.56 mmol) was added to a solution of methyl 6,8-dimethyl-[1,2,4]triazolo[4,3-a]pyridine-7-carboxylate (24.0 mg, 0.110 mmol) in MeOH (2 mL) and the reaction was stirred at room temperature for 18 hrs and heated at 50° C. for 1 hr. The mixture was then cooled to room temperature and MeOH was remove under reduced pressure. 2M HCl_{aq} was added until pH 1, and the mixture was evaporated to dryness. The solid obtained was triturated with EtOH, the salts precipitated were filtered off and the filtrate was evaporated to afford the title compound (22 mg, 0.104 mmol, 98% yield). ¹H NMR (400 MHz, DMSO-d₆) δ 14.13 (s, 1H), 9.24 (s, 1H), 8.83 (s, 1H), 2.56 (s, 3H). MS-ESI (m/z) calculated for C₈H₇ClN₃O₂ [M+H]⁺: 212.0, 214.0. Found 212.1, 214.2.

### Step 3. 6-Chloro-8-methyl-N-(3-phenyl-1H-indazol-5-yl)-[1,2,4]triazolo[4,3-a]pyridine-7-carboxamide

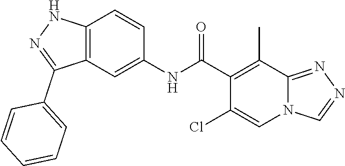 [SMILES: Cc1c(C(=O)Nc2ccc3[nH]nc(-c4ccccc4)c3c2)c(Cl)cn2cnnc12]

6-Chloro-8-methyl-[1,2,4]triazolo[4,3-a]pyridine-7-carboxylic acid (22 mg, 0.100 mmol) and 3-phenyl-1H-indazol-5-amine (33 mg, 0.160 mmol) were dissolved in dry DMF (1 mL). Then the solution was cooled to 0° C. with an ice-water bath and TEA (0.03 mL, 0.210 mmol) and HATU (47 mg, 0.120 mmol) were added. The mixture was stirred at 0° C. for 5 minutes and then at room temperature for 18 hrs. The mixture was concentrated to give a crude which was purified by C-18 chromatography (from 100% water+0.1% formic acid to 60/40 water+0.1% formic acid/ACN+0.1% formic acid in 12CV). The solid obtained was purified again by NH-chromatography (from 100% EtOAc to 90/10 EtOAc/MeOH) to afford the title compound (10 mg, 0.026 mmol, 25% yield). ¹H NMR (400 MHz, DMSO-d₆) δ 13.28 (s, 1H), 10.81 (s, 1H), 9.28 (s, 1H), 8.88 (d, J=0.9 Hz, 1H), 8.57 (t, J=1.3 Hz, 1H), 7.98-7.90 (m, 2H), 7.64-7.60 (m, 2H), 7.56 (t, J=7.7 Hz, 2H), 7.48-7.38 (m, 1H), 2.62-2.59 (m, 3H). MS-ESI (m/z) calculated for C₂₁H₁₆ClN₆O [M+H]⁺: 403.1, 405.1. Found 403.1, 405.1.

### Example 60. 5,7-Dimethyl-N-(3-phenyl-1H-indazol-5-yl)-[1,2,4]triazolo[4,3-a]pyridine-6-carboxamide

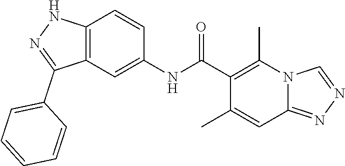 [SMILES: Cc1cc2nncn2c(C)c1C(=O)Nc1ccc2[nH]nc(-c3ccccc3)c2c1]

### Step 1. 6-Hydroxy-2,4-dimethylnicotinic acid

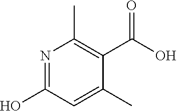

6-Chloro-2,4-dimethylnicotinonitrile (5.0 g, 30.01 mmol, 1 eq) was dissolved in a mixture of sulfuric acid (10.4 mL, 195.07 mmol, 6.5 eq) and water (9 mL). The mixture was heated at 120° C. for 16 hrs. Then it was then cooled to 90° C. and sodium nitrite (14.49 g, 210.07 mmol, 7 eq) was added in small portions over 10 minutes. The reaction was heated at 90° C. for an additional hour. Then other 3.5 eq. of NaNO₂ were added portion-wise and the mixture was stirred at 90° C. for additional 2 hrs. The mixture was cooled to r.t. and poured into an ice-water mix. The chilled mixture was basified to pH=12 using NaOH aq. 10N. The aqueous mixture was then washed with DCM (2×) to remove impurities. The pH was then adjusted to 2 by adding HCl aq. 6N and a mixture 4:1 of DCM/MeOH was added. The off-white solid at the interface was recovered by filtration to afford the title compound (1.3 g, 7.77 mmol, 25.91% yield). ¹H NMR (400 MHz, DMSO-d₆) δ 12.90-11.00 (m, 2H), 6.05 (s, 1H), 2.30 (s, 3H), 2.18 (s, 3H). MS-ESI (m/z) calc'd for C₈H₁₀NO₃ [M+H]⁺: 168.1. Found 168.2. The filtrate was also recovered, the 2 phases separated, the aqueous layer extracted with DCM/MeOH 4/1 (2×) and the combined extracts were washed with water, dried over anhydrous Na₂SO₄ and concentrated to dryness to afford the title compound (2.9 g, 15.62 mmol, 52% yield). ¹H NMR (400 MHz, DMSO-d₆) δ 13.84-13.55 (bs, 1H), 7.34 (s, 1H), 2.45 (s, 3H), 2.31 (s, 3H). MS-ESI (m/z) calc'd for C₈H₉ClNO₂ [M+H]⁺: 186.0. Found 186.2.

### Step 2. Ethyl 6-chloro-2,4-dimethylnicotinate

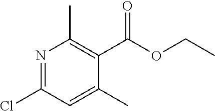

A suspension of 6-hydroxy-2,4-dimethylnicotinic acid (1.3 g, 7.78 mmol, 1 eq) in Phosphorus(V) oxychloride (13.09 mL, 139.99 mmol, 18 eq) was stirred at 100° C. overnight. Then the mixture was cooled to room temperature and evaporated to dryness. The residue was cooled to 0° C. and EtOH (7 mL) was added dropwise. The mixture was stirred at r.t. for 1.5 hrs, then partitioned between EtOAc and H₂O. The organic phase was concentrated in vacuo and the residue was purified by normal phase chromatography on a 50 g silica gel column, using as eluent a gradient of EtOAc in cyclohexane, from 0 to 20% to afford the title compound (710 mg, 3.323 mmol, 42.73% yield) as a colorless oil. ¹H NMR (400 MHz, CDCl₃) δ 7.06 (s, 1H), 4.44 (q, J=7.1 Hz, 2H), 2.55 (s, 3H), 2.34 (s, 3H), 1.42 (t, J=7.2 Hz, 3H). MS-ESI (m/z) calc'd for C₁₀H₁₃ClNO₂ [M+H]⁺: 214.1. Found 214.2.

### Step 3. Ethyl 6-hydrazinyl-2,4-dimethylnicotinate

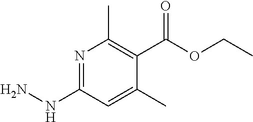

A mixture of ethyl 6-chloro-2,4-dimethylnicotinate (100.0 mg, 0.420 mmol, 1 eq), hydrazine hydrate (0.13 mL, 6.32 mmol, 15 eq) and EtOH (1 mL) was stirred at 80° C. for 12 hrs. The mixture was allowed to cool to r.t. and evaporated under vacuum. To strip hydrazine, the residue was dissolved in EtOH and the solvent removed under reduced pressure three times to afford the title compound (0.420 mmol theoretical) as a yellow solid without further purification. ¹H NMR (400 MHz, DMSO-d₆) δ 7.70 (br. s., 1H), 6.42 (s, 1H), 4.25 (q, J=7.0 Hz, 2H), 2.31 (s, 3H), 2.20 (s, 3H), 1.29 (t, J=7.2 Hz, 3H). MS-ESI (m/z) calc'd for C₁₀H₁₆N₃O₂ [M+H]⁺: 210.1. Found 210.3.

### Step 4. Ethyl 5,7-dimethyl-[1,2,4]triazolo[4,3-a]pyridine-6-carboxylate

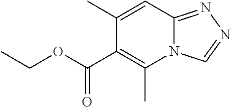

Ethyl 6-hydrazinyl-2,4-dimethylnicotinate (0.420 mmol theoretical, 1 eq), was suspended in trimethoxymethane (1.0 mL, 9.14 mmol) and the mixture was stirred at 60° C. for 1 hr and then at 100° C. overnight. The mixture was cooled to room temperature and concentrated. The brown residue was purified by normal phase chromatography on a 11 g NH-silica gel column, using as eluent a gradient of EtOAc in cyclohexane from 0 to 80% to afford the title compound (30 mg, 0.137 mmol, 32.58% yield) as a white solid. ¹H NMR (400 MHz, DMSO-d₆) δ=9.31 (d, J=0.9 Hz, 1H), 7.59 (s, 1H), 4.40 (q, J=7.0 Hz, 2H), 2.68 (s, 3H), 2.33 (d, J=1.1 Hz, 3H), 1.35 (t, J=7.2 Hz, 3H). MS-ESI (m/z) calc'd for C₁₁H₁₄N₃O₂ [M+H]⁺: 220.1. Found 220.3.

### Step 5. 5,7-Dimethyl-[1,2,4]triazolo[4,3-a]pyridine-6-carboxylic acid

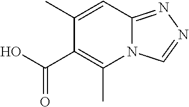

Ethyl 5,7-dimethyl-[1,2,4]triazolo[4,3-a]pyridine-6-carboxylate (30.0 mg, 0.137 mmol, 1 eq) was dissolved in EtOH (0.700 mL) and a 2M aqueous solution of sodium hydroxide (1.5 mL, 3 mmol) was added. The solution was stirred at r.t. overnight. Ethanol was removed under reduced pressure and then the aqueous residue acidified with HCl 2M until pH=1. The mixture was partitioned between water and EtOAc, the phases were separated, the aqueous layer was extracted with EtOAc (2×) and the combined organic phases washed with water (1×), dried over anhydrous Na₂SO₄ and evaporated to dryness. The aqueous layer was evaporated to dryness, and the obtained solid triturated with EtOH. The insoluble salts were removed by filtration, while the filtrate was collected and the solvent evaporated under reduced pressure to afford the title compound (20 mg, 0.105 mmol, 76.45% yield) as an off-white solid. ¹H NMR (400 MHz, DMSO-d₆) δ 9.55 (s, 1H), 7.74 (s, 1H), 2.75 (s, 3H), 2.45 (d, J=0.9 Hz, 3H). MS-ESI (m/z) calc'd for C₉H₁₀N₃O₂ [M+H]⁺: 192.1. Found 192.2.

### Step 6. 5,7-Dimethyl-N-(3-phenyl-1H-indazol-5-yl)-[1,2,4]triazolo[4,3-a]pyridine-6-carboxamide

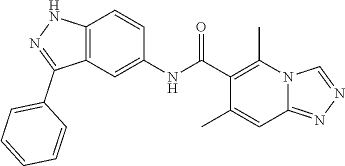 [SMILES: Cc1cc2nncn2c(C)c1C(=O)Nc1ccc2[nH]nc(-c3ccccc3)c2c1]

5,7-Dimethyl-[1,2,4]triazolo[4,3-a]pyridine-6-carboxylic acid (20.0 mg, 0.105 mmol, 1 eq) and 3-phenyl-1H-indazol-5-amine (43.78 mg, 0.210 mmol, 2 eq) were dissolved in dry DMF (1.5 mL). Then the solution was cooled to 0° C. with an ice-water bath and triethylamine (21.87 uL, 0.160 mmol, 1.5 eq) and HATU (47.73 mg, 0.130 mmol, 1.2 eq) were added. The mixture was stirred at 0° C. for 5 minutes and then at room temperature overnight. The reaction mixture was partitioned between water and EtOAc, the phases were separated, the aqueous layer was extracted with EtOAc (2×) and the combined organic phases washed with water (1×), dried over anhydrous Na₂SO₄ and evaporated to dryness. The crude was purified by normal phase chromatography on a 11 g NH-silica gel column, using as eluent a gradient of EtOAc in cyclohexane from 0 to 100% and then isocratic elution with EtOAc/MeOH 9:1. The purest fractions were combined and evaporated to dryness to afford a crude that was purified again by reverse phase chromatography on a 12 g C18 column, using as eluent a gradient of CH₃CN in H₂O from 5 to 40% in presence of 0.1% formic acid. The title compound (7 mg, 0.018 mmol, 17.5% yield) was obtained as a white solid. ¹H NMR (400 MHz, DMSO-d₆) δ 13.28 (br. s., 1H), 10.66 (s, 1H), 9.35 (d, J=0.88 Hz, 1H), 8.59 (s, 1H), 7.92-7.98 (m, 2H), 7.61-7.67 (m, 3H), 7.56 (t, J=7.59 Hz, 2H), 7.39-7.47 (m, 1H), 2.70 (s, 4H), 2.39 (d, J=0.88 Hz, 3H). MS-ESI (m/z) calc'd for C₂₂H₁₉N₆O [M+H]⁺: 383.2. Found 383.4.

### Example 61. 5,7-Dimethyl-N-(3-methyl-1H-indazol-5-yl)-[1,2,4]triazolo[4,3-a]pyridine-6-carboxamide

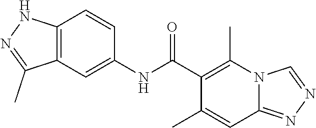 [SMILES: Cc1cc2nncn2c(C)c1C(=O)Nc1ccc2[nH]nc(C)c2c1]

5,7-Dimethyl-[1,2,4]triazolo[4,3-a]pyridine-6-carboxylic acid (30.0 mg, 0.157 mmol, 1 eq) and 3-methyl-1H-indazol-5-amine (46.19 mg, 0.314 mmol, 2 eq) were dissolved in dry DMF (1.5 mL). Then the solution was cooled to 0° C. with an ice-water bath and triethylamine (32.81 μL, 0.240 mmol, 1.5 eq) and HATU (71.6 mg, 0.190 mmol, 1.2 eq) were added. The mixture was stirred at 0° C. for 5 minutes and then at room temperature overnight. The crude was loaded directly on a 12 g C18 cartridge and purified by reverse phase chromatography using as eluent a gradient of CH₃CN in H₂O from 5 to 25% in presence of 0.1% formic acid. Fractions containing product were combined and purified again by normal phase chromatography on a 11 g NH-silica gel column, using as eluent a gradient of MeOH in EtOAc from 0 to 10% to afford the title compound (5 mg, 0.016 mmol, 10% yield) as a white solid. ¹H NMR (400 MHz, DMSO-d₆) δ 12.63 (s, 1H), 10.56 (s, 1H), 9.34 (s, 1H), 8.21 (s, 1H), 7.63 (s, 1H), 7.46 (s, 2H), 2.69 (s, 3H), 2.49 (s, 3H), 2.39 (d, J=0.66 Hz, 3H). MS-ESI (m/z) calc'd for C₁₇H₁₇N₆O [M+H]⁺: 320.1. Found 321.3.

### Example 62. 5-Methyl-N-(3-phenyl-1H-indazol-5-yl)imidazo[1,5-a]pyridine-6-carboxamide

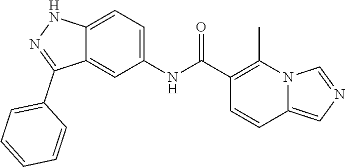 [SMILES: Cc1c(C(=O)Nc2ccc3[nH]nc(-c4ccccc4)c3c2)ccc2cncn12]

5-methylimidazo[1,5-a]pyridine-6-carboxylic acid (50.0 mg, 0.280 mmol) and 3-phenyl-1H-indazol-5-amine (89.08 mg, 0.430 mmol) were dissolved in dry DMF (0.701 mL). Then the solution was cooled to 0° C. with an ice-water bath and triethylamine (0.08 mL, 0.570 mmol) and [dimethylamino(3-triazolo[4,5-b]pyridinyloxy)methylidene]-dimethylammonium hexafluorophosphate (129.5 mg, 0.340 mmol) were added. The mixture was stirred at 0° C. for 5 minutes and then at room temperature for 18 hrs. The mixture was concentrated to give a crude which was purified by reverse phase column chromatography on C-18-silica gel column (from 100% water+0.1% formic acid to 60/40 water+0.10% formic acid/acetonitrile+0.10% formic acid) then the crude was re-purified by NH-chromatography (from 100% EtOAc to 90/10 EtOAc/MeOH) to afford the title compound (18.1 mg, 0.049 mmol, 17.36% yield). ¹H NMR (400 MHz, DMSO-d₆) δ 13.23 (br. S, 1H), 10.55 (s, 1H), 8.99 (s, 1H), 8.59 (d, J=1.18 Hz, 1H), 7.93-7.99 (m, 2H), 7.82 (br. S, 1H), 7.74 (d, J=9.39 Hz, 1H), 7.67-7.71 (m, 1H), 7.52-7.63 (m, 3H), 7.38-7.46 (m, 1H), 7.17 (d, J=9.37 Hz, 1H), 2.77 (s, 3H). MS-ESI (m/z) calc'd for C₂₂H₁₈N₅O [M+H]⁺: 368.1. Found 368.4.

### Example 63. 6,8-dichloro-N-(3-phenyl-1H-indazol-5-yl)-[1,2,4]triazolo[4,3-a]pyridine-7-carboxamide

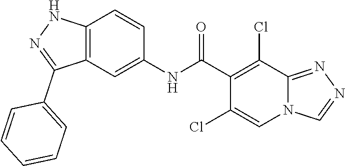 [SMILES: O=C(Nc1ccc2[nH]nc(-c3ccccc3)c2c1)c1c(Cl)cn2cnnc2c1Cl]

To a solution of 3-phenyl-1H-indazol-5-amine (67.6 mg, 0.32 mmol), 6,8-dichloro-[1,2,4]triazolo[4,3-a]pyridine-7-carboxylic acid (50 mg, 0.22 mmol) and triethylamine (0.04 mL, 0.26 mmol) in DMF (2.5 mL), HATU (98.33 mg, 0.260 mmol) was added at 0° C. The resulting mixture was allowed to reach room temperature and left stirring for 4 hrs. Cooled water was added and the mixture was extracted with EtOAc (2×). The organic phases were collected and washed with brine, dried over Na₂SO₄, filtered and concentrated under reduced pressure to afford a crude (202 mg) which was purified via preparative HPLC (acid conditions) to afford the title compound (11.1 mg, 0.026 mmol, 12.2% yield) as pink solid. ¹H NMR (400 MHz, DMSO-d₆) δ 13.32 (s, 1H), 11.02 (s, 1H), 9.40 (s, 1H), 9.06 (s, 1H), 8.54 (s, 1H), 7.94 (dd, J=8.25, 1.21 Hz, 2H), 7.54-7.67 (m, 4H), 7.41-7.46 (m, 1H). MS-ESI (m/z) calc'd for C₂₀H₁₃Cl₂N₆O [M+H]⁺: 423.0, 425.0. Found 423.2; 425.2.

### Example 64. 3,5,7-Trimethyl-N-(3-phenyl-1H-indazol-5-yl)-[1,2,3]triazolo[1,5-a]pyridine-6-carboxamide

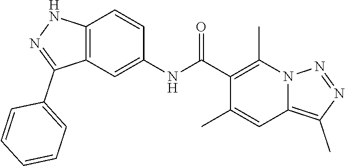 [SMILES: Cc1cc2c(C)nnn2c(C)c1C(=O)Nc1ccc2[nH]nc(-c3ccccc3)c2c1]

3,5,7-trimethyltriazolo[1,5-a]pyridine-6-carboxylic acid (36.5 mg, 0.180 mmol) and 3-phenyl-1H-indazol-5-amine (55.83 mg, 0.270 mmol) were dissolved in dry DMF (1 mL). Then the solution was cooled to 0° C. with an ice-water bath and triethylamine (37.19 uL, 0.270 mmol) and [dimethylamino(3-triazolo[4,5-b]pyridinyloxy)methylidene]-dimethylammonium hexafluorophosphate (81.16 mg, 0.210 mmol) were added. The mixture was stirred at 0° C. for 5 minutes and then at r.t. for 3 days. The crude was loaded directly into a 12 g C18 cartridge and purified by reverse phase column chromatography, using as eluent a gradient of CH₃CN in H₂O from 5 to 40% in presence of 0.1% formic acid. The product containing fractions were combined, evaporated to dryness and purified again by flash chromatography on a silica gel column, using a 50 to 100% gradient of EtOAc in cyclohexane as eluent to afford the title compound (5.9 mg, 0.015 mmol, 8.4% yield) as a white solid. ¹H NMR (400 MHz, DMSO-d₆) δ 13.28 (br. s., 1H), 10.72 (s, 1H), 8.61 (s, 1H), 7.93-7.98 (m, 2H), 7.77 (s, 1H), 7.60-7.67 (m, 2H), 7.52-7.59 (m, 2H), 7.40-7.45 (m, 1H), 2.81 (s, 3H), 2.57 (s, 3H), 2.41 (d, J=0.75 Hz, 3H). MS-ESI (m/z) calc'd for C₂₃H₂₁N₆O [M+H]⁺: 397.2. Found 397.4.

### Example 65. 3,5,7-Trimethyl-N-(3-methyl-1H-indazol-5-yl)-[1,2,3]triazolo[1,5-a]pyridine-6-carboxamide

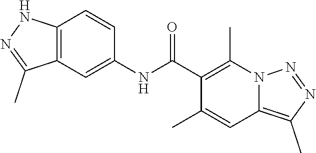 [SMILES: Cc1cc2c(C)nnn2c(C)c1C(=O)Nc1ccc2[nH]nc(C)c2c1]

### Step 1. 6-Chloro-2,4-dimethylnicotinic acid

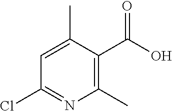

6-chloro-2,4-dimethylnicotinonitrile (3 g, 18.01 mmol) was dissolved in a mixture of sulfuric acid (25.0 mL, 118.23 mmol) and water (6 mL) The mixture was heated at 120° C. for 16 hrs. It was then cooled to 90° C. and sodium nitrite (8.82 g, 126.04 mmol) was added in small portions over 10 minutes. The reaction was heated at 90° C. for an additional hour and then cooled to r.t. and poured into an ice-water mixture (˜20 mL). The chilled mixture was basified to pH=12 using 10N NaOH. The aqueous mixture was then washed with CH₂Cl₂ (30 mL×2) to remove impurities. It was then acidified to pH=2. After being concentrated under high vacuum to dryness, the residue was extracted with CH₂Cl₂/CH₃OH (4/1, 70 mL×3). The combined extracts were concentrated to afford the title compound (4 g, 21.55 mmol). The product was used without further purification. ¹H NMR (400 MHz, DMSO-d₆) δ 9.57-10.12 (m, 1H), 7.33 (s, 1H), 2.44 (s, 3H), 2.31 (s, 3H). MS-ESI (m/z) calc'd for C₈H₉ClNO₂ [M+H]⁺: 186.0. Found 186.1.

### Step 2. Ethyl 6-chloro-2,4-dimethylnicotinate

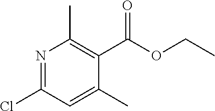

A suspension of 6-chloro-2,4-dimethylnicotinic acid (4.0 g, 21.55 mmol) in phosphorus(V) oxychloride (36.27 mL, 387.91 mmol), was stirred at 80° C. for 2 hrs, cooled to room temperature and then evaporated. The residue was cooled to 0° C. and EtOH (15 mL) was added dropwise. The mixture was stirred at room temperature for 1.5 hrs and then partitioned between EtOAc and H₂O. The organic phase was concentrated in vacuo and the residue was purified by normal phase column chromatography (cyclohexane/EtOAc, from 1:0 to 8:2, as eluent) to afford the title compound (2.44 g, 11.42 mmol, 53% yield). ¹H NMR (400 MHz, DMSO-d₆) δ 7.37 (s, 1H), 4.38 (q, J=7.19 Hz, 2H), 2.43 (s, 3H), 2.29 (s, 3H), 1.33 (t, J=7.15 Hz, 3H). MS-ESI (m/z) calc'd for C₁₀H₁₃ClNO₂ [M+H]⁺: 214.1, 216.1. Found 214.2, 216.2.

### Step 3. Ethyl 6-acetyl-2,4-dimethylnicotinate

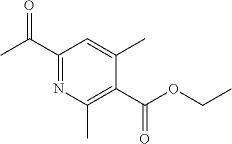 [SMILES: CCOC(=O)c1c(C)cc(C(C)=O)nc1C]

Ethyl 6-chloro-2,4-dimethylnicotinate (0.96 g, 4.47 mmol) and triphenylphosphine (0.12 g, 0.450 mmol) were dissolved in toluene (13 mL) in a previously nitrogen-filled sealed vessel. The solution was degassed with nitrogen, tributyl(1-ethoxyethenyl)stannane (1.96 mL, 5.82 mmol) palladium triphenylphosphine (258.52 mg, 0.220 mmol) and were added. The mixture was heated at 95° C. for 2 hrs under nitrogen atmosphere. The reaction mixture was concentrated and the crude enol-ether was treated with a 2:1 mixture of MeOH/concentrated HCl (3.3 mL/1.6 mL) and allowed to shake for 4 hrs. The reaction mixture was diluted with EtOAc and H₂O and basified by the slow addition of solid Na₂CO₃. The layers were separated and the aqueous layer was extracted several times with EtOAc. The combined organic extracts were dried over Na₂SO₄, filtered through a filtered funnel, and concentrated in vacuo. The crude residue was purified by normal phase column chromatography (cyclohexane/EtOAc, from 1:0 to 8:2, as eluent) to afford the title compound (0.96 g, 4.47 mmol). This product was used without further purification in the next step. MS-ESI (m/z) calc'd for C₁₂H₁₆NO₃ [M+H]⁺: 222.1. Found 222.1.

### Step 4. Ethyl (E)-2,4-dimethyl-6-(1-(2-tosylhydrazineylidene)ethyl)nicotinate

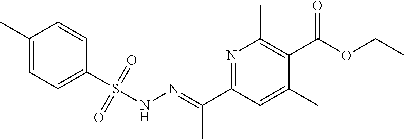 [SMILES: CCOC(=O)c1c(C)cc(/C(C)=N/NS(=O)(=O)c2ccc(C)cc2)nc1C]

To a mixture of 4-methylbenzenesulfonohydrazide (792.56 mg, 4.26 mmol) and EtOH (14 mL) was rapidly added ethyl 6-acetyl-2,4-dimethylnicotinate (856 mg, 3.87 mmol). The reaction was stirred at r.t. for 2 hrs. The mixture was then concentrated in vacuo to afford the title compound (1.62 g, 4.16 mmol) without further purification. MS-ESI (m/z) calc'd for C₁₉H₂₄N₃O₄S [M+H]⁺: 390.1. Found 390.4.

### Step 5. Ethyl 3,5,7-trimethyl-[1,2,3]triazolo[1,5-a]pyridine-6-carboxylate

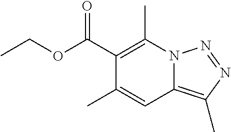 [SMILES: CCOC(=O)c1c(C)cc2c(C)nnn2c1C]

Ethyl (E)-2,4-dimethyl-6-(1-(2-tosylhydrazineylidene)ethyl)nicotinate (1.62 g, 4.16 mmol) was dissolved in morpholine 10 mL and the solution was stirred at 95° C. for 1 hr. The mixture was then concentrated in vacuo and the residue was taken up with DCM and washed with water. The aqueous layer was then extracted (3×) with DCM. The organic layers were combined and concentrated in vacuo. The crude material obtained was purified by normal phase silica gel column chromatography (cyclohexane/EtOAc, 1:0 to 3:7, as eluent), to afford the title compound (485 mg, 2.079 mmol, 50% yield) as a pale yellow oil. ¹H NMR (400 MHz, CDCl₃) δ 7.34 (s, 1H), 4.49 (q, J=7.04 Hz, 2H), 2.91 (s, 3H), 2.62 (s, 3H), 2.43 (s, 3H), 1.46 (t, J=7.15 Hz, 3H). MS-ESI (m/z) calc'd for C₁₂H₁₆N₃O₂ [M+H]⁺: 234.1. Found 234.3.

### Step 6. 3,5,7-trimethyl-[1,2,3]triazolo[1,5-a]pyridine-6-carboxylic acid

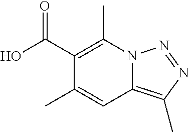

A 2M aq. solution of sodium hydroxide (3.13 mL, 6.27 mmol) was added to a solution of ethyl 3,5,7-trimethyl-[1,2,3]triazolo[1,5-a]pyridine-6-carboxylate (100 mg, 0.430 mmol) in MeOH (2 mL) and the reaction was stirred at room temperature for 18 hrs. The mixture was then cooled to room temperature and MeOH was remove under reduced pressure. HCl 2M aq. solution was added until pH 1. The precipitate was filtered and washed with water. The white solid was dried under reduced pressure at 50° C., to afford the title compound (73 mg, 0.356 mmol, 83% yield). MS-ESI (m/z) calc'd for C₁₀H₁₁N₃O₂ [M+H]⁺: 206.1. Found 206.2.

### Step 7. 3,5,7-Trimethyl-N-(3-methyl-1H-indazol-5-yl)-[1,2,3]triazolo[1,5-a]pyridine-6-carboxamide

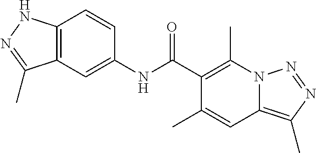 [SMILES: Cc1cc2c(C)nnn2c(C)c1C(=O)Nc1ccc2[nH]nc(C)c2c1]

3,5,7-Trimethyl-[1,2,3]triazolo[1,5-a]pyridine-6-carboxylic acid (42.52 mg, 0.210 mmol) and 3-methyl-1H-indazol-5-amine (61 mg, 0.410 mmol) were dissolved in dry DMF (1 mL). The solution was cooled to 0° C. with an ice-water bath and triethylamine (31.45 mg, 0.310 mmol) and [dimethylamino(3-triazolo[4,5-b]pyridinyloxy)methylidene]-dimethylammonium hexafluorophosphate (94.53 mg, 0.250 mmol) were added. The mixture was stirred at 0° C. for 5 minutes and then at room temperature overnight. The crude was loaded directly into a 12 g C18 cartridge and purified by reverse phase column chromatography, using as eluent a gradient of CH₃CN in H₂O from 5 to 40% in presence of 0.1% formic acid. The product containing fractions were combined, evaporated to dryness and purified again by NH silica phase column chromatography (cyclohexane/EtOAc, from 1:1 to 0:1, as eluent) to afford the title compound (28 mg, 0.084 mmol, 40% yield) as a white solid. ¹H NMR (400 MHz, DMSO-d₆) δ 12.62 (s, 1H), 10.61 (s, 1H), 8.21 (d, J=1.3 Hz, 1H), 7.76 (t, J=1.0 Hz, 1H), 7.47-7.44 (m, 2H), 2.79 (s, 3H), 2.55 (s, 3H), 2.49 (s, 3H), 2.40 (d, J=1.1 Hz, 3H). MS-ESI (m/z) calc'd for C₁₈H₁₉N₆O [M+H]⁺: 335.2. Found 335.4.

### Example 66. 5,7-Dimethyl-N-(3-(3-morpholinophenyl)-1H-indazol-5-yl)-[1,2,3]triazolo[1,5-a]pyridine-6-carboxamide

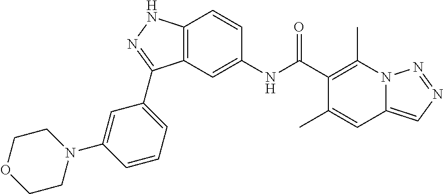 [SMILES: Cc1cc2cnnn2c(C)c1C(=O)Nc1ccc2[nH]nc(-c3cccc(N4CCOCC4)c3)c2c1]

5,7-Dimethyltriazolo[1,5-a]pyridine-6-carboxylic acid (40.0 mg, 0.210 mmol, 1 eq) and 3-(3-morpholin-4-ylphenyl)-1H-indazol-5-amine (92.37 mg, 0.310 mmol, 1.5 eq) were dissolved in dry DMF (1.5 mL). Then the solution was cooled to 0° C. with an ice-water bath and triethylamine (43.74 uL, 0.310 mmol, 1.5 eq) and HATU (95.46 mg, 0.250 mmol, 1.2 eq) were added. The mixture was stirred at 0° C. for 5 minutes and then at room temperature for 3 days. The crude was loaded directly on a 12 g C18 column and purified by reverse phase chromatography, using as eluent a gradient of CH₃CN in H₂O from 5 to 40% in presence of 0.1% formic acid. The purest fractions were combined, evaporated to dryness and purified again by normal phase column chromatography on a 11 g NH-silica gel column, using as eluent a gradient of MeOH in EtOAc 0 to 10% to afford the title compound (46 mg, 0.098 mmol, 47% yield) as a white solid. ¹H NMR (400 MHz, DMSO-d₆) δ 13.21 (s, 1H), 10.73 (s, 1H), 8.59 (s, 1H), 8.22 (s, 1H), 7.82 (s, 1H), 7.58-7.67 (m, 2H), 7.48 (s, 1H), 7.35-7.45 (m, 2H), 6.93-7.08 (m, 1H), 3.71-3.85 (m, 4H), 3.17-3.27 (m, 4H), 2.86 (s, 3H), 2.42 (d, J=0.88 Hz, 3H). MS-ESI (m/z) calc'd for C₂₆H₂₆N₇O₂ [M+H]⁺: 468.2. Found 468.3.

### Example 67. 4-Methyl-N-(3-phenyl-1H-indazol-5-yl)-[1,2,3]triazolo[1,5-a]pyridine-5-carboxamide

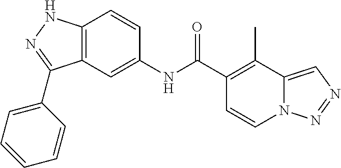 [SMILES: Cc1c(C(=O)Nc2ccc3[nH]nc(-c4ccccc4)c3c2)ccn2nncc12]

### Step 1. Ethyl 2,3,5-trichloroisonicotinate

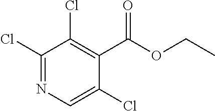

To a solution of 2,3,5-trichloropyridine-4-carboxylic acid (2.26 g, 10 mmol) in DMF (20 mL) was added potassium carbonate (5.53 g, 40 mmol) and iodoethane (1.61 mL, 20 mmol). The mixture was stirred at room temperature for 2 hrs. Water was added and the compound was extracted with EtOAc (3×), the combined organic layers were washed with water (2×), passed through a phase separator and evaporated to afford the title compound (2.55 g, 10 mmol, 100% yield) as a brown oil. ¹H NMR (400 MHz, DMSO-d₆) δ 8.70 (s, 1H), 4.48 (q, J=7.1 Hz, 2H), 1.34 (t, J=7.1 Hz, 3H). MS-ESI (m/z) calculated for C₈H₇Cl₃NO₂ [M+H]⁺: 254.0, 256.0. Found 254.0, 256.0.

### Step 2. Ethyl 3,5-dichloro-2-vinylisonicotinate

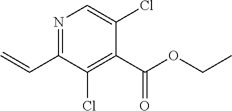

To a solution of ethyl 2,3,5-trichloroisonicotinate (2.55 g, 10 mmol) in 1,4-Dioxane (50 mL) was added 2-ethenyl-4,4,5,5-tetramethyl-1,3,2-dioxaborolane (2.54 mL, 15 mmol) and palladium tetrakis (1.16 g, 1.00 mmol). The mixture was stirred at r.t. for 5 minutes then a solution of potassium carbonate (1.38 g, 10 mmol) in water (50 mL) was added and the mixture was stirred at 80° C. for 2 hrs. The mixture was taken up in water and extracted with DCM (3×), the combined organic layers were passed through a phase separator and evaporated to obtain a residue which was purified chromatography (SiO₂, 100 g, EtOAc in cyclohexane [0%, 10%, 10 CV]) to afford the title compound (2.07 g, 8.42 mmol, 84% yield) as an orange oil. ¹H NMR (400 MHz, DMSO-d₆) δ 8.77 (s, 1H), 7.17 (dd, J=16.9, 10.7 Hz, 1H), 6.47 (dd, J=16.9, 2.0 Hz, 1H), 5.76 (dd, J=10.7, 2.0 Hz, 1H), 4.46 (q, J=7.0 Hz, 2H), 1.34 (t, J=7.1 Hz, 3H). MS-ESI (m/z) calculated for C₁₀H₁₀Cl₂NO₂ [M+H]⁺: 246.0, 248.0. Found 246.0, 248.0.

### Step 3. Ethyl 3,5-dichloro-2-formylisonicotinate

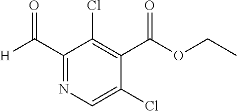

A solution of ethyl 3,5-dichloro-2-vinylisonicotinate (2.07 g, 8.42 mmol) in DCM (100 mL) was cooled to −78° C. and ozone was bubbled for 30 minutes. Triphenylphosphine (2.21 g, 8.42 mmol) was added portionwise and the mixture was stirred for 30 minutes. The solvent was evaporated and the residue was purified chromatography (SiO₂, 50 g, EtOAc in cyclohexane [0%, 20%, 1° C.V]) to obtain ethyl 3,5-dichloro-2-formylisonicotinate (0.81 g, 3.265 mmol, 39% yield) as a brown oil. ¹H NMR (400 MHz, DMSO-d₆) δ 10.06 (s, 1H), 9.02 (s, 1H), 4.49 (q, J=7.1 Hz, 2H), 1.35 (t, J=7.1 Hz, 3H). MS-ESI (m/z) calculated for C₉H₈Cl2NO₃ [M+H]⁺:248.0, 250.0. Found 248.0, 250.0.

### Step 4: Ethyl 4,6-dichloro-[1,2,3]triazolo[1,5-a]pyridine-5-carboxylate

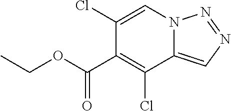 [SMILES: CCOC(=O)c1c(Cl)cn2nncc2c1Cl]

To a solution of ethyl 3,5-dichloro-2-formylisonicotinate (0.81 g, 3.27 mmol) in MeOH (16 mL) was added hydrazine hydrate (0.3 mL, 9.81 mmol) and the mixture was stirred at room temperature for 15 hrs. The solvent was evaporated and the residue was taken up in DCM (16 mL), then manganese (IV) oxide (568 mg, 6.54 mmol) was added and the mixture was stirred at room temperature for 1 hr. The black solid was filtered through a Celite pad and the filtrate was evaporated to afford the title compound (640 mg, 2.461 mmol, 75% yield) as a yellow solid. ¹H NMR (400 MHz, DMSO-d₆) δ 9.70 (d, J=1.0 Hz, 1H), 8.52 (d, J=1.0 Hz, 1H), 4.47 (q, J=7.1 Hz, 2H), 1.36 (t, J=7.1 Hz, 3H). MS-ESI (m/z) calculated for C₉H₈Cl₂N₃O₂ [M+H]⁺: 260.0, 262.0. Found 260.0 262.0.

### Step 5: Ethyl 6-chloro-4-methyl-[1,2,3]triazolo[1,5-a]pyridine-5-carboxylate

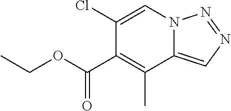 [SMILES: CCOC(=O)c1c(Cl)cn2nncc2c1C]

To a solution of ethyl 4,6-dichloro-[1,2,3]triazolo[1,5-a]pyridine-5-carboxylate (611 mg, 2.36 mmol) in 1,4-dioxane (24 mL) was added 2,4,6-trimethyl-1,3,5,2,4,6-trioxatriborinane (3.00 mL, 21.25 mmol), palladium triphenylphosphine (0.55 g, 0.470 mmol) and potassium carbonate (0.98 g, 7.08 mmol) in water (12 mL). The mixture was stirred at 90° C. for 3 hrs. The organic solvent was evaporated, the mixture was dilutes with water and extracted with EtOAc (3×), the combined organic layers were washed with brine, dried over Na₂SO₄ and evaporated to obtain a residue which was purified chromatography (SiO₂, 50 g, EtOAc in cyclohexane [0%, 50%, 10 CV; 50%, 50%, 5 CV]) to afford the title compound (405 mg, 1.69 mmol, 72% yield) as a yellow solid. ¹H NMR (400 MHz, DMSO-d₆) δ 9.47 (p, J=1.0 Hz, 1H), 8.50 (d, J=1.0 Hz, 1H), 4.43 (q, J=7.1 Hz, 2H), 2.54 (d, J=0.8 Hz, 3H), 1.35 (t, J=7.1 Hz, 3H). MS-ESI (m/z) calculated for C₁₀H₁₁ClN₃O₂ [M+H]⁺: 240.1, 242.1. Found 204.0, 242.0.

### Step 6: Ethyl 4-methyl-[1,2,3]triazolo[1,5-a]pyridine-5-carboxylate

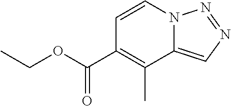

To a solution of ethyl 6-chloro-4-methyl-[1,2,3]triazolo[1,5-a]pyridine-5-carboxylate (400.0 mg, 1.67 mmol) in MeOH (17 mL) was added ammonium formate (421 mg, 6.68 mmol) and 10% palladium on carbon (178 mg, 0.170 mmol). The mixture was stirred 65° C. for 2 hrs. The catalyst was filtered through a Celite pad and the filtrate was evaporated. The residue was taken up in water and extracted with EtOAc (3×), the combined organic layers were passed through a phase separator and evaporated to afford the title compound (250 mg, 1.218 mmol, 73% yield) as a yellow solid. ¹H NMR (400 MHz, DMSO-d₆) δ 8.98 (dt, J=7.3, 1.0 Hz, 1H), 8.59 (d, J=1.1 Hz, 1H), 7.43 (d, J=7.3 Hz, 1H), 4.35 (q, J=7.1 Hz, 2H), 2.80-2.79 (m, 3H), 1.35 (t, J=7.1 Hz, 3H). MS-ESI (m/z) calculated for C₁₀H₁₂N₃O₂ [M+H]⁺: 206.1. Found 206.0.

### Step 7: 4-Methyl-[1,2,3]triazolo[1,5-a]pyridine-5-carboxylic acid

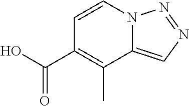

To a solution of ethyl 4-methyl-[1,2,3]triazolo[1,5-a]pyridine-5-carboxylate (60 mg, 0.290 mmol) in THF (1.5 mL) was added a solution of lithium hydroxide (21 mg, 0.880 mmol) in water (1.5 mL) and the mixture was stirred at room temperature for 4 hrs. The pH was adjusted to 1 by addition of 2M HCl and the solvent was evaporated to afford the title compound (89 mg, 0.292 mmol, 100% yield) as a yellow solid, which was used for the next step without any further purification. ¹H NMR (400 MHz, DMSO-d₆) δ 13.55 (s, 1H), 8.94 (d, J=7.3 Hz, 1H), 8.56 (s, 1H), 7.44 (d, J=7.3 Hz, 1H), 2.80 (s, 3H). MS-ESI (m/z) calculated for C₈H₈N₃O₂ [M+H]⁺: 178.1. Found 178.0.

### Step 8: 4-Methyl-N-(3-phenyl-1H-indazol-5-yl)-[1,2,3]triazolo[1,5-a]pyridine-5-carboxamide

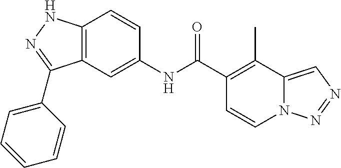 [SMILES: Cc1c(C(=O)Nc2ccc3[nH]nc(-c4ccccc4)c3c2)ccn2nncc12]

To a solution of 4-methyl-[1,2,3]triazolo[1,5-a]pyridine-5-carboxylic acid (52 mg, 0.290 mmol) in ACN (3 mL) was added triethylamine (41 uL, 0.290 mmol) and HATU (111 mg, 0.290 mmol). The mixture was stirred at room temperature for 15 minutes then 3-phenyl-1H-indazol-5-amine (63 mg, 0.290 mmol) was added and stirring was carried on for 8 hrs. The mixture was diluted with water and extracted with EtOAc (3×), the combined organic layers were washed with water (2×), passed through a phase separator and evaporated to obtain a residue which was purified by chromatography (SiO₂, 25 g, MeOH in DCM [0%, 10%, 15CV]) to afford the title compound (78 mg, 0.212 mmol, 72% yield) as an orange solid. ¹H NMR (400 MHz, DMSO-d₆) δ 13.26 (s, 1H), 10.58 (s, 1H), 9.06 (d, J=7.1 Hz, 1H), 8.60 (d, J=1.8 Hz, 1H), 8.48 (d, J=1.0 Hz, 1H), 8.02-7.88 (m, 2H), 7.68 (dd, J=9.0, 1.8 Hz, 1H), 7.61 (d, J=8.9 Hz, 1H), 7.55 (t, J=7.7 Hz, 2H), 7.46-7.38 (m, 1H), 7.31 (d, J=7.1 Hz, 1H), 2.65 (s, 3H). MS-ESI (m/z) calculated for C₂₁H₁₇N₆O [M+H]⁺: 369.1. Found 369.1.

### Example 68. N-(3-(Furan-3-yl)-1H-indazol-5-yl)-5,7-dimethyl-[1,2,3]triazolo[1,5-a]pyridine-6-carboxamide

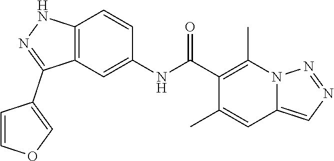 [SMILES: Cc1cc2cnnn2c(C)c1C(=O)Nc1ccc2[nH]nc(-c3ccoc3)c2c1]

### Step 1: 3-(Furan-3-yl)-1H-indazol-5-amine

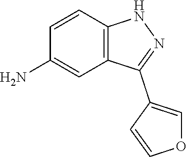 [SMILES: Nc1ccc2[nH]nc(-c3ccoc3)c2c1]

3-Furanylboronic acid (158.3 mg, 1.41 mmol), 3-bromo-1H-indazol-5-amine (Intermediate 1; 200 mg, 0.94 mmol) and K₃PO₄ (600.6 mg, 2.83 mmol) were dissolved in a mixture of THF (3 mL) and water (1 mL) in a capped microwave vial. The reaction mixture was degassed with nitrogen for 15 minutes and then SPhos Pd G2 (102 mg, 0.14 mmol) was added. The mixture was heated to 80° C. and stirred for 18 hrs. The reaction was cooled to room temperature, diluted with water and then extracted with EtOAc. The organic phase was filtered through a Celite pad and then concentrated under reduced pressure. The crude obtained was purified by reverse phase column chromatography, eluting with a gradient of ACN in water from 2% to 50% in presence of 0.1% ammonia aqueous solution) to afford the title compound (104 mg, 0.522 mmol, 55% yield) as a beige solid. ¹H NMR (400 MHz, DMSO-d₆) δ 12.61 (s, 1H), 8.15 (dd, J=1.43, 0.77 Hz, 1H), 7.78 (t, J=1.65 Hz, 1H), 7.26 (d, J=8.58 Hz, 1H), 6.99 (d, J=1.32 Hz, 1H), 6.94 (dd, J=1.76, 0.66 Hz, 1H), 6.83 (dd, J=8.80, 1.98 Hz, 1H), 4.80 (s, 2H). MS-ESI (m/z) calculated for C₁₁H₁₀N₃O [M+H]⁺: 200.1. Found 200.1.

### Step 2: N-(3-(Furan-3-yl)-1H-indazol-5-yl)-5,7-dimethyl-[1,2,3]triazolo[1,5-a]pyridine-6-carboxamide

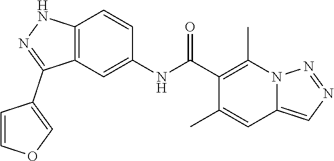 [SMILES: Cc1cc2cnnn2c(C)c1C(=O)Nc1ccc2[nH]nc(-c3ccoc3)c2c1]

To a solution of 3-(furan-3-yl)-1H-indazol-5-amine (52.1 mg, 0.26 mmol) and 5,7-dimethyltriazolo[1,5-a]pyridine-6-carboxylic acid (50 mg, 0.26 mmol) in DMF (2 mL), were added triethylamine (0.04 mL, 0.31 mmol) and HATU (109.4 mg, 0.29 mmol) at 0° C. The reaction was allowed to reach room temperature and stirring for further 18 hrs. The reaction mixture was diluted with water and extracted with EtOAc. The organic phase was separated, washed with brine, dried over Na₂SO₄, filtered and then concentrated under reduced pressure. The crude obtained was purified by normal phase column chromatography, eluting with a gradient of EtOAc in cyclohexane from 0% to 100% to afford the title compound (14.5 mg, 0.039 mmol, 15% yield) as a beige solid. ¹H NMR (400 MHz, DMSO-d₆) δ 13.14 (s, 1H), 10.71 (s, 1H), 8.41 (s, 1H), 8.25 (s, 1H), 8.22 (s, 1H), 7.77-7.93 (m, 2H), 7.53-7.66 (m, 2H), 6.95-7.06 (m, 1H), 2.87 (s, 3H), 2.43 (d, J=0.66 Hz, 3H). LC-MS: m/z=373.12 [M+H]⁺, 0.79 min. MS-ESI (m/z) calculated for C₂₀H₁₇N₆O₂ [M+H]⁺: 373.1. Found 373.1.

### Example 69. 5,7-Dimethyl-N-(3-phenyl-1H-indazol-5-yl)imidazo[1,5-a]pyridine-6-carboxamide

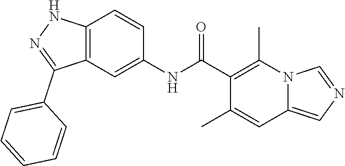 [SMILES: Cc1cc2cncn2c(C)c1C(=O)Nc1ccc2[nH]nc(-c3ccccc3)c2c1]

### Step 1: 6-Chloro-2,4-dimethylnicotinic acid

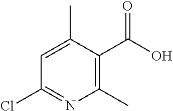

6-Chloro-2,4-dimethylnicotinonitrile (5.0 g, 30.01 mmol, 1 eq) was dissolved in a mixture of sulfuric acid (10.4 mL, 195.07 mmol, 6.5 eq) and water (9 mL). The mixture was heated at 120° C. for 16 hrs. Then it was then cooled to 90° C. and sodium nitrite (14.49 g, 210.07 mmol, 7 eq) was added in small portions over 10 minutes. The reaction was heated at 90° C. for an additional hour. Then other 3.5 eq. of NaNO₂ were added portionwise and the mixture was stirred at 90° C. for additional 2 hrs. The mixture was cooled to r.t. and poured into an ice-water mix. The chilled mixture was basified to pH=12 using NaOH aq. 10N. The aqueous mixture was then washed with DCM (2×) to remove impurities. The pH was then adjusted to 2 by adding HCl aq. 6N and a mixture 4:1 of DCM/MeOH was added. The off-white solid at the interphase was recovered by filtration and proved to be 6-hydroxy-2,4-dimethylpyridine-3-carboxylic acid (1.3 g, 7.77 mmol, 26% yield). ¹H NMR (400 MHz, DMSO-d₆) δ 12.90-11.00 (m, 2H), 6.05 (s, 1H), 2.30 (s, 3H), 2.18 (s, 3H). MS-ESI (m/z) calculated for C₈H₁₀NO₃ [M+H]⁺: 168.1. Found 168.2. The filtrate was also recovered, the 2 phases separated, the aqueous layer extracted with DCM/MeOH 4/1 (2×) and the combined extracts were washed with water, dried over anhydrous Na₂SO₄ and concentrated to dryness to afford the title compound (2.9 g, 15.62 mmol, 52% yield). ¹H NMR (400 MHz, DMSO-d₆) δ 13.84-13.55 (bs, 1H), 7.34 (s, 1H), 2.45 (s, 3H), 2.31 (s, 3H). MS-ESI (m/z) calculated for C₈H₉ClNO₂ [M+H]⁺: 186.0. Found 186.2.

### Step 2: Ethyl 6-chloro-2,4-dimethylnicotinate

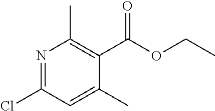

A suspension of 6-chloro-2,4-dimethylnicotinic acid (2.9 g, 15.62 mmol, 1 eq) in phosphorus(V) oxychloride (26.29 mL, 281.23 mmol, 18 eq) was stirred at 80° C. for 2 hrs, cooled to room temperature and then evaporated to dryness. The residue was cooled to 0° C. and EtOH (15 mL) was added dropwise. The mixture was stirred at room temperature for 1.5 hrs and then portioned between EtOAc and H₂O. The organic phase was concentrated in vacuo and the residue was purified by normal phase chromatography on a 100 g silica gel column, using as eluent a gradient of EtOAc in cyclohexane from 0 to 20% to afford the title compound (2.33 g, 10.91 mmol, 70% yield) as colorless oil. ¹H NMR (400 MHz, CDCl₃) δ 7.07 (s, 1H), 4.44 (q, J=7.2 Hz, 2H), 2.55 (s, 3H), 2.35 (s, 3H), 1.42 (t, J=7.2 Hz, 3H). MS-ESI (m/z) calculated for C₁₀H₁₃ClNO₂ [M+H]⁺: 214.1. Found 214.2.

### Step 3: Ethyl 2,4-dimethyl-6-vinylnicotinate

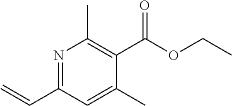

Ethyl 6-chloro-2,4-dimethylnicotinate (1.0 g, 4.21 mmol, 1 eq) was dissolved in toluene (12 mL) and the solution was degassed by bubbling nitrogen for 5 minutes. Then palladium triphenylphosphine (243.38 mg, 0.210 mmol, 0.05 eq), triphenylphosphine (110.48 mg, 0.420 mmol, 0.1 eq) and tributyl(ethenyl)stannane (1.6 mL, 5.48 mmol, 1.3 eq) were added. The mixture was heated at 100° C. overnight under nitrogen atmosphere. The reaction mixture was concentrated in vacuo and the crude purified by normal phase chromatography on a 100 g silica gel column, using as eluent a gradient of EtOAc in cyclohexane from 0 to 15%. The target compound (940 mg, 3.98 mmol, 94% yield) was obtained as a colorless oil. ¹H NMR (400 MHz, CDCl₃) δ 7.06 (s, 1H), 6.77 (dd, J=10.9, 17.5 Hz, 1H), 6.22 (dd, J=1.3, 17.4 Hz, 1H), 5.52 (dd, J=1.3, 10.8 Hz, 1H), 4.43 (q, J=7.0 Hz, 2H), 2.58 (s, 3H), 2.35 (s, 3H), 1.42 (t, J=7.2 Hz, 3H). MS-ESI (m/z) calculated for C₁₂H₁₆NO₂ [M+H]⁺: 206.1. Found 206.2.

### Step 4: Ethyl 6-formyl-2,4-dimethylnicotinate

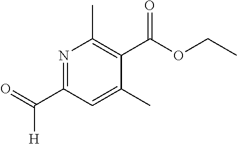

To a solution of ethyl 2,4-dimethyl-6-vinylnicotinate (500.0 mg, 87% pure by UPLC-UV, 2.12 mmol, 1 eq) in 1,4-dioxane (10 mL) was added a solution of sodium periodate (906.62 mg, 4.24 mmol, 2 eq) in water (10 mL). To the mixture, 4% osmium tetroxide (0.2 mL, 0.030 mmol, 0.015 eq) in water was added and the mixture was stirred at room temperature for 16 hrs. Then 1 additional equivalent of NaIO₄ was added and the mixture was stirred at room temperature for 1 hr. After that time the reaction was complete. The suspension was diluted with water and extracted with DCM (3×). The combined organic layers were passed through a phase separator and evaporated to dryness to afford the title compound (540 mg crude) as a dark oil. This material was used without further purification. ¹H NMR (400 MHz, CDCl₃) δ 10.04 (s, 1H), 7.68 (s, 1H), 4.48 (q, J=7.3 Hz, 2H), 2.65 (s, 3H), 2.43 (s, 3H), 1.44 (t, J=7.0 Hz, 3H). MS-ESI (m/z) calculated for C₁₁H₁₄NO₃ [M+H]⁺: 208.1. Found 208.2.

### Step 5: Ethyl (E)-6-((hydroxyimino)methyl)-2,4-dimethylnicotinate

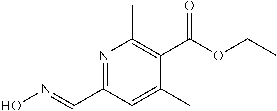 [SMILES: CCOC(=O)c1c(C)cc(/C=N/O)nc1C]

A mixture of ethyl 6-formyl-2,4-dimethylnicotinate (540 mg as crude, 2.12 mmol theoretical, 1 eq), potassium carbonate (351.44 mg, 2.54 mmol, 1.2 eq) and hydroxylamine hydrochloride (176.7 mg, 2.54 mmol, 1.2 eq) in MeOH (15 mL) was stirred at room temperature for 1 hr. Volatiles were removed under reduced pressure. The residue was portioned between water and EtOAc, the phases were separated, the aqueous layer was extracted with EtOAc (2×) and the combined organic phases washed with water (1×), dried over anhydrous Na₂SO₄ and evaporated to dryness. Target compound ethyl (E)-6-((hydroxyimino)methyl)-2,4-dimethylnicotinate (380 mg, 1.71 mmol, 81% yield) was obtained as a light brown solid. ¹H NMR (400 MHz, CDCl₃) δ 8.21 (s, 1H), 7.86 (s, 1H), 7.49 (s, 1H), 4.45 (q, J=7.3 Hz, 2H), 2.59 (s, 3H), 2.38 (s, 3H), 1.43 (t, J=7.2 Hz, 3H). MS-ESI (m/z) calculated for C₁₁H₁₅N₂O₃ [M+H]⁺: 223.1. Found 223.2.

### Step 6: Ethyl 6-(aminomethyl)-2,4-dimethylnicotinate

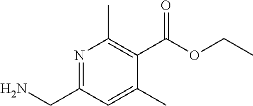

A solution of ethyl (E)-6-((hydroxyimino)methyl)-2,4-dimethylnicotinate (330 mg, 1.48 mmol, 1 eq) in EtOH (2.5 mL), water (4.5 mL) and acetic acid (4.5 mL) at 0° C. was treated slowly with zinc dust (485.41 mg, 7.42 mmol, 5 eq) and stirred for 20 minutes at the same temperature. The solids were filtered off and the solution concentrated under reduced pressure. The crude material was partitioned between DCM and sat. aqueous NaHCO₃. The phases were separated, the aqueous layer was washed with DCM (2×) and the combined organic phases extracted with water (1×). The target compound was present in the aqueous phase, which was evaporated to dryness. The residue was suspended in EtOH and the salts removed by filtration to afford the title compound (1.48 mmol theoretical). This was used without further purification in the next reaction. ¹H NMR (400 MHz, DMSO-d₆) δ 7.22 (s, 1H), 4.36 (q, J=7.0 Hz, 2H), 3.82 (s, 2H), 2.44 (s, 3H), 2.28 (s, 3H), 1.32 (t, J=7.0 Hz, 3H). MS-ESI (m/z) calculated for C₁₁H₁₇N₂O₂ [M+H]⁺: 209.1. Found 209.2.

### Step 7: Ethyl 6-(formamidomethyl)-2,4-dimethylnicotinate

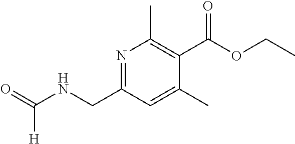 [SMILES: CCOC(=O)c1c(C)cc(CNC=O)nc1C]

A solution of ethyl 6-(aminomethyl)-2,4-dimethylnicotinate (1.48 mmol theoretical, 1 eq) in formic acid (7.0 mL) was refluxed (100° C.) for 1 hr. Volatiles were removed under reduced pressure. The residue was re-dissolved in DCM, and then washed with 10% NaHCO₃aq. solution (1×), water (1×) and dried over Na₂SO₄, filtered and concentrated to dryness to afford the title compound (180 mg, 0.762 mmol, 51% yield over two steps) as a yellow oil. ¹H NMR (400 MHz, DMSO-d₆) δ 8.57 (br. s., 1H), 8.18 (d, J=1.5 Hz, 1H), 7.07 (s, 1H), 4.43-4.31 (m, 4H), 2.43 (s, 3H), 2.27 (s, 3H), 1.32 (t, J=7.0 Hz, 3H). MS-ESI (m/z) calculated for C₁₂H₁₇N₂O₃ [M+H]⁺: 237.1. Found 237.1.

### Step 8: Ethyl 5,7-dimethylimidazo[1,5-a]pyridine-6-carboxylate

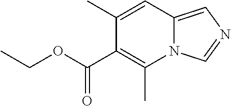 [SMILES: CCOC(=O)c1c(C)cc2cncn2c1C]

Ethyl 6-(formamidomethyl)-2,4-dimethylnicotinate (180.0 mg, 0.760 mmol, 1 eq) was dissolved in toluene (5 mL) and phosphorus(V) oxychloride (0.7 mL, 7.49 mmol, 10 eq) was added. The reaction was stirred at 65° C. for 1 hr. Volatiles were removed under reduced pressure. The residue was taken up in DCM and washed with saturated aqueous NaHCO₃ (1×), water (1×), passed through a phase separator and concentrated to dryness to afford the title compound (155 mg, 0.710 mmol, 93% yield) as a brown oil. ¹H NMR (400 MHz, DMSO-d₆) δ 8.35 (s, 1H), 7.40-7.32 (m, 2H), 4.38 (q, J=7.0 Hz, 2H), 2.59 (s, 3H), 2.22 (d, J=0.9 Hz, 3H), 1.34 (t, J=7.0 Hz, 3H). MS-ESI (m/z) calculated for C₁₂H₁₅N₂O₂ [M+H]⁺: 219.1. Found 219.1.

### Step 9: 5,7-Dimethylimidazo[1,5-a]pyridine-6-carboxylic acid

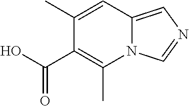

Ethyl 5,7-dimethylimidazo[1,5-a]pyridine-6-carboxylate (155.0 mg, 0.710 mmol, 1 eq) was dissolved in EtOH (1.5 mL) and a 2M aqueous solution of sodium hydroxide (4.0 mL, 8 mmol, 11 eq) was added. The solution was stirred at room temperature for 2 hrs and then at 50° C. overnight. Ethanol was removed under reduced pressure and then the aqueous residue acidified with HCl 2M until pH=1. The mixture was portioned between water and EtOAc, the phases were separated, the organic layer was extracted with water (1×) and the combined aqueous phases washed with EtOAc (1×) and evaporated to dryness. The product remained in the aqueous layer, which was therefore evaporated to dryness. The solid obtained was triturated in EtOH. The insoluble salts were removed by filtration, while the filtrate was collected and the solvent evaporated under reduced pressure to afford the title compound (140 mg, 0.662 mmol, 93% yield) as a light yellow solid. ¹H NMR (400 MHz, DMSO-d₆) δ 9.46 (s, 1H), 7.97 (s, 1H), 7.61 (s, 1H), 2.66 (s, 3H), 2.32 (s, 3H). MS-ESI (m/z) calculated for C₁₀H₁₁N₂O₂ [M+H]⁺: 191.1. Found 191.0.

### Step 10: 5,7-Dimethyl-N-(3-phenyl-1H-indazol-5-yl)imidazo[1,5-a]pyridine-6-carboxamide

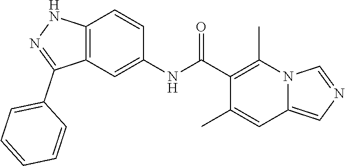 [SMILES: Cc1cc2cncn2c(C)c1C(=O)Nc1ccc2[nH]nc(-c3ccccc3)c2c1]

5,7-Dimethylimidazo[1,5-a]pyridine-6-carboxylic acid (35.0 mg, 0.180 mmol, 1 eq) and 3-phenyl-1H-indazol-5-amine (61.44 mg, 0.280 mmol, 1.5 eq) were dissolved in dry DMF (2 mL). Then the solution was cooled to 0° C. with an ice-water bath and HATU (83.96 mg, 0.220 mmol, 1.2 eq) and triethylamine (38.47 uL, 0.280 mmol, 1.5 eq) were added. The mixture was stirred at 0° C. for 5 minutes and then at r.t. overnight. The reaction mixture was portioned between water and EtOAc, the phases were separated, the aqueous layer was extracted with EtOAc (2×) and the combined organic phases washed with water (1×), dried over anhydrous Na₂SO₄ and evaporated to dryness. The crude was purified by reverse phase chromatography on a 12 g C18 column, using as eluent a gradient of CH₃CN in H₂O from 5 to 30% in presence of 0.1% formic acid. The purest fractions were combined and evaporated to dryness to afford crude material that was purified by normal phase chromatography on a 11 g NH-silica gel column, using as eluent a gradient of MeOH in EtOAc from 0 to 10%. The material was purified again by reverse phase chromatography on a 12 g C18 cartridge using as eluent a gradient of CH₃CN in H₂O from 5 to 45% in presence of 0.1% NH₃ to afford the title compound (10 mg, 0.026 mmol, 14% yield) as a white solid. ¹H NMR (400 MHz, DMSO-d₆) δ 13.26 (br. s., 1H), 10.60 (s, 1H), 8.62 (s, 1H), 8.37 (s, 1H), 8.03-7.90 (m, 2H), 7.67-7.51 (m, 4H), 7.46-7.36 (m, 3H), 2.61 (s, 3H), 2.28 (s, 3H). MS-ESI (m/z) calculated for C₂₃H₂₀N₅O [M+H]⁺: 382.2. Found 382.2.

### Example 70: N-(3-Phenyl-1H-indazol-5-yl)-5-(trifluoromethyl)-1H-indazole-6-carboxamide

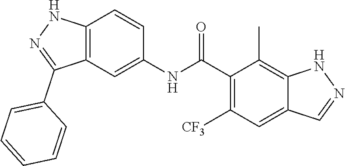 [SMILES: Cc1c(C(=O)Nc2ccc3[nH]nc(-c4ccccc4)c3c2)c(C(F)(F)F)cc2cn[nH]c12]

### Step 1: 5-Bromo-2-iodo-4-(trifluoromethyl)aniline

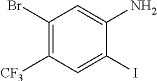 [SMILES: Nc1cc(Br)c(C(F)(F)F)cc1I]

To a solution of 3-bromo-4-(trifluoromethyl)aniline (1 g, 4.17 mmol) in AcOH (12 mL) was added NIS (937.35 mg, 4.17 mmol). The mixture was stirred at 25° C. for 2 hrs. The reaction mixture was concentrated and purified by flash silica gel chromatography (ISCO®; 12 g SepaFlash® Silica Flash Column, eluent of 0-3% EtOAc/petroleum ether gradient at 100 mL/min) to afford the title compound (1.45 g, 3.95 mmol, 95% yield) as a pink solid.

### Step 2: 5-Bromo-2-methyl-4-(trifluoromethyl)aniline

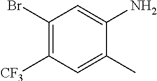

To a solution of 5-bromo-2-iodo-4-(trifluoromethyl)aniline (450 mg, 1.23 mmol) in dioxane (10 mL) was added methylboronic acid (88.34 mg, 1.48 mmol), Cs₂CO₃ (1.60 g, 4.92 mmol), Pd(dppf)Cl₂ (89.98 mg, 122.98 umol). The mixture was stirred at 90° C. for 12 hrs under N₂ atmosphere. The reaction mixture was concentrated and purified by flash silica gel chromatography (ISCO®; 12 g SepaFlash® Silica Flash Column, eluent of 0-8% EtOAc/petroleum ether gradient at 100 mL/min) to afford the title compound (120 mg, 472.35 umol, 38% yield) as an orange solid.

### Step 3: 6-Bromo-5-(trifluoromethyl)-1H-indazole

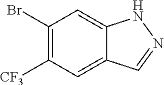 [SMILES: FC(F)(F)c1cc2cn[nH]c2cc1Br]

To a solution of 5-bromo-2-methyl-4-(trifluoromethyl)aniline (120 mg, 472.35 umol) in AcOH (2 mL) was added NaNO₂ (32.59 mg, 472.35 umol) in H₂O (0.1 mL) at 0° C. The mixture was stirred at 25° C. for 12 hrs. The reaction mixture was concentrated under reduced pressure to remove solvent. The residue was purified by preparative TLC (SiO₂, petroleum ether/EtOAc=3/1) to afford the title compound (100 mg, 377.32 umol, 80% yield) as an orange solid.

### Step 4: Methyl 5-(trifluoromethyl)-1H-indazole-6-carboxylate

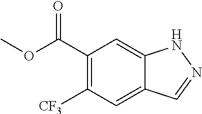 [SMILES: COC(=O)c1cc2[nH]ncc2cc1C(F)(F)F]

A mixture of 6-bromo-5-(trifluoromethyl)-1H-indazole (100 mg, 377.32 umol), Pd(dppf)Cl₂ (27.61 mg, 37.73 umol), TEA (1.53 g, 15.09 mmol) in MeOH (5 mL) was degassed and purged with CO (3×) and then the mixture was stirred at 120° C. for 12 hrs under CO atmosphere (4 MPa). The reaction mixture was concentrated under reduced pressure to remove solvent. The residue was purified by preparative HPLC (TFA condition) to afford the title compound (20 mg, 55.84 umol, 15% yield) as a red solid.

### Step 5: 5-(Trifluoromethyl)-1H-indazole-6-carboxylic acid

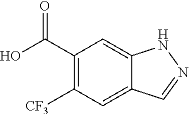 [SMILES: O=C(O)c1cc2[nH]ncc2cc1C(F)(F)F]

To a solution of methyl 5-(trifluoromethyl)-1H-indazole-6-carboxylate (60 mg, 245.73 umol) in MeOH (1 mL) and H₂O (1 mL) was added NaOH (49.14 mg, 1.23 mmol). The mixture was stirred at 40° C. for 12 hrs. The reaction mixture was diluted with H₂O (5 mL) and extracted with EtOAc (4 mL×2). The combined organic layers were discarded. Then the aqueous phase was acidified to pH=3 with 1 M HCl and extracted with EtOAc (3 mL×5). The combined organic layers were dried over Na₂SO₄, filtered and concentrated under reduced pressure to afford the title compound (54 mg, crude) as a white solid.

### Step 6: N-(3-Phenyl-1H-indazol-5-yl)-5-(trifluoromethyl)-1H-indazole-6-carboxamide

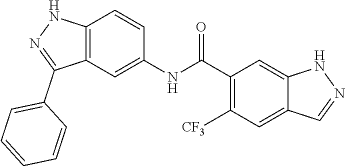 [SMILES: O=C(Nc1ccc2[nH]nc(-c3ccccc3)c2c1)c1cc2[nH]ncc2cc1C(F)(F)F]

To a solution of 5-(trifluoromethyl)-1H-indazole-6-carboxylic acid (77 mg, 334.57 umol) in DMF (5 mL) was added 3-phenyl-1H-indazol-5-amine (84.01 mg, 401.49 umol), HOBt (54.25 mg, 401.49 umol), EDCI (76.97 mg, 401.49 umol) and TEA (101.57 mg, 1.00 mmol). The mixture was stirred at 25° C. for 12 hrs. The reaction mixture was concentrated under reduced pressure to remove solvent. The residue was purified by preparative HPLC (TFA condition) to afford the title compound (1.82 mg, 3.25 umol, 1% yield) as a yellow gum. ¹H NMR (400 MHz, DMSO-d₆) δ 13.74 (br s, 1H) 13.26 (br s, 1H) 10.64 (br s, 1H) 8.56 (br s, 1H) 8.37 (br d, J=12.47 Hz, 2H) 7.87-8.00 (m, 3H) 7.38-7.69 (m, 5H). MS-ESI (m/z) calc'd for C₂₂H₁₅F₃N₅O [M+H]⁺: 422.1. Found 422.1.

### Example 71: 4,6-Difluoro-1-methyl-N-(3-(6-methylpyridin-2-yl)-1H-indazol-5-yl)-1H-indazole-5-carboxamide

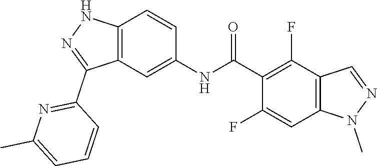 [SMILES: Cc1cccc(-c2n[nH]c3ccc(NC(=O)c4c(F)cc5c(cnn5C)c4F)cc23)n1]

To a solution of N-(3-bromo-1H-indazol-5-yl)-4,6-difluoro-1-methyl-1H-indazole-5-carboxamide (50 mg, 123.10 umol) and 2-methyl-6-(tributylstannyl)pyridine (61.16 mg, 160.03 umol) in dioxane (2 mL) was added Pd(PPh₃)₂Cl₂ (86.40 mg, 123.10 umol) and the reaction mixture was stirred at 120° C. for 12 hrs under N₂. The reaction mixture was filtered and the filtrate was concentrated. The residue was purified by preparative HPLC (TFA condition) and further purified by preparative HPLC (neutral condition) to afford the title compound (4.43 mg, 8.32 umol, 7% yield) as a yellow solid. ¹H NMR (400 MHz, DMSO-d₆) δ 13.32 (br s, 1H) 10.81 (s, 1H) 9.05 (d, J=1.34 Hz, 1H) 8.35 (d, J=0.73 Hz, 1H) 7.96 (d, J=7.82 Hz, 1H) 7.73-7.84 (m, 2H) 7.67 (d, J=9.54 Hz, 1H) 7.59 (d, J=8.93 Hz, 1H) 7.22 (d, J=7.58 Hz, 1H) 4.09 (s, 3H) 2.62 (s, 3H). MS-ESI (m/z) calc'd for C₂₂H₁₇F₂N₆O [M+H]⁺: 419.1. Found 419.1.

### Example 72: N-(3-(4,5-Dihydrofuran-2-yl)-1H-indazol-5-yl)-4,6-difluoro-1-methyl-1H-indazole-5-carboxamide

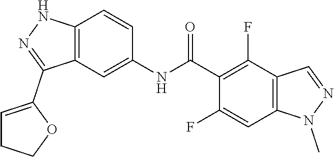 [SMILES: Cn1ncc2c(F)c(C(=O)Nc3ccc4[nH]nc(C5=CCCO5)c4c3)c(F)cc21]

Prepared as described for 4,6-difluoro-1-methyl-N-(3-(6-methylpyridin-2-yl)-1H-indazol-5-yl)-1H-indazole-5-carboxamide using tributyl(4,5-dihydrofuran-2-yl)stannane in place of 2-methyl-6-(tributylstannyl)pyridine. ¹H NMR (400 MHz, DMSO-d₆) δ 10.87 (s, 1H) 8.69 (s, 1H) 8.34 (s, 1H) 7.65-7.69 (m, 3H) 4.52 (s, 1H) 4.09 (s, 3H) 3.16 (t, J=7 Hz, 2H) 1.85 (quin, J=7 Hz, 2H). MS-ESI (m/z) calc'd for C₂₀H₁₆F₂N₅O [M+H]⁺: 396.1. Found 396.0.

### Example 73: 4,6-Difluoro-1-methyl-N-(3-(pyrimidin-4-yl)-1H-indazol-5-yl)-1H-indazole-5-carboxamide

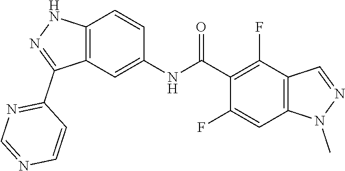 [SMILES: Cn1ncc2c(F)c(C(=O)Nc3ccc4[nH]nc(-c5ccncn5)c4c3)c(F)cc21]

Prepared as described for 4,6-difluoro-1-methyl-N-(3-(6-methylpyridin-2-yl)-1H-indazol-5-yl)-1H-indazole-5-carboxamide using 4-(tributylstannyl)pyrimidine in place of 2-methyl-6-(tributylstannyl)pyridine. ¹H NMR (400 MHz, DMSO-d₆) δ 13.81 (br s, 1H), 10.91 (s, 1H), 9.30 (s, 1H), 8.99 (s, 1H), 8.84 (d, J=5.3 Hz, 1H), 8.34 (s, 1H), 8.17 (br d, J=5.1 Hz, 1H), 7.80-7.75 (m, 1H), 7.67 (br dd, J=6.0, 9.0 Hz, 2H), 4.09 (s, 3H). MS-ESI (m/z) calc'd for C₂₀H₁₆F₂N₅O [M+H]⁺: 406.1. Found 406.1.

### Example 74: N-(3-(2-Cyanophenyl)-1H-indazol-5-yl)-4,6-difluoro-1-methyl-1H-indazole-5-carboxamide

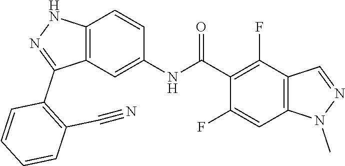 [SMILES: Cn1ncc2c(F)c(C(=O)Nc3ccc4[nH]nc(-c5ccccc5C#N)c4c3)c(F)cc21]

To a solution of N-(3-bromo-1H-indazol-5-yl)-4,6-difluoro-1-methyl-1H-indazole-5-carboxamide (50 mg, 123.10 umol) and (2-cyanophenyl)boronic acid (21.71 mg, 147.72 umol) in EtOH (2 mL) and H₂O (0.4 mL) was added Pd(Amphos)C₁₂ (8.72 mg, 12.31 umol) and KOAc (36.24 mg, 369.29 umol). The mixture was then stirred at 100° C. for 12 hrs under N₂. The mixture was extracted with EtOAc (3 mL×3) and water (3 mL). The organic layers were combined and dried over anhydrous Na₂SO₄, filtered and the filtrate was concentrated under vacuum. The residue was purified by preparative HPLC (TFA condition) to afford the title compound (5.08 mg, 9.26 umol, 8% yield) as a white solid. ¹H NMR (400 MHz, DMSO-d₆) δ 8.35 (s, 1H) 8.18 (s, 1H) 7.92-7.96 (m, 2H) 7.82-7.87 (m, 1H) 7.61-7.66 (m, 3H) 7.34 (d, J=9.54 Hz, 1H) 4.07 (s, 3H). MS-ESI (m/z) calc'd for C₂₃H₁₅F₂N₆O [M+H]⁺: 429.1. Found 429.0.

### Example 75: 4,6-Difluoro-N-(3-(2-hydroxyphenyl)-1H-indazol-5-yl)-1-methyl-1H-indazole-5-carboxamide

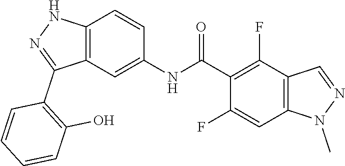 [SMILES: Cn1ncc2c(F)c(C(=O)Nc3ccc4[nH]nc(-c5ccccc5O)c4c3)c(F)cc21]

Prepared as described for N-(3-(2-cyanophenyl)-1H-indazol-5-yl)-4,6-difluoro-1-methyl-1H-indazole-5-carboxamide using (2-hydroxyphenyl)boronic acid in place of (2-cyanophenyl)boronic acid. ¹H NMR (400 MHz, DMSO-d₆) δ 13.40 (br s, 1H) 10.85 (s, 1H) 10.58 (br s, 1H) 8.61 (s, 1H) 8.35 (d, J=0.73 Hz, 1H) 7.80 (br d, J=6.48 Hz, 1H) 7.60-7.72 (m, 3H) 7.25-7.34 (m, 1H) 6.98-7.08 (m, 2H) 4.09 (s, 3H). MS-ESI (m/z) calc'd for C₂₂H₁₆F₂N₅O₂ [M+H]⁺: 420.1. Found 420.1.

### Example 76: 4,6-Difluoro-1-methyl-N-(3-(m-tolyl)-1H-indazol-5-yl)-1H-indazole-5-carboxamide

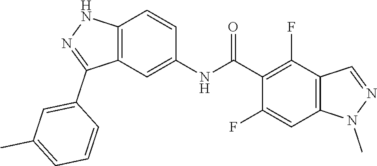 [SMILES: Cc1cccc(-c2n[nH]c3ccc(NC(=O)c4c(F)cc5c(cnn5C)c4F)cc23)c1]

Prepared as described for N-(3-(2-cyanophenyl)-1H-indazol-5-yl)-4,6-difluoro-1-methyl-1H-indazole-5-carboxamide using m-tolylboronic acid in place of (2-cyanophenyl)boronic acid. ¹H NMR (400 MHz, DMSO-d₆) δ 13.25 (s, 1H) 10.84 (s, 1H) 8.55 (s, 1H) 8.34 (s, 1H) 7.64-7.78 (m, 4H) 7.57-7.62 (m, 1H) 7.43 (t, J=7.61 Hz, 1H) 7.23 (d, J=7.50 Hz, 1H) 4.08 (s, 3H) 2.42 (s, 3H). MS-ESI (m/z) calc'd for C₂₃H₁₈F₂N₅O [M+H]⁺: 418.1. Found 418.1.

### Example 77: 4,6-Difluoro-1-methyl-N-(3-(o-tolyl)-1H-indazol-5-yl)-1H-indazole-5-carboxamide

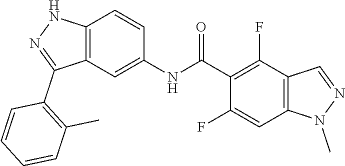 [SMILES: Cc1ccccc1-c1n[nH]c2ccc(NC(=O)c3c(F)cc4c(cnn4C)c3F)cc12]

Prepared as described for N-(3-(2-cyanophenyl)-1H-indazol-5-yl)-4,6-difluoro-1-methyl-1H-indazole-5-carboxamide using o-tolylboronic acid in place of (2-cyanophenyl)boronic acid. ¹H NMR (400 MHz, DMSO-d₆) δ 13.22 (br s, 1H) 10.79 (s, 1H) 8.33 (d, J=0.73 Hz, 1H) 8.18 (s, 1H) 7.65 (d, J=9.54 Hz, 1H) 7.55-7.63 (m, 2H) 7.46-7.52 (m, 1H) 7.38-7.44 (m, 1H) 7.34-7.38 (m, 2H) 4.08 (s, 3H) 2.37 (s, 3H). MS-ESI (m/z) calc'd for C₂₃H₁₈F₂N₅O [M+H]⁺: 418.1. Found 418.1.

### Example 78: 4,6-Difluoro-N-(3-(2-fluorophenyl)-1H-indazol-5-yl)-1-methyl-1H-indazole-5-carboxamide

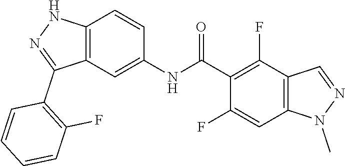 [SMILES: Cn1ncc2c(F)c(C(=O)Nc3ccc4[nH]nc(-c5ccccc5F)c4c3)c(F)cc21]

Prepared as described for N-(3-(2-cyanophenyl)-1H-indazol-5-yl)-4,6-difluoro-1-methyl-1H-indazole-5-carboxamide using (2-fluorophenyl)boronic acid in place of (2-cyanophenyl)boronic acid. ¹H NMR (400 MHz, DMSO-d₆) δ 13.42 (br s, 1H), 10.81 (s, 1H), 8.33 (d, J=0.7 Hz, 1H), 8.32-8.29 (m, 1H), 7.78 (dt, J=1.8, 7.6 Hz, 1H), 7.67-7.60 (m, 3H), 7.55-7.48 (m, 1H), 7.46-7.34 (m, 2H), 4.08 (s, 3H). MS-ESI (m/z) calc'd for C₂₂H₁₅F₃N₅O [M+H]⁺: 422.1. Found 422.1.

### Example 79: N-(3-(1,3-Dimethyl-1H-pyrazol-5-yl)-1H-indazol-5-yl)-4,6-difluoro-1-methyl-1H-indazole-5-carboxamide

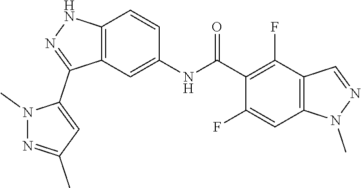 [SMILES: Cc1cc(-c2n[nH]c3ccc(NC(=O)c4c(F)cc5c(cnn5C)c4F)cc23)n(C)n1]

Prepared as described for N-(3-(2-cyanophenyl)-1H-indazol-5-yl)-4,6-difluoro-1-methyl-1H-indazole-5-carboxamide using (1,3-dimethyl-1H-pyrazol-5-yl)boronic acid in place of (2-cyanophenyl)boronic acid. ¹H NMR (400 MHz, DMSO-d₆) δ 13.46 (br s, 1H), 10.86 (s, 1H), 8.40 (d, J=0.7 Hz, 1H), 8.34 (d, J=0.9 Hz, 1H), 7.72-7.60 (m, 3H), 6.51 (s, 1H), 4.08 (s, 3H), 4.03 (s, 3H), 2.24 (s, 3H). MS-ESI (m/z) calc'd for C₂₁H₁₈F₂N₇O [M+H]⁺: 422.2. Found 422.2.

### Example 80: N-(3-(1,3-Dimethyl-1H-pyrazol-4-yl)-1H-indazol-5-yl)-4,6-difluoro-1-methyl-1H-indazole-5-carboxamide

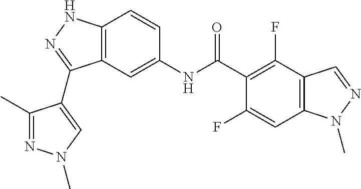 [SMILES: Cc1nn(C)cc1-c1n[nH]c2ccc(NC(=O)c3c(F)cc4c(cnn4C)c3F)cc12]

Prepared as described for N-(3-(2-cyanophenyl)-1H-indazol-5-yl)-4,6-difluoro-1-methyl-1H-indazole-5-carboxamide using (1,3-dimethyl-1H-pyrazol-4-yl)boronic acid in place of (2-cyanophenyl)boronic acid. ¹H NMR (400 MHz, DMSO-d₆) δ 12.97 (s, 1H) 10.76 (s, 1H) 8.30 (d, J=9.26 Hz, 2H) 8.04 (s, 1H) 7.64 (d, J=9.70 Hz, 1H) 7.49-7.59 (m, 2H) 4.06 (s, 3H) 3.85 (s, 3H) 2.38 (s, 3H). MS-ESI (m/z) calc'd for C₂₁H₁₈F₂N₇O [M+H]⁺: 422.2. Found 422.1.

### Example 81: 4,6-Difluoro-N-(3-(2-methoxypyridin-3-yl)-1H-indazol-5-yl)-1-methyl-1H-indazole-5-carboxamide

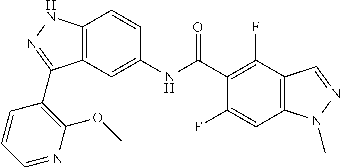 [SMILES: COc1ncccc1-c1n[nH]c2ccc(NC(=O)c3c(F)cc4c(cnn4C)c3F)cc12]

Prepared as described for N-(3-(2-cyanophenyl)-1H-indazol-5-yl)-4,6-difluoro-1-methyl-1H-indazole-5-carboxamide using (2-methoxypyridin-3-yl)boronic acid in place of (2-cyanophenyl)boronic acid. ¹H NMR (400 MHz, DMSO-d₆) δ 13.28 (br s, 1H) 10.74 (s, 1H) 8.31 (s, 1H) 8.21-8.29 (m, 2H) 7.97 (dd, J=7.39, 1.87 Hz, 1H) 7.63 (d, J=9.70 Hz, 1H) 7.52-7.60 (m, 2H) 7.13 (dd, J=7.39, 4.96 Hz, 1H) 4.06 (s, 3H) 3.94 (s, 3H). MS-ESI (m/z) calc'd for C₂₂H₁₇F₂N₆O₂ [M+H]⁺: 435.1. Found 435.0.

### Example 82: 4,6-Difluoro-N-(3-(5-methoxypyridin-3-yl)-1H-indazol-5-yl)-1-methyl-1H-indazole-5-carboxamide

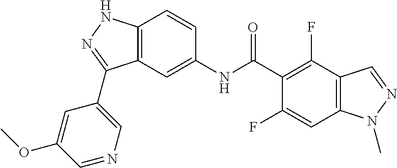 [SMILES: COc1cncc(-c2n[nH]c3ccc(NC(=O)c4c(F)cc5c(cnn5C)c4F)cc23)c1]

Prepared as described for N-(3-(2-cyanophenyl)-1H-indazol-5-yl)-4,6-difluoro-1-methyl-1H-indazole-5-carboxamide using (5-methoxypyridin-3-yl)boronic acid in place of (2-cyanophenyl)boronic acid. ¹H NMR (400 MHz, DMSO-d₆) δ 13.48 (br s, 1H) 10.87 (s, 1H) 8.75 (s, 1H) 8.61 (s, 1H) 8.35 (br s, 2H) 7.82 (br s, 1H) 7.63-7.72 (m, 3H) 4.08 (s, 3H) 3.94 (s, 3H). MS-ESI (m/z) calc'd for C₂₂H₁₇F₂N₆O₂ [M+H]⁺: 435.1. Found 435.1.

### Example 83: N-(3-(2,6-Dimethylpyridin-4-yl)-1H-indazol-5-yl)-4,6-difluoro-1-methyl-1H-indazole-5-carboxamide

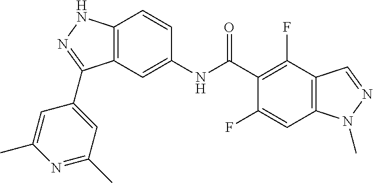 [SMILES: Cc1cc(-c2n[nH]c3ccc(NC(=O)c4c(F)cc5c(cnn5C)c4F)cc23)cc(C)n1]

Prepared as described for N-(3-(2-cyanophenyl)-1H-indazol-5-yl)-4,6-difluoro-1-methyl-1H-indazole-5-carboxamide using (2,6-dimethylpyridin-4-yl)boronic acid in place of (2-cyanophenyl)boronic acid. ¹H NMR (400 MHz, DMSO-d₆) δ 14.23 (s, 1H) 11.01 (s, 1H) 8.71 (s, 1H) 8.34 (s, 1H) 8.17 (s, 2H) 7.77 (s, 2H) 7.64-7.71 (m, 1H) 4.09 (s, 3H) 2.76 (s, 6H). MS-ESI (m/z) calc'd for C₂₃H₁₉F₂N₆O [M+H]⁺: 433.2. Found 433.1.

### Example 84: 4,6-Difluoro-N-(3-(3-methoxyphenyl)-1H-indazol-5-yl)-1-methyl-1H-indazole-5-carboxamide

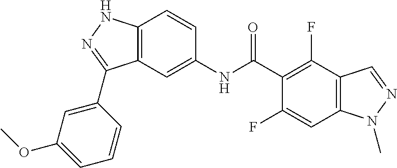 [SMILES: COc1cccc(-c2n[nH]c3ccc(NC(=O)c4c(F)cc5c(cnn5C)c4F)cc23)c1]

Prepared as described for N-(3-(2-cyanophenyl)-1H-indazol-5-yl)-4,6-difluoro-1-methyl-1H-indazole-5-carboxamide using (3-methoxyphenyl)boronic acid in place of (2-cyanophenyl)boronic acid. ¹H NMR (400 MHz, DMSO-d₆) δ 13.13-13.41 (m, 1H) 10.85 (s, 1H) 8.60 (s, 1H) 8.34 (s, 1H) 7.58-7.70 (m, 3H) 7.41-7.54 (m, 3H) 6.95-7.02 (m, 1H) 4.08 (s, 3H) 3.85 (s, 3H). MS-ESI (m/z) calc'd for C₂₃H₁₈F₂N₅O₂ [M+H]⁺: 434.1. Found 434.1.

### Example 85: 4,6-Difluoro-N-(3-(2-methoxyphenyl)-1H-indazol-5-yl)-1-methyl-1H-indazole-5-carboxamide

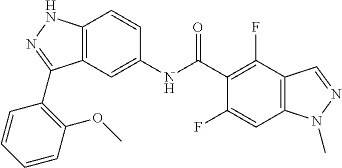 [SMILES: COc1ccccc1-c1n[nH]c2ccc(NC(=O)c3c(F)cc4c(cnn4C)c3F)cc12]

Prepared as described for N-(3-(2-cyanophenyl)-1H-indazol-5-yl)-4,6-difluoro-1-methyl-1H-indazole-5-carboxamide using (2-methoxyphenyl)boronic acid in place of (2-cyanophenyl)boronic acid. ¹H NMR (400 MHz, DMSO-d₆) δ 13.15 (br s, 1H), 10.71 (s, 1H), 8.32 (d, J=0.9 Hz, 1H), 8.17 (s, 1H), 7.64 (d, J=9.7 Hz, 1H), 7.56-7.52 (m, 3H), 7.47-7.41 (m, 1H), 7.20 (d, J=7.9 Hz, 1H), 7.07 (dt, J=0.8, 7.4 Hz, 1H), 4.07 (s, 3H), 3.83 (s, 3H). MS-ESI (m/z) calc'd for C₂₃H₁₈F₂N₅O₂ [M+H]⁺: 434.1. Found 434.1.

### Example 86: N-(3-(1-(Difluoromethyl)-1H-pyrazol-4-yl)-1H-indazol-5-yl)-4,6-difluoro-1-methyl-1H-indazole-5-carboxamide

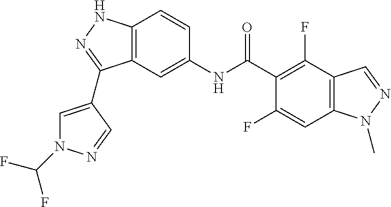 [SMILES: Cn1ncc2c(F)c(C(=O)Nc3ccc4[nH]nc(-c5cnn(C(F)F)c5)c4c3)c(F)cc21]

Prepared as described for N-(3-(2-cyanophenyl)-1H-indazol-5-yl)-4,6-difluoro-1-methyl-1H-indazole-5-carboxamide using (1-(difluoromethyl)-1H-pyrazol-4-yl)boronic acid in place of (2-cyanophenyl)boronic acid. ¹H NMR (400 MHz, DMSO-d₆) δ 13.23 (s, 1H), 10.83 (s, 1H), 8.72 (s, 1H), 8.43 (s, 1H), 8.34 (s, 1H), 8.28 (s, 1H), 8.09-7.79 (m, 1H), 7.67 (d, J=9.7 Hz, 1H), 7.63-7.57 (m, 2H), 4.08 (s, 3H). MS-ESI (m/z) calc'd for C₂₀H₁₄F₄N₇O [M+H]⁺: 444.1. Found 444.1.

### Example 87: N-(3-(4-(Dimethylamino)phenyl)-1H-indazol-5-yl)-4,6-difluoro-1-methyl-1H-indazole-5-carboxamide

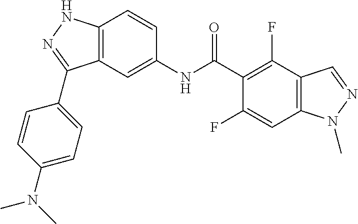 [SMILES: CN(C)c1ccc(-c2n[nH]c3ccc(NC(=O)c4c(F)cc5c(cnn5C)c4F)cc23)cc1]

Prepared as described for N-(3-(2-cyanophenyl)-1H-indazol-5-yl)-4,6-difluoro-1-methyl-1H-indazole-5-carboxamide using (4-(dimethylamino)phenyl)boronic acid in place of (2-cyanophenyl)boronic acid. ¹H NMR (400 MHz, DMSO-d₆) δ 13.00 (s, 1H) 10.81 (s, 1H) 8.58 (s, 1H) 8.35 (s, 1H) 7.79 (br d, J=8 Hz, 2H) 7.67 (d, J=10 Hz, 1H) 7.57 (s, 2H) 6.96 (br s, 2H) 4.09 (s, 3H) 2.99 (s, 6H). MS-ESI (m/z) calc'd for C₂₄H₂₀F2N₆O [M+H]⁺: 447.2. Found 447.1.

### Example 88: N-(3-(3-(Dimethylamino)phenyl)-1H-indazol-5-yl)-4,6-difluoro-1-methyl-1H-indazole-5-carboxamide

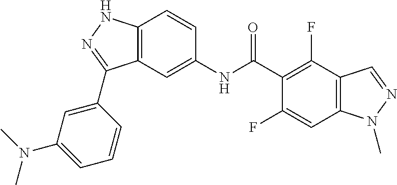 [SMILES: CN(C)c1cccc(-c2n[nH]c3ccc(NC(=O)c4c(F)cc5c(cnn5C)c4F)cc23)c1]

Prepared as described for N-(3-(2-cyanophenyl)-1H-indazol-5-yl)-4,6-difluoro-1-methyl-1H-indazole-5-carboxamide using (3-(dimethylamino)phenyl)boronic acid in place of (2-cyanophenyl)boronic acid. ¹H NMR (400 MHz, DMSO-d₆) δ 13.17 (br s, 1H) 10.84 (s, 1H) 8.65 (s, 1H) 8.35 (d, J=1 Hz, 1H) 7.67 (d, J=10 Hz, 1H) 7.61 (s, 2H) 7.33-7.41 (m, 2H) 7.29 (br d, J=7 Hz, 1H) 6.88 (br s, 1H) 4.09 (s, 3H) 3.02 (s, 6H). MS-ESI (m/z) calc'd for C₂₄H₂₀F₂N₆O [M+H]⁺: 447.2. Found 447.1.

### Example 89: 4,6-Difluoro-N-(3-(2-methoxy-5-methylphenyl)-1H-indazol-5-yl)-1-methyl-1H-indazole-5-carboxamide

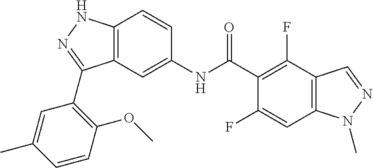 [SMILES: COc1ccc(C)cc1-c1n[nH]c2ccc(NC(=O)c3c(F)cc4c(cnn4C)c3F)cc12]

Prepared as described for N-(3-(2-cyanophenyl)-1H-indazol-5-yl)-4,6-difluoro-1-methyl-1H-indazole-5-carboxamide using (2-methoxy-5-methylphenyl)boronic acid in place of (2-cyanophenyl)boronic acid. ¹H NMR (400 MHz, DMSO-d₆) δ 10.72 (s, 1H) 8.32 (s, 1H) 8.15 (s, 1H) 7.65 (d, J=9.48 Hz, 1H) 7.50-7.58 (m, 2H) 7.34 (d, J=1.76 Hz, 1H) 7.20-7.27 (m, 1H) 7.08 (d, J=8.38 Hz, 1H) 4.07 (s, 3H) 3.78 (s, 3H) 2.31 (s, 3H). MS-ESI (m/z) calc'd for C₂₄H₂₀F₂N₅O₂ [M+H]⁺: 448.2. Found 448.1.

### Example 90: 4,6-Difluoro-N-(3-(5-methoxy-2-methylphenyl)-1H-indazol-5-yl)-1-methyl-1H-indazole-5-carboxamide

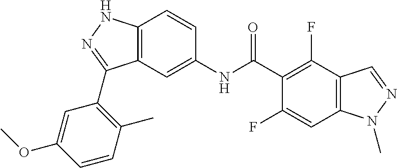 [SMILES: COc1ccc(C)c(-c2n[nH]c3ccc(NC(=O)c4c(F)cc5c(cnn5C)c4F)cc23)c1]

Prepared as described for N-(3-(2-cyanophenyl)-1H-indazol-5-yl)-4,6-difluoro-1-methyl-1H-indazole-5-carboxamide using (5-methoxy-2-methylphenyl)boronic acid in place of (2-cyanophenyl)boronic acid. ¹H NMR (400 MHz, DMSO-d₆) δ 8.17 (s, 1H) 8.12 (s, 1H) 7.58-7.63 (m, 2H) 7.34 (d, J=9.48 Hz, 1H) 7.29 (d, J=8.16 Hz, 1H) 7.05 (d, J=2.65 Hz, 1H) 6.94 (dd, J=8.49, 2.76 Hz, 1H) 4.07 (s, 3H) 3.83 (s, 3H) 2.28 (s, 3H). MS-ESI (m/z) calc'd for C₂₄H₂₀F₂N₅O₂ [M+H]⁺: 448.2. Found 448.1.

### Example 91: 4,6-Difluoro-1-methyl-N-(3-(4-(trifluoromethyl)pyridin-3-yl)-1H-indazol-5-yl)-1H-indazole-5-carboxamide

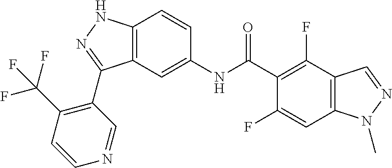 [SMILES: Cn1ncc2c(F)c(C(=O)Nc3ccc4[nH]nc(-c5cnccc5C(F)(F)F)c4c3)c(F)cc21]

Prepared as described for N-(3-(2-cyanophenyl)-1H-indazol-5-yl)-4,6-difluoro-1-methyl-1H-indazole-5-carboxamide using (4-(trifluoromethyl)pyridin-3-yl)boronic acid in place of (2-cyanophenyl)boronic acid. ¹H NMR (400 MHz, DMSO-d₆) δ 13.57 (s, 1H), 10.83 (s, 1H), 9.02-8.93 (m, 2H), 8.36-8.29 (m, 1H), 8.21-8.17 (m, 1H), 7.98 (d, J=5.1 Hz, 1H), 7.67-7.57 (m, 3H), 4.09-4.06 (m, 3H). MS-ESI (m/z) calc'd for C₂₂H₁₄F₅N₆O [M+H]⁺: 473.1. Found 473.0.

### Example 92: 4,6-Difluoro-1-methyl-N-(3-(1-methyl-3-(trifluoromethyl)-1H-pyrazol-4-yl)-1H-indazol-5-yl)-1H-indazole-5-carboxamide

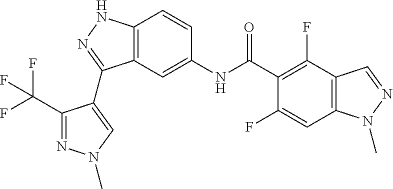 [SMILES: Cn1cc(-c2n[nH]c3ccc(NC(=O)c4c(F)cc5c(cnn5C)c4F)cc23)c(C(F)(F)F)n1]

Prepared as described for N-(3-(2-cyanophenyl)-1H-indazol-5-yl)-4,6-difluoro-1-methyl-1H-indazole-5-carboxamide using (1-methyl-3-(trifluoromethyl)-1H-pyrazol-4-yl)boronic acid in place of (2-cyanophenyl)boronic acid. ¹H NMR (400 MHz, DMSO-d₆) δ 13.25 (br s, 1H) 10.81 (br s, 1H) 8.36 (br d, J=17.42 Hz, 2H) 8.27 (s, 1H) 7.66 (br d, J=9.70 Hz, 1H) 7.54-7.60 (m, 2H) 4.08 (s, 3H) 4.04 (s, 3H). MS-ESI (m/z) calc'd for C₂₁H₁₅F₅N₇O [M+H]⁺: 476.1. Found 476.1.

### Example 93: 4,6-Difluoro-1-methyl-N-(3-(2-(trifluoromethoxy)phenyl)-1H-indazol-5-yl)-1H-indazole-5-carboxamide

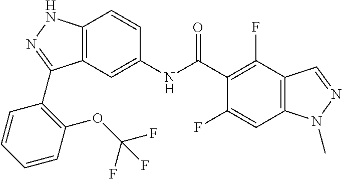 [SMILES: Cn1ncc2c(F)c(C(=O)Nc3ccc4[nH]nc(-c5ccccc5OC(F)(F)F)c4c3)c(F)cc21]

Prepared as described for N-(3-(2-cyanophenyl)-1H-indazol-5-yl)-4,6-difluoro-1-methyl-1H-indazole-5-carboxamide using (2-(trifluoromethoxy)phenyl)boronic acid in place of (2-cyanophenyl)boronic acid. ¹H NMR (400 MHz, DMSO-d₆) δ 8.24-8.15 (m, 2H), 7.79 (d, J=7.0 Hz, 1H), 7.71-7.64 (m, 1H), 7.64-7.48 (m, 4H), 7.34 (d, J=9.2 Hz, 1H), 4.07 (s, 3H). MS-ESI (m/z) calc'd for C₂₃H₁₅F₅N₅O₂ [M+H]⁺: 488.1. Found 488.0.

### Example 94: 4,6-Difluoro-1-methyl-N-(3-(4-morpholinophenyl)-1H-indazol-5-yl)-1H-indazole-5-carboxamide

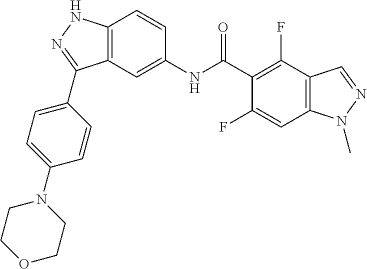 [SMILES: Cn1ncc2c(F)c(C(=O)Nc3ccc4[nH]nc(-c5ccc(N6CCOCC6)cc5)c4c3)c(F)cc21]

Prepared as described for N-(3-(2-cyanophenyl)-1H-indazol-5-yl)-4,6-difluoro-1-methyl-1H-indazole-5-carboxamide using (4-morpholinophenyl)boronic acid in place of (2-cyanophenyl)boronic acid. ¹H NMR (400 MHz, DMSO-d₆) δ 13.04 (br s, 1H), 10.79 (s, 1H), 8.54 (s, 1H), 8.32 (d, J=0.7 Hz, 1H), 7.78 (d, J=8.8 Hz, 2H), 7.64 (d, J=9.7 Hz, 1H), 7.60-7.50 (m, 2H), 7.09 (d, J=8.9 Hz, 2H), 4.06 (s, 3H), 3.80-3.69 (m, 4H), 3.22-3.11 (m, 4H). MS-ESI (m/z) calc'd for C₂₆H₂₃F₂N₆O₂ [M+H]⁺: 489.2. Found 489.1.

### Example 95: 4,6-Difluoro-1-methyl-N-(3-(2-methylthiazol-5-yl)-1H-indazol-5-yl)-1H-indazole-5-carboxamide

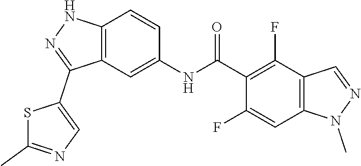 [SMILES: Cc1ncc(-c2n[nH]c3ccc(NC(=O)c4c(F)cc5c(cnn5C)c4F)cc23)s1]

Prepared as described for N-(3-(2-cyanophenyl)-1H-indazol-5-yl)-4,6-difluoro-1-methyl-1H-indazole-5-carboxamide using (2-methylthiazol-5-yl)boronic acid in place of (2-cyanophenyl)boronic acid. ¹H NMR (400 MHz, DMSO-d₆) δ 13.37 (s, 1H) 10.87 (s, 1H) 8.55 (s, 1H) 8.34 (s, 1H) 8.08 (s, 1H) 7.64-7.69 (m, 2H) 7.60-7.64 (m, 1H) 4.08 (s, 3H) 2.72 (s, 3H). MS-ESI (m/z) calc'd for C₂₀H₁₅F₂N₆OS [M+H]⁺: 425.1. Found 425.1.

### Example 96: 4,6-Difluoro-1-methyl-N-(3-(1-methyl-1H-pyrazol-5-yl)-1H-indazol-5-yl)-1H-indazole-5-carboxamide

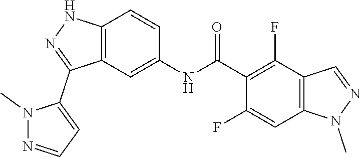 [SMILES: Cn1nccc1-c1n[nH]c2ccc(NC(=O)c3c(F)cc4c(cnn4C)c3F)cc12]

Prepared as described for N-(3-(2-cyanophenyl)-1H-indazol-5-yl)-4,6-difluoro-1-methyl-1H-indazole-5-carboxamide using (1-methyl-1H-pyrazol-5-yl)boronic acid in place of (2-cyanophenyl)boronic acid. ¹H NMR (400 MHz, DMSO-d₆) δ 8.39 (s, 1H), 8.18 (s, 1H), 7.68-7.59 (m, 3H), 7.35 (d, J=9.5 Hz, 1H), 6.82 (d, J=2.1 Hz, 1H), 4.17 (s, 3H), 4.08 (s, 3H). MS-ESI (m/z) calc'd for C₂₀H₁₆F₂N₇O [M+H]⁺: 408.1. Found 408.1.

### Example 97: 4,6-Difluoro-1-methyl-N-(3-(5-methylthiophen-2-yl)-1H-indazol-5-yl)-1H-indazole-5-carboxamide

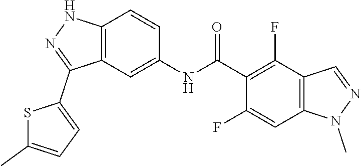 [SMILES: Cc1ccc(-c2n[nH]c3ccc(NC(=O)c4c(F)cc5c(cnn5C)c4F)cc23)s1]

Prepared as described for N-(3-(2-cyanophenyl)-1H-indazol-5-yl)-4,6-difluoro-1-methyl-1H-indazole-5-carboxamide using (5-methylthiophen-2-yl)boronic acid in place of (2-cyanophenyl)boronic acid. ¹H NMR (400 MHz, DMSO-d₆) δ 13.14 (br s, 1H), 10.83 (s, 1H), 8.59 (s, 1H), 8.34 (s, 1H), 7.67 (d, J=9.7 Hz, 1H), 7.63-7.54 (m, 2H), 7.38 (d, J=3.5 Hz, 1H), 6.91 (d, J=2.4 Hz, 1H), 4.09 (s, 3H). MS-ESI (m/z) calc'd for C₂₁H₁₆F₂N₅OS [M+H]⁺: 424.1. Found 424.1.

### Example 98: 4,6-Difluoro-1-methyl-N-(3-(5-morpholinopyridin-3-yl)-1H-indazol-5-yl)-1H-indazole-5-carboxamide

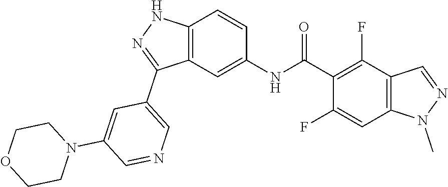 [SMILES: Cn1ncc2c(F)c(C(=O)Nc3ccc4[nH]nc(-c5cncc(N6CCOCC6)c5)c4c3)c(F)cc21]

Prepared as described for N-(3-(2-cyanophenyl)-1H-indazol-5-yl)-4,6-difluoro-1-methyl-1H-indazole-5-carboxamide using (5-morpholinopyridin-3-yl)boronic acid in place of (2-cyanophenyl)boronic acid. ¹H NMR (400 MHz, DMSO-d₆) δ 13.41 (br s, 1H), 10.85 (s, 1H), 8.57 (br d, J=3.2 Hz, 2H), 8.39 (d, J=2.6 Hz, 1H), 8.34 (s, 1H), 7.74 (br s, 1H), 7.71-7.56 (m, 3H), 4.08 (s, 3H), 3.82-3.75 (m, 4H), 3.30-3.26 (m, 4H). MS-ESI (m/z) calc'd for C₂₅H₂₂F₂N₇O₂ [M+H]⁺: 490.2. Found 490.1.

### Example 99: 4,6-Difluoro-1-methyl-N-(3-propyl-1H-indazol-5-yl)-1H-indazole-5-carboxamide

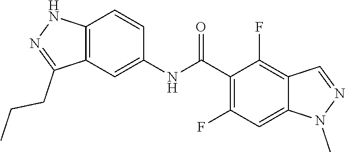 [SMILES: CCCc1n[nH]c2ccc(NC(=O)c3c(F)cc4c(cnn4C)c3F)cc12]

Prepared as described for N-(3-(2-cyanophenyl)-1H-indazol-5-yl)-4,6-difluoro-1-methyl-1H-indazole-5-carboxamide using propylboronic acid in place of (2-cyanophenyl)boronic acid. ¹H NMR (400 MHz, DMSO-d₆) δ 12.64 (s, 1H), 10.73 (s, 1H), 8.33 (s, 1H), 8.24 (s, 1H), 7.65 (d, J=9.7 Hz, 1H), 7.53-7.42 (m, 2H), 4.08 (s, 3H), 2.87 (t, J=7.5 Hz, 2H), 1.83-1.71 (m, 2H), 0.96 (t, J=7.3 Hz, 3H). MS-ESI (m/z) calc'd for C₁₉H₁₈F₂N₅O [M+H]⁺: 370.2. Found 370.1.

### Example 100: 4,6-Difluoro-1-methyl-N-(3-(thiophen-3-yl)-1H-indazol-5-yl)-1H-indazole-5-carboxamide

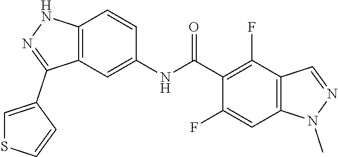 [SMILES: Cn1ncc2c(F)c(C(=O)Nc3ccc4[nH]nc(-c5ccsc5)c4c3)c(F)cc21]

Prepared as described for N-(3-(2-cyanophenyl)-1H-indazol-5-yl)-4,6-difluoro-1-methyl-1H-indazole-5-carboxamide using 3-thienylboronic acid in place of (2-cyanophenyl)boronic acid. ¹H NMR (400 MHz, DMSO-d₆) δ 13.16 (br s, 1H) 10.83 (s, 1H) 8.57 (s, 1H) 8.34 (d, J=0.73 Hz, 1H) 7.92 (dd, J=2.87, 1.16 Hz, 1H) 7.73 (dd, J=5.01, 2.93 Hz, 1H) 7.65-7.69 (m, 2H) 7.57-7.63 (m, 2H) 4.09 (s, 3H). MS-ESI (m/z) calc'd for C₂₀H₁₄F₂N₅OS [M+H]⁺: 410.1. Found 410.0.

### Example 101: N-(3-(2,5-Dimethylfuran-3-yl)-1H-indazol-5-yl)-4,6-difluoro-1-methyl-1H-indazole-5-carboxamide

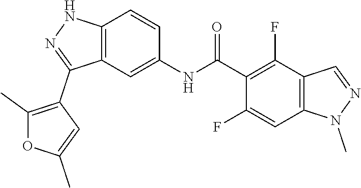 [SMILES: Cc1cc(-c2n[nH]c3ccc(NC(=O)c4c(F)cc5c(cnn5C)c4F)cc23)c(C)o1]

Prepared as described for N-(3-(2-cyanophenyl)-1H-indazol-5-yl)-4,6-difluoro-1-methyl-1H-indazole-5-carboxamide using (2,5-dimethylfuran-3-yl)boronic acid in place of (2-cyanophenyl)boronic acid. ¹H NMR (400 MHz, DMSO-d₆) δ 8.33 (s, 1H) 8.18 (s, 1H) 7.50-7.59 (m, 2H) 7.35 (d, J=9.41 Hz, 1H) 6.40 (s, 1H) 4.08 (s, 3H) 2.47 (s, 3H) 2.33 (s, 3H). MS-ESI (m/z) calc'd for C₂₂H₁₈F₂N₅O₂ [M+H]⁺: 422.1. Found 422.1.

### Example 102: 4,6-Difluoro-N-(3-(4-methoxypyridin-3-yl)-1H-indazol-5-yl)-1-methyl-1H-indazole-5-carboxamide

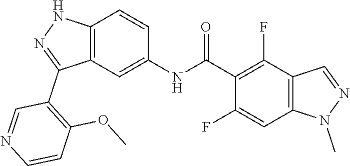 [SMILES: COc1ccncc1-c1n[nH]c2ccc(NC(=O)c3c(F)cc4c(cnn4C)c3F)cc12]

Prepared as described for N-(3-(2-cyanophenyl)-1H-indazol-5-yl)-4,6-difluoro-1-methyl-1H-indazole-5-carboxamide using (4-methoxypyridin-3-yl)boronic acid in place of (2-cyanophenyl)boronic acid. ¹H NMR (400 MHz, DMSO-d₆) δ 13.30 (s, 1H) 10.75 (s, 1H) 8.61 (s, 1H) 8.53 (d, J=5.73 Hz, 1H) 8.33 (s, 1H) 8.21 (s, 1H) 7.65 (d, J=9.70 Hz, 1H) 7.58 (s, 2H) 7.26 (d, J=5.73 Hz, 1H) 4.08 (s, 3H) 3.93 (s, 3H). MS-ESI (m/z) calc'd for C₂₂H₁₇F₂N₆O₂ [M+H]⁺: 435.1. Found 435.0.

### Example 103: 4,6-Difluoro-N-(3-(3-methoxypyridin-4-yl)-1H-indazol-5-yl)-1-methyl-1H-indazole-5-carboxamide

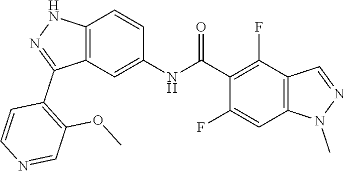 [SMILES: COc1cnccc1-c1n[nH]c2ccc(NC(=O)c3c(F)cc4c(cnn4C)c3F)cc12]

Prepared as described for N-(3-(2-cyanophenyl)-1H-indazol-5-yl)-4,6-difluoro-1-methyl-1H-indazole-5-carboxamide using (3-methoxypyridin-4-yl)boronic acid in place of (2-cyanophenyl)boronic acid. ¹H NMR (400 MHz, DMSO-d₆) δ 8.67 (br s, 1H) 8.63 (s, 1H) 8.47 (br s, 1H) 8.27 (br d, J=5.29 Hz, 1H) 8.18 (s, 1H) 7.64 (d, J=9.04 Hz, 1H) 7.54 (br d, J=9.04 Hz, 1H) 7.36 (d, J=9.48 Hz, 1H) 4.20 (s, 3H) 4.08 (s, 3H). MS-ESI (m/z) calc'd for C₂₂H₁₇F₂N₆O₂ [M+H]⁺: 435.1. Found 435.0.

### Example 104: 4,6-Difluoro-1-methyl-N-(3-(tetrahydrofuran-3-yl)-1H-indazol-5-yl)-1H-indazole-5-carboxamide

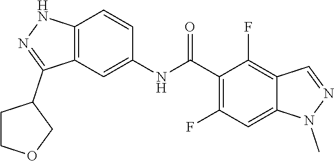 [SMILES: Cn1ncc2c(F)c(C(=O)Nc3ccc4[nH]nc(C5CCOC5)c4c3)c(F)cc21]

To a solution of 4,6-difluoro-N-(3-(furan-3-yl)-1H-indazol-5-yl)-1-methyl-1H-indazole-5-carboxamide (40 mg, 101.69 umol) in MeOH (1 mL) was added Pd/C (30 mg, 10% purity). The mixture was stirred at 20° C. for 2 hrs under H₂ atmosphere (15 psi) and monitored by LCMS. The reaction was filtered and the filtrate was concentrated under reduced pressure to remove solvent. Another 20 mg batch of crude material was combined with this reaction for further purification. The residue was purified by preparative HPLC (neutral condition) to afford the title compound (1.63 mg) as a colorless gum. ¹H NMR (400 MHz, DMSO-d₆) δ 8.26-8.31 (m, 1H) 8.19 (d, J=0.73 Hz, 1H) 7.55-7.60 (m, 1H) 7.48-7.52 (m, 1H) 7.36 (d, J=9.41 Hz, 1H) 4.24 (t, J=7.95 Hz, 1H) 4.11-4.16 (m, 1H) 4.09 (s, 3H) 3.89-4.07 (m, 3H) 2.45-2.55 (m, 1H) 2.32-2.42 (m, 1H). MS-ESI (m/z) calc'd for C₂₀H₁₈F₂N₅O₂ [M+H]⁺:398.1. Found 398.1.

### Example 105: N-(3-Cyano-1H-indazol-5-yl)-4,6-difluoro-1-methyl-1H-indazole-5-carboxamide

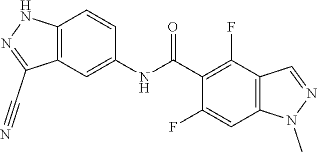 [SMILES: Cn1ncc2c(F)c(C(=O)Nc3ccc4[nH]nc(C#N)c4c3)c(F)cc21]

### Step 1: 5-Amino-1H-indazole-3-carbonitrile

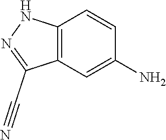 [SMILES: N#Cc1n[nH]c2ccc(N)cc12]

To a solution of 5-nitro-1H-indazole-3-carbonitrile (100 mg, 531.51 umol) in EtOAc (5 mL) was added Pd/C (30 mg, 10% purity) under N₂. The suspension was degassed under vacuum and purged with H₂ several times. The mixture was stirred under H₂ (15 psi) at 15° C. for 16 hrs. The reaction mixture was filtered and the filtrate was concentrated to afford the title compound (81 mg, crude) as a red brown solid.

### Step 2: N-(3-Cyano-1H-indazol-5-yl)-4,6-difluoro-1-methyl-1H-indazole-5-carboxamide

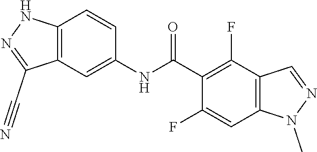 [SMILES: Cn1ncc2c(F)c(C(=O)Nc3ccc4[nH]nc(C#N)c4c3)c(F)cc21]

To a solution of 4,6-difluoro-1-methyl-1H-indazole-5-carboxylic acid (42.92 mg, 202.33 umol) and 5-amino-1H-indazole-3-carbonitrile (40 mg, 252.91 umol) in pyridine (3 mL) was added EDCI (96.97 mg, 505.82 umol). The mixture was stirred at 15° C. for 12 hrs. The reaction mixture was concentrated under reduced pressure to remove solvent. The residue was purified by preparative HPLC (TFA condition) and further purified by preparative HPLC (neutral condition) to afford the title compound (8.13 mg, 17.37 umol, 7% yield) as a white solid. ¹H NMR (400 MHz, DMSO-d₆) δ 14.34 (br s, 1H), 11.04 (s, 1H), 8.46 (d, J=1.0 Hz, 1H), 8.35 (s, 1H), 7.83-7.76 (m, 1H), 7.71-7.62 (m, 2H), 4.08 (s, 3H).). MS-ESI (m/z) calc'd for C₁₇H₁₁F₂N₆O [M+H]⁺:353.1. Found 353.0.

### Example 106: 4,6-Difluoro-N-(3-methoxy-1H-indazol-5-yl)-1-methyl-1H-indazole-5-carboxamide

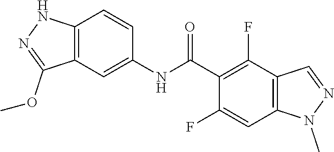 [SMILES: COc1n[nH]c2ccc(NC(=O)c3c(F)cc4c(cnn4C)c3F)cc12]

Prepared as described for N-(3-cyano-1H-indazol-5-yl)-4,6-difluoro-1-methyl-1H-indazole-5-carboxamide using 3-methoxy-1H-indazol-5-amine in place of 5-amino-1H-indazole-3-carbonitrile. ¹H NMR (400 MHz, DMSO-d₆) δ 11.92 (s, 1H) 10.74 (s, 1H) 8.33 (s, 1H) 8.13 (s, 1H) 7.66 (d, J=9.70 Hz, 1H) 7.49 (dd, J=9.04, 1.76 Hz, 1H) 7.36 (d, J=8.82 Hz, 1H) 4.08 (s, 3H) 4.00 (s, 3H). MS-ESI (m/z) calc'd for C₁₇H₁₄F₂N₅O₂ [M+H]⁺: 358.1. Found 358.1.

### Example 107: 1-Methyl-N-(3-phenyl-1H-indazol-5-yl)-1H-indazole-5-carboxamide

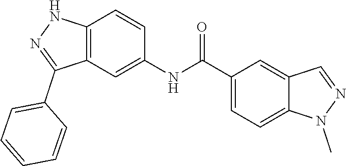 [SMILES: Cn1ncc2cc(C(=O)Nc3ccc4[nH]nc(-c5ccccc5)c4c3)ccc21]

### Step 1: 3-Bromo-5-nitro-1-((2-(trimethylsilyl)ethoxy)methyl)-1H-indazole

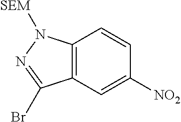 [SMILES: C[Si](C)(C)CCOCn1nc(Br)c2cc([N+](=O)[O-])ccc21]

To a stirred solution of 3-bromo-5-nitro-1H-indazole (2 g, 8.26 mmol) in THF (20 mL) was added NaH (661.01 mg, 16.53 mmol, 60% purity) at 0° C., then SEM-Cl (1.65 g, 9.92 mmol, 1.76 mL) was added and the reaction mixture was warmed to 15° C. and stirred for 12 hrs. The reaction mixture was quenched by saturated aqueous NH₄Cl (20 mL) and extracted with EtOAc (20 mL×3). The combined organic phases were washed with brine (20 mL×1), dried over anhydrous Na₂SO₄, filtered and concentrated in vacuum. The residue was purified by column chromatography (SiO₂) using a gradient of 0-10% EtOAc/petroleum ether to afford the title compound (3 g, 8.06 mmol, 98% yield) as a yellow solid.

### Step 2: 5-Nitro-3-phenyl-1-((2-(trimethylsilyl)ethoxy)methyl)-1H-indazole

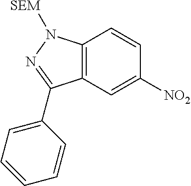 [SMILES: C[Si](C)(C)CCOCn1nc(-c2ccccc2)c2cc([N+](=O)[O-])ccc21]

A mixture of 3-bromo-5-nitro-1-((2-(trimethylsilyl)ethoxy)methyl)-1H-indazole (1 g, 2.69 mmol), phenylboronic acid (393.01 mg, 3.22 mmol), KOAc (790.86 mg, 8.06 mmol) and Pd(Amphos)C₁₂ (190.19 mg, 268.61 umol) in EtOH (10 mL) and H₂O (2.5 mL) was de-gassed and heated to 100° C. for 12 hrs under N₂. After cooling to 20° C., the reaction mixture was concentrated. The residue was poured into water (20 mL). The aqueous phase was extracted with EtOAc (20 mL×3). The combined organic phases were washed with brine (20 mL×1), dried over anhydrous Na₂SO₄, filtered and concentrated in vacuum. The residue was purified by column chromatography (SiO₂) using a gradient of 0-10% EtOAc/petroleum ether to afford the title compound (740 mg, 2.00 mmol, 75% yield) as a yellow oil.

### Step 3: 3-Phenyl-1-((2-(trimethylsilyl)ethoxy)methyl)-1H-indazol-5-amine

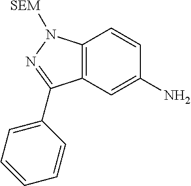 [SMILES: C[Si](C)(C)CCOCn1nc(-c2ccccc2)c2cc(N)ccc21]

To a stirred solution of 5-nitro-3-phenyl-1-((2-(trimethylsilyl)ethoxy)methyl)-1H-indazole (600 mg, 1.62 mmol) in EtOH (10 mL) and H₂O (10 mL) was added Fe (453.42 mg, 8.12 mmol) and NH₄Cl (434.31 mg, 8.12 mmol). The reaction mixture was stirred at 80° C. for 2 hrs and monitored by TLC (petroleum ether/EtOAc=2/1, Rf=0.24). After cooling to 20° C., the reaction mixture was filtered and the filtrate was concentrated. The residue was poured into water (10 mL). The aqueous phase was extracted with EtOAc (10 mL×3). The combined organic phases were washed with brine (10 mL×1), dried over anhydrous Na₂SO₄, filtered and concentrated in vacuum. The residue was purified by column chromatography (SiO₂, petroleum ether/EtOAc=50/1 to 5/1) to afford the title compound (510 mg, 1.50 mmol, 93% yield) as a brown oil.

### Step 4: 1-Methyl-N-(3-phenyl-1-((2-(trimethylsilyl)ethoxy)methyl)-1H-indazol-5-yl)-1H-indazole-5-carboxamide

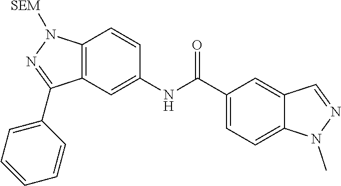 [SMILES: Cn1ncc2cc(C(=O)Nc3ccc4c(c3)c(-c3ccccc3)nn4COCC[Si](C)(C)C)ccc21]

To a solution of 1-methyl-1H-indazole-5-carboxylic acid (90 mg, 510.87 umol) and 3-phenyl-1-((2-(trimethylsilyl)ethoxy)methyl)-1H-indazol-5-amine (173.44 mg, 510.87 umol) in DCM (2 mL) was added T₃P (50 wt. % in EtOAc, 487.64 mg, 766.30 umol) and the reaction mixture was stirred at 20° C. for 0.5 hr. Then TEA (155.08 mg, 1.53 mmol) was added and the reaction mixture was stirred at 20° C. for 12 hrs. The reaction mixture was concentrated. The residue was purified by preparative TLC (SiO₂, petroleum ether/EtOAc=1/1, Rf=0.30) to afford the title compound (140 mg, 281.32 umol, 55% yield) as a yellow solid.

### Step 5: N-(1-(Hydroxymethyl)-3-phenyl-1H-indazol-5-yl)-1-methyl-1H-indazole-5-carboxamide

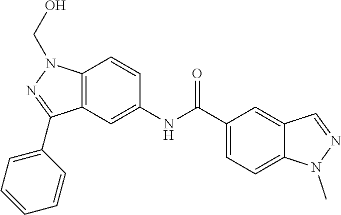 [SMILES: Cn1ncc2cc(C(=O)Nc3ccc4c(c3)c(-c3ccccc3)nn4CO)ccc21]

1-Methyl-N-(3-phenyl-1-((2-(trimethylsilyl)ethoxy)methyl)-1H-indazol-5-yl)-1H-indazole-5-carboxamide (140 mg, 281.32 umol) was dissolved into TFA (2 mL) and the reaction mixture was stirred at 20° C. for 12 hrs. The reaction mixture was concentrated to afford the title compound (100 mg, crude) as a yellow gum which was used without further purification.

### Step 6: 1-Methyl-N-(3-phenyl-1H-indazol-5-yl)-1H-indazole-5-carboxamide

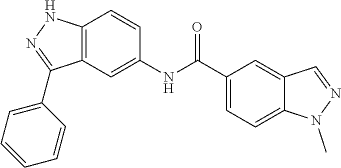 [SMILES: Cn1ncc2cc(C(=O)Nc3ccc4[nH]nc(-c5ccccc5)c4c3)ccc21]

To a solution of N-(1-(hydroxymethyl)-3-phenyl-1H-indazol-5-yl)-1-methyl-1H-indazole-5-carboxamide (100 mg, 251.62 umol) in MeOH (2 mL) and DMF (2 mL) was added K₂CO₃ (104.33 mg, 754.85 umol) and the reaction mixture was stirred at 20° C. for 12 hrs. The reaction mixture was filtered and the filtrate was concentrated. The residue was purified by preparative HPLC (neutral condition) to afford the title compound (17.81 mg, 47.92 umol, 19% yield) as a colorless gum. ¹H NMR (400 MHz, DMSO-d₆) δ 13.24 (br s, 1H) 10.38 (s, 1H) 8.61 (d, J=0.98 Hz, 1H) 8.52 (s, 1H) 8.26 (s, 1H) 8.06 (dd, J=8.86, 1.41 Hz, 1H) 7.98 (d, J=7.34 Hz, 2H) 7.84 (dd, J=9.05, 1.59 Hz, 1H) 7.78 (d, J=8.80 Hz, 1H) 7.61 (d, J=8.93 Hz, 1H) 7.56 (t, J=7.70 Hz, 2H) 7.39-7.46 (m, 1H) 4.12 (s, 3H). MS-ESI (m/z) calc'd for C₂₂H₁₈N₅O [M+H]⁺: 368.1. Found 368.1.

### Example 108: 4,6-Difluoro-1-methyl-N-(3-(pyrrolidin-1-yl)-1H-indazol-5-yl)-1H-indazole-5-carboxamide

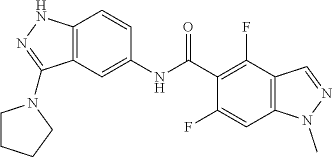 [SMILES: Cn1ncc2c(F)c(C(=O)Nc3ccc4[nH]nc(N5CCCC5)c4c3)c(F)cc21]

### Step 1: 3-Bromo-5-nitro-1-((2-(trimethylsilyl)ethoxy)methyl)-1H-indazole

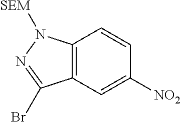 [SMILES: C[Si](C)(C)CCOCn1nc(Br)c2cc([N+](=O)[O-])ccc21]

To a stirred solution of 3-bromo-5-nitro-1H-indazole (5 g, 20.66 mmol) in THF (50 mL) was added NaH (826.35 mg, 20.66 mmol) at 0° C. and the reaction mixture was stirred at 0° C. for 0.5 hr. Then SEM-Cl (3.44 g, 20.66 mmol) was added and the reaction mixture was warmed to 20° C. and stirred for 11.5 hrs. The reaction mixture was quenched by saturated aqueous NH₄Cl (50 mL) and extracted with EtOAc (50 mL×3). The combined organic phases were washed with brine (50 mL×1), dried over anhydrous Na₂SO₄, filtered and concentrated under reduced pressure. The residue was purified by column chromatography (SiO₂, petroleum ether/EtOAc=90/10 to 0/100) to afford the title compound (5 g, 13.43 mmol, 65% yield) as a yellow solid.

### Step 2: 5-Nitro-3-(pyrrolidin-1-yl)-1-((2-(trimethylsilyl)ethoxy)methyl)-1H-indazole

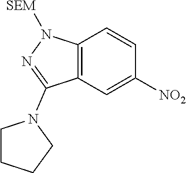 [SMILES: C[Si](C)(C)CCOCn1nc(N2CCCC2)c2cc([N+](=O)[O-])ccc21]

To a solution of 3-bromo-5-nitro-1-((2-(trimethylsilyl)ethoxy)methyl)-1H-indazole (500 mg, 1.34 mmol) and pyrrolidine (238.79 mg, 3.36 mmol) in toluene (10 mL) was added Cs₂CO₃ (1.31 g, 4.03 mmol), BINAP (83.63 mg, 134.30 umol) and Pd₂(dba)₃ (122.98 mg, 134.30 umol) under N₂. The mixture was stirred at 100° C. for 12 hrs under N₂. The reaction mixture was concentrated under reduced pressure and purified by column chromatography (SiO₂, petroleum ether/EtOAc=1/0 to 9/1) to afford the title compound (420 mg, 1.16 mmol, 86% yield) as a red solid.

### Step 3: 3-(Pyrrolidin-1-yl)-1H-indazol-5-amine

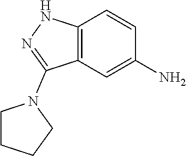 [SMILES: Nc1ccc2[nH]nc(N3CCCC3)c2c1]

To a solution of 5-nitro-3-(pyrrolidin-1-yl)-1-((2-(trimethylsilyl)ethoxy)methyl)-1H-indazole (420 mg, 1.16 mmol) in EtOH (20 mL) was added SnCl₂.2H₂O (1.57 g, 6.95 mmol) at 0° C. The mixture was then stirred at 80° C. for 12 hrs. The reaction mixture was concentrated under reduced pressure to remove solvent. The residue was diluted with NaHCO₃ (20 mL) to adjust the pH to 7, then filtered and the filtrate was extracted with EtOAc (20 mL×3). The combined organic layers were dried over Na₂SO₄, filtered and concentrated under reduced pressure to afford the title compound (260 mg, crude) as a yellow solid, which was used without further purification.

### Step 4: 4,6-Difluoro-1-methyl-N-(3-(pyrrolidin-1-yl)-1H-indazol-5-yl)-1H-indazole-5-carboxamide

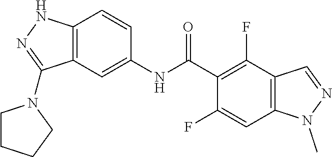 [SMILES: Cn1ncc2c(F)c(C(=O)Nc3ccc4[nH]nc(N5CCCC5)c4c3)c(F)cc21]

To a solution of 3-(pyrrolidin-1-yl)-1H-indazol-5-amine (100 mg, 494.43 umol) and 4,6-difluoro-1-methyl-2,3-dihydro-1H-indazole-5-carboxylic acid (104.89 mg, 494.43 umol) in pyridine (5 mL) was added EDCI (189.56 mg, 988.85 umol). The mixture was stirred at 20° C. for 12 hrs. The reaction mixture was concentrated under reduced pressure and purified by preparative HPLC three times (twice neutral conditions and the last TFA condition) to afford the title compound (9.34 mg, 17.08 umol) as a gray solid. ¹H NMR (400 MHz, DMSO-d₆) δ 11.98 (br s, 1H) 10.70 (br s, 1H) 8.41 (br s, 1H) 8.32 (s, 1H) 7.65 (br d, J=9.48 Hz, 1H) 7.55 (br d, J=9.04 Hz, 1H) 7.34 (br d, J=8.38 Hz, 1H) 4.08 (s, 3H) 3.57 (br s, 4H) 1.98 (br s, 4H). MS-ESI (m/z) calc'd for C₂₀H₁₉F₂N₆O [M+H]⁺: 397.2. Found 397.2.

### Example 109: 4,6-Difluoro-N-(3-(isoindolin-2-yl)-1H-indazol-5-yl)-1-methyl-1H-indazole-5-carboxamide

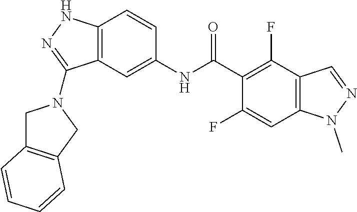 [SMILES: Cn1ncc2c(F)c(C(=O)Nc3ccc4[nH]nc(N5Cc6ccccc6C5)c4c3)c(F)cc21]

Prepared as described for 4,6-difluoro-1-methyl-N-(3-(pyrrolidin-1-yl)-1H-indazol-5-yl)-1H-indazole-5-carboxamide using isoindoline in place of pyrrolidine. ¹H NMR (400 MHz, DMSO-d₆) 11.89 (s, 1H) 10.73 (s, 1H) 8.44 (s, 1H) 8.34 (s, 1H) 7.67 (d, J=9.48 Hz, 1H) 7.52-7.58 (m, 1H) 7.45 (dd, J=5.40, 3.20 Hz, 2H) 7.29-7.38 (m, 3H) 4.92 (s, 4H) 4.09 (s, 3H). MS-ESI (m/z) calc'd for C₂₄H₁₉F₂N₆O₂ [M+H]⁺: 445.2. Found 445.2.

### Example 110: N-(3-(Benzylamino)-1H-indazol-5-yl)-4,6-difluoro-1-methyl-1H-indazole-5-carboxamide

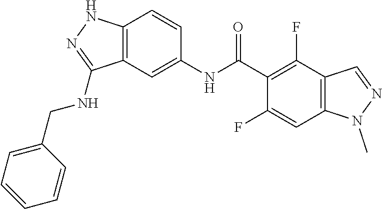 [SMILES: Cn1ncc2c(F)c(C(=O)Nc3ccc4[nH]nc(NCc5ccccc5)c4c3)c(F)cc21]

Prepared as described for 4,6-difluoro-1-methyl-N-(3-(pyrrolidin-1-yl)-1H-indazol-5-yl)-1H-indazole-5-carboxamide using benzylamine in place of pyrrolidine. ¹H NMR (400 MHz, DMSO-d₆) 11.39 (br s, 1H) 10.61 (s, 1H) 8.30 (br d, J=14.99 Hz, 2H) 7.64 (br d, J=9.48 Hz, 2H) 7.41 (br d, J=7.28 Hz, 2H) 7.26-7.34 (m, 3H) 7.18-7.25 (m, 2H) 6.64 (br t, J=5.95 Hz, 1H) 4.46 (br d, J=5.73 Hz, 2H) 4.07 (s, 3H). MS-ESI (m/z) calc'd for C₂₃H₁₉F₂N₆O [M+H]⁺: 433.2. Found 433.2.

### Example 111: N-(3-Iodo-1H-indazol-5-yl)-2,4-dimethylimidazo[1,5-a]pyrimidine-3-carboxamide

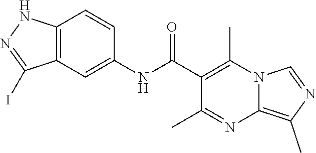 [SMILES: Cc1nc2c(C)ncn2c(C)c1C(=O)Nc1ccc2[nH]nc(I)c2c1]

### Step 1: 1H-Imidazol-4-amine

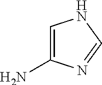

To a solution of 4-nitro-1H-imidazole (3 g, 26.53 mmol) in MeOH (24 mL) and NH₃.H₂O (3 mL) (purity: 25%) was added Raney-Ni (454.61 mg) under N₂ atmosphere. The mixture was degassed and purged with H₂ (15 psi) 3 times. The mixture was stirred under H₂ (15 psi) at 15° C. for 6 hrs and monitored by TLC (DCM/MeOH=5/1, Rf=0.32). The reaction mixture was filtered and the filtrate was concentrated to afford the title compound (1.6 g, crude) as a black solid.

### Step 2: Ethyl 2,4-dimethylimidazo[1,5-a]pyrimidine-3-carboxylate

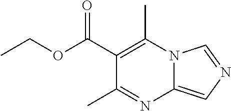 [SMILES: CCOC(=O)c1c(C)nc2cncn2c1C]

A solution of ethyl 2-acetyl-3-oxobutanoate (600 mg, 3.48 mmol) in AcOH (6 mL) and MeOH (6 mL) was added 1H-imidazol-4-amine (434.33 mg, 5.23 mmol). The mixture was stirred at 80° C. for 12 hrs and monitored by TLC (petroleum ether/EtOAc=0/1). The reaction mixture was concentrated under reduced pressure to remove solvent. The residue was diluted with EtOAc (20 mL), filtered and the filtrate was concentrated under reduced pressure to give a residue. The residue was purified by column chromatography (SiO₂) using a gradient of 0-100% EtOAc/petroleum ether to afford the title compound (50 mg, 228.06 umol, 7% yield) as a yellow solid.

### Step 3: N-(3-Iodo-1H-indazol-5-yl)-2,4-dimethylimidazo[1,5-a]pyrimidine-3-carboxamide

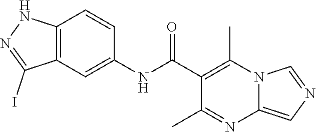 [SMILES: Cc1nc2cncn2c(C)c1C(=O)Nc1ccc2[nH]nc(I)c2c1]

To a solution of ethyl 2,4-dimethylimidazo[1,5-a]pyrimidine-3-carboxylate (45 mg, 205.26 umol) and 3-methyl-1H-indazol-5-amine (63.80 mg, 246.31 umol) in toluene (3 mL) was added Al(CH₃)₃ (2 M, 410.51 uL). The mixture was stirred at 90° C. for 12 hrs and monitored by LC-MS. The reaction mixture was concentrated under reduced pressure to remove solvent. The residue was diluted with H₂O (10 mL) and extracted with EtOAc (10 mL×5). The combined organic phases were dried over anhydrous Na₂SO₄, filtered and the filtrate was concentrated under reduced pressure to give a residue. The residue was purified by preparative HPLC (TFA condition) to afford the title compound (21.67 mg, 38.95 umol, 19% yield) as a yellow solid. ¹H NMR (400 MHz, DMSO-d₆) δ 9.34 (br s, 1H), 8.07 (d, J=1.1 Hz, 1H), 7.96 (s, 1H), 7.65-7.55 (m, 2H), 2.81 (s, 3H), 2.66 (s, 3H). MS-ESI (m/z) calc'd for C₁₆H₁₄₁N₆O [M+H]⁺: 433.0. Found 433.0.

### Example 112: N-(3-(Furan-3-yl)-1H-indazol-5-yl)-2,4-dimethylimidazo[1,5-a]pyrimidine-3-carboxamide

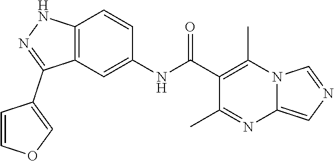 [SMILES: Cc1nc2cncn2c(C)c1C(=O)Nc1ccc2[nH]nc(-c3ccoc3)c2c1]

To a mixture of N-(3-iodo-1H-indazol-5-yl)-2,4-dimethylimidazo[1,5-a]pyrimidine-3-carboxamide (158 mg, 365.56 umol), 3-furylboronic acid (49.08 mg, 438.67 umol) and KOAc (107.63 mg, 1.10 mmol) in EtOH (2 mL) and H₂O (0.5 mL) was added Pd(Amphos)C₁₂ (25.88 mg, 36.56 umol). The mixture was degassed and purged with N₂ (3×) and stirred at 100° C. for 12 hrs under N₂ atmosphere. The reaction mixture was concentrated under reduced pressure and purified by preparative HPLC (TFA condition) to afford the title compound (30.38 mg, 59.34 umol, 16% yield) as a white solid. ¹H NMR (400 MHz, DMSO-d₆) δ 9.14 (s, 1H), 8.45 (s, 1H), 8.18 (s, 1H), 7.87 (s, 1H), 7.67 (t, J=1.5 Hz, 1H), 7.60 (s, 2H), 7.03 (d, J=1.6 Hz, 1H), 2.82 (s, 3H), 2.66 (s, 3H). MS-ESI (m/z) calc'd for C₂₀H₁₇N₆O₂ [M+H]⁺: 373.1. Found 373.1.

### Example 113: N-(3-(Furan-3-yl)-1H-indazol-5-yl)-4-methylimidazo[1,5-a]pyrimidine-3-carboxamide

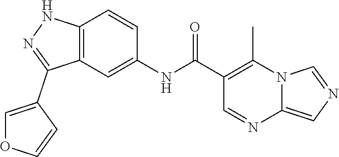 [SMILES: Cc1c(C(=O)Nc2ccc3[nH]nc(-c4ccoc4)c3c2)cnc2cncn12]

### Step 1: Methyl 5-methylimidazo[1,2-a]pyrimidine-6-carboxylate

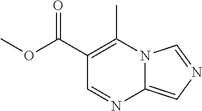

To a solution of 1H-imidazol-4-amine (334.90 mg, 4.03 mmol) in AcOH (43 mL) was added methyl (E)-2-((dimethylamino)methylene)-3-oxobutanoate (460 mg, 2.69 mmol). The mixture was stirred at 120° C. for 8 hrs and monitored by TLC (petroleum ether/EtOAc=0/1, Rf=0.25). The reaction mixture was concentrated under reduced pressure to remove solvent. The residue was diluted with EtOAc (20 mL), filtered and the filtrate was concentrated under reduced pressure to remove solvent. The residue was purified by column chromatography (SiO₂) using a gradient of 0-100% EtOAc/petroleum to afford the title compound (136 mg, 711.35 umol, 26% yield) as a yellow solid.

### Step 2: tert-Butyl 5-(4-methylimidazo[1,5-a]pyrimidine-3-carboxamido)-1H-indazole-3-carboxylate

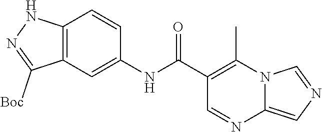 [SMILES: Cc1c(C(=O)Nc2ccc3[nH]nc(C(=O)OC(C)(C)C)c3c2)cnc2cncn12]

To a solution of methyl 5-methylimidazo[1,2-a]pyrimidine-6-carboxylate (136 mg, 711.35 umol) and 3-bromo-1H-indazol-5-amine (181.01 mg, 853.62 umol) in toluene (7 mL) was added Al(CH₃)₃ (2 M, 1.42 mL). The mixture was stirred at 90° C. for 12 hrs and monitored LC-MS. The reaction mixture was concentrated under reduced pressure to remove solvent. The residue was diluted with H₂O (20 mL) and extracted with EtOAc (20 mL×5). The combined organic phases were dried with anhydrous Na₂SO₄, filtered and the filtrate was concentrated under reduced pressure to afford the title compound (180 mg, crude) as a red solid.

### Step 3: N-(3-(Furan-3-yl)-1H-indazol-5-yl)-4-methylimidazo[1,5-a]pyrimidine-3-carboxamide

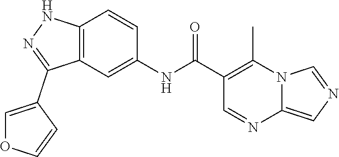 [SMILES: Cc1c(C(=O)Nc2ccc3[nH]nc(-c4ccoc4)c3c2)cnc2cncn12]

A mixture of N-(3-bromo-1H-indazol-5-yl)-4-methylimidazo[1,5-a]pyrimidine-3-carboxamide (180 mg, 484.93 umol), 3-furylboronic acid (65.11 mg, 581.91 umol) and KOAc (142.77 mg, 1.45 mmol) in EtOH (2 mL) and H₂O (0.5 mL) was added Pd(Amphos)C₁₂ (34.34 mg, 48.49 umol), then the mixture was degassed and purged with N₂ (3×). The mixture was then stirred at 100° C. for 12 hrs under N₂ atmosphere and monitored by LC-MS. The reaction mixture was concentrated under reduced pressure and purified by preparative HPLC (TFA condition) to afford the title compound (13.22 mg, 26.72 umol, 6% yield) as a yellow solid. ¹H NMR (400 MHz, DMSO-d₆) δ 13.13 (br s, 1H), 10.65 (s, 1H), 8.73 (br s, 1H), 8.48 (s, 1H), 8.40 (s, 1H), 8.25 (s, 1H), 7.86 (s, 1H), 7.77 (s, 1H), 7.67-7.62 (m, 1H), 7.61-7.56 (m, 1H), 7.01 (s, 1H), 2.86 (s, 3H). MS-ESI (m/z) calc'd for C₁₉H₁₅N₆O₂ [M+H]⁺: 359.1. Found 359.1.

### Example 114: N-(3-(Furan-3-yl)-1H-indazol-5-yl)-4-methylimidazo[1,5-a]pyrimidine-3-carboxamide

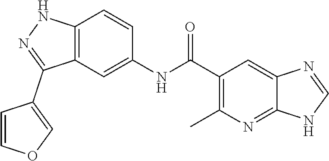 [SMILES: Cc1nc2[nH]cnc2cc1C(=O)Nc1ccc2[nH]nc(-c3ccoc3)c2c1]

### Step 1: Methyl 5-methyl-3H-imidazo[4,5-b]pyridine-6-carboxylate

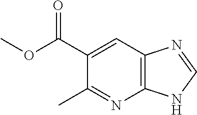

To a solution of methyl (Z)-2-((dimethylamino)methylene)-3-oxobutanoate (700 mg, 4.09 mmol) in AcOH (14 mL) was added 1H-imidazol-4-amine (509.64 mg, 6.13 mmol). The mixture was stirred at 120° C. for 8 hrs and monitored by LC-MS. The reaction mixture was concentrated under reduced pressure to remove solvent. The residue was diluted with MeOH (20 mL) and the mixture was filtered. The filtrate was concentrated under reduced pressure to remove solvent. The residue was purified by column chromatography (SiO₂) using a gradient of 0-100% EtOAc/petroleum ether to afford the title compound (480 mg, 2.51 mmol, 61% yield) as a yellow solid.

### Step 2: N-(3-(Furan-3-yl)-1H-indazol-5-yl)-4-methylimidazo[1,5-a]pyrimidine-3-carboxamide

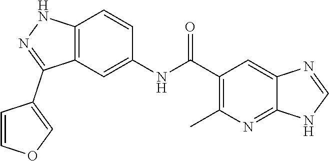 [SMILES: Cc1nc2[nH]cnc2cc1C(=O)Nc1ccc2[nH]nc(-c3ccoc3)c2c1]

Prepared as described for N-(3-(furan-3-yl)-1H-indazol-5-yl)-4-methylimidazo[1,5-a]pyrimidine-3-carboxamide using methyl 5-methyl-3H-imidazo[4,5-b]pyridine-6-carboxylate in place of methyl 5-methylimidazo[1,2-a]pyrimidine-6-carboxylate. ¹H NMR (400 MHz, DMSO-d₆) δ 13.12 (br s, 1H), 10.51 (s, 1H), 8.88-8.79 (m, 1H), 8.42 (s, 1H), 8.32 (s, 1H), 8.22 (s, 1H), 7.85 (s, 1H), 7.68 (br d, J=9.0 Hz, 1H), 7.56 (br d, J=8.8 Hz, 1H), 7.00 (s, 1H), 2.72 (s, 3H). MS-ESI (m/z) calc'd for C₂₃H₁₉F₂N₆O [M+H]⁺: 359.0. Found 359.0.

### Example 115: 4,6-Difluoro-1-methyl-N-(3-(trifluoromethyl)-1H-indazol-5-yl)-1H-indazole-5-carboxamide

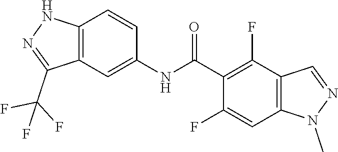 [SMILES: Cn1ncc2c(F)c(C(=O)Nc3ccc4[nH]nc(C(F)(F)F)c4c3)c(F)cc21]

### Step 1: 2,2,2-Trifluoro-1-(2-fluoro-5-nitrophenyl)ethan-1-ol

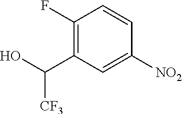 [SMILES: O=[N+]([O-])c1ccc(F)c(C(O)C(F)(F)F)c1]

To a solution of 2-fluoro-5-nitrobenzaldehyde (2 g, 11.83 mmol) in THF (50 mL) was added TBAF (1 M, 591.33 uL) and TMSCF3 (3.36 g, 23.65 mmol) under N₂ at 0° C. The mixture was stirred at 25° C. for 12 hrs and monitored by LC-MS. The reaction mixture was diluted with 2 N HCl (100 mL) and extracted with EtOAc (30 mL×3). The combined organic layers were washed with brine (50 mL×1), dried over Na₂SO₄, filtered and concentrated under reduced pressure to give a residue. The residue was purified by column chromatography (SiO₂) using a 0-25% gradient of EtOAc/petroleum ether to afford the title compound (800 mg, 3.35 mmol, 28% yield) as a yellow oil.

### Step 2: 2,2,2-Trifluoro-1-(2-fluoro-5-nitrophenyl)ethan-1-one

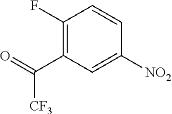 [SMILES: O=C(c1cc([N+](=O)[O-])ccc1F)C(F)(F)F]

To a solution of 2,2,2-trifluoro-1-(2-fluoro-5-nitrophenyl)ethan-1-ol (500 mg, 2.09 mmol) in DCM (20 mL) was added Dess-Martin periodinane (3.55 g, 8.36 mmol) and the mixture was stirred at 20° C. for 1.5 hrs and monitored by TLC (petroleum ether/EtOAc=3/1, Rf=0.2). The reaction mixture was diluted with saturated aqueous Na₂CO₃ (20 mL) and the organic phase was separated. The aqueous phase was extracted with EtOAc (10 mL×3). The combined organic layers were washed with brine (20 mL×1), dried over Na₂SO₄, filtered and concentrated under reduced pressure to give a residue. The residue was purified by column chromatography (SiO₂, petroleum ether/EtOAc=1/0 to 3/1) to afford the title compound (380 mg, 1.60 mmol, 77% yield) as a yellow oil.

### Step 3: 5-Nitro-3-(trifluoromethyl)-1H-indazole

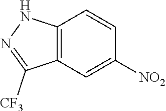 [SMILES: O=[N+]([O-])c1ccc2[nH]nc(C(F)(F)F)c2c1]

To a solution of 2,2,2-trifluoro-1-(2-fluoro-5-nitrophenyl)ethan-1-one (200 mg, 843.50 umol) in EtOH (5 mL) was added N₂H₄.H₂O (64.63 mg, 1.27 mmol, purity: 98%). The mixture was stirred at 90° C. for 12 hrs and monitored by LC-MS. The reaction mixture was concentrated under reduced pressure to remove solvent. The residue was purified by column chromatography (SiO₂) using a 0-25% gradient of EtOAc/petroleum ether to afford the title compound (180 mg, 778.78 umol, 92% yield) as a light yellow solid.

### Step 4: 3-(Trifluoromethyl)-1H-indazol-5-amine

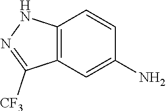 [SMILES: Nc1ccc2[nH]nc(C(F)(F)F)c2c1]

To a solution of 5-nitro-3-(trifluoromethyl)-1H-indazole (180 mg, 778.78 umol) in EtOH (3 mL) was added Pd/C (200 mg, 10% purity). The mixture was stirred at 20° C. for 2 hrs under H₂ at 15 psi and monitored by TLC (petroleum ether/EtOAc=3/1, Rf=0.15). The reaction mixture was filtered and the filtrate was concentrated under reduced pressure to afford the title compound (150 mg, crude) as a red solid.

### Step 5: 4,6-Difluoro-1-methyl-N-(3-(trifluoromethyl)-1H-indazol-5-yl)-1H-indazole-5-carboxamide

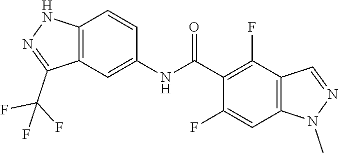 [SMILES: Cn1ncc2c(F)c(C(=O)Nc3ccc4[nH]nc(C(F)(F)F)c4c3)c(F)cc21]

To a solution of 3-(trifluoromethyl)-1H-indazol-5-amine (100 mg, 497.15 umol) and 4,6-difluoro-1-methyl-1H-indazole-5-carboxylic acid (105.47 mg, 497.15 umol) in pyridine (1 mL) was added EDCI (142.96 mg, 745.72 umol). The mixture was stirred at 25° C. for 12 hrs and monitored by LC-MS. The reaction mixture was concentrated under reduced pressure and purified by preparative HPLC (basic condition) to afford the title compound (105.01 mg, 255.03 umol, 51% yield) as a white solid. ¹H NMR (400 MHz, DMSO-d₆) δ 10.95 (s, 1H) 8.41 (s, 1H) 8.34 (s, 1H) 7.71-7.76 (m, 1H) 7.62-7.70 (m, 2H) 4.08 (s, 3H). MS-ESI (m/z) calc'd for C₁₇H₁₁F₅N₅O [M+H]⁺: 396.1. Found 396.1.

### Example 116: N-(3-(furan-3-yl)-1H-indazol-5-yl)benzo[d]oxazole-6-carboxamide

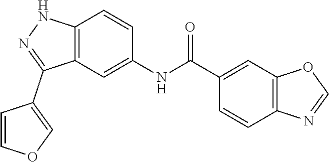 [SMILES: O=C(Nc1ccc2[nH]nc(-c3ccoc3)c2c1)c1ccc2ncoc2c1]

### Step 1: 3-Bromo-1H-indazol-5-amine

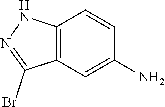 [SMILES: Nc1ccc2[nH]nc(Br)c2c1]

To a solution of 3-bromo-5-nitro-1H-indazole (15 g, 61.98 mmol) in EtOH (300 mL) was added SnCl₂.2H₂O (69.92 g, 309.88 mmol) at 15° C. The mixture was then stirred at 90° C. for 13 hrs and monitored by LC-MS. The reaction mixture was concentrated under reduced pressure to remove solvent. The residue was diluted with 1 M NaOH (700 mL) and extracted with EtOAc (300 mL×4). The combined organic layers were dried over Na₂SO₄, filtered and concentrated under reduced pressure to afford the title compound (13 g, crude) as a brown solid.

### Step 2: 3-(Furan-3-yl)-1H-indazol-5-amine

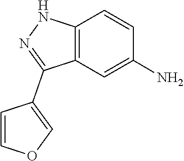 [SMILES: Nc1ccc2[nH]nc(-c3ccoc3)c2c1]

A mixture of 3-bromo-1H-indazol-5-amine (12 g, 56.59 mmol), furan-3-ylboronic acid (9.50 g, 84.89 mmol), Na₂CO₃ (29.99 g, 282.96 mmol), Pd(dppf)Cl₂ (4.14 g, 5.66 mmol) in dioxane (160 mL) and H₂O (160 mL) was degassed and purged with N₂ (3×). The mixture was then stirred at 120° C. for 2 hrs under N₂ atmosphere and monitored by LCMS. The reaction mixture was concentrated under reduced pressure to remove solvent. The residue was diluted with H₂O (500 mL) and EtOAc (300 mL) and then the mixture was filtered and the filtrate was extracted with EtOAc (200 mL×3). The combined organic layers were dried over Na₂SO₄, filtered and concentrated under reduced pressure to afford the title compound (7 g, crude) as a brown solid.

### Step 3: N-(3-(Furan-3-yl)-1H-indazol-5-yl)benzo[d]oxazole-6-carboxamide

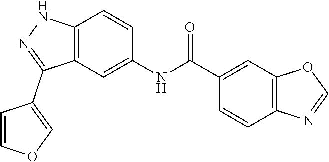 [SMILES: O=C(Nc1ccc2[nH]nc(-c3ccoc3)c2c1)c1ccc2ncoc2c1]

To a stirred solution of 3-(furan-3-yl)-1H-indazol-5-amine (476.26 mg, 2.39 mmol) and benzo[d]oxazole-6-carboxylic acid (300 mg, 1.84 mmol) in pyridine (3 mL) was added EDCI (528.82 mg, 2.76 mmol). The reaction mixture was stirred at 20° C. for 12 hrs and monitored by LC-MS. The reaction mixture was concentrated and purified by column chromatography (SiO₂) using a gradient of 0-100% EtOAc/petroleum ether to afford the title compound (86.28 mg, 245.79 umol, 13% yield) as a white solid. ¹H NMR (400 MHz, DMSO-d₆) δ 13.11 (s, 1H), 10.43 (s, 1H), 8.94 (s, 1H), 8.44 (s, 1H), 8.41 (s, 1H), 8.26 (s, 1H), 8.10 (d, J=8.4 Hz, 1H), 7.97 (d, J=8.3 Hz, 1H), 7.85 (s, 1H), 7.77 (dd, J=1.0, 8.9 Hz, 1H), 7.58 (d, J=8.9 Hz, 1H), 7.01 (s, 1H). MS-ESI (m/z) calc'd for C₁₉H₁₃N₄O₃ [M+H]⁺: 345.1. Found 345.0.

### Example 117: N-(3-(Furan-3-yl)-1H-indazol-5-yl)pyrazolo[1,5-a]pyridine-5-carboxamide

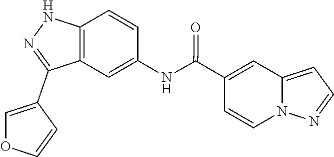 [SMILES: O=C(Nc1ccc2[nH]nc(-c3ccoc3)c2c1)c1ccn2nccc2c1]

Prepared as described for N-(3-(furan-3-yl)-1H-indazol-5-yl)benzo[d]oxazole-6-carboxamide using pyrazolo[1,5-a]pyridine-5-carboxylic acid in place of benzo[d]oxazole-6-carboxylic acid. ¹H NMR (400 MHz, DMSO-d₆) δ 13.10 (br s, 1H) 10.48 (s, 1H) 8.84 (d, J=7.25 Hz, 1H) 8.33-8.49 (m, 2H) 8.26 (s, 1H) 8.14 (d, J=2.25 Hz, 1H) 7.86 (t, J=1.56 Hz, 1H) 7.76 (dd, J=9.01, 1.75 Hz, 1H) 7.58 (d, J=9.01 Hz, 1H) 7.42 (dd, J=7.25, 1.88 Hz, 1H) 7.01 (d, J=1.13 Hz, 1H) 6.90 (d, J=2.13 Hz, 1H). MS-ESI (m/z) calc'd for C₁₉H₁₄N₅O₂ [M+H]⁺: 344.1. Found 344.1.

### Example 118: N-(3-(Furan-3-yl)-1H-indazol-5-yl)imidazo[1,2-a]pyridine-7-carboxamide

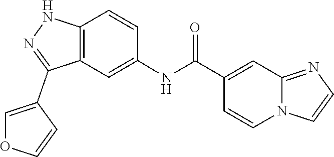 [SMILES: O=C(Nc1ccc2[nH]nc(-c3ccoc3)c2c1)c1ccn2ccnc2c1]

Prepared as described for N-(3-(furan-3-yl)-1H-indazol-5-yl)benzo[d]oxazole-6-carboxamide using 1,8a-dihydroimidazo[1,2-a]pyridine-7-carboxylic acid in place of benzo[d]oxazole-6-carboxylic acid. ¹H NMR (400 MHz, DMSO-d₆) δ 13.31-13.03 (m, 1H), 10.84 (br s, 1H), 9.03 (br d, J=6.6 Hz, 1H), 8.52 (br s, 1H), 8.46 (br s, 1H), 8.41 (s, 1H), 8.31 (br s, 1H), 8.26 (s, 1H), 7.95 (br d, J=5.0 Hz, 1H), 7.86 (s, 1H), 7.74 (br d, J=9.4 Hz, 1H), 7.61 (d, J=8.9 Hz, 1H), 7.01 (s, 1H). MS-ESI (m/z) calc'd for C₁₉H₁₄N₅O₂ [M+H]⁺: 344.1. Found 344.1.

### Example 119: N-(3-(Furan-3-yl)-1H-indazol-5-yl)imidazo[1,2-a]pyridine-6-carboxamide

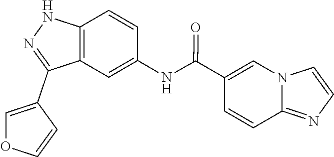 [SMILES: O=C(Nc1ccc2[nH]nc(-c3ccoc3)c2c1)c1ccc2nccn2c1]

Prepared as described for N-(3-(furan-3-yl)-1H-indazol-5-yl)benzo[d]oxazole-6-carboxamide using imidazo[1,2-a]pyridine-6-carboxylic acid in place of benzo[d]oxazole-6-carboxylic acid. ¹H NMR (400 MHz, DMSO-d₆) δ 13.31-13.03 (m, 1H), 10.84 (br s, 1H), 9.03 (br d, J=6.6 Hz, 1H), 8.52 (br s, 1H), 8.46 (br s, 1H), 8.41 (s, 1H), 8.31 (br s, 1H), 8.26 (s, 1H), 7.95 (br d, J=5.0 Hz, 1H), 7.86 (s, 1H), 7.74 (br d, J=9.4 Hz, 1H), 7.61 (d, J=8.9 Hz, 1H), 7.01 (s, 1H). MS-ESI (m/z) calc'd for C₁₉H₁₄N₅O₂ [M+H]⁺: 344.1. Found 344.1.

### Example 120: N-(3-(Furan-3-yl)-1H-indazol-5-yl)benzo[c][1,2,5]thiadiazole-5-carboxamide

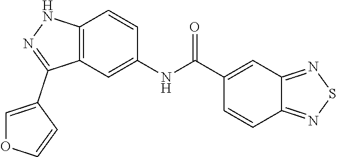 [SMILES: O=C(Nc1ccc2[nH]nc(-c3ccoc3)c2c1)c1ccc2nsnc2c1]

Prepared as described for N-(3-(furan-3-yl)-1H-indazol-5-yl)benzo[d]oxazole-6-carboxamide using benzo[c][1,2,5]thiadiazole-5-carboxylic acid in place of benzo[d]oxazole-6-carboxylic acid. ¹H NMR (400 MHz, DMSO-d₆) δ 13.12 (br s, 1H), 10.65 (br s, 1H), 8.80 (s, 1H), 8.44 (d, J=1.1 Hz, 1H), 8.26 (s, 1H), 8.26-8.22 (m, 2H), 7.86 (t, J=1.6 Hz, 1H), 7.79 (dd, J=1.7, 8.9 Hz, 1H), 7.59 (d, J=8.9 Hz, 1H), 7.01 (d, J=1.4 Hz, 1H). MS-ESI (m/z) calc'd for C₁₈H₁₂N₅O₂S [M+H]⁺: 362.1. Found 362.0.

### Example 121: N-(3-(Furan-3-yl)-1H-indazol-5-yl)benzo[d][1,2,3]thiadiazole-5-carboxamide

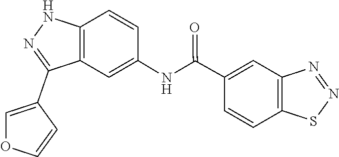 [SMILES: O=C(Nc1ccc2[nH]nc(-c3ccoc3)c2c1)c1ccc2snnc2c1]

Prepared as described for N-(3-(furan-3-yl)-1H-indazol-5-yl)benzo[d]oxazole-6-carboxamide using benzo[c][1,2,5]thiadiazole-5-carboxylic acid in place of benzo[d]oxazole-6-carboxylic acid. ¹H NMR (400 MHz, DMSO-d₆) δ 13.14 (br s, 1H), 10.67 (br s, 1H), 9.39 (s, 1H), 8.57 (br d, J=8.4 Hz, 1H), 8.45 (s, 1H), 8.39 (br d, J=8.4 Hz, 1H), 8.28 (s, 1H), 7.86 (s, 1H), 7.81 (br d, J=8.8 Hz, 1H), 7.60 (br d, J=8.9 Hz, 1H), 7.02 (s, 1H). MS-ESI (m/z) calc'd for C₁₈H₁₂N₅O₂S [M+H]⁺: 362.1. Found 362.0.

### Example 122: N-(3-(Furan-3-yl)-1H-indazol-5-yl)thiazolo[5,4-b]pyridine-5-carboxamide

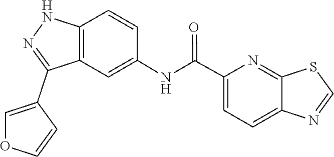 [SMILES: O=C(Nc1ccc2[nH]nc(-c3ccoc3)c2c1)c1ccc2ncsc2n1]

Prepared as described for N-(3-(furan-3-yl)-1H-indazol-5-yl)benzo[d]oxazole-6-carboxamide using thiazolo[5,4-b]pyridine-5-carboxylic acid in place of benzo[d]oxazole-6-carboxylic acid. ¹H NMR (400 MHz, DMSO-d₆) δ 13.12 (s, 1H) 10.87 (s, 1H) 9.78 (s, 1H) 8.72 (d, J=8.44 Hz, 1H) 8.53 (s, 1H) 8.38 (d, J=8.44 Hz, 1H) 8.32 (s, 1H) 8.04 (d, J=8.93 Hz, 1H) 7.86 (s, 1H) 7.57 (d, J=8.93 Hz, 1H) 7.03 (s, 1H). MS-ESI (m/z) calc'd for C₁₈H₁₂N₅O₂S [M+H]⁺: 362.1. Found 362.0.

### Example 123: N-(3-(Furan-3-yl)-1H-indazol-5-yl)benzo[d]thiazole-6-carboxamide

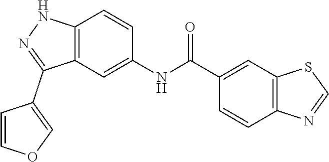 [SMILES: O=C(Nc1ccc2[nH]nc(-c3ccoc3)c2c1)c1ccc2ncsc2c1]

Prepared as described for N-(3-(furan-3-yl)-1H-indazol-5-yl)benzo[d]oxazole-6-carboxamide using thiazolo[5,4-b]pyridine-5-carboxylic acid in place of benzo[d]oxazole-6-carboxylic acid. ¹H NMR (400 MHz, DMSO-d₆) δ 13.12 (s, 1H), 10.49 (s, 1H), 9.58 (s, 1H), 8.84 (s, 1H), 8.43 (s, 1H), 8.29-8.25 (m, 1H), 8.23 (s, 1H), 8.20. MS-ESI (m/z) calc'd for C₁₉H₁₃N₄O₂S [M+H]⁺: 361.1. Found 361.0.

### Example 124: N-(3-(Furan-3-yl)-1H-indazol-5-yl)benzo[d]thiazole-5-carboxamide

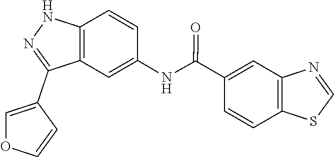 [SMILES: O=C(Nc1ccc2[nH]nc(-c3ccoc3)c2c1)c1ccc2scnc2c1]

Prepared as described for N-(3-(furan-3-yl)-1H-indazol-5-yl)benzo[d]oxazole-6-carboxamide using thiazolo[4,5-b]pyridine-5-carboxylic acid in place of benzo[d]oxazole-6-carboxylic acid. ¹H NMR (400 MHz, DMSO-d₆) δ 13.10 (br s, 1H), 10.49 (s, 1H), 9.54 (s, 1H), 8.79 (s, 1H), 8.43 (s, 1H), 8.35 (d, J=8.4 Hz, 1H), 8.27 (s, 1H), 8.16-8.09 (m, 1H), 7.85 (s, 1H), 7.81 (br d, J=8.8 Hz, 1H), 7.57 (d, J=8.8 Hz, 1H), 7.01 (s, 1H). MS-ESI (m/z) calc'd for C₁₉H₁₃N₄O₂S [M+H]⁺: 361.1. Found 361.0.

### Example 125: N-(3-(Furan-3-yl)-1H-indazol-5-yl)thieno[3,2-b]pyridine-2-carboxamide

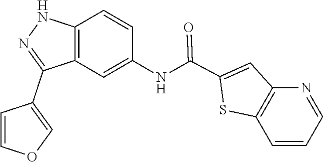 [SMILES: O=C(Nc1ccc2[nH]nc(-c3ccoc3)c2c1)c1cc2ncccc2s1]

Prepared as described for N-(3-(furan-3-yl)-1H-indazol-5-yl)benzo[d]oxazole-6-carboxamide using thieno[3,2-b]pyridine-2-carboxylic acid in place of benzo[d]oxazole-6-carboxylic acid. ¹H NMR (400 MHz, DMSO-d₆) δ 10.70 (s, 1H) 8.79 (dd, J=4.52, 1.34 Hz, 1H) 8.60 (br d, J=8.19 Hz, 1H) 8.55 (s, 1H) 8.39 (s, 1H) 8.28 (s, 1H) 7.84-7.87 (m, 1H) 7.75 (dd, J=8.93, 1.71 Hz, 1H) 7.60 (d, J=8.93 Hz, 1H) 7.51 (dd, J=8.31, 4.52 Hz, 1H) 7.01 (d, J=1.34 Hz, 1H). MS-ESI (m/z) calc'd for C₁₉H₁₃N₄O₂S [M+H]⁺: 361.1. Found 361.0.

### Example 126: N-(3-(Furan-3-yl)-1H-indazol-5-yl)benzo[d]oxazole-5-carboxamide

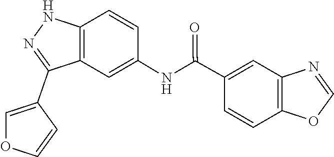 [SMILES: O=C(Nc1ccc2[nH]nc(-c3ccoc3)c2c1)c1ccc2ocnc2c1]

Prepared as described for N-(3-(furan-3-yl)-1H-indazol-5-yl)benzo[d]oxazole-6-carboxamide using benzo[d]oxazole-5-carboxylic acid in place of benzo[d]oxazole-6-carboxylic acid. ¹H NMR (400 MHz, DMSO-d₆) δ 13.07 (br s, 1H), 10.39 (s, 1H), 8.88 (s, 1H), 8.48 (s, 1H), 8.38 (s, 1H), 8.24 (s, 1H), 8.10 (dd, J=1.4, 8.5 Hz, 1H), 7.92 (d, J=8.6 Hz, 1H), 7.83 (d, J=1.3 Hz, 1H), 7.76 (dd, J=1.3, 8.8 Hz, 1H), 7.55 (d, J=9.0 Hz, 1H), 6.99 (s, 1H). MS-ESI (m/z) calc'd for C₁₉H₁₃N₄O₃ [M+H]⁺: 345.1. Found 345.0.

### Example 127: N-(3-(Furan-3-yl)-1H-indazol-5-yl)pyrazolo[1,5-a]pyrimidine-5-carboxamide

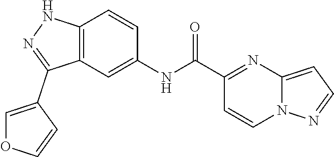 [SMILES: O=C(Nc1ccc2[nH]nc(-c3ccoc3)c2c1)c1ccn2nccc2n1]

Prepared as described for N-(3-(furan-3-yl)-1H-indazol-5-yl)benzo[d]oxazole-6-carboxamide using pyrazolo[1,5-a]pyrimidine-5-carboxylic acid in place of benzo[d]oxazole-6-carboxylic acid. ¹H NMR (400 MHz, DMSO-d₆) δ 13.11 (s, 1H), 10.83 (s, 1H), 9.35 (d, J=7.1 Hz, 1H), 8.52 (s, 1H), 8.43 (d, J=2.4 Hz, 1H), 8.31 (s, 1H), 8.01 (br d, J=9.0 Hz, 1H), 7.86 (s, 1H), 7.68 (d, J=7.3 Hz, 1H), 7.58 (d, J=8.8 Hz, 1H), 7.08-6.95 (m, 2H). MS-ESI (m/z) calc'd for C₁₈H₁₃N₆O₂ [M+H]⁺: 345.1. Found 345.0.

### Example 128: N-(3-(Furan-3-yl)-1H-indazol-5-yl)imidazo[1,2-a]pyrimidine-2-carboxamide

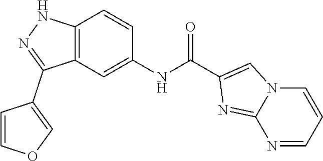 [SMILES: O=C(Nc1ccc2[nH]nc(-c3ccoc3)c2c1)c1cn2cccnc2n1]

Prepared as described for N-(3-(furan-3-yl)-1H-indazol-5-yl)benzo[d]oxazole-6-carboxamide using imidazo[1,2-a]pyrimidine-2-carboxylic acid in place of benzo[d]oxazole-6-carboxylic acid. ¹H NMR (400 MHz, DMSO-d₆) δ 12.98-13.19 (m, 1H) 10.55 (s, 1H) 9.22 (s, 1H) 8.59-8.76 (m, 2H) 8.52 (s, 1H) 8.30 (s, 1H) 7.93-8.09 (m, 2H) 7.78-7.91 (m, 1H) 7.55 (d, J=9.04 Hz, 1H) 7.02 (d, J=0.88 Hz, 1H). MS-ESI (m/z) calc'd for C₁₈H₁₃N₆O₂ [M+H]⁺: 345.1. Found 345.0.

### Example 129: N-(3-(Furan-3-yl)-1H-indazol-5-yl)imidazo[1,2-a]pyrimidine-6-carboxamide

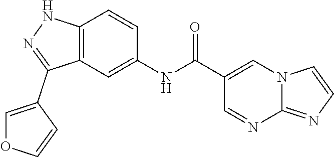 [SMILES: O=C(Nc1ccc2[nH]nc(-c3ccoc3)c2c1)c1cnc2nccn2c1]

Prepared as described for N-(3-(furan-3-yl)-1H-indazol-5-yl)benzo[d]oxazole-6-carboxamide using imidazo[1,2-a]pyrimidine-6-carboxylic acid in place of benzo[d]oxazole-6-carboxylic acid. ¹H NMR (400 MHz, DMSO-d₆) δ 13.15 (br s, 1H) 10.66 (s, 1H) 9.72 (s, 1H) 9.21 (br s, 1H) 8.37 (s, 1H) 8.25 (s, 1H) 8.18 (s, 1H) 8.01 (s, 1H) 7.86 (s, 1H) 7.71 (br d, J=8.56 Hz, 1H) 7.61 (d, J=8.44 Hz, 1H) 7.00 (s, 1H). MS-ESI (m/z) calc'd for C₁₈H₁₃N₆O₂ [M+H]⁺: 345.1. Found 345.0.

### Example 130: N-(3-(Furan-3-yl)-1H-indazol-5-yl)pyrazolo[1,5-a]pyrimidine-6-carboxamide

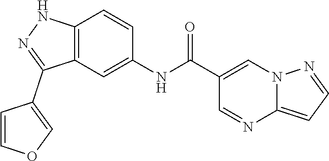 [SMILES: O=C(Nc1ccc2[nH]nc(-c3ccoc3)c2c1)c1cnc2ccnn2c1]

Prepared as described for N-(3-(furan-3-yl)-1H-indazol-5-yl)benzo[d]oxazole-6-carboxamide using pyrazolo[1,5-a]pyrimidine-6-carboxylic acid in place of benzo[d]oxazole-6-carboxylic acid. ¹H NMR (400 MHz, DMSO-d₆) δ 13.13 (br s, 1H) 10.53 (br s, 1H) 9.77 (d, J=1.32 Hz, 1H) 9.05 (d, J=1.98 Hz, 1H) 8.36-8.45 (m, 2H) 8.24 (s, 1H) 7.83-7.89 (m, 1H) 7.72 (dd, J=8.93, 1.65 Hz, 1H) 7.59 (d, J=8.82 Hz, 1H) 7.01 (d, J=1.10 Hz, 1H) 6.88 (d, J=2.21 Hz, 1H). MS-ESI (m/z) calc'd for C₁₈H₁₃N₆O₂ [M+H]⁺: 345.1. Found 345.0.

### Example 131: N-(3-(Furan-3-yl)-1H-indazol-5-yl)-1-methyl-1H-benzo[d]imidazole-6-carboxamide

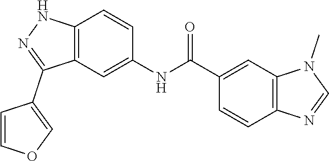 [SMILES: Cn1cnc2ccc(C(=O)Nc3ccc4[nH]nc(-c5ccoc5)c4c3)cc21]

Prepared as described for N-(3-(furan-3-yl)-1H-indazol-5-yl)benzo[d]oxazole-6-carboxamide using 1-methyl-1H-benzo[d]imidazole-6-carboxylic acid in place of benzo[d]oxazole-6-carboxylic acid. ¹H NMR (400 MHz, DMSO-d₆) δ 13.09 (br s, 1H) 10.32 (br s, 1H) 8.38 (br d, J=12.96 Hz, 2H) 8.28 (br d, J=14.67 Hz, 2H) 7.92 (br d, J=8.19 Hz, 1H) 7.85 (br s, 1H) 7.78 (br d, J=7.95 Hz, 2H) 7.57 (br d, J=8.68 Hz, 1H) 7.01 (s, 1H) 3.94 (s, 3H). MS-ESI (m/z) calc'd for C₂₀H₁₆N₅O₂ [M+H]⁺: 358.1. Found 358.1.

### Example 132: N-(3-(Furan-3-yl)-1H-indazol-5-yl)-1-methyl-1H-benzo[d]imidazole-5-carboxamide

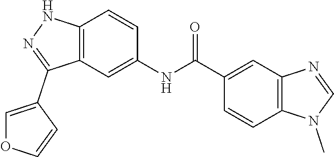 [SMILES: Cn1cnc2cc(C(=O)Nc3ccc4[nH]nc(-c5ccoc5)c4c3)ccc21]

Prepared as described for N-(3-(furan-3-yl)-1H-indazol-5-yl)benzo[d]oxazole-6-carboxamide using 1-methyl-1H-benzo[d]imidazole-5-carboxylic acid in place of benzo[d]oxazole-6-carboxylic acid. ¹H NMR (400 MHz, DMSO-d₆) δ 13.08 (br s, 1H), 10.29 (s, 1H), 8.43 (br d, J=3.1 Hz, 2H), 8.34 (s, 1H), 8.26 (s, 1H), 7.99 (dd, J=1.0, 8.5 Hz, 1H), 7.85 (s, 1H), 7.83 (dd, J=1.3, 9.0 Hz, 1H), 7.71 (d, J=8.6 Hz, 1H), 7.56 (d, J=9.0 Hz, 1H), 7.02 (d, J=0.9 Hz, 1H), 3.90 (s, 3H). MS-ESI (m/z) calc'd for C₂₀H₁₆N₅O₂ [M+H]⁺: 358.1. Found 358.1.

### Example 133: N-(3-(Furan-3-yl)-1H-indazol-5-yl)-1-methyl-1H-indazole-5-carboxamide

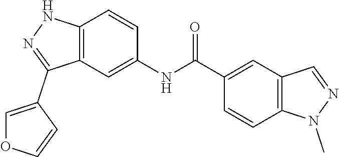 [SMILES: Cn1ncc2cc(C(=O)Nc3ccc4[nH]nc(-c5ccoc5)c4c3)ccc21]

Prepared as described for N-(3-(furan-3-yl)-1H-indazol-5-yl)benzo[d]oxazole-carboxamide using 1-methyl-1H-indazole-5-carboxylic acid in place of benzo[d]oxazole-6-carboxylic acid. ¹H NMR (400 MHz, DMSO-d₆) δ 13.07 (s, 1H), 10.33 (s, 1H), 8.51 (s, 1H), 8.39 (s, 1H), 8.26 (s, 2H), 8.06-8.03 (m, 1H), 7.85 (s, 1H), 7.78 (d, J=8.8 Hz, 2H), 7.55 (d, J=8.8 Hz, 1H), 7.00 (s, 1H), 4.11 (s, 3H). MS-ESI (m/z) calc'd for C₂₀H₁₆N₅O₂ [M+H]⁺: 358.1. Found 358.1.

### Example 134: N-(3-(Furan-3-yl)-1H-indazol-5-yl)-2-methylbenzo[d]oxazole-6-carboxamide

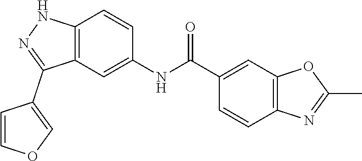 [SMILES: Cc1nc2ccc(C(=O)Nc3ccc4[nH]nc(-c5ccoc5)c4c3)cc2o1]

Prepared as described for N-(3-(furan-3-yl)-1H-indazol-5-yl)benzo[d]oxazole-6-carboxamide using 2-methylbenzo[d]oxazole-6-carboxylic acid in place of benzo[d]oxazole-6-carboxylic acid. ¹H NMR (400 MHz, DMSO-d₆) δ 13.09 (s, 1H), 10.37 (s, 1H), 8.39 (s, 1H), 8.30 (s, 1H), 8.25 (s, 1H), 8.03 (br d, J=8.4 Hz, 1H), 7.85 (s, 1H), 7.81 (d, J=8.4 Hz, 1H), 7.75 (br d, J=8.8 Hz, 1H), 7.56 (d, J=8.8 Hz, 1H), 7.00 (s, 1H), 2.68 (s, 3H). MS-ESI (m/z) calc'd for C₂₀H₁₅N₄O₃ [M+H]⁺: 359.1. Found 359.0.

### Example 135: 4,6-Difluoro-N-(3-(isoxazol-3-yl)-1H-indazol-5-yl)-1-methyl-1H-indazole-5-carboxamide

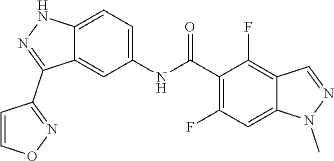 [SMILES: Cn1ncc2c(F)c(C(=O)Nc3ccc4[nH]nc(-c5ccon5)c4c3)c(F)cc21]

### Step 1: 2-(5-Nitro-1H-indazol-3-yl)ethen-1-ol

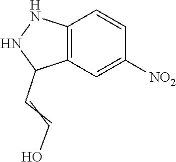 [SMILES: O=[N+]([O-])c1ccc2c(c1)C(C=CO)NN2]

To a stirred solution of 5-nitro-1H-indazole-3-carbaldehyde (400 mg, 2.09 mmol) in EtOH (5 mL) was added NH₂OH.HCl (290.84 mg, 4.19 mmol) and AcONa (343.32 mg, 4.19 mmol). The reaction mixture was stirred at 20° C. for 2 hrs and monitored by LC-MS. The reaction mixture was filtered and the filtrate was concentrated. The residue was poured into water (5 mL) and extracted with EtOAc (5 mL×3). The combined organic phase was washed with brine (5 mL×1), dried over anhydrous Na₂SO₄, filtered and concentrated to afford the title compound (450 mg, crude) as a light yellow solid, which was used without further purification.

### Step 2: 3-(5-Nitro-1H-indazol-3-yl)-5-(trimethylsilyl)isoxazole

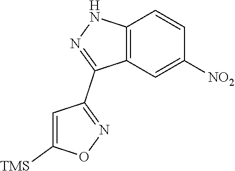 [SMILES: C[Si](C)(C)c1cc(-c2n[nH]c3ccc([N+](=O)[O-])cc23)no1]

To a solution of 2-(5-nitro-1H-indazol-3-yl)ethen-1-ol (450 mg, 2.18 mmol) and ethynyl(trimethyl)silane (321.58 mg, 3.27 mmol) in MeOH (10 mL) and H₂O (2 mL) was added PIFA (1.41 g, 3.27 mmol). The reaction mixture was stirred at 20° C. for 12 hrs. TLC (ether/=1/1, Rf=0.77) indicated the starting material was consumed and one major new spot with lower polarity was detected. The reaction mixture was concentrated and purified by column chromatography (SiO₂) using a 5-50% gradient of EtOAc/petroleum ether to afford the title compound (550 mg, 1.82 mmol, 83% yield) as a yellow solid.

### Step 3: 3-(5-(Trimethylsilyl)isoxazol-3-yl)-1H-indazol-5-amine

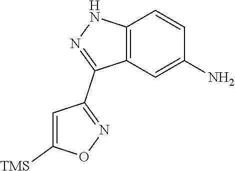 [SMILES: C[Si](C)(C)c1cc(-c2n[nH]c3ccc(N)cc23)no1]

To a solution of 3-(5-nitro-1H-indazol-3-yl)-5-(trimethylsilyl)isoxazole (543 mg, 1.80 mmol) in THF (5 mL) and H₂O (1.2 mL) was added Zn (939.46 mg, 14.37 mmol) and NH₄Cl (576.37 mg, 10.78 mmol). The reaction mixture was stirred at 25° C. for 1 hr. LCMS showed the starting material was consumed completely and one main peak with desired mass was detected. The reaction mixture was filtered and the filtrate was concentrated. The residue was poured into water (5 mL) and extracted with (5 mL×3). The combined organic phase was washed with brine (5 mL×1), dried over anhydrous Na₂SO₄, filtered and concentrated in vacuum. The residue was purified by column chromatography (SiO₂) using a 5-30% gradient of EtOAc/petroleum ether to afford the title compound (480 mg, 1.76 mmol, 98% yield) as a yellow solid.

### Step 4: 4,6-Difluoro-1-methyl-N-(3-(5-(trimethylsilyl)isoxazol-3-yl)-1H-indazol-5-yl)-1H-indazole-5-carboxamide

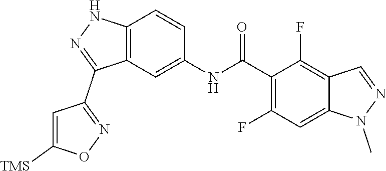 [SMILES: Cn1ncc2c(F)c(C(=O)Nc3ccc4[nH]nc(-c5cc([Si](C)(C)C)on5)c4c3)c(F)cc21]

To a solution of 3-(5-(trimethylsilyl)isoxazol-3-yl)-1H-indazol-5-amine (480 mg, 1.76 mmol) and 4,6-difluoro-1-methyl-1H-indazole-5-carboxylic acid (373.87 mg, 1.76 mmol) in pyridine (5 mL) was added EDCI (506.74 mg, 2.64 mmol). The reaction mixture was stirred at 25° C. for 12 hrs and monitored by LCMS. The reaction mixture was concentrated. The residue was poured into water (10 mL) and extracted with EtOAc (10 mL×3). The combined organic phase was washed with brine (10 mL×1), dried over anhydrous Na₂SO₄, filtered and concentrated in vacuum. The residue was purified by column chromatography (SiO₂) using a gradient of 5-100% EtOAc/petroleum ether to afford the title compound (650 mg, 1.39 mmol, 79% yield) as a yellow solid.

### Step 5: 4,6-Difluoro-N-(3-(isoxazol-3-yl)-1H-indazol-5-yl)-1-methyl-1H-indazole-5-carboxamide

To a solution of 4,6-difluoro-1-methyl-N-(3-(5-(trimethylsilyl)isoxazol-3-yl)-1H-indazol-5-yl)-1H-indazole-5-carboxamide (100 mg, 214.36 umol) in ACN (1 mL) and EtOH (0.5 mL) was added CsF (65.12 mg, 428.71 umol). The reaction mixture was stirred at 25° C. for 12 hrs and monitored by LCMS. The reaction mixture was filtered and the filtrate was concentrated. The residue was purified by preparative HPLC (neutral condition) to afford the title compound (17.67 mg, 44.81 umol, 21% yield) as a white solid. ¹H NMR (400 MHz, DMSO-d₆) δ 13.66 (br s, 1H), 11.06-10.88 (m, 1H), 9.07 (d, J=1.8 Hz, 1H), 8.71 (s, 1H), 8.35 (s, 1H), 7.82-7.62 (m, 3H), 7.08 (d, J=1.8 Hz, 1H), 4.09 (s, 3H). MS-ESI (m/z) calc'd for C₁₉H₁₃F₂N₆O₂ [M+H]⁺: 395.1. Found 395.1.

### Example 136: 4,6-Difluoro-1-methyl-N-(3-(oxazol-4-yl)-1H-indazol-5-yl)-1H-indazole-5-carboxamide

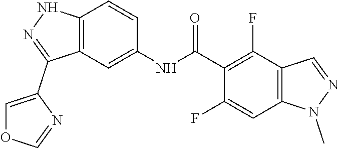 [SMILES: Cn1ncc2c(F)c(C(=O)Nc3ccc4[nH]nc(-c5cocn5)c4c3)c(F)cc21]

### Step 1: 3-Bromo-5-nitro-1-((2-(trimethylsilyl)ethoxy)methyl)-1H-indazole

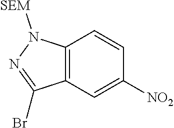 [SMILES: C[Si](C)(C)CCOCn1nc(Br)c2cc([N+](=O)[O-])ccc21]

To a solution of 3-bromo-5-nitro-1H-indazole (5 g, 20.66 mmol) in THF (50 mL) was added NaH (1.24 g, 30.99 mmol, 60% purity) at 0° C. The mixture was stirred at 0° C. for 0.5 hr. Then SEM-Cl (4.13 g, 24.79 mmol) was added and the mixture was stirred at 15° C. for 12 hrs. The reaction was monitored by TLC. The reaction mixture was quenched by addition NH₄Cl (30 mL) at 15° C. and then extracted with EtOAc (50 mL×3). The combined organic layers were dried over Na₂SO₄, filtered and concentrated under reduced pressure to give a residue. The residue was purified by flash silica gel chromatography (ISCO®; 20 g SepaFlash® Silica Flash Column, eluent of 0-16% EtOAc/ether gradient @ 100 mL/min) to afford the title compound (9 g, 16.92 mmol, 82% yield) as an orange solid.

### Step 2: 3-(1-Methoxyvinyl)-5-nitro-1-((2-(trimethylsilyl)ethoxy)methyl)-1H-indazole

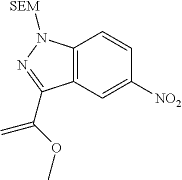 [SMILES: C=C(OC)c1nn(COCC[Si](C)(C)C)c2ccc([N+](=O)[O-])cc12]

A mixture of 3-bromo-5-nitro-1-((2-(trimethylsilyl)ethoxy)methyl)-1H-indazole (1 g, 2.69 mmol), tributyl(1-ethoxyvinyl)stannane (1.46 g, 4.04 mmol), Pd(PPh₃)₄ (310.85 mg, 269.00 umol) in toluene (15 mL) was degassed and purged with N₂ (3×) and then the mixture was stirred at 100° C. for 4 hrs under N₂ atmosphere. The reaction mixture was concentrated under reduced pressure to remove solvent. The residue was purified by flash silica gel chromatography (ISCO®; 12 g SepaFlash® Silica Flash Column, eluent of 04% EtOAc/petroleum ether gradient at 100 mL/min) to afford the title compound (870 mg, 2.39 mmol, 89% yield) as a yellow oil.

### Step 3: 1-(5-Nitro-1-((2-(trimethylsilyl)ethoxy)methyl)-1H-indazol-3-yl)ethan-1-one

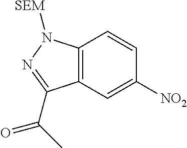 [SMILES: CC(=O)c1nn(COCC[Si](C)(C)C)c2ccc([N+](=O)[O-])cc12]

To a solution of 3-(1-methoxyvinyl)-5-nitro-1-((2-(trimethylsilyl)ethoxy)methyl)-1H-indazole (870 mg, 2.39 mmol) in EtOAc (9 mL) was added HCl (4M, 9.28 mL). The mixture was sonicated and stirred at 20° C. for 10 min. The reaction mixture was extracted with EtOAc (15 mL×3). The combined organic layers were dried over Na₂SO₄, filtered and concentrated under reduced pressure to afford the title compound (700 mg, crude) as a yellow solid.

### Step 4: 2-Bromo-1-(5-nitro-1-((2-(trimethylsilyl)ethoxy)methyl)-1H-indazol-3-yl)ethan-1-one

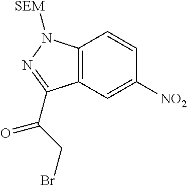 [SMILES: C[Si](C)(C)CCOCn1nc(C(=O)CBr)c2cc([N+](=O)[O-])ccc21]

To a solution of 1-(5nitro-1-((2-(trimethylsilyl)ethoxy)methyl)-1H-indazol-3-yl)ethan-1-one (660 mg, 1.97 mmol) in dioxane (21 mL) was added Br₂ (408.78 mg, 2.56 mmol) in dioxane (7 mL) at 0° C. The mixture was stirred at 20° C. for 12 hrs and monitored by TLC (ether/EtOAc=3/1, Rf=0.45). The reaction mixture was concentrated under reduced pressure and purified by flash silica gel chromatography (ISCO®; 12 g SepaFlash® Silica Flash Column, eluent of 0-10% EtOAc/petroleum ether gradient at 100 mL/min) to afford the title compound (350 mg, 844.75 umol, 43% yield) as a white solid.

### Step 5: 2-(5-Nitro-1-((2-(trimethylsilyl)ethoxy)methyl)-1H-indazol-3-yl)-2-oxoethyl formate

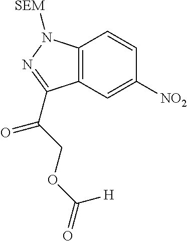 [SMILES: C[Si](C)(C)CCOCn1nc(C(=O)COC=O)c2cc([N+](=O)[O-])ccc21]

To a solution of 2-bromo-1-(5-nitro-1-((2-(trimethylsilyl)ethoxy)methyl)-1H-indazol-3-yl)ethan-1-one (485 mg, 1.17 mmol) in DMF (6 mL) was added sodium formate (103.49 mg, 1.52 mmol). The mixture was stirred at 15° C. for 12 hrs. TLC (petroleum ether/EtOAc=1/1, Rf=0.43) showed the starting material was consumed and one new spot with larger polarity was detected. The reaction mixture was poured into H₂O (10 mL) and extracted with EtOAc (10 mL×5), the combined organic layers were washed with brine (10 mL×2), dried over Na₂SO₄, filtered and concentrated to give a residue. The residue was purified by column chromatography (SiO₂, ether/EtOAc=1/1, Rf=0.43) to afford the title compound (370 mg, 975.12 umol, 83% yield) as a yellow solid.

### Step 6: 4-(5-Nitro-1-((2-(trimethylsilyl)ethoxy)methyl)-1H-indazol-3-yl)oxazole

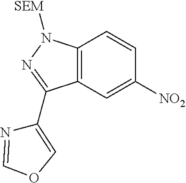 [SMILES: C[Si](C)(C)CCOCn1nc(-c2cocn2)c2cc([N+](=O)[O-])ccc21]

To a solution of 2-(5-nitro-1-((2-(trimethylsilyl)ethoxy)methyl)-1H-indazol-3-yl)-2-oxoethyl formate (500 mg, 1.32 mmol) in AcOH (5 mL) was added AcONH₄ (101.57 mg, 1.32 mmol), the mixture was stirred at 100° C. for 0.5 hr, then it was stirred at 120° C. for another 3 hrs. The reaction mixture was concentrated and purified by column chromatography (SiO₂, petroleum ether/EtOAc=1/0 to 4/1, Rf=0.43) to afford the title compound (160 mg, 97.66 umol, 7% yield) as a yellow solid.

### Step 7: 3-(Oxazol-4-yl)-1-((2-(trimethylsilyl)ethoxy)methyl)-1H-indazol-5-amine

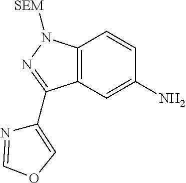 [SMILES: C[Si](C)(C)CCOCn1nc(-c2cocn2)c2cc(N)ccc21]

To a solution of 4-(5-nitro-1-((2-(trimethylsilyl)ethoxy)methyl)-1H-indazol-3-yl)oxazole (150 mg, 91.55 umol) in MeOH (6 mL) was added 10% Pd/C (150 mg). The mixture was degassed and purged with H₂ (3×) and then it was stirred at 15° C. for 2 hrs under H₂ atmosphere (15 psi). The reaction mixture was filtered and the filtrate was concentrated to give a residue. The residue was purified by preparative TLC (SiO₂, ether/EtOAc=1/1) to afford the title compound (34 mg, 61.73 umol, 67% yield) as an orange solid.

### Step 8: 4,6-Difluoro-1-methyl-N-(3-(oxazol-4-yl)-1-((2-(trimethylsilyl)ethoxy)methyl)-1H-indazol-5-yl)-1H-indazole-5-carboxamide

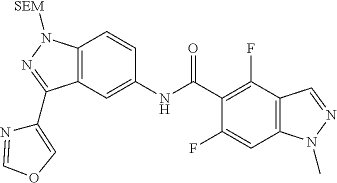 [SMILES: Cn1ncc2c(F)c(C(=O)Nc3ccc4c(c3)c(-c3cocn3)nn4COCC[Si](C)(C)C)c(F)cc21]

To a solution of 4,6-difluoro-1-methyl-1H-indazole-5-carboxylic acid (26.19 mg, 123.47 umol) in pyridine (2 mL) was added EDCI (29.59 mg, 154.33 umol) and 3-(oxazol-4-yl)-1-((2-(trimethylsilyl)ethoxy)methyl)-1H-indazol-5-amine (34 mg, 102.89 umol). The mixture was stirred at 15° C. for 12 hrs and monitored by LC-MS. The reaction mixture was concentrated and purified by preparative TLC (ether/EtOAc=0/1, Rf=0.43) to afford the title compound (24 mg, 45.75 umol, 44% yield) as a red solid.

### Step 9: 4,6-Difluoro-N-(1-(hydroxymethyl)-3-(oxazol-4-yl)-1H-indazol-5-yl)-1-methyl-1H-indazole-5-carboxamide

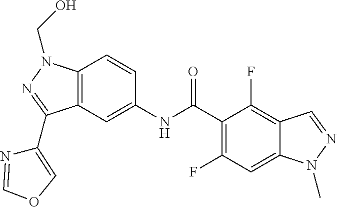 [SMILES: Cn1ncc2c(F)c(C(=O)Nc3ccc4c(c3)c(-c3cocn3)nn4CO)c(F)cc21]

To a solution of 4,6-difluoro-1-methyl-N-(3-(oxazol-4-yl)-1-((2-(trimethylsilyl)ethoxy)methyl)-1H-indazol-5-yl)-1H-indazole-5-carboxamide (20 mg, 38.12 umol) in DCM (1 mL) was added TFA (1 mL). The mixture was stirred at 15° C. for 3 hrs. LC-MS showed the starting material was consumed completely and one main peak with desired mass was detected. The reaction mixture was concentrated to give a residue. The residue was basified by addition of saturated aqueous NaHCO₃ (3 mL) and then the mixture was extracted with EtOAc (3 mL×3). The combined organic layers were dried over Na₂SO₄, filtered and concentrated to afford the title compound (15 mg, crude) as a yellow solid.

### Step 10: 4,6-Difluoro-1-methyl-N-(3-(oxazol-4-yl)-1H-indazol-5-yl)-1H-indazole-5-carboxamide

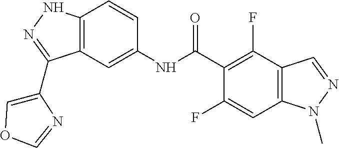 [SMILES: Cn1ncc2c(F)c(C(=O)Nc3ccc4[nH]nc(-c5cocn5)c4c3)c(F)cc21]

To a solution of 4,6-difluoro-N-(1-(hydroxymethyl)-3-(oxazol-4-yl)-1H-indazol-5-yl)-1-methyl-1H-indazole-5-carboxamide (15 mg, 35.35 umol) in DCM (0.5 mL) and H₂O (0.5 mL) was added NH₃.H₂O (455.00 mg, 3.25 mmol, 25% purity). The mixture was stirred at 15° C. for 2 hrs and monitored by LC-MS. The reaction mixture was extracted with DCM (3 mL×3). The combined organic layers were dried over Na₂SO₄, filtered and concentrated to give a residue. The residue was purified by preparative HPLC (Method K) to afford the title compound (2.53 mg, 4.63 umol, 13% yield) as a yellow solid. ¹H NMR (400 MHz, DMSO-d₆) δ 13.26 (br s, 1H) 10.81 (s, 1H) 8.71 (s, 1H) 8.60 (s, 2H) 8.34 (s, 1H) 7.54-7.69 (m, 3H) 4.09 (s, 3H). MS-ESI (m/z) calc'd for C₁₉H₁₃F₂N₆O₂ [M+H]⁺: 395.1. Found 395.1.

### Example 137: 4,6-Difluoro-N-(3-(isoxazol-5-yl)-1H-indazol-5-yl)-1-methyl-1H-indazole-5-carboxamide

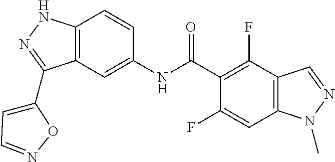 [SMILES: Cn1ncc2c(F)c(C(=O)Nc3ccc4[nH]nc(-c5ccno5)c4c3)c(F)cc21]

### Step 1: (E)-3-(Dimethylamino)-1-(5-nitro-1-((2-(trimethylsilyl)ethoxy)methyl)-1H-indazol-3-yl)prop-2-en-1-one

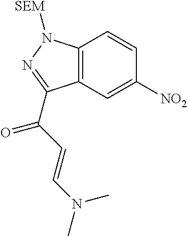 [SMILES: CN(C)/C=C/C(=O)c1nn(COCC[Si](C)(C)C)c2ccc([N+](=O)[O-])cc12]

1-(5-nitro-1-((2-(trimethylsilyl)ethoxy)methyl)-1H-indazol-3-yl)ethan-1-one (1 g, 2.98 mmol) and DMF-DMA (7.11 g, 59.62 mmol) were added to a microwave vial and the vial was sealed. The reaction mixture was heated to 160° C. for 0.25 hr in a reaction microwave and monitored by TLC (ether/=0/1, Rf=0.43). The reaction mixture was concentrated and purified by column chromatography (SiO₂, petroleum ether/EtOAc=1/0 to 0/1) to afford the title compound (750 mg, 1.92 mmol, 64% yield) as a yellow solid.

### Step 2: 5-(5-Nitro-1-((2-(trimethylsilyl)ethoxy)methyl)-1H-indazol-3-yl)isoxazole

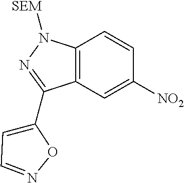 [SMILES: C[Si](C)(C)CCOCn1nc(-c2ccno2)c2cc([N+](=O)[O-])ccc21]

To a solution of (E)-3-(dimethylamino)-1-(5-nitro-1-((2-(trimethylsilyl)ethoxy)methyl)-1H-indazol-3-yl)prop-2-en-1-one (500 mg, 1.28 mmol) in EtOH (5 mL) was added NH₂OH.HCl (222.44 mg, 3.20 mmol). The reaction mixture was stirred at 100° C. for 12 hrs. The reaction mixture was concentrated under reduced pressure to remove solvent. The residue was diluted with H₂O (10 mL) and extracted with EtOAc (10 mL×3). The combined organic phase was washed with brine (10 mL×1), dried over anhydrous Na₂SO₄, filtered and concentrated. The residue was purified by column chromatography (SiO₂, petroleum ether/EtOAc=20/1 to 0/1) to afford the title compound (230 mg, 638.11 umol, 50% yield) as a yellow solid.

### Step 3: 3-(Isoxazol-5-yl)-1-((2-(trimethylsilyl)ethoxy)methyl)-1H-indazol-5-amine

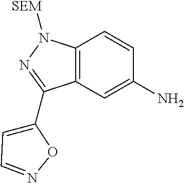 [SMILES: C[Si](C)(C)CCOCn1nc(-c2ccno2)c2cc(N)ccc21]

To a solution of 5-(5-nitro-1-((2-(trimethylsilyl)ethoxy)methyl)-1H-indazol-3-yl)isoxazole (230 mg, 638.11 umol) in THF (2 mL) and H₂O (0.5 mL) was added Zn (333.81 mg, 5.10 mmol) and NH₄Cl (204.80 mg, 3.83 mmol). The reaction mixture was stirred at 25° C. for 1 hr. The reaction mixture was filtered and the filtrate was poured into water (5 mL). The aqueous phase was extracted with EtOAc (10 mL×3). The combined organic phase was washed with brine (10 mL×1), dried over anhydrous Na₂SO₄, filtered and concentrated to afford the title compound (198 mg, 599.17 umol, 94% yield) as a yellow solid, which was used without further purification.

### Step 4: 4,6-Difluoro-N-(3-(isoxazol-5-yl)-1-((2-(trimethylsilyl)ethoxy)methyl)-1H-indazol-5-yl)-1-methyl-1H-indazole-5-carboxamide

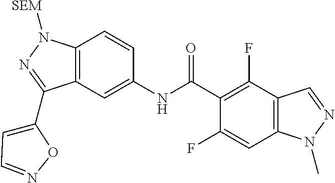 [SMILES: Cn1ncc2c(F)c(C(=O)Nc3ccc4c(c3)c(-c3ccno3)nn4COCC[Si](C)(C)C)c(F)cc21]

To a solution of 3-(isoxazol-5-yl)-1-((2-(trimethylsilyl)ethoxy)methyl)-1H-indazol-5-amine (196 mg, 593.12 umol) and 4,6-difluoro-N-(3-(isoxazol-5-yl)-1H-indazol-5-yl)-1-methyl-1H-indazole-5-carboxamide (125.83 mg, 593.12 umol) in pyridine (2 mL) was added EDCI (170.55 mg, 889.68 umol). The reaction mixture was stirred at 25° C. for 12 hrs. The reaction mixture was concentrated and purified by preparative HPLC (neutral condition) to afford the title compound (160 mg, 305.00 umol, 51% yield) as a white solid.

### Step 5: 4,6-Difluoro-N-(3-(isoxazol-5-yl)-1H-indazol-5-yl)-1-methyl-1H-indazole-5-carboxamide

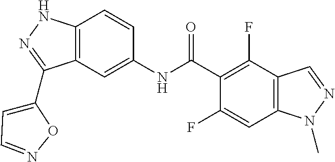 [SMILES: Cn1ncc2c(F)c(C(=O)Nc3ccc4[nH]nc(-c5ccno5)c4c3)c(F)cc21]

4,6-Difluoro-N-(3-(isoxazol-5-yl)-1-((2-(trimethylsilyl)ethoxy)methyl)-1H-indazol-5-yl)-1-methyl-1H-indazole-5-carboxamide (150 mg, 285.94 umol) was dissolved into TFA (3 mL). The reaction mixture was stirred at 70° C. for 2 hrs. The reaction mixture was concentrated and purified by preparative HPLC (neutral condition) to afford the title compound (6.11 mg, 12.02 umol, 4% yield) as a white solid. ¹H NMR (400 MHz, DMSO-d₆) δ 13.79 (br s, 1H), 10.94 (s, 1H), 8.75 (d, J=1.1 Hz, 1H), 8.68 (s, 1H), 8.35 (s, 1H), 7.71-7.65 (m, 3H), 6.95 (d, J=1.1 Hz, 1H), 4.09 (s, 3H). MS-ESI (m/z) calc'd for C₁₉H₁₃F₂N₆O₂ [M+H]⁺: 395.1. Found 395.1.

### Example 138: 4,6-Difluoro-1-methyl-N-(3-(oxazol-2-yl)-1H-indazol-5-yl)-1H-indazole-5-carboxamide

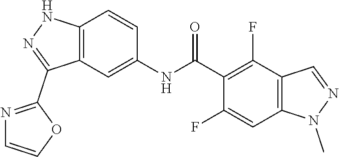 [SMILES: Cn1ncc2c(F)c(C(=O)Nc3ccc4[nH]nc(-c5ncco5)c4c3)c(F)cc21]

### Step 1: Oxazol-2-ylzinc(II) chloride

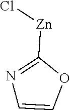

To a solution of oxazole (1 g, 14.48 mmol) in THF (20 mL) was added n-BuLi (2.5 M, 5.79 mL) at −78° C., the mixture was stirred for 0.5 hr, then ZnCl₂ (0.7 M, 20.69 mL) was added and the resulting mixture was warmed to 0° C. and stirred for 15 min to afford a yellow liquid. This mixture was used without further purification.

### Step 2: 2-(5-Nitro-1H-indazol-3-yl)oxazole

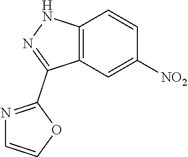 [SMILES: O=[N+]([O-])c1ccc2[nH]nc(-c3ncco3)c2c1]

A mixture of 3-iodo-5-nitro-1H-indazole (50 mg, 172.99 umol), Pd(PPh₃)₄ (19.99 mg, 17.30 umol) in THF (1 mL) was degassed and purged with N₂ (3×). Oxazol-2-ylzinc(II) chloride (292.18 mg, 1.73 mmol) (in 6 mL THF) was then added and the mixture was stirred at 70° C. for 2 hrs under N₂. 5 additional preparations were combined and these reaction mixtures were quenched by addition of H₂O (20 mL) and EtOAc (20 mL) at 20° C. and then diluted with 1 N HCl (8 mL) and extracted with EtOAc (100 mL×3). The combined organic layers were dried over Na₂SO₄, filtered and concentrated under reduced pressure to give a residue. The residue was purified by flash silica gel chromatography (ISCO®; 4 g SepaFlash® Silica Flash Column, eluent of 0-36%/petroleum ether gradient at 100 mL/min) to afford the title compound (180 mg, 414.46 umol, 40% yield) as a yellow solid.

### Step 3: tert-Butyl 5-nitro-3-(oxazol-2-yl)-1H-indazole-1-carboxylate

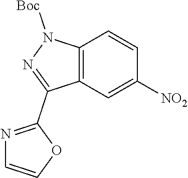 [SMILES: CC(C)(C)OC(=O)n1nc(-c2ncco2)c2cc([N+](=O)[O-])ccc21]

To a solution of 2-(5-nitro-1H-indazol-3-yl)oxazole (180 mg, 782.00 umol) in ACN (6 mL) was added (Boc)₂O (221.87 mg, 1.02 mmol), TEA (118.70 mg, 1.17 mmol) and DMAP (9.55 mg, 78.20 umol). The mixture was stirred at 20° C. for 12 hrs. The reaction mixture was concentrated under reduced pressure to remove solvent. The residue was diluted with EtOAc (5 mL) and an off-white solid precipitated. The precipitate was filtered and collected and the solid was washed with EtOAc (15 mL) and dried under vacuum to afford the title compound (100 mg, crude) as a pale yellow solid.

### Step 4: tert-Butyl 5-amino-3-(oxazol-2-yl)-1H-indazole-1-carboxylate

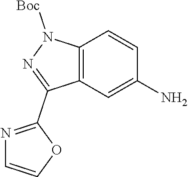 [SMILES: CC(C)(C)OC(=O)n1nc(-c2ncco2)c2cc(N)ccc21]

To a solution of tert-butyl 5-nitro-3-(oxazol-2-yl)-1H-indazole-1-carboxylate (60 mg, 199.79 umol) in pyridine (2 mL) was added EDCI (57.45 mg, 299.69 umol) and 4,6-difluoro-1-methyl-1H-indazole-5-carboxylic acid (42.39 mg, 199.79 umol). The mixture was stirred at 20° C. for 15 hrs. The reaction mixture was concentrated under reduced pressure to remove solvent. The residue was diluted with H₂O (5 mL) and extracted with EtOAc (5 mL×3). The combined organic layers were dried over Na₂SO₄, filtered and concentrated under reduced pressure to give a residue. The residue was purified by preparative TLC (SiO₂, ether/EtOAc=0/1, Rf=0.64) to afford the title compound (15 mg, 30.34 umol, 15% yield) as a pale yellow solid.

### Step 5: 4,6-Difluoro-1-methyl-N-(3-(oxazol-2-yl)-1H-indazol-5-yl)-1H-indazole-5-carboxamide

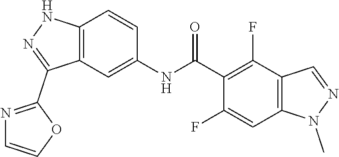 [SMILES: Cn1ncc2c(F)c(C(=O)Nc3ccc4[nH]nc(-c5ncco5)c4c3)c(F)cc21]

A solution of tert-butyl 5-amino-3-(oxazol-2-yl)-1H-indazole-1-carboxylate (15 mg, 30.34 umol) in DCM (0.8 mL) and TFA (0.2 mL) was stirred at 20° C. for 2 hrs. The reaction mixture was concentrated under reduced pressure to remove solvent. The residue was purified by preparative HPLC (TFA condition) to afford the title compound (2.27 mg, 4.27 umol, 14% yield) as a white solid. ¹H NMR (400 MHz, DMSO-d₆) δ 13.69 (br s, 1H) 10.91 (s, 1H) 8.80 (s, 1H) 8.20-8.41 (m, 2H) 7.62-7.75 (m, 3H) 7.49 (s, 1H) 4.09 (s, 3H). MS-ESI (m/z) calc'd for C₁₉H₁₃F₂N₆O₂ [M+H]⁺: 395.1. Found 395.1.

### Example 139: N-(3-(Azetidin-1-yl)-1H-indazol-5-yl)-4,6-difluoro-1-methyl-1H-indazole-5-carboxamide

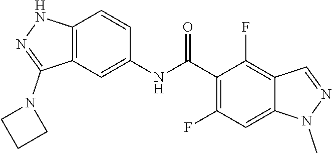 [SMILES: Cn1ncc2c(F)c(C(=O)Nc3ccc4[nH]nc(N5CCC5)c4c3)c(F)cc21]

### Step 1: 3-(Azetidin-1-yl)-5-nitro-1-((2-(trimethylsilyl)ethoxy)methyl)-1H-indazole

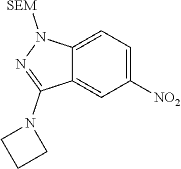 [SMILES: C[Si](C)(C)CCOCn1nc(N2CCC2)c2cc([N+](=O)[O-])ccc21]

To a mixture of azetidine (1.5 g, 11.54 mmol, HCl salt) in toluene (100 mL) was added Cs₂CO₃ (4.51 g, 13.84 mmol) and the mixture was stirred at 15° C. for 30 min. 3-Bromo-5-nitro-1-((2-(trimethylsilyl)ethoxy)methyl)-1H-indazole (1.72 g, 4.61 mmol) was then added followed by BINAP (287.35 mg, 461.48 umol) and Pd₂(dba)₃ (422.59 mg, 461.48 umol). The mixture was degassed and purged with N₂ (3×) and stirred at 100° C. for 12 hrs under N₂ atmosphere. The reaction mixture was concentrated under reduced pressure to remove solvent. The residue was diluted with MeOH (30 mL) and filtered. The filtrate was concentrated to give a residue. The residue was purified by column chromatography (SiO₂, ether/EtOAc=0/1 to 3/1) to afford the title compound (1.1 g, 3.16 mmol, 68% yield) as a yellow solid.

### Step 2: 3-(Azetidin-1-yl)-1-((2-(trimethylsilyl)ethoxy)methyl)-1H-indazol-5-amine

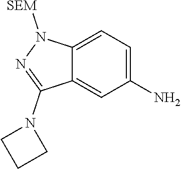 [SMILES: C[Si](C)(C)CCOCn1nc(N2CCC2)c2cc(N)ccc21]

To a solution of 3-(azetidin-1-yl)-5-nitro-1-((2-(trimethylsilyl)ethoxy)methyl)-1H-indazole (500 mg, 1.43 mmol) in THF (50 mL) was added NH₄Cl (1.23 g, 22.96 mmol) in H₂O (12.5 mL) and Zn (750.59 mg, 11.48 mmol). The mixture was stirred at 15° C. for 0.5 hr. The reaction mixture was filtered and the filtrate was diluted with H₂O (10 mL) and extracted with EtOAc (20 mL×3). The combined organic phases were dried over anhydrous Na₂SO₄, filtered and the filtrate was concentrated under reduced pressure to afford the title compound (495 mg, crude) as a yellow oil.

### Step 3: N-(3-(Azetidin-1-yl)-1-((2-(trimethylsilyl)ethoxy)methyl)-1H-indazol-5-yl)-4,6-difluoro-1-methyl-1H-indazole-5-carboxamide

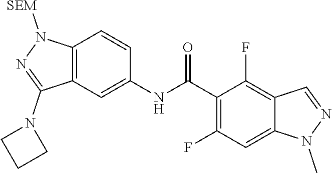 [SMILES: Cn1ncc2c(F)c(C(=O)Nc3ccc4c(c3)c(N3CCC3)nn4COCC[Si](C)(C)C)c(F)cc21]

To a solution of 3-(azetidin-1-yl)-1-((2-(trimethylsilyl)ethoxy)methyl)-1H-indazol-5-amine (495 mg, 1.55 mmol) and 4,6-difluoro-1-methyl-1H-indazole-5-carboxylic acid (274.78 mg, 1.30 mmol) in pyridine (10 mL) was added EDCI (372.43 mg, 1.94 mmol). The mixture was stirred at 15° C. for 12 hrs. The reaction mixture was concentrated under reduced pressure to remove solvent. The residue was purified by preparative HPLC (neutral condition) to afford the title compound (205 mg, 399.90 umol, 31% yield) as a yellow solid.

### Step 4: N-(3-(Azetidin-1-yl)-1H-indazol-5-yl)-4,6-difluoro-1-methyl-1H-indazole-5-carboxamide

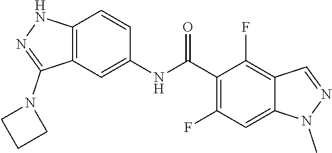 [SMILES: Cn1ncc2c(F)c(C(=O)Nc3ccc4[nH]nc(N5CCC5)c4c3)c(F)cc21]

To a solution of N-(3-(azetidin-1-yl)-1-((2-(trimethylsilyl)ethoxy)methyl)-1H-indazol-5-yl)-4,6-difluoro-1-methyl-1H-indazole-5-carboxamide (50 mg, 97.54 umol) in DCM (1 mL) was added TFA (1 mL). The mixture was stirred at 15° C. for 12 hrs. The mixture was basified with saturated aqueous NaHCO₃ to pH=8, then the mixture was extracted with DCM (5 mL×3). The organic layer was dried over anhydrous Na₂SO₄, filtered and the filtrate was concentrated. The residue was purified by preparative HPLC (neutral condition) to afford the title compound (12.25 mg) as a white solid. ¹H NMR (400 MHz, DMSO-d₆) δ 11.80 (s, 1H), 10.66 (s, 1H), 8.32 (s, 1H), 8.04 (s, 1H), 7.64 (d, J=9.7 Hz, 1H), 7.49 (dd, J=1.8, 9.0 Hz, 1H), 7.31 (d, J=8.9 Hz, 1H), 4.08 (s, 3H), 4.03 (t, J=7.3 Hz, 4H), 2.38 (td, J=7.3, 14.6 Hz, 2H). MS-ESI (m/z) calc'd for C₁₉H₁₇F₂N₆O [M+H]⁺: 383.1. Found 383.1.

### Example 140: N-(3-Benzyl-1H-indazol-5-yl)-4,6-difluoro-1-methyl-1H-indazole-5-carboxamide

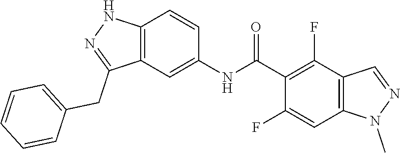 [SMILES: Cn1ncc2c(F)c(C(=O)Nc3ccc4[nH]nc(Cc5ccccc5)c4c3)c(F)cc21]

### Step 1: 3-Benzyl-5-nitro-1-((2-(trimethylsilyl)ethoxy)methyl)-1H-indazole

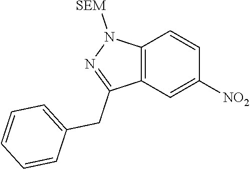 [SMILES: C[Si](C)(C)CCOCn1nc(Cc2ccccc2)c2cc([N+](=O)[O-])ccc21]

A mixture of 3-bromo-5-nitro-1-((2-(trimethylsilyl)ethoxy)methyl)-1H-indazole (500 mg, 1.34 mmol), 2-benzyl-4,4,5,5-tetramethyl-1,3,2-dioxaborolane (439.37 mg, 2.01 mmol), Pd(dppf)Cl₂ (98.27 mg, 134.30 umol) and K₂C₀₃ (556.85 mg, 4.03 mmol) in dioxane (2 mL) and H₂O (0.5 mL) was degassed and purged with N₂ (3×). The mixture was then stirred at 100° C. for 12 hrs under N₂ atmosphere and monitored by LC-MS. The reaction mixture was concentrated and purified by column chromatography (SiO₂, petroleum ether/EtOAc=20/1 to 2/1) to afford the title compound (500 mg, 1.30 mmol, 97% yield) as a yellow solid.

### Step 2: 3-Benzyl-1-((2-(trimethylsilyl)ethoxy)methyl)-1H-indazol-5-amine

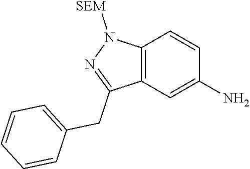 [SMILES: C[Si](C)(C)CCOCn1nc(Cc2ccccc2)c2cc(N)ccc21]

To a solution of 3-benzyl-5-nitro-1-((2-(trimethylsilyl)ethoxy)methyl)-1H-indazole (300 mg, 782.24 umol) in THF (2 mL) and H₂O (0.5 mL) was added Zn (409.20 mg, 6.26 mmol) and NH₄Cl (251.06 mg, 4.69 mmol). The reaction mixture was stirred at 25° C. for 1 hr and monitored by TLC (petroleum ether/=2/1, Rf=0.42). The reaction mixture was filtered and the filtrate was poured into water (5 mL). The aqueous phase was extracted with EtOAc (10 mL×3). The combined organic phases were washed with brine (10 mL×1), dried over anhydrous Na₂SO₄, filtered and concentrated in vacuum to afford the title compound (200 mg, 565.72 umol, 72% yield) as a yellow solid, which was used without further purification.

### Step 3: N-(3-Benzyl-1-((2-(trimethylsilyl)ethoxy)methyl)-1H-indazol-5-yl)-4,6-difluoro-1-methyl-1H-indazole-5-carboxamide

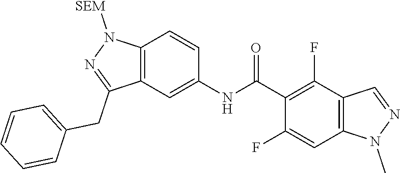 [SMILES: Cn1ncc2c(F)c(C(=O)Nc3ccc4c(c3)c(Cc3ccccc3)nn4COCC[Si](C)(C)C)c(F)cc21]

To a solution of 3-benzyl-1-((2-(trimethylsilyl)ethoxy)methyl)-1H-indazol-5-amine (150 mg, 424.29 umol) and 4,6-difluoro-1-methyl-1H-indazole-5-carboxylic acid (90.01 mg, 424.29 umol) in pyridine (2 mL) was added EDCI (122.01 mg, 636.43 umol). The reaction mixture was stirred at 25° C. for 12 hrs and monitored by LC-MS. The reaction mixture was concentrated and purified by column chromatography (SiO₂, petroleum ether/=20/1 to 0/1) to afford the title compound (130 mg, 237.37 umol, 56% yield) as a yellow solid.

### Step 4: N-(3-Benzyl-1H-indazol-5-yl)-4,6-difluoro-1-methyl-1H-indazole-5-carboxamide

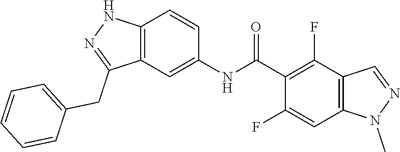 [SMILES: Cn1ncc2c(F)c(C(=O)Nc3ccc4[nH]nc(Cc5ccccc5)c4c3)c(F)cc21]

N-(3-Benzyl-1-((2-(trimethylsilyl)ethoxy)methyl)-1H-indazol-5-yl)-4,6-difluoro-1-methyl-1H-indazole-5-carboxamide (100 mg, 182.59 umol) was dissolved in TFA (3 mL). The reaction mixture was stirred at 70° C. for 2 hrs and monitored by LC-MS. The reaction mixture was concentrated and the residue was purified by preparative HPLC (TFA condition) and further purified by preparative HPLC (neutral condition) to afford the title compound (5.66 mg, 10.45 umol, 6% yield, TFA salt) as a yellow solid. ¹H NMR (400 MHz, DMSO-d₆) δ 12.78 (s, 1H), 10.69 (s, 1H), 8.32 (s, 1H), 8.09 (s, 1H), 7.63 (d, J=9.6 Hz, 1H), 7.55-7.45 (m, 2H), 7.28 (d, J=4.3 Hz, 4H), 7.21-7.14 (m, 1H), 4.26 (s, 2H), 4.07 (s, 3H). MS-ESI (m/z) calc'd for C₂₃H₁₈F₂N₅O [M+H]⁺: 418.1. Found 418.1.

### Example 141: N-(3-(1H-Imidazol-1-yl)-1H-indazol-5-yl)-4,6-difluoro-1-methyl-1H-indazole-5-carboxamide

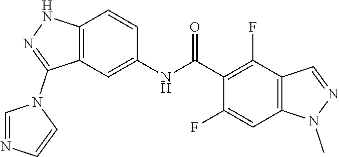 [SMILES: Cn1ncc2c(F)c(C(=O)Nc3ccc4[nH]nc(-n5ccnc5)c4c3)c(F)cc21]

### Step 1: 2,5-Dinitro-2H-indazole

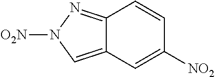 [SMILES: O=[N+]([O-])c1ccc2nn([N+](=O)[O-])cc2c1]

To a solution of HNO₃ (4.83 g, 75.16 mmol, 3.45 mL) in Ac₂O (8.76 g, 85.82 mmol) was added 5-nitroindazole (2 g, 12.26 mmol) in AcOH (18 mL) at −5° C. for 2 min. The mixture was poured onto ice and stirred at 0° C. for 30 min. The reaction mixture was filtered and the cake was dried under vacuum to afford the title compound (2.5 g, crude) as a yellow solid.

### Step 2: 3-(1H-Imidazol-1-yl)-5-nitro-1H-indazole

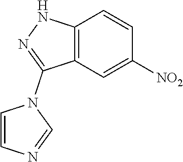 [SMILES: O=[N+]([O-])c1ccc2[nH]nc(-n3ccnc3)c2c1]

To a solution of 2,5-dinitro-2H-indazole (2.5 g, 12.01 mmol) in THF (54 mL) and H₂O (72 mL) was added imidazole (1.64 g, 24.02 mmol). The mixture was stirred at 20° C. for 12 hrs and monitored by LC-MS. The reaction mixture was extracted with EtOAc (100 mL×5). The combined organic layers were dried over Na₂SO₄, filtered and concentrated under reduced pressure to afford the title compound (2.9 g, crude) as an orange solid.

### Step 3: 3-(1H-Imidazol-1-yl)-1H-indazol-5-amine

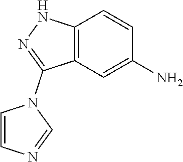 [SMILES: Nc1ccc2[nH]nc(-n3ccnc3)c2c1]

To a solution of 3-(1H-imidazol-1-yl)-5-nitro-1H-indazole (1.4 g, 6.11 mmol) in EtOH (7 mL) and H₂O (7 mL) was added Fe (1.71 g, 30.55 mmol) and NH₄Cl (1.63 g, 30.55 mmol). The mixture was stirred at 80° C. for 1 hr and monitored by LC-MS. The reaction mixture was filtered, the filtrate was concentrated under reduced pressure to afford 1.2 g crude product as a brown solid. 500 mg of the crude was purified by preparative HPLC (TFA condition) to afford the title compound (70 mg, TFA salt) as a brown solid.

### Step 4: N-(3-(1H-Imidazol-1-yl)-1H-indazol-5-yl)-4,6-difluoro-1-methyl-1H-indazole-5-carboxamide

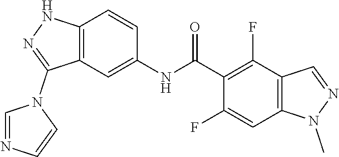 [SMILES: Cn1ncc2c(F)c(C(=O)Nc3ccc4[nH]nc(-n5ccnc5)c4c3)c(F)cc21]

To a solution of 3-(1H-imidazol-1-yl)-1H-indazol-5-amine (70 mg, 351.39 umol) in pyridine (2 mL) was added EDCI (122.47 mg, 638.88 umol) and 4,6-difluoro-1-methyl-1H-indazole-5-carboxylic acid (67.77 mg, 319.44 umol). The mixture was stirred at 40° C. for 12 hrs and monitored by LC-MS. The reaction mixture was concentrated under reduced pressure to remove solvent and purified by preparative HPLC (basic condition) to afford the title compound (33.86 mg, 85.69 umol, 27% yield) as a white solid. ¹H NMR (400 MHz, DMSO-d₆) δ 10.89 (br s, 1H) 8.34 (d, J=3.31 Hz, 2H) 8.23 (s, 1H) 7.74 (s, 1H) 7.64-7.68 (m, 3H) 7.22 (s, 1H) 4.08 (s, 3H). MS-ESI (m/z) calc'd for C₁₉H₁₄F₂N₇O [M+H]⁺: 394.1. Found 394.0.

### Example 142: 1-(5-(4,6-difluoro-1-methyl-1H-indazole-5-carboxamido)-1H-indazol-3-yl)azetidine-3-carboxylic acid

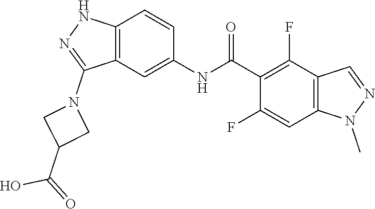 [SMILES: Cn1ncc2c(F)c(C(=O)Nc3ccc4[nH]nc(N5CC(C(=O)O)C5)c4c3)c(F)cc21]

### Step 1: Methyl 1-(5-nitro-1H-indazol-3-yl)azetidine-3-carboxylate

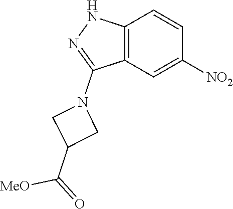 [SMILES: COC(=O)C1CN(c2n[nH]c3ccc([N+](=O)[O-])cc23)C1]

A stirred mixture of 3-bromo-5-nitro-1-((2-(trimethylsilyl)ethoxy)methyl)-1H-indazole (1.0 g, 2.69 mmol), methyl azetidine-3-carboxylate (610.78 mg, 4.03 mmol, HCl), Pd₂(dba)₃ (245.97 mg, 268.61 umol), Xantphos (310.84 mg, 537.22 umol) and Cs₂CO₃ (2.63 g, 8.06 mmol) in dioxane (10 mL) was stirred at 100° C. for 18 hrs under N₂ and monitored by LC-MS. The mixture was filtered through Celite and the filtrate was concentrated. The material was purified by silica gel chromatography using a step gradient of 3%, 10%, and 20% EtOAc/petroleum ether to afford the title compound (1 g, 2.25 mmol, 84% yield) as a brown oil.

### Step 2: Methyl 1-(5-amino-1H-indazol-3-yl)azetidine-3-carboxylate

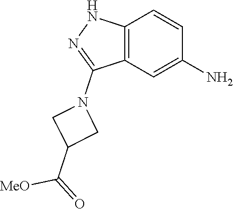 [SMILES: COC(=O)C1CN(c2n[nH]c3ccc(N)cc23)C1]

To a stirred solution of methyl 1-(5-nitro-1H-indazol-3-yl)azetidine-3-carboxylate (0.9 g, 2.21 mmol) in THF (8 mL) and H₂O (2 mL) was added Zn (1.16 g, 17.71 mmol) and NH₄Cl (710.57 mg, 13.28 mmol) in one portion. Then the mixture was stirred at 15° C. for 1 hr and monitored by TLC (petroleum ether/EtOAc=2/1, Rf=0.03). The mixture was filtered and the filtrate was dried over anhydrous Na₂SO₄ and concentrated under a stream of N₂ at 15° C. to afford the title compound (833 mg, crude) as a red oil which was used without further purification.

### Step 3: Methyl 1-(5-(4,6-difluoro-1-methyl-1H-indazole-5-carboxamido)-1-((2-(trimethylsilyl)ethoxy)methyl)-1H-indazol-3-yl)azetidine-3-carboxylate

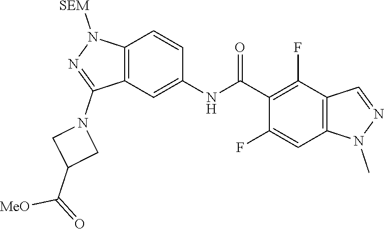 [SMILES: COC(=O)C1CN(c2nn(COCC[Si](C)(C)C)c3ccc(NC(=O)c4c(F)cc5c(cnn5C)c4F)cc23)C1]

To a stirred solution of methyl 1-(5-amino-1H-indazol-3-yl)azetidine-3-carboxylate (0.833 g, 2.21 mmol) in pyridine (10 mL) was added 4,6-difluoro-1-methyl-1H-indazole-5-carboxylic acid (704.03 mg, 3.32 mmol) followed by addition of EDCI (636.17 mg, 3.32 mmol) in one portion. The mixture was then stirred at 15° C. for 3 hrs and monitored by LC-MS. The mixture was diluted with EtOAc (30 mL) and washed with saturated aqueous NH₄Cl (10 mL×3). The organic layer was dried over anhydrous Na₂SO₄ and purified by silica gel chromatography using a step gradient of 10%, 30%, and 75% EtOAc/petroleum ether to afford the title compound (750 mg, 1.03 mmol, 47% yield) as a brown solid.

### Step 4: 1-(5-(4,6-Difluoro-1-methyl-1H-indazole-5-carboxamido)-1H-indazol-3-yl)azetidine-3-carboxylic acid

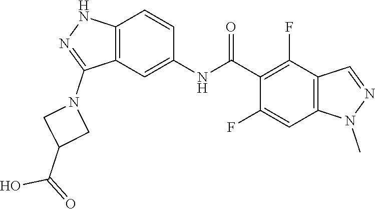 [SMILES: Cn1ncc2c(F)c(C(=O)Nc3ccc4[nH]nc(N5CC(C(=O)O)C5)c4c3)c(F)cc21]

To a stirred solution of methyl 1-(5-(4,6-difluoro-1-methyl-1H-indazole-5-carboxamido)-1-((2-(trimethylsilyl)ethoxy)methyl)-1H-indazol-3-yl)azetidine-3-carboxylate (250 mg, 343.46 umol) in DCM (4 mL) was added TFA (3.08 g, 27.01 mmol) dropwise at 15° C. The mixture was stirred at 15° C. for 12 hrs. Then the mixture was adjusted to pH=11 by dropwise addition of 10% aqueous NaOH at 0° C. This mixture was then stirred at 15° C. for 2 hrs. NH₃.H₂O (5 mL) was added to the mixture and the mixture was stirred at 15° C. for another 12 hrs and monitored by LC-MS. The mixture was concentrated by lyophilization and purified by preparative HPLC (Method L), then further purified by preparative HPLC (Method M) to afford the title compound (9.41 mg, 15.18 umol, 4% yield) as an off-white solid. ¹H NMR (400 MHz, DMSO-d₆) δ 11.90 (s, 1H) 10.67 (s, 1H) 8.32 (s, 1H) 8.07 (s, 1H) 7.63-7.66 (d, J=9.70 Hz, 1H) 7.48-7.50 (dd, 1H) 7.32-7.34 (d, 1H) 4.22-4.26 (t, J=7.60 Hz, 2H) 4.09-4.13 (br t, J=6.84 Hz, 2H) 4.08 (s, 3H) 3.58-3.66 (m, 1H). MS-ESI (m/z) calc'd for C₂₀H₁₇F₂N₆O₃ [M+H]⁺: 427.1. Found 427.1.

### Example 143: 4,6-Difluoro-N-(6-fluoro-3-phenyl-1H-indazol-5-yl)-1-methyl-1H-indazole-5-carboxamide

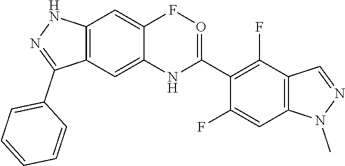

### Step 1: 6-Fluoro-3-iodo-5-nitro-1H-indazole

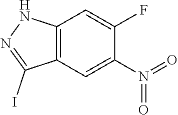 [SMILES: O=[N+]([O-])c1cc2c(I)n[nH]c2cc1F]

6-Fluoro-5-nitro-1H-indazole (0.4 g, 2.22 mmol) was dissolved in DMF (10 mL). The solution was cooled to 0° C. and 1-iodopyrrolidine-2,5-dione (0.55 g, 2.44 mmol) was added in portions. The mixture was stirred at room temperature for 1 hr. The mixture was quenched with water and extracted with DCM (2×). The combined organic layers were washed with water (1×), passed through a phase separator and evaporated to dryness. The residue was purified by column chromatography on a 100 g silica gel column using a 0-20% gradient of EtOAc in cyclohexane as eluent to afford the title compound (0.510 g, 1.661 mmol, 75% yield) as a white solid. ¹H NMR (400 MHz, CDCl₃) δ ppm 8.38 (d, J=6.82 Hz, 1H), 7.40 (d, J=10.56 Hz, 1H), 7.28 (s, 2H). MS-ESI (m/z) calc'd for C₇H₄FIN₃O₂ [M+H]⁺: 307.9. Found 308.0.

### Step 2: 6-Fluoro-3-iodo-1H-indazol-5-amine

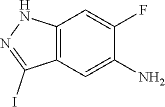 [SMILES: Nc1cc2c(I)n[nH]c2cc1F]

Iron powder (181.91 mg, 3.26 mmol) was added slowly to a solution of 6-fluoro-3-iodo-5-nitro-1H-indazole (200.0 mg, 0.650 mmol) in acetic acid (6 mL). The mixture was stirred at room temperature overnight. The residue was filtered, concentrated and partitioned between saturated aqueous NaHCO₃ and EtOAc. The phases were separated, the aqueous layer was extracted with EtOAc (2×), and the combined organic phases washed with water (1×), dried over anhydrous Na₂SO₄ and evaporated to dryness to give the title compound (180 mg, 0.650 mmol, 99% yield) as a beige solid. ¹H NMR (400 MHz, MeOD) δ ppm 7.12-7.25 (m, 1H), 6.83 (d, J=8.14 Hz, 1H). MS-ESI (m/z) calc'd for C₇H₆FIN₃ [M+H]⁺: 278.0. Found 278.0.

### Step 3: 6-Fluoro-3-phenyl-1H-indazol-5-amine

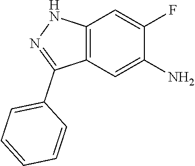 [SMILES: Nc1cc2c(-c3ccccc3)n[nH]c2cc1F]

A mixture of 6-fluoro-3-iodo-1H-indazol-5-amine (50 mg, 0.18 mmol), phenylboronic acid (44 mg, 0.36 mmol) and K₃PO₄ (115 mg, 0.54 mmol) in THF/water (1.9/0.6 mL) was prepared and N₂ was bubbled through the mixture for 10 minutes. SPhos-Pd-G2 (19 mg, 0.027 mmol) was added and the mixture was heated at 80° C. overnight. The organic solvent was evaporated and the residue was taken up in DCM and washed with sat. aq. NaHCO₃. The organic phase was separated, filtered through a hydrophobic phase separator and evaporated at reduced pressure. Purification by flash chromatography on Biotage SNAP-NH 28 g cartridge (DCM:MeOH from 100:0 to 97:3) afforded the title compound (41.2 mg, 0.181 mmol) as a white solid. ¹H NMR (400 MHz, DMSO-d₆) δ ppm 12.86 (br. s., 1H), 7.89 (d, J=7.04 Hz, 2H), 7.46-7.60 (m, 2H), 7.32-7.40 (m, 2H), 7.25 (d, J=11.22 Hz, 1H), 4.94 (s, 2H). MS-ESI (m/z) calc'd for C₁₃H₁₁FN₃ [M+H]⁺: 228.1. Found 228.2.

### Step 4: tert-Butyl 5-((tert-butoxycarbonyl)amino)-6-fluoro-3-phenyl-1H-indazole-1-carboxylate

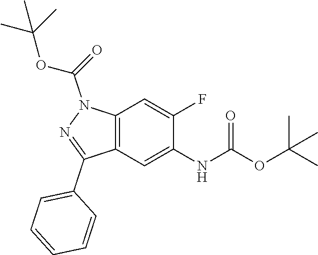 [SMILES: CC(C)(C)OC(=O)Nc1cc2c(-c3ccccc3)nn(C(=O)OC(C)(C)C)c2cc1F]

6-Fluoro-3-phenyl-1H-indazol-5-amine (41.0 mg, 0.180 mmol) was dissolved in THF (0.30 mL) and N,N-diisopropylethylamine (0.03 mL, 0.200 mmol) was added. The reaction mixture was cooled to 0° C. followed by the addition of di-tert-butyl dicarbonate (0.04 mL, 0.180 mmol); the reaction was then stirred at room temperature overnight. Additional N,N-diisopropylethylamine (0.03 mL, 0.200 mmol) and di-tert-butyl dicarbonate (0.04 mL, 0.180 mmol) were added and stirring was continued for an additional 24 hrs. The reaction was concentrated under reduced pressure and the residue was purified on a Biotage 11 g silica-NH cartridge (cyclohexane/EtOAc 0:0 to 70:30) to afford the title compound (27 mg, 0.063 mmol, 35% yield). MS-ESI (m/z) calc'd for C₂₃H₂₇FN₃O₄ [M+H]⁺: 428.2. Found 428.1.

### Step 5: 6-Fluoro-3-phenyl-1H-indazol-5-amine

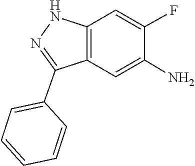 [SMILES: Nc1cc2c(-c3ccccc3)n[nH]c2cc1F]

Tert-Butyl 6-fluoro-5-[(2-methylpropan-2-yl)oxycarbonylamino]-3-phenylindazole-1-carboxylate (27.0 mg, 0.060 mmol), DCM (0.3 mL) and TFA (0.05 mL, 0.63 mmol) were stirred at room temperature overnight. The reaction mixture was concentrated to afford the title compound as yellow oil which was used without further purification.

### Step 6: 4,6-Difluoro-N-(6-fluoro-3-phenyl-1H-indazol-5-yl)-1-methyl-1H-indazole-5-carboxamide

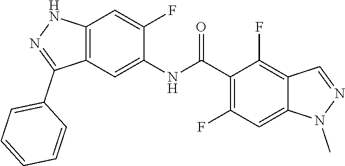 [SMILES: Cn1ncc2c(F)c(C(=O)Nc3cc4c(-c5ccccc5)n[nH]c4cc3F)c(F)cc21]

Trimethylaluminum (0.077 mL, 2 M in toluene), 6-fluoro-3-phenyl-1H-indazol-5-amine (17.5 mg, 0.080 mmol) and methyl 4,6-difluoro-1-methyl-1H-indazole-5-carboxylate (22.64 mg, 0.100 mmol) were dissolved in toluene (2 mL). The reaction mixture was stirred at 100° C. for 1 hr. A further amount of trimethylaluminum (0.077 mL, 2 M in toluene) was added and the reaction mixture was stirred at 100° C. for 3 hrs. The reaction was cooled to room temperature. Water was added followed by EtOAc. The organic layer was separated, dried over Na₂SO₄, filtered and concentrated to give crude material which was purified by preparative HPLC (Method A) to afford the title compound as a white solid (3.0 mg, 0.007 mmol, 9% yield). ¹H NMR (400 MHz, DMSO-d₆) δ ppm 13.41 (br. s., 1H), 10.59 (br. s., 1H), 8.47 (d, J=7.26 Hz, 1H), 8.34 (d, J=0.66 Hz, 1H), 7.87-8.00 (m, 2H), 7.65 (d, J=9.90 Hz, 1H), 7.53-7.60 (m, 3H), 7.41-7.48 (m, 1H), 4.09 (s, 3H). MS-ESI (m/z) calc'd for C₂₂H₁₅F₃N₅O [M+H]⁺: 422.1. Found 422.0.

### Example 144: 4,6-Difluoro-N-(6-fluoro-3-(furan-3-yl)-1H-indazol-5-yl)-1-methyl-1H-indazole-5-carboxamide

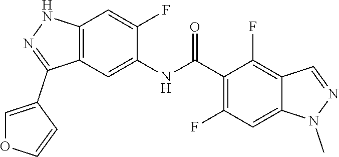 [SMILES: Cn1ncc2c(F)c(C(=O)Nc3cc4c(-c5ccoc5)n[nH]c4cc3F)c(F)cc21]

### Step 1: 6-Fluoro-3-iodo-5-nitro-1H-indazole

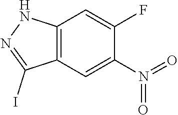 [SMILES: O=[N+]([O-])c1cc2c(I)n[nH]c2cc1F]

6-Fluoro-5-nitro-1H-indazole (0.6 g, 3.31 mmol) was dissolved in DMF (14.91 mL). The solution was cooled to 0° C. and 1-iodopyrrolidine-2,5-dione (0.82 g, 3.64 mmol) was added in portions. The mixture was stirred at r.t. overnight. An additional amount of 1-iodopyrrolidine-2,5-dione (0.82 g, 3.64 mmol) was added and stirring was continued for an additional 24 hrs. The mixture was quenched with sat. aq. Na₂S₂O₃ and extracted with DCM (2×). The combined organic layers were washed with water, passed through a phase separator and evaporated to dryness. The residue was purified by column chromatography on a 50 g silica gel column, using a 0-20% gradient of EtOAc in cyclohexane to afford the title compound (1 g, 3.257 mmol, 98% yield) as a yellow solid. ¹H NMR (400 MHz, DMSO-d₆) δ ppm 14.15 (br. s., 1H), 8.29 (d, J=7.04 Hz, 1H), 7.65-7.93 (m, 1H). MS-ESI (m/z) calc'd for C₇H₄IFN₃O₂ [M+H]⁺: 307.9. Found 307.9.

### Step 2: 6-Fluoro-3-iodo-5-nitro-1-((2-(trimethylsilyl)ethoxy)methyl)-1H-indazole

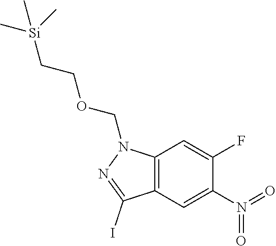 [SMILES: C[Si](C)(C)CCOCn1nc(I)c2cc([N+](=O)[O-])c(F)cc21]

To a solution of NaH (60% mineral oil, 130.28 mg, 3.26 mmol) in THF (2.7 mL) at 0° C. was added 6-fluoro-3-iodo-5-nitro-1H-indazole (500.0 mg, 1.63 mmol) dropwise and the mixture was stirred for 20 minutes. Then 2-(chloromethoxy)ethyl-trimethylsilane (352.97 mg, 2.12 mmol) was added slowly to the mixture and stirring continued for 1 hr at 0° C. The mixture was diluted with NH₄Cl and the aqueous layer was extracted with EtOAc. The combined organic layers were washed with brine, dried over Na₂SO₄, filtered and concentrated under reduced pressure. The residue was purified by flash chromatography on a 25 g Biotage silica gel cartridge (from cyclohexane:EtOAc=100:0 to cyclohexane:EtOAc=70:30) to afford the title compound (0.226 g, 0.52 mmol, 32% yield). ¹H NMR (400 MHz, DMSO-d₆) δ ppm 8.40-8.20 (m, 1H) 8.20-8.10 (m, 1H) 5.79 (s, 2H) 3.59-3.55 (m, 2H) 0.84-0.79 (m, 2H) 0.03-0.02 (m, 2H) −0.08-−0.09 (m, 9H). MS-ESI (m/z) calc'd for C₁₃H₁₈IFN₃O₃Si [M+H]⁺: 438.0. Found 438.0.

### Step 3: 6-Fluoro-3-iodo-1-((2-(trimethylsilyl)ethoxy)methyl)-1H-indazol-5-amine

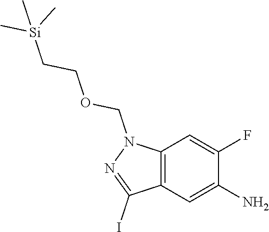 [SMILES: C[Si](C)(C)CCOCn1nc(I)c2cc(N)c(F)cc21]

A mixture of 6-fluoro-3-iodo-5-nitro-1-((2-(trimethylsilyl)ethoxy)methyl)-1H-indazole (0.479 g, 1.09 mmol), NH₄Cl (63 mg, 1.17 mmol), and iron powder (0.263 g, 4.7 mmol) in EtOH/H₂O (1.8 mL/2 mL) was heated to reflux for 45 min. The mixture was cooled and filtered through a Celite pad. The filtrate was evaporated and the resulting residue was diluted with EtOAc and H₂O. The aqueous layer was separated and extracted with EtOAc. The combined organic extracts were washed with brine, dried over Na₂SO₄, filtered, and evaporated. The residue was purified by flash chromatography on a 55 g Biotage silica-NH cartridge (from cyclohexane:EtOAc=100:0 to cyclohexane:EtOAc=70:30) to afford the title compound (0.295 g, 0.72 mmol, 66% yield). ¹H NMR (400 MHz, DMSO-d₆) δ ppm 7.48-7.60 (m, 1H), 6.69 (d, J=8.36 Hz, 1H), 5.55-5.66 (m, 2H), 5.16 (s, 2H), 3.40-3.55 (m, 2H), 0.70-0.83 (m, 2H), −0.16-−0.05 (m, 9H). MS-ESI (m/z) calc'd for C₁₃H₂₀IFN₃OSi [M+H]⁺: 408.0. Found 408.1.

### Step 4: 6-Fluoro-3-(furan-3-yl)-1-((2-(trimethylsilyl)ethoxy)methyl)-1H-indazol-5-amine

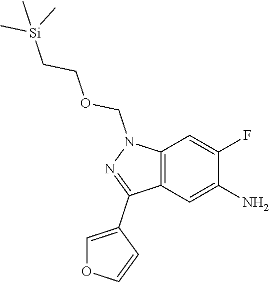 [SMILES: C[Si](C)(C)CCOCn1nc(-c2ccoc2)c2cc(N)c(F)cc21]

A mixture of 6-fluoro-3-iodo-1-((2-(trimethylsilyl)ethoxy)methyl)-1H-indazol-5-amine (295.0 mg, 0.720 mmol), 3-furanylboronic acid (162.08 mg, 1.45 mmol) and K₃PO₄ (0.459 g, 2.16 mmol) in THF/H₂O (8.4 mL/2.5 mL) was prepared and N₂ was bubbled through the mixture for 10 minutes. Then SPhos-Pd-G2 (0.078 g, 0.108 mmol) was added and the mixture was heated at 80° C. overnight. The organic solvent was evaporated, and the resulting residue was diluted with DCM and washed with sat. aq. NaHCO₃. The organic phase was separated, filtered through a hydrophobic phase separator and evaporated at reduced pressure. The crude material was purified by flash chromatography on a 55 g Biotage silica-NH cartridge (from cyclohexane:EtOAc=100:0 to cyclohexane:EtOAc=60:40) to afford the title compound (209 mg, 0.602 mmol, 83% yield) as a yellow oil. ¹H NMR (400 MHz, DMSO-d₆) δ ppm 8.21 (dd, J=1.54, 0.88 Hz, 1H), 7.83 (t, J=1.65 Hz, 1H), 7.48-7.61 (m, 1H), 7.24 (d, J=8.36 Hz, 1H), 6.93 (dd, J=1.87, 0.77 Hz, 1H), 5.58-5.68 (m, 2H), 3.48-3.57 (m, 2H), 0.76-0.87 (m, 2H), −0.18-−0.07 (m, 9H). MS-ESI (m/z) calc'd for C₁₇H₂₃FN₃O₂Si [M+H]⁺: 348.2. Found 348.2.

### Step 5: 4,6-Difluoro-N-(6-fluoro-3-(furan-3-yl)-1-((2-(trimethylsilyl)ethoxy)methyl)-1H-indazol-5-yl)-1-methyl-1H-indazole-5-carboxamide

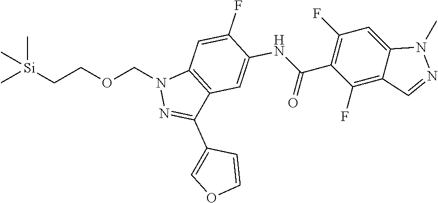

Methyl 4,6-difluoro-1-methyl-1H-indazole-5-carboxylate (42.31 mg, 0.190 mmol), 6-fluoro-3-(furan-3-yl)-1-((2-(trimethylsilyl)ethoxy)methyl)-1H-indazol-5-amine (50.0 mg, 0.140 mmol) and trimethylaluminum (2.0 M in toluene, 0.28 mL, 0.56 mmol) were dissolved in toluene (1 mL) and the reaction was stirred at 100° C. for 1 hr. The reaction was cooled to room temperature, diluted with water and extracted with EtOAc. The organic phase was separated, dried over Na₂SO₄, filtered and concentrated. The crude material was purified by flash chromatography on a 10 g Biotage silica gel cartridge (from cyclohexane:EtOAc=100:0 to cyclohexane:EtOAc=50:50) to afford the title compound (62 mg, 0.114 mmol, 80% yield) as a white solid. ¹H NMR (400 MHz, DMSO-d₆) δ ppm 10.59 (s, 1H), 8.43-8.53 (m, 1H), 8.27-8.39 (m, 2H), 7.81-7.92 (m, 2H), 7.65 (d, J=9.46 Hz, 1H), 7.00 (d, J=1.10 Hz, 1H), 5.77 (s, 2H), 4.03-4.14 (m, 3H), 3.50-3.64 (m, 2H), 0.75-0.88 (m, 2H), −0.17-−0.02 (m, 9H). MS-ESI (m/z) calc'd for C₂₆H₂₇F₃N₅O₃Si [M+H]⁺: 542.2. Found 542.2.

### Step 6: 4,6-Difluoro-N-(6-fluoro-3-(furan-3-yl)-1H-indazol-5-yl)-1-methyl-1H-indazole-5-carboxamide

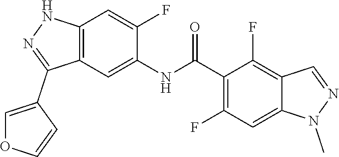 [SMILES: Cn1ncc2c(F)c(C(=O)Nc3cc4c(-c5ccoc5)n[nH]c4cc3F)c(F)cc21]

4,6-Difluoro-N-(6-fluoro-3-(furan-3-yl)-1-((2-(trimethylsilyl)ethoxy)methyl)-1H-indazol-5-yl)-1-methyl-1H-indazole-5-carboxamide (0.054 g, 0.1 mmol) was dissolved in DCM (6.4 mL) and trifluoroacetic acid was added (6.4 mL). The reaction mixture was stirred at room temperature for 1 hr. The mixture was then evaporated, diluted with saturated aqueous NaHCO₃ and extracted with DCM. The organic phase was separated, dried over Na₂SO₄, filtered, concentrated and added to a second batch of crude material prepared in a similar manner. The combined preparations (50 mg) were then submitted to preparative HPLC under acid conditions (method B′) to afford the title compound (9.8 mg, 0.024 mmol) as a white solid. ¹H NMR (400 MHz, DMSO-d₆) δ ppm 13.24 (br. s., 2H), 10.53 (s, 1H), 8.40 (s, 1H), 8.34 (d, J=0.88 Hz, 1H), 8.30 (d, J=7.04 Hz, 1H), 7.85 (t, J=1.65 Hz, 1H), 7.65 (d, J=9.90 Hz, 1H), 7.51 (d, J=10.34 Hz, 1H), 6.96-7.05 (m, 1H), 4.06-4.12 (m, 3H). MS-ESI (m/z) calc'd for C₂₀H₁₃F₃N₅O₂ [M+H]⁺: 412.1. Found 412.0.

### Example 145: 7-Methyl-N-(3-methyl-1H-indazol-5-yl)imidazo[1,5-a]pyridine-6-carboxamide

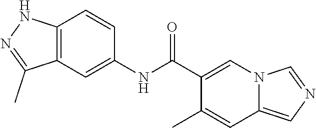 [SMILES: Cc1cc2cncn2cc1C(=O)Nc1ccc2[nH]nc(C)c2c1]

### Step 1: Methyl 6-chloro-4-methylnicotinate

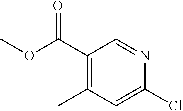

A suspension of 6-chloro-4-methylpyridine-3-carboxylic acid (2.19 g, 12.76 mmol) in phosphorus oxychloride (21.9 mL, 234.24 mmol) was heated at 100° C. for 15 hrs. The excess of POCl₃ was evaporated and the residue was carefully quenched with methanol (21.41 mL, 541.35 mmol) at 0° C. The solvent was evaporated and the residue was taken up in sat. aq. NaHCO₃ and stirred for 15 minutes. The solid formed was extracted with DCM (3×) and the combined organic layers were passed through a phase separator and evaporated to obtain the title compound (2.369 g, 12.76 mmol, 100% yield) as a dark solid. ¹H NMR (400 MHz, DMSO-d₆) δ ppm 8.75 (s, 1H), 7.59 (t, J=0.7 Hz, 1H), 3.86 (s, 3H), 2.54 (s, 3H). MS-ESI (m/z) calc'd for C₈H₉ClNO₂ [M+H]⁺: 186.1. Found 186.1.

### Step 2: Methyl 4-methyl-6-vinylnicotinate

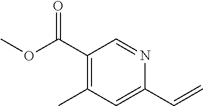

A solution of methyl 6-chloro-4-methylnicotinate (2.37 g, 11.75 mmol) and tetrakis(triphenylphosphine)palladium(0) (0.68 g, 0.590 mmol) in toluene (60 mL) was degassed with N₂ for 15 minutes. Tributyl(ethenyl)stannane (4.12 mL, 14.1 mmol) was added and the mixture was stirred at 100° C. for 15 hrs. The solvent was evaporated and the residue was purified by column chromatography (SiO₂, 100 g) using cyclohexane as eluant for 3 CV followed by a 0-50% gradient of EtOAc in cyclohexane for 10 CV to obtain the title compound (1.37 g, 7.731 mmol, 66% yield) as a yellow oil. ¹H NMR (400 MHz, DMSO-d₆) δ ppm 8.89 (s, 1H), 7.49 (s, 1H), 6.83 (dd, J=17.4, 10.7 Hz, 1H), 6.36 (dd, J=17.5, 1.6 Hz, 1H), 5.60 (dd, J=10.7, 1.6 Hz, 1H), 3.86 (s, 3H), 2.55 (s, 3H). MS-ESI (m/z) calc'd for C₁₀H₁₂NO₂ [M+H]⁺: 178.1. Found 178.0.

### Step 3: Methyl 6-formyl-4-methylnicotinate

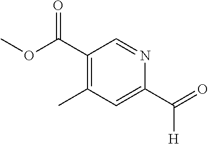

To a solution of methyl 4-methyl-6-vinylnicotinate carboxylate (1.37 g, 7.73 mmol) in 1,4-dioxane (23 mL) was added 4% osmium tetroxide (2.46 mL, 0.390 mmol) and a solution of sodium periodate (3.31 g, 15.46 mmol) in water (23 mL). The mixture was stirred at 25° C. for 15 hrs. The mixture was diluted with water and extracted with DCM (3×), the combined organic layers were passed through a phase separator and evaporated to obtain the title compound (1.385 g, 7.732 mmol, 100% yield) as a dark solid which was used for the next step without further purification. ¹H NMR (400 MHz, CDCl₃) δ ppm 10.11 (s, 1H), 9.19 (s, 1H), 7.83 (t, J=0.8 Hz, 1H), 3.98 (s, 3H), 2.70 (s, 3H). MS-ESI (m/z) calc'd for C₉H₁₀NO₃ [M+H]⁺: 180.1. Found 180.0.

### Step 4: Methyl (E)-6-((hydroxyimino)methyl)-4-methylnicotinate

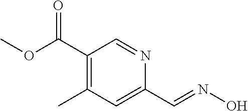

To a solution of methyl 6-formyl-4-methylpyridine-3-carboxylate (0.8 g, 4.47 mmol) in MeOH (30 mL) was added hydroxylamine hydrochloride (310 mg, 4.47 mmol) and potassium carbonate (0.62 g, 4.47 mmol), the mixture was stirred at 25° C. for 15 hrs. The solvent was evaporated, the residue was taken up in water and extracted with EtOAc (3×). The combined organic layers were passed through a phase separator and evaporated to obtain the title compound (560 mg, 2.884 mmol, 65% yield). ¹H NMR (400 MHz, DMSO-d₆) δ ppm 11.95 (s, 1H), 8.92 (s, 1H), 8.10 (s, 1H), 7.74 (s, 1H), 3.87 (s, 3H), 2.57 (s, 3H). MS-ESI (m/z) calc'd for C₉H₁₁N₂O₃ [M+H]⁺: 195.1. Found 195.0.

### Step 5: Methyl 6-(aminomethyl)-4-methylnicotinate

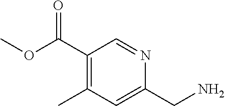

To a solution of methyl (E)-6-((hydroxyimino)methyl)-4-methylnicotinate (560 mg, 2.88 mmol) in acetic acid (7 mL) was added zinc (943 mg, 14.42 mmol) at 0° C. and the mixture was stirred at 0° C. for 6 hrs. The zinc was filtered through a Celite pad and the solution was evaporated to dryness. The residue was taken up in DCM and the solid formed was filtered. The filtrate was evaporated to obtain the title compound (520 mg, 2.884 mmol, 100% yield) as a dark oil which was used without further purification. ¹H NMR (400 MHz, DMSO-d₆) δ ppm 8.94 (s, 1H), 7.55 (s, 1H), 5.75 (s, 2H), 4.07 (s, 2H), 3.90 (s, 3H), 2.62 (s, 3H). MS-ESI (m/z) calc'd for C₉H₁₃N₂O₂ [M+H]⁺: 181.1. Found 181.0.

### Step 6: Methyl 6-(formamidomethyl)-4-methylnicotinate

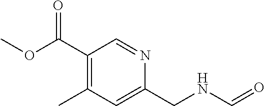

A mixture of methyl 6-(aminomethyl)-4-methylnicotinate (520 mg, 2.88 mmol) in formic acid (10 mL, 265.04 mmol) was heated at 100° C. for 3 hrs. The solvent was evaporated to dryness to obtain the title compound (600.48 mg, 2.884 mmol, 100% yield) as a dark oil which was used without further purification. ¹H NMR (400 MHz, DMSO-d₆) δ ppm 8.86 (s, 1H), 8.62 (s, 1H), 8.18 (d, J=1.7 Hz, 1H), 7.27 (s, 1H), 4.41 (d, J=6.0 Hz, 2H), 3.85 (s, 3H), 2.53 (s, 3H). MS-ESI (m/z) calc'd for C₁₀H₁₃N₂O₃ [M+H]⁺: 209.1. Found 209.0.

### Step 7: Methyl 7-methylimidazo[1,5-a]pyridine-6-carboxylate

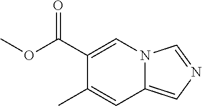

To a solution of methyl 6-(formamidomethyl)-4-methylnicotinate (600 mg, 2.88 mmol) in toluene (19 mL) was added phosphorus oxychloride (3 mL, 31.72 mmol) and the mixture was stirred at 65° C. for 2 hrs. The solvent was evaporated to dryness, and the residue was taken up in DCM and washed with sat. aq. NaHCO₃, passed through a phase separator and evaporated to obtain the title compound (470 mg, 2.471 mmol, 86% yield) as a dark solid which was used without further purification. ¹H NMR (400 MHz, DMSO-d₆) δ ppm 9.06 (s, 1H), 8.47 (s, 1H), 7.37 (d, J=1.3 Hz, 1H), 7.26 (s, 1H), 3.84 (s, 3H), 2.42 (s, 3H). MS-ESI (m/z) calc'd for C₁₀H₁₁N₂O₂ [M+H]⁺: 191.1. Found 191.1.

### Step 8: 7-Methylimidazo[1,5-a]pyridine-6-carboxylic acid

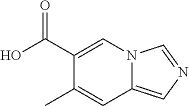

To a solution of methyl 7-methylimidazo[1,5-a]pyridine-6-carboxylate (470 mg, 2.47 mmol) in THF (20 mL) was added LiOH.H₂O (311 mg, 7.41 mmol) in water (5 mL) and the mixture was stirred at 25° C. for 15 hrs. The organic solvent was evaporated and the pH was adjusted to ˜4 by addition of HCl. Evaporation to dryness gave the title compound (435 mg, 2.471 mmol, 100% yield) as a brown solid. ¹H NMR (400 MHz, DMSO-d₆) δ ppm 13.62 (s, 1H), 9.46 (s, 1H), 9.18 (s, 1H), 7.84 (s, 1H), 7.59 (d, J=1.6 Hz, 1H), 2.48 (s, 3H). MS-ESI (m/z) calc'd for C₉H₉N₂O₂ [M+H]⁺: 177.1. Found 177.0.

### Step 9: 7-Methyl-N-(3-methyl-1H-indazol-5-yl)imidazo[1,5-a]pyridine-6-carboxamide

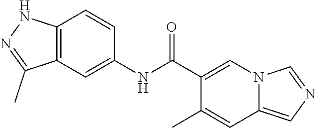 [SMILES: Cc1cc2cncn2cc1C(=O)Nc1ccc2[nH]nc(C)c2c1]

7-Methylimidazo[1,5-a]pyridine-6-carboxylic acid (20.0 mg, 0.110 mmol) and 3-methyl-1H-indazol-5-amine (20.05 mg, 0.140 mmol) were dissolved in dry DMF (1.5 mL). Then the solution was cooled to 0° C. with an ice-water bath and HATU (51.8 mg, 0.140 mmol) and triethylamine (23.74 uL, 0.170 mmol) were added. The mixture was stirred at 0° C. for 5 min and then at room temperature for 3 hrs. The reaction mixture was partitioned between water and EtOAc and the phases were separated. The aqueous layer was extracted with EtOAc (2×) and the combined organic phases washed with water (1×), dried over anhydrous Na₂SO₄ and evaporated to dryness. The crude material was purified by normal phase chromatography on an 11 g NH silica gel column, using a gradient of MeOH in EtOAc from 0 to 10% as eluent to afford the title compound (11 mg, 0.036 mmol, 32% yield) as a light yellow solid. ¹H NMR (400 MHz, DMSO-d₆) δ ppm 12.60 (br. s., 1H), 10.47 (s, 1H), 8.69 (s, 1H), 8.40 (s, 1H), 8.22 (s, 1H), 7.55-7.39 (m, 3H), 7.29 (s, 1H), 2.48 (s, 3H), 2.34 (s, 3H). MS-ESI (m/z) calc'd for C₁₇H₁₆N₅O [M+H]⁺: 306.1. Found 306.1.

### Example 146: N-(3-(Furan-3-yl)-1H-indazol-5-yl)-7-methylimidazo[1,5-a]pyridine-6-carboxamide

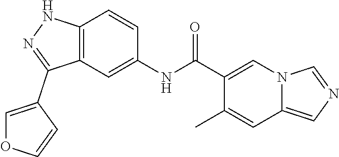 [SMILES: Cc1cc2cncn2cc1C(=O)Nc1ccc2[nH]nc(-c3ccoc3)c2c1]

7-Methylimidazo[1,5-a]pyridine-6-carboxylic acid (20.0 mg, 0.110 mmol) and 3-(furan-3-yl)-1H-indazol-5-amine (27.14 mg, 0.140 mmol) were dissolved in dry DMF (1.5 mL). Then the solution was cooled to 0° C. with an ice-water bath and HATU (51.8 mg, 0.140 mmol) and triethylamine (23.74 uL, 0.170 mmol) were added. The mixture was stirred at 0° C. for 5 min and then at r.t. for 3 hrs. The reaction mixture was partitioned between water and EtOAc, the phases were separated, the aqueous layer was extracted with EtOAc (2×) and the combined organic phases washed with water (1×), dried over anhydrous Na₂SO₄ and evaporated to dryness. The crude was purified by direct phase chromatography on a 11 g NH silica gel column, using as eluent a gradient of MeOH in EtOAc from 0 to 10% to afford the title compound (13 mg, 0.036 mmol, 32% yield) as a white solid. ¹H NMR (400 MHz, DMSO-d₆) δ ppm 13.11 (br. s., 1H), 10.53 (s, 1H), 8.72 (s, 1H), 8.46-8.36 (m, 2H), 8.21 (s, 1H), 7.86 (t, J=1.7 Hz, 1H), 7.73-7.63 (m, 1H), 7.59-7.54 (m, 1H), 7.43 (s, 1H), 7.29 (s, 1H), 7.00 (d, J=1.1 Hz, 1H), 2.35 (s, 3H). MS-ESI (m/z) calc'd for C₂₀H₁₆N₅O₂ [M+H]⁺: 358.1. Found 358.1.

### Example 147: N-(3-(Furan-3-yl)-1H-indazol-5-yl)-1,5,7-trimethylimidazo[1,5-a]pyridine-6-carboxamide

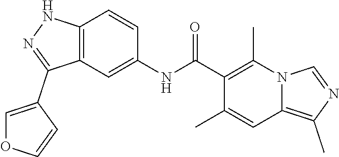 [SMILES: Cc1cc2c(C)ncn2c(C)c1C(=O)Nc1ccc2[nH]nc(-c3ccoc3)c2c1]

### Step 1: 6-Chloro-2,4-dimethylnicotinic acid

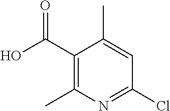

6-Chloro-2,4-dimethylnicotinonitrile (10.0 g 60.02 mmol) was dissolved in a mixture of sulfuric acid (21.01 mL, 394.1 mmol) and water (20 mL). The mixture was heated at 120° C. for 16 hrs. It was then cooled to 90° C. and sodium nitrite (29.41 g, 420.14 mmol) was added in small portions over 10 min. The reaction was heated at 90° C. for an additional 18 hrs and then cooled to room temperature and poured into an ice-water mixture (˜20 mL). The chilled mixture was filtered and the white solid was washed with water. The white powder was dried under high vacuum to give the title compound (8.4 g, 45.26 mmol. 75% yield). ¹H NMR (400 MHz, DMSO-d₆) δ ppm 13.51 (br. s., 1H) 7.34 (s, 1H) 2.45 (s, 3H) 2.31 (s, 3H). MS-ESI (m/z) calc'd for C₈H₈ClNO₂ [M+H]⁺: 186.0. Found 186.0, 188.0.

### Step 2: Ethyl 6-chloro-2,4-dimethylnicotinate

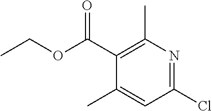

A suspension of 6-chloro-2,4-dimethylnicotinic acid (8.4 g, 45.26 mmol) in phosphorus(V) oxychloride (77.41 mL, 830.54 mmol) was heated at 80° C. for 6 hrs. Excess POCl₃ was removed by evaporation and the residue was carefully quenched with EtOH (112 mL) at 0° C. The solvent was evaporated and the residue was taken up in sat. aq. NaHCO₃ and stirred for 15 minutes. The solid that formed was extracted with DCM (3×) and the combined organic layers were passed through a phase separator and evaporated to dryness under reduced pressure. The crude material was purified by normal phase chromatography on a 100 g silica gel column, using a gradient of EtOAc in cyclohexane (from 0 to 20%) as eluent to afford the title compound (3.4 g, 15.91 mmol, 99% yield) a colorless oil. ¹H NMR (400 MHz, CDCl₃) δ ppm 7.07 (s, 1H), 4.44 (q, J=7.0 Hz, 2H), 2.55 (s, 3H), 2.35 (s, 3H), 1.42 (t, J=7.2 Hz, 3H). MS-ESI (m/z) calc'd for C₁₀H₁₃ClNO₂ [M+H]⁺: 214.1. Found 214.0, 216.0.

### Step 3: Ethyl 6-acetyl-2,4-dimethylnicotinate

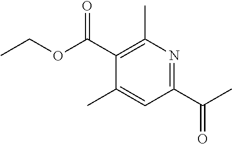 [SMILES: CCOC(=O)c1c(C)cc(C(C)=O)nc1C]

Ethyl 6-chloro-2,4-dimethylnicotinate (1.5 g, 7.02 mmol) and triphenylphosphine (0.18 g, 0.700 mmol) were dissolved in toluene (20 mL). The solution was degassed with nitrogen and tributyl(1-ethoxyethenyl)stannane (3.08 mL, 9.13 mmol) and tetrakis(triphenylphosphine)palladium(0) (0.41 g, 0.350 mmol) were added. The mixture was heated at 95° C. for 16 hrs under a nitrogen atmosphere. The reaction mixture was concentrated under reduced pressure and the crude enol-ether was treated with a 2:1 mixture of EtOH/conc. HCl (5 mL/2.5 mL) and allowed to stir for 4 hrs. The reaction mixture was diluted with EtOAc and basified with sat. aq. NaHCO₃. The layers were separated and the aqueous layer was extracted with EtOAc (2×). The combined organic layers were washed with water, dried over anhydrous Na₂SO₄, filtered and concentrated in vacuo. The crude material was purified by normal phase chromatography on a 100 g silica gel column, using a gradient of EtOAc in cyclohexane (from 0 to 20%) as eluent to afford the title compound (1.38 g, 6.237 mmol, 89% yield) as a light brown oil. ¹H NMR (400 MHz, CDCl₃) δ ppm 7.74 (s, 1H), 4.46 (q, J=7.0 Hz, 2H), 2.72 (s, 3H), 2.61 (s, 3H), 2.40 (s, 3H), 1.43 (t, J=7.2 Hz, 3H). MS-ESI (m/z) calc'd for C₁₂H₁₆NO₃ [M+H]⁺: 222.1. Found 222.1.

### Step 4: Ethyl (E)-6-(1-(hydroxyimino)ethyl)-2,4-dimethylnicotinate

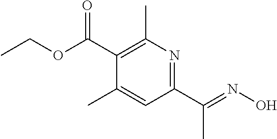 [SMILES: CCOC(=O)c1c(C)cc(/C(C)=N/O)nc1C]

A mixture of ethyl 6-acetyl-2,4-dimethylnicotinate (1.38 g, 6.24 mmol), potassium carbonate (1.03 g, 7.48 mmol) and hydroxylamine hydrochloride (520.11 mg, 7.48 mmol) in MeOH (40 mL) was stirred at room temperature for 2 hrs. Volatiles were removed under reduced pressure. The residue was partitioned between water and EtOAc. The phases were then separated and the aqueous layer was extracted with EtOAc (2×). The combined organic phases were washed with water (1×), dried over anhydrous Na₂SO₄ and evaporated to dryness. The title compound (1.44 g, 6.095 mmol, 98% yield) was obtained as a light yellow oil. ¹H NMR (400 MHz, DMSO-d₆) δ ppm 11.55 (s, 1H), 7.60 (s, 1H), 4.38 (q, J=7.2 Hz, 2H), 2.47 (s, 3H), 2.30 (s, 3H), 2.19 (s, 3H), 1.33 (t, J=7.0 Hz, 3H). MS-ESI (m/z) calc'd for C₁₂H₁₇N₂O₃ [M+H]⁺: 237.1. Found 237.1.

### Step 5: Ethyl 6-(I-aminoethyl)-2,4-dimethylnicotinate

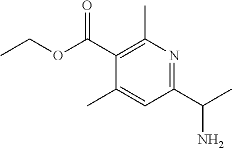 [SMILES: CCOC(=O)c1c(C)cc(C(C)N)nc1C]

A solution of ethyl (E)-6-(1-(hydroxyimino)ethyl)-2,4-dimethylnicotinate (1.44 g, 6.09 mmol) in water (18 mL) and acetic acid (18 mL) at 0° C. was treated slowly with zinc dust (1.99 g, 30.47 mmol) and stirred for 1 hr at the same temperature. The solids were removed by filtration through a Celite pad. The cake was washed with EtOH and the filtrate was recovered and concentrated to dryness. The crude material was partitioned between DCM and sat. aq. NaHCO₃. The phases were separated; the aqueous layer was washed with DCM (2×) and the combined organic phases were extracted with water (1×). The desired compound was found in the aqueous phase which was evaporated to dryness. The residue was suspended in EtOH and the salts removed by filtration. The filtrate was collected and evaporated to dryness. The title compound (2.1 g, 6.09 mmol theoretical) was obtained as a white solid. ¹H NMR (400 MHz, DMSO-d₆) δ ppm 7.26 (s, 1H), 4.36 (q, J=7.0 Hz, 2H), 4.05 (q, J=6.7 Hz, 1H), 2.45 (s, 3H), 2.28 (s, 3H), 1.37-1.26 (m, 6H). MS-ESI (m/z) calc'd for C₁₂H₁₉N₂O₂ [M+H]⁺: 223.1. Found 223.1.

### Step 6: Ethyl 6-(1-formamidoethyl)-2,4-dimethylnicotinate

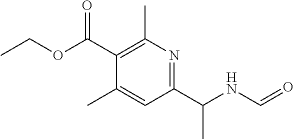 [SMILES: CCOC(=O)c1c(C)cc(C(C)NC=O)nc1C]

A solution of ethyl 6-(1-aminoethyl)-2,4-dimethylnicotinate (1.35 g, 6.1 mmol) in formic acid (20.0 mL, 530.09 mmol) was refluxed at 100° C. for 1 hr. Volatiles were removed under reduced pressure. The residue was re-dissolved in DCM and then washed with sat. aq. NaHCO₃ and water, passed through a phase separator, and concentrated. The title compound (675 mg, 2.697 mmol, 44% yield) was obtained as a yellow oil. ¹H NMR (400 MHz, DMSO-d₆) δ ppm 8.55 (d, J=7.5 Hz, 1H), 8.06 (s, 1H), 7.12 (s, 1H), 4.93 (quin, J=7.2 Hz, 1H), 4.36 (q, J=7.0 Hz, 2H), 2.44 (s, 3H), 2.27 (s, 3H), 1.39-1.27 (m, 6H). MS-ESI (m/z) calc'd for C₁₃H₁₉N₂O₃ [M+H]⁺: 251.1. Found 251.1.

### Step 7: Ethyl 1,5,7-trimethylimidazo[1,5-a]pyridine-6-carboxylate

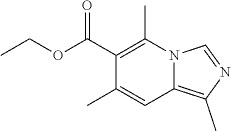 [SMILES: CCOC(=O)c1c(C)cc2c(C)ncn2c1C]

Ethyl 6-(1-formamidoethyl)-2,4-dimethylnicotinate (675.0 mg, 2.7 mmol) was dissolved in toluene (15 mL) and phosphorus(V) oxychloride (2.5 mL, 26.74 mmol) was added. The reaction was stirred at 65° C. for 1 hr. Volatiles were removed under reduced pressure. The residue was taken up in DCM and washed with saturated aqueous NaHCO₃ (1×), water (1×), passed through a phase separator and concentrated to dryness to give the title compound (580 mg, 2.497 mmol, 93% yield) as a yellow oil. ¹H NMR (400 MHz, DMSO-d₆) δ ppm 8.28 (s, 1H), 7.33 (s, 1H), 4.37 (q, J=7.0 Hz, 2H), 2.53 (s, 3H), 2.41 (s, 3H), 2.20 (s, 3H), 1.33 (t, J=7.0 Hz, 3H). MS-ESI (m/z) calc'd for C₁₃H₁₇N₂O₂ [M+H]⁺: 233.1. Found 233.1.

### Step 8: 1,5,7-Trimethylimidazo[1,5-a]pyridine-6-carboxylic acid

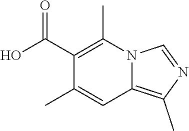

Ethyl 1,5,7-trimethylimidazo[1,5-a]pyridine-6-carboxylate (580.0 mg, 2.5 mmol) was dissolved in EtOH (5 mL) and a 2 M aqueous solution of NaOH (12.0 mL, 24 mmol) was added. The solution was stirred at 50° C. for 16 hrs. EtOH was removed under reduced pressure and then the aqueous residue acidified with HCl (2M) until pH 1. The mixture was partitioned between water and EtOAc and the phases were separated. The organic layer was extracted with water (1×) and the combined aqueous phases washed with EtOAc (1×) and evaporated to dryness. The product remained in the aqueous layer. The solid obtained after evaporation of water was triturated with EtOH. The insoluble salts were removed by filtration and the filtrate was collected and the solvent evaporated under reduced pressure to afford the title compound (625 mg, 3.06 mmol) as a brown oil. ¹H NMR (400 MHz, DMSO-d₆) δ ppm 8.82 (br. s., 1H), 7.48 (s, 1H), 2.58 (s, 3H), 2.48 (s, 3H), 2.27 (d, J=0.9 Hz, 3H). MS-ESI (m/z) calc'd for C₁₁H₁₃N₂O₂ [M+H]⁺: 205.1. Found 205.1.

### Step 9: N-(3-(Furan-3-yl)-1H-indazol-5-yl)-1,5,7-trimethylimidazo[1,5-a]pyridine-6-carboxamide

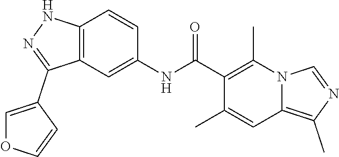 [SMILES: Cc1cc2c(C)ncn2c(C)c1C(=O)Nc1ccc2[nH]nc(-c3ccoc3)c2c1]

1,5,7-Trimethylimidazo[1,5-a]pyridine-6-carboxylic acid (20.0 mg, 0.100 mmol) and 3-(furan-3-yl)-1H-indazol-5-amine (23.41 mg, 0.120 mmol) were dissolved in dry DMF (1.5 mL). Then the solution was cooled to 0° C. with an ice-water bath followed by addition of HATU (44.68 mg, 0.120 mmol) and triethylamine (20.47 uL, 0.150 mmol). The mixture was stirred at 0° C. for 5 min, at room temperature overnight and then at 50° C. for 10 hrs. The reaction mixture was partitioned between water and EtOAc and the phases were separated. The aqueous layer was extracted with EtOAc (2×) and the combined organic phases washed with water (1×), dried over anhydrous Na₂SO₄ and evaporated to dryness. The crude material was purified first by normal phase chromatography on an 11 g NH silica gel column using a gradient of MeOH in EtOAc (from 0 to 10%) as eluent, then via preparative HPLC (method C) to afford the title compound (5.7 mg, 0.015 mmol, 15% yield) as a white solid. ¹H NMR (400 MHz, DMSO-d₆) δ ppm 13.11 (s, 1H), 10.53 (s, 1H), 8.41 (s, 1H), 8.21-8.24 (in, 2H), 7.86 (t, J=1.4 Hz, 1H), 7.67-7.52 (m, 2H), 7.36 (s, 1H), 7.00 (d, J=1.3 Hz, 1H), 2.57 (s, 3H), 2.43 (s, 3H), 2.27 (s, 3H). MS-ESI (m/z) calc'd for C₁₁H₁₃N₂O₂ [M+H]⁺: 385.1. Found 385.2.

### Example 148: 1,5,7-Trimethyl-N-(3-methyl-1H-indazol-5-yl)imidazo[1,5-a]pyridine-6-carboxamide

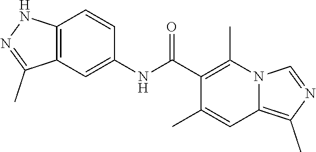 [SMILES: Cc1cc2c(C)ncn2c(C)c1C(=O)Nc1ccc2[nH]nc(C)c2c1]

1,5,7-trimethylimidazo[1,5-a]pyridine-6-carboxylic acid (20.0 mg, 0.100 mmol) and 3-methyl-1H-indazol-5-amine (17.3 mg, 0.120 mmol) were dissolved in dry DMF (1.5 mL). The solution was cooled to 0° C. with an ice-water bath and HATU (44.68 mg, 0.120 mmol) and triethylamine (20.47 uL, 0.150 mmol) were added. The mixture was stirred at 0° C. for 5 min., then at room temperature overnight and then at 50° C. for 5 hrs. The reaction mixture was partitioned between water and EtOAc, the phases were separated, the aqueous layer was extracted with EtOAc (2×) and the combined organic phases washed with water (1×), dried over anhydrous Na₂SO₄ and evaporated to dryness. The crude material was purified first by normal phase flash chromatography on an 11 g NH silica gel column, using a gradient of MeOH in EtOAc (from 0 to 10%) as eluent followed by preparative HPLC (method D) to afford the title compound (4.2 mg, 0.011 mmol, 110% yield) as a white solid. ¹H NMR (400 MHz, DMSO-d₆) δ ppm 12.61 (br. s., 1H), 10.48 (s, 1H), 8.32 (s, 1H), 8.22 (s, 2H), 7.50-7.41 (m, 2H), 7.35 (s, 1H), 2.54 (s, 3H), 2.49 (s, 3H), 2.43 (s, 3H), 2.26 (s, 3H). MS-ESI (m/z) calc'd for C₁₉H₂₀N₅O [M+H]⁺: 334.2. Found 334.2.

### Example 149: 4-Methyl-N-(3-phenyl-1H-indazol-5-yl)-[1,2,3]triazolo[1,5-a]pyridine-5-carboxamide

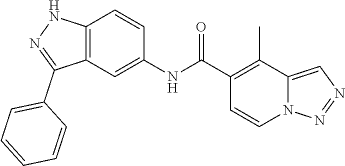 [SMILES: Cc1c(C(=O)Nc2ccc3[nH]nc(-c4ccccc4)c3c2)ccn2nncc12]

### Step 1: Ethyl 2,3,5-trichloroisonicotinate

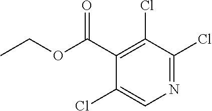

To a solution of 2,3,5-trichloropyridine-4-carboxylic acid (2.26 g, 10 mmol) in DMF (20 mL) was added K₂CO₃ (5.53 g, 40 mmol) and iodoethane (1.61 mL, 20 mmol). The mixture was stirred at 25° C. for 2 hrs. Water was added and the compound was extracted with EtOAc (3×). The combined organic layers were washed with water (2×), passed through a phase separator and evaporated to afford the title compound (2.545 g, 10 mmol, 100% yield) as a brown oil. ¹H NMR (400 MHz, DMSO-d₆) δ 8.70 (s, 1H), 4.48 (q, J=7.1 Hz, 2H), 1.34 (t, J=7.1 Hz, 3H). MS-ESI (m/z) calc'd for C₈H₇Cl₃NO₂ [M+H]⁺: 254.0. Found 254.0.

### Step 2: Ethyl 3,5-dichloro-2-vinylisonicotinate

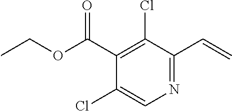

To a solution of ethyl 2,3,5-trichloroisonicotinate (2.55 g, 10 mmol) in 1,4-dioxane (50 mL) was added 2-ethenyl-4,4,5,5-tetramethyl-1,3,2-dioxaborolane (2.54 mL, 15 mmol) and tetrakis(triphenylphosphine)palladium(0) (1.16 g, 1 mmol). The mixture was stirred at 25° C. for 5 minutes then a solution of K₂CO₃ (1.38 g, 10 mmol) in water (50 mL) was added and the mixture was stirred at 80° C. for 2 hrs. The mixture was taken up in water and extracted with DCM (3×). The combined organic layers were passed through a phase separator and evaporated to obtain a residue which was purified by column chromatography (SiO₂, 100 g) using a 0-10% gradient of EtOAc in cyclohexane for 10 CV to afford the title compound (2.072 g, 8.42 mmol, 84% yield) as an orange oil. ¹H NMR (400 MHz, DMSO-d₆) δ 8.77 (s, 1H), 7.17 (dd, J=16.9, 10.7 Hz, 1H), 6.47 (dd, J=16.9, 2.0 Hz, 1H), 5.76 (dd, J=10.7, 2.0 Hz, 1H), 4.46 (q, J=7.0 Hz, 2H), 1.34 (t, J=7.1 Hz, 3H). MS-ESI (m/z) calc'd for C₁₀H₁₀Cl₂NO₂ [M+H]⁺: 246.0. Found 246.0, 248.0.

### Step 3: Ethyl 3,5-dichloro-2-formylisonicotinate

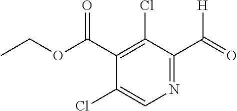

A solution of ethyl 3,5-dichloro-2-vinylisonicotinate (2.07 g, 8.42 mmol) in DCM (100 mL) was cooled to −78° C. and ozone was bubbled through the mixture for 30 minutes. Triphenylphosphine (2.21 g, 8.42 mmol) was added portionwise and the mixture was stirred for 30 minutes. The solvent was evaporated and the residue was purified by column chromatography (SiO₂, 50 g) using a 0-20% gradient of EtOAc in cyclohexane for 10 CV to afford the title compound (0.810 g, 3.265 mmol, 39% yield) as a brown oil. ¹H NMR (400 MHz, DMSO-d₆) δ 10.06 (s, 1H), 9.02 (s, 1H), 4.49 (q, J=7.1 Hz, 2H), 1.35 (t, J=7.1 Hz, 3H). MS-ESI (m/z) calc'd for C₉H₈Cl₂NO₃ [M+H]⁺: 248.0. Found 248.0, 250.0.

### Step 4: Ethyl 4,6-dichloro-[1,2,3]triazolo[1,5-a]pyridine-5-carboxylate

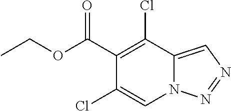 [SMILES: CCOC(=O)c1c(Cl)cn2nncc2c1Cl]

To a solution of ethyl 3,5-dichloro-2-formylisonicotinate (0.81 g, 3.27 mmol) in MeOH (16 mL) was added hydrazine hydrate (0.3 mL, 9.8 mmol) and the mixture was stirred at 25° C. for 15 hrs. The solvent was evaporated and the residue was taken up in DCM (16 mL), then manganese(IV) oxide (568 mg, 6.53 mmol) was added and the mixture was stirred at 25° C. for 1 hr. The black solid was filtered through a Celite pad and the filtrate was evaporated to afford the title compound (640 mg, 2.461 mmol, 75% yield) as a yellow solid. ¹H NMR (400 MHz, DMSO-d₆) δ 9.70 (d, J=1.0 Hz, 1H), 8.52 (d, J=1.0 Hz, 1H), 4.47 (q, J=7.1 Hz, 2H), 1.36 (t, J=7.1 Hz, 3H). MS-ESI (m/z) calc'd for C₉H₈Cl₂N₃O₂ [M+H]⁺: 260.0. Found 259.7, 262.0.

### Step 5: Ethyl 6-chloro-4-methyl-[1,2,3]triazolo[1,5-a]pyridine-5-carboxylate

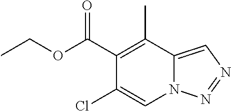 [SMILES: CCOC(=O)c1c(Cl)cn2nncc2c1C]

To a solution of ethyl 4,6-dichloro-[1,2,3]triazolo[1,5-a]pyridine-5-carboxylate (614 mg, 2.36 mmol) in 1,4-dioxane (24 mL) was added trimethylboroxine (3 mL, 21.25 mmol), tetrakis(triphenylphosphine)palladium(0) (0.55 g, 0.470 mmol) and K₂CO₃ (0.98 g, 7.08 mmol) in water (12 mL). The mixture was stirred at 90° C. for 3 hrs. The organic solvent was evaporated and the residue was diluted with water and extracted with EtOAc (3×). The combined organic layers were washed with brine, dried over Na₂SO₄ and evaporated to obtain a residue which was purified by column chromatography (SiO₂, 50 g) using a 0-50% gradient of EtOAc in cyclohexane for 10 CV followed by straight EtOAc for 5 CV to afford the title compound (405 mg, 1.69 mmol, 72% yield) as a yellow solid. LC-MS: m/z=240.04 242.04 [M+H]⁺, 0.87 min. ¹H NMR (400 MHz, DMSO-d₆) δ 9.47 (t, J=0.9 Hz, 1H), 8.50 (d, J=1.0 Hz, 1H), 4.43 (q, J=7.1 Hz, 2H), 2.54 (d, J=0.8 Hz, 3H), 1.35 (t, J=7.1 Hz, 3H). MS-ESI (m/z) calc'd for C₁₀H₁₁₁ClN₃O₂ [M+H]⁺: 240.1. Found 240.0, 242.0.

### Step 6: Ethyl 4-methyl-[1,2,3]triazolo[1,5-a]pyridine-5-carboxylate

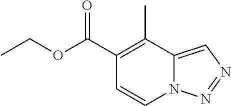

To a solution of ethyl 6-chloro-4-methyl-[1,2,3]triazolo[1,5-a]pyridine-5-carboxylate (400.0 mg, 1.67 mmol) in methanol (17 mL) was added ammonium formate (421 mg, 6.68 mmol) and 10% palladium (178 mg, 0.170 mmol) on carbon. The mixture was stirred at 65° C. for 2 hrs. The catalyst was filtered through a Celite pad and the filtrate was evaporated. The residue was taken up in water and extracted with EtOAc (3×). The combined organic layers were passed through a phase separator and evaporated to afford the title compound (250 mg, 1.218 mmol, 73% yield) as a yellow solid. ¹H NMR (400 MHz, DMSO-d₆) δ 8.98 (dt, J=7.3, 1.0 Hz, 1H), 8.59 (d, J=1.1 Hz, 1H), 7.43 (d, J=7.3 Hz, 1H), 4.35 (q, J=7.1 Hz, 2H), 2.87-2.72 (m, 3H), 1.35 (t, J=7.1 Hz, 3H). MS-ESI (m/z) calc'd for C₁₀H₁₂N₃O₂ [M+H]⁺: 206.1. Found 206.0.

### Step 7: 4-Methyl-[1,2,3]triazolo[1,5-a]pyridine-5-carboxylic acid

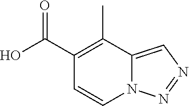

To a solution of ethyl 4-methyltriazolo[1,5-a]pyridine-5-carboxylate (60.0 mg, 0.290 mmol) in THF (1.5 mL) was added a solution LiOH (21 mg, 0.880 mmol) in water (1.5 mL) and the mixture was stirred at 25° C. for 4 hrs. The pH was adjusted to 1 by addition of HCl and the solvent was evaporated to afford the title compound (51 mg, 0.292 mmol, 100% yield) as a yellow solid. ¹H NMR (400 MHz, DMSO-d₆) δ 13.55 (s, 1H), 8.94 (d, J=7.3 Hz, 1H), 8.56 (d, J=1.0 Hz, 1H), 7.44 (d, J=7.3 Hz, 1H), 2.80 (s, 3H). MS-ESI (m/z) calc'd for C₈H₈N₃O₂ [M+H]⁺: 178.1. Found 178.0.

### Step 8: 4-Methyl-N-(3-phenyl-1H-indazol-5-yl)-[1,2,3]triazolo[1,5-a]pyridine-5-carboxamide

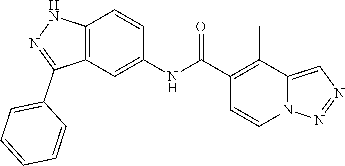 [SMILES: Cc1c(C(=O)Nc2ccc3[nH]nc(-c4ccccc4)c3c2)ccn2nncc12]

To a solution of 4-methyl-[1,2,3]triazolo[1,5-a]pyridine-5-carboxylic acid (51 mg, 0.290 mmol) in MeCN (3 mL) was added triethylamine (41 uL, 0.290 mmol) and HATU (111 mg, 0.290 mmol). The mixture was stirred at 25° C. for 15 minutes, then 3-phenyl-1H-indazol-5-amine (63.07 mg, 0.290 mmol) was added and stirring was continued for 8 hrs. The mixture was diluted with water and extracted with EtOAc (3×), the combined organic layers were washed with water (2×), passed through a phase separator and evaporated to obtain a residue which was purified by column chromatography (SiO₂, 25 g) using a 0-10% gradient of MeOH in DCM for 15 CV to afford the title compound (78 mg, 0.212 mmol, 72% yield) as an orange solid. ¹H NMR (400 MHz, DMSO-d₆) δ 13.26 (s, 1H), 10.58 (s, 1H), 9.06 (d, J=7.1 Hz, 1H), 8.60 (d, J=1.8 Hz, 1H), 8.48 (d, J=1.0 Hz, 1H), 8.02-7.88 (m, 2H), 7.68 (dd, J=9.0, 1.8 Hz, 1H), 7.61 (d, J=8.9 Hz, 1H), 7.55 (t, J=7.7 Hz, 2H), 7.46-7.38 (m, 1H), 7.31 (d, J=7.1 Hz, 1H), 2.65 (s, 3H). MS-ESI (m/z) calc'd for C₂₁H₁₇N₆O [M+H]⁺: 369.1. Found 369.1.

### Example 150: N-(3-(Furan-3-yl)-1H-indazol-5-yl)-5,7-dimethyl-[1,2,3]triazolo[1,5-a]pyridine-6-carboxamide

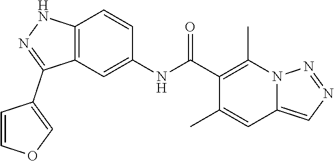 [SMILES: Cc1cc2cnnn2c(C)c1C(=O)Nc1ccc2[nH]nc(-c3ccoc3)c2c1]

To a solution of 3-(furan-3-yl)-1H-indazol-5-amine (52.1 mg, 0.26 mmol) and 5,7-dimethyltriazolo[1,5-a]pyridine-6-carboxylic acid (50.0 mg, 0.26 mmol) in DMF (2 mL), was added triethylamine (0.04 mL, 0.31 mmol) and HATU (109.4 mg, 0.29 mmol) at 0° C. The reaction mixture was stirred at r.t. for 18 hrs. Water was added and the mixture was extracted with EtOAc. The phases were separated and the organic layer was washed with brine, dried over Na₂SO₄ and concentrated under reduced pressure. The residue was purified by normal phase column chromatography using a 0-100% gradient of EtOAc in cyclohexane for 10 CV. Product-containing fractions were combined and concentrated under reduced pressure to afford the title compound (14.5 mg, 0.039 mmol, 15% yield), as a beige solid. ¹H NMR (400 MHz, DMSO-d₆) δ ppm 13.14 (s, 1H), 10.71 (s, 1H), 8.41 (s, 1H), 8.25 (s, 1H), 8.22 (s, 1H), 7.77-7.93 (m, 2H), 7.53-7.66 (m, 2H), 6.95-7.06 (m, 1H), 2.87 (s, 3H), 2.43 (d, J=0.66 Hz, 3H). MS-ESI (m/z) calc'd for C₂₀H₁₇N₆O₂ [M+H]⁺: 373.1. Found 371.2.

### Example 151: N-(3-(Furan-3-yl)-1H-indazol-5-yl)-5,7-dimethylimidazo[1,5-a]pyridine-6-carboxamide

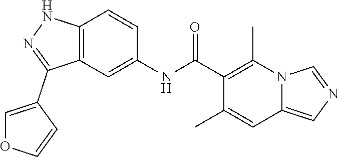 [SMILES: Cc1cc2cncn2c(C)c1C(=O)Nc1ccc2[nH]nc(-c3ccoc3)c2c1]

To a solution of 3-(furan-3-yl)-1H-indazol-5-amine (41.89 mg, 0.21 mmol) and 5,7-dimethylimidazo[1,5-a]pyridine-6-carboxylic acid (40.0 mg, 0.21 mmol) in DMF (1.5 mL) was added triethylamine (0.04 mL, 0.320 mmol) and HATU (87.96 mg, 0.230 mmol). The resulting mixture was stirred for 18 hrs at r.t. and then at 50° C. for 4 hrs. The mixture was diluted with water and then extracted with EtOAc (2×). The organic phase was collected, dried over Na₂SO₄, filtered and concentrated under reduced pressure. The crude material was purified by semi-preparative HPLC (method E) to obtain the title compound (18.5 mg, 0.05 mmol, 24% yield) as a beige solid. ¹H NMR (400 MHz, DMSO-d₆) δ 13.12 (br. s., 1H), 10.57 (s, 1H), 8.41 (s, 1H), 8.37 (s, 1H), 8.23 (s, 1H), 7.86 (t, J=1.54 Hz, 1H), 7.59-7.64 (m, 1H), 7.55-7.59 (m, 1H), 7.41 (s, 1H), 7.38 (s, 1H), 7.00 (d, J=1.10 Hz, 1H), 2.62 (s, 3H), 2.29 (s, 3H). MS-ESI (m/z) calc'd for C₂₁H₁₈N₅O₂ [M+H]⁺: 372.1. Found 372.1.

### Example 152: N-(3-(Furan-3-yl)-1H-indazol-5-yl)-6-methylimidazo[1,5-a]pyridine-7-carboxamide

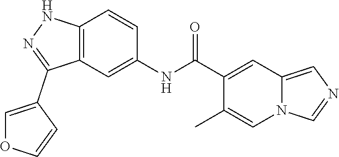 [SMILES: Cc1cn2cncc2cc1C(=O)Nc1ccc2[nH]nc(-c3ccoc3)c2c1]

To a solution of ethyl 6-methylimidazo[1,5-a]pyridine-7-carboxylate (50 mg, 0.24 mmol) in toluene (10 mL) was added 3-(furan-3-yl)-1H-indazol-5-amine (64.7 mg, 0.32 mmol) followed by trimethylaluminum (2 M in hexane) (0.24 mL, 0.49 mmol). The reaction mixture was stirred for 2 hrs at 90° C. An additional portion of trimethylaluminum (2 M in hexane) (0.24 mL, 0.49 mmol) was added and the reaction was stirred for 18 hrs at 90° C. The reaction was cooled to room temperature and diluted with water (20 mL) and EtOAc (30 mL). The organic layer was separated, dried over Na₂SO₄, filtered and concentrated under reduced pressure. The residue was purified by semi-preparative HPLC (method F) to obtain the title compound (13.3 mg, 0.037 mmol, 15% yield) as a yellow solid. ¹H NMR (400 MHz, DMSO-d₆) δ ppm 13.10 (s, 1H), 10.45 (s, 1H), 8.39 (s, 2H), 8.19-8.28 (m, 2H), 7.83-7.91 (m, 2H), 7.69 (d, J=8.80 Hz, 1H), 7.48-7.59 (m, 2H), 7.00 (d, J=1.10 Hz, 1H), 2.31 (d, J=0.88 Hz, 3H). MS-ESI (m/z) calc'd for C₂₀H₁₆N₅O₂ [M+H]⁺: 358.1. Found 358.1.

### Example 153: N-(3-(Furan-3-yl)-1H-indazol-5-yl)-5,7-dimethyl-[1,2,4]triazolo[4,3-a]pyridine-6-carboxamide

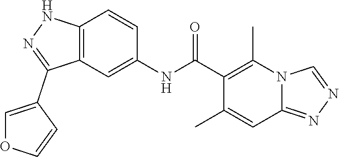 [SMILES: Cc1cc2nncn2c(C)c1C(=O)Nc1ccc2[nH]nc(-c3ccoc3)c2c1]

To a solution of 3-(furan-3-yl)-1H-indazol-5-amine (40.93 mg, 0.20 mmol), 5,7-dimethyl-[1,2,4]triazolo[4,3-a]pyridine-6-carboxylic acid (35.0 mg, 0.18 mmol) and triethylamine (0.04 mL, 0.27 mmol) in DMF (1 mL) was added HATU (76.57 mg, 0.200 mmol) and the mixture was left stirring at room temperature for 18 hrs. Water was added and the mixture was extracted with EtOAc (2×). The organic phases were combined and then concentrated under reduced pressure. The residue was purified by semi-preparative HPLC (method G) to afford the title compound (16.4 mg, 0.044 mmol, 24% yield) as a pink solid. ¹H NMR (400 MHz, DMSO-d₆) δ ppm 13.13 (s, 1H), 10.62 (s, 1H), 9.35 (d, J=0.88 Hz, 1H), 8.39 (s, 1H), 8.24 (dd, J=1.43, 0.77 Hz, 1H), 7.86 (t, J=1.65 Hz, 1H), 7.64 (s, 1H), 7.56-7.63 (m, 2H), 7.54-7.66 (m, 3H), 7.00 (dd, J=1.76, 0.66 Hz, 1H), 2.72 (s, 3H), 2.41 (d, J=1.10 Hz, 3H). MS-ESI (m/z) calc'd for C₂₀H₁₇N₆O₂ [M+H]⁺: 373.1. Found 373.2.

### Example 154: N-(3-(Furan-2-yl)-1H-indazol-5-yl)-6-methylimidazo[1,5-a]pyridine-7-carboxamide

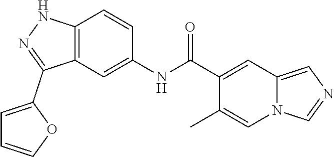 [SMILES: Cc1cn2cncc2cc1C(=O)Nc1ccc2[nH]nc(-c3ccco3)c2c1]

To a solution of ethyl 6-methylimidazo[1,5-a]pyridine-7-carboxylate (50.0 mg, 0.24 mmol) in toluene (10 mL) was added 3-(furan-2-yl)-1H-indazol-5-amine (66.04 mg, 0.320 mmol) followed by trimethylaluminum (2M in hexane) (0.24 mL, 0.49 mmol). The reaction mixture was stirred for 18 hrs at 90° C. An additional portion of trimethylaluminum (2M in hexane) (0.24 mL, 0.49 mmol) was added and stirring was continued for 3 hrs at 90° C. The reaction was cooled to room temperature and diluted with water (20 mL) and EtOAc (30 mL). The organic layer was separated, dried over Na₂SO₄, filtered and concentrated under reduced pressure. The residue was purified by reverse phase column chromatography using a 2-60% gradient of CH₃CN in H₂O (+0.1% HCOOH) to afford the title compound (15.5 mg, 0.043 mmol, 18% yield) as a yellow solid. ¹H NMR (400 MHz, DMSO-d₆) δ ppm 13.24 (s, 1H), 10.50 (s, 1H), 8.60 (s, 1H), 8.39 (s, 1H), 8.25 (d, J=0.88 Hz, 1H), 7.88 (s, 2H), 7.69 (d, J=7.48 Hz, 1H), 7.57 (d, J=9.02 Hz, 1H), 7.52 (s, 1H), 6.89 (d, J=3.30 Hz, 1H), 6.69 (dd, J=3.30, 1.98 Hz, 1H), 2.30 (d, J=0.66 Hz, 3H). MS-ESI (m/z) calc'd for C₂₀H₁₆N₅O₂ [M+H]⁺: 358.1. Found 358.1.

### Example 155: N-(3-(Furan-2-yl)-1H-indazol-5-yl)-5,7-dimethyl-[1,2,3]triazolo[1,5-a]pyridine-6-carboxamide

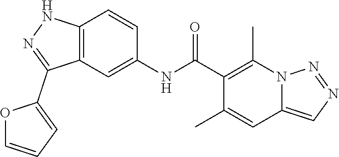 [SMILES: Cc1cc2cnnn2c(C)c1C(=O)Nc1ccc2[nH]nc(-c3ccco3)c2c1]

To a mixture of 3-(furan-2-yl)-1H-indazol-5-amine (41.79 mg, 0.200 mmol), 5,7-dimethyltriazolo[1,5-a]pyridine-6-carboxylic acid (35.0 mg, 0.180 mmol) and triethylamine (0.04 mL, 0.270 mmol) in DMF (1 mL) was added HATU (76.57 mg, 0.200 mmol) and the mixture was stirred at room temperature for 18 hrs. The mixture was then diluted with water and extracted with EtOAc (2×). The organic phases were combined and then concentrated under reduced pressure. The residue (110 mg) was purified by semi-preparative HPLC (method H) to afford the title compound (16.1 mg, 0.043 mmol, 24% yield) as a pink solid. ¹H NMR (400 MHz, DMSO-d₆) δ ppm 13.29 (s, 1H), 10.76 (s, 1H), 8.63 (s, 1H), 8.22 (s, 1H), 7.89 (dd, J=1.76, 0.66 Hz, 1H), 7.82 (s, 1H), 7.56-7.68 (m, 2H), 6.91 (dd, J=3.30, 0.66 Hz, 1H), 6.70 (dd, J=3.30, 1.76 Hz, 1H), 2.86 (s, 3H), 2.43 (d, J=0.88 Hz, 3H). MS-ESI (m/z) calc'd for C₂₀H₁₇N₆O₂ [M+H]⁺: 373.1. Found 373.1.

### Example 156: N-(3-(Furan-3-yl)-1H-indazol-5-yl)benzo[d]isothiazole-6-carboxamide

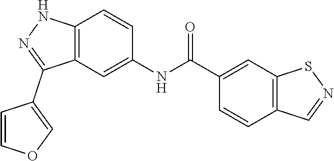 [SMILES: O=C(Nc1ccc2[nH]nc(-c3ccoc3)c2c1)c1ccc2cnsc2c1]

To a solution of 3-(furan-3-yl)-1H-indazol-5-amine (47.64 mg, 0.17 mmol) and 1,2-benzothiazole-6-carboxylic acid (30.0 mg, 0.17 mmol) in DMF (1 mL), triethylamine (0.03 mL, 0.20 mmol) and HATU (70.02 mg, 0.18 mmol) were added. The mixture was stirred at r.t. for 18 hrs. Water was added and the solution was extracted with EtOAc. The organic phase was dried over Na₂SO₄, filtered and concentrated under reduced pressure. The residue was purified by reverse phase column chromatography using a 2-100% gradient of CH₃CN in H₂O (+0.1% HCOOH) over 10 CV. The product was then dried under vacuum at 50° C. for 18 hrs. The title compound (12.7 mg, 0.035 mmol, 21% yield) was obtained as a white solid. ¹H NMR (400 MHz, DMSO-d₆) δ ppm 13.11 (br. s., 1H), 10.56 (s, 1H), 9.26 (d, J=0.88 Hz, 1H), 8.85 (s, 1H), 8.42 (s, 1H), 8.38 (d, J=8.36 Hz, 1H), 8.25 (dd, J=1.43, 0.77 Hz, 1H), 8.08 (dd, J=8.47, 1.43 Hz, 1H), 7.86 (t, J=1.65 Hz, 1H), 7.78 (dd, J=8.91, 1.65 Hz, 1H), 7.59 (d, J=8.80 Hz, 1H), 7.02 (dd, J=1.76, 0.66 Hz, 1H). MS-ESI (m/z) calc'd for C₁₉H₁₃N₄O₂S [M+H]⁺: 361.1. Found 361.1.

### Example 157: N-(3-(Furan-3-yl)-1H-indazol-5-yl)-1,4,6-trimethyl-1H-indazole-5-carboxamide

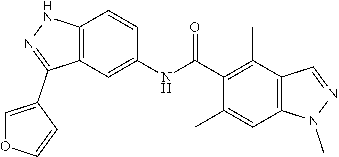 [SMILES: Cc1cc2c(cnn2C)c(C)c1C(=O)Nc1ccc2[nH]nc(-c3ccoc3)c2c1]

To a solution of methyl 1,4,6-trimethylindazole-5-carboxylate (28.0 mg, 0.120 mmol) in toluene (5 mL) was added 3-(furan-2-yl)-1H-indazol-5-amine (32.18 mg, 0.150 mmol) followed by trimethylaluminum (2 M in hexane) (0.12 mL, 0.240 mmol). The reaction mixture was stirred for 6 hrs at 90° C. An additional portion of trimethylaluminum (2M in hexane) (0.12 mL, 0.240 mmol) was added and the reaction was stirred for another 18 hrs at 90° C. The reaction was cooled to room temperature and extracted with water (20 mL) and EtOAc (30 mL). The organic layer was separated, dried over Na₂SO₄, filtered and concentrated under reduced pressure. The residue (57 mg) was purified by semi-preparative HPLC (method I) to obtain the title compound (7.2 mg, 0.019 mmol, 16% yield) as a yellow solid. ¹H NMR (400 MHz, DMSO-d₆) δ ppm 13.07 (s, 1H), 10.36 (s, 1H), 8.44 (d, J=1.32 Hz, 1H), 8.20 (dd, J=1.43, 0.77 Hz, 1H), 8.14 (d, J=0.88 Hz, 1H), 7.85 (t, J=1.65 Hz, 1H), 7.68 (dd, J=8.91, 1.65 Hz, 1H), 7.55 (d, J=8.80 Hz, 1H), 7.39 (s, 1H), 6.99 (dd, J=1.76, 0.66 Hz, 1H), 4.03 (s, 3H), 2.56 (s, 3H), 2.47 (s, 3H). MS-ESI (m/z) calc'd for C₂₂H₂₀N₅O₂ [M+H]⁺: 386.2. Found 386.1.

### Example 158: N-(3-(Furan-3-yl)-1H-indazol-5-yl)-1,6-dimethyl-1H-indazole-5-carboxamide

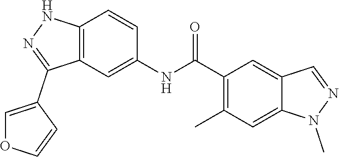 [SMILES: Cc1cc2c(cnn2C)cc1C(=O)Nc1ccc2[nH]nc(-c3ccoc3)c2c1]

### Step 1: 1,6-Dimethyl-1H-indazole-5-carbaldehyde

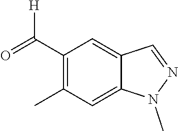

n-BuLi (0.49 mL, 1.22 mmol) was added to a cooled (−78° C.) solution of 5-bromo-1,6-dimethylindazole (250 mg, 1.11 mmol) in dry THF (4 mL). After stirring at the same temperature for 30 min, the reaction mixture was treated with dry DMF (0.33 mL, 4.44 mmol) and then allowed to rise to room temperature. After 1 hr at room temperature the reaction mixture was combined with an additional preparation, and treated with a saturated aqueous solution of NH₄Cl. The mixture was extracted with EtOAc and the organic layer was washed with brine, dried over Na₂SO₄, filtered and concentrated in vacuo. The residue was purified by normal phase column chromatography to obtain the title compound (77 mg, 0.442 mmol, 40% yield), as a pale yellow solid. ¹H NMR (400 MHz, DMSO-d₆) δ ppm 10.18 (s, 1H), 8.34 (s, 1H), 8.25 (d, J=0.88 Hz, 1H), 7.56 (s, 1H), 4.06 (s, 3H), 2.73 (d, J=0.66 Hz, 3H). MS-ESI (m/z) calc'd for C₁₀H₁₀N₂O [M+H]⁺: 175.1. Found 175.0.

### Step 2: 1,6-Dimethyl-1H-indazole-5-carboxylic acid

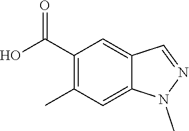

A mixture of 1,6-dimethyl-1H-indazole-5-carbaldehyde (47 mg, 0.27 mmol) and KMnO₄ (27.7 mg, 0.18 mmol) in CH₃CN/H₂O (4:1, 0.5 mL) was stirred for 2 hrs at room temperature. Another equivalent of KMnO₄ (27.7 mg, 0.18 mmol) was added and stirring was continued for another 4 hrs. The mixture was acidified with a 2 N HCl aq. sol. and extracted with EtOAc, dried over Na₂SO₄, filtered and concentrated in vacuo. The residue was combined with an additional preparation (50 mg) obtained in a similar manner and the desired product was purified by reverse phase column chromatography using a 2-60% gradient of CH₃CN in water (+0.1% HCOOH) over 10 CV to obtain the title compound (37.7 mg, 0.198 mmol, 73% yield) as a pale yellow solid. ¹H NMR (400 MHz, DMSO-d₆) δ ppm 12.42-12.82 (m, 1H), 8.36 (s, 1H), 8.12 (d, J=0.66 Hz, 1H), 7.52 (s, 1H), 4.03 (s, 3H), 2.67 (s, 3H). MS-ESI (m/z) calc'd for C₁₀H₁₁N₂O₂ [M+H]⁺: 191.1. Found 191.0.

### Step 3: N-(3-(Furan-3-yl)-1H-indazol-5-yl)-1,6-dimethyl-1H-indazole-5-carboxamide

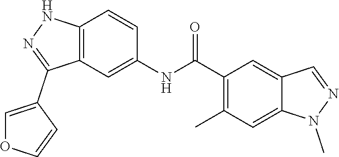 [SMILES: Cc1cc2c(cnn2C)cc1C(=O)Nc1ccc2[nH]nc(-c3ccoc3)c2c1]

To a solution of 1,6-dimethyl-1H-indazole-5-carboxylic acid (37.7 mg, 0.190 mmol), 3-(furan-3-yl)-1H-indazol-5-amine (59.23 mg, 0.280 mmol) and triethylamine (0.04 mL, 0.280 mmol) in DMF (1 mL) was added HATU (78.76 mg, 0.210 mmol) and the reaction mixture was stirred at room temperature for 2 hrs. Water (10 mL) was added and the resulting mixture was extracted with EtOAc (2×15 mL). The organic phases were combined, dried over Na₂SO₄, filtered and then concentrated under reduced pressure. The residue was purified by reverse phase column chromoatography using a 2-70% gradient of CH₃CN in water (+0.1% HCOOH) over 10 CV to afford the title compound (32.5 mg, 0.088 mmol, 46% yield) a beige solid. ¹H NMR (400 MHz, DMSO-d₆) δ ppm 13.07 (s, 1H), 10.35 (s, 1H), 8.43 (s, 1H), 8.21 (s, 1H), 8.11 (s, 1H), 7.98 (s, 1H), 7.86 (s, 1H), 7.72 (d, J=8.14 Hz, 1H), 7.50-7.61 (m, 2H), 7.00 (s, 1H), 4.06 (s, 3H), 2.58 (s, 3H). MS-ESI (m/z) calc'd for C₂₁H₁₈N₅O₂ [M+H]⁺: 172.1. Found 372.1.

### Example 159: 2-Methyl-N-(3-(oxazol-5-yl)-1H-indazol-5-yl)-2H-pyrazolo[3,4-c]pyridine-5-carboxamide

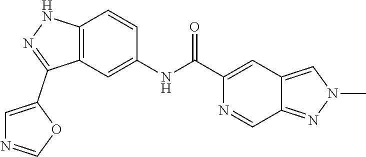 [SMILES: Cn1cc2cc(C(=O)Nc3ccc4[nH]nc(-c5cnco5)c4c3)ncc2n1]

The title compound was prepared according to the methods described above. ¹H NMR (400 MHz, DMSO-d₆) δ ppm 13.46 (br s, 1H) 10.71 (s, 1H), 9.26 (s, 1H) 8.73 (s, 1H) 8.69 (d, J=1.1 Hz, 1H) 8.59 (d, J=1.1 Hz, 1H) 8.58 (s, 1H) 7.99 (dd, J=1.8, 9.0 Hz, 1H) 7.71 (s, 1H) 7.62 (d, J=9.0 Hz, 1H), 4.32 (3H). MS-ESI (m/z) calc'd for C₁₈H₁₄N₇O₂ [M+H]⁺: 360.1. Found 360.2.

### Example 160: 1,6-Dimethyl-N-(3-(oxazol-5-yl)-1H-indazol-5-yl)-1H-pyrazolo[4,3-b]pyridine-5-carboxamide

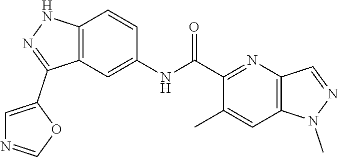 [SMILES: Cc1cc2c(cnn2C)nc1C(=O)Nc1ccc2[nH]nc(-c3cnco3)c2c1]

The title compound was prepared according to the methods described above. ¹H NMR (400 MHz, DMSO-d₆) δ ppm 13.48 (br s, 1H) 10.68 (s, 1H), 8.64 (s, 1H) 8.59 (s, 1H) 8.38 (s, 1H) 8.15 (s, 1H) 7.86 (br d, J=9.26 Hz, 1H) 7.65 (s, 1H) 7.62 (d, J=9.04 Hz, 1H) 4.11 (s, 3H) 2.71 (s, 3H). MS-ESI (m/z) calc'd for C₁₉H₁₆N₇O₂ [M+H]⁺: 374.1. Found 374.1.

### Example 161a: rel-(R)-N-(3-(Furan-3-yl)-1H-indazol-5-yl)-1-methyl-4,5,6,7-tetrahydro-1H-indazole-5-carboxamide, Example 161b: rel-(S)-N-(3-(Furan-3-yl)-1H-indazol-5-yl)-1-methyl-4,5,6,7-tetrahydro-1H-indazole-5-carboxamide, Example 161c: rel-(R)-N-(3-(furan-3-yl)-1H-indazol-5-yl)-2-methyl-4,5,6,7-tetrahydro-2H-indazole-5-carboxamide, and Example 161d: rel-(S)-N-(3-(furan-3-yl)-1H-indazol-5-yl)-2-methyl-4,5,6,7-tetrahydro-2H-indazole-5-carboxamide

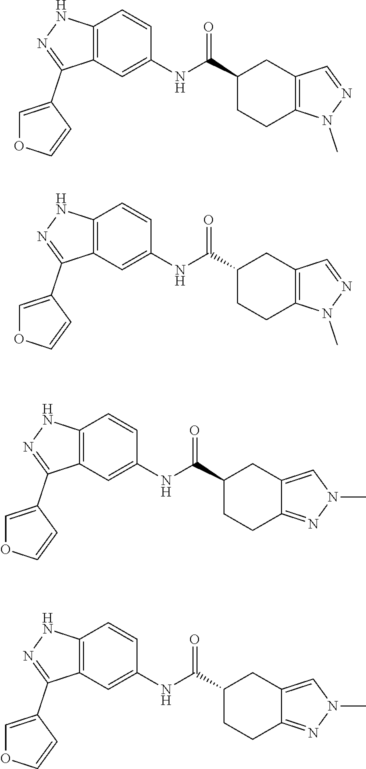 [SMILES: Cn1ncc2c1CC[C@@H](C(=O)Nc1ccc3[nH]nc(-c4ccoc4)c3c1)C2 ; Cn1ncc2c1CC[C@H](C(=O)Nc1ccc3[nH]nc(-c4ccoc4)c3c1)C2 ; Cn1cc2c(n1)CC[C@@H](C(=O)Nc1ccc3[nH]nc(-c4ccoc4)c3c1)C2 ; Cn1cc2c(n1)CC[C@H](C(=O)Nc1ccc3[nH]nc(-c4ccoc4)c3c1)C2]

### Step 1: Ethyl 1-methyl-4,5,6,7-tetrahydro-1H-indazole-5-carboxylate and Ethyl 2-methyl-4,5,6,7-tetrahydro-2H-indazole-5-carboxylate

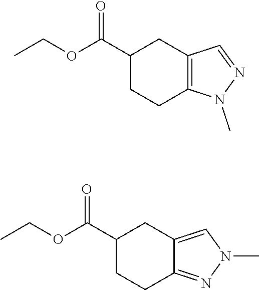

To a solution of ethyl 4,5,6,7-tetrahydro-1H-indazole-5-carboxylate (200 mg, 1.03 mmol) in DMF (2 mL) was added Cs₂CO₃ (671.00 mg, 2.06 mmol). A solution of Mel (175.39 mg, 1.24 mmol) in DMF (1 mL) was added and the mixture was stirred at 15° C. for 12 hrs. The reaction mixture was concentrated and purified by flash silica gel chromatography (ISCO; 4 g SepaFlash Silica Flash Column, eluent of 0-100% EtOAc/Petroleum ether gradient @ 80 mL/min) to afford a mixture of the title compounds (170 mg, 79%) as a pale yellow oil.

### Step 2: 1-Methyl-4,5,6,7-tetrahydro-1H-indazole-5-carboxylic acid and 2-Methyl-4,5,6,7-tetrahydro-2H-indazole-5-carboxylic acid

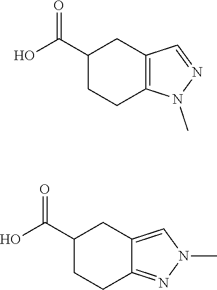

To a solution of a mixture of ethyl 1-methyl-4,5,6,7-tetrahydro-1H-indazole-5-carboxylate and ethyl 2-methyl-4,5,6,7-tetrahydro-2H-indazole-5-carboxylate (170.00 mg, 816.30 umol) in MeOH (6 mL) was added H₂O (3 mL) and LiOH.H₂O (102.77 mg, 2.45 mmol). The mixture was stirred at 15° C. for 12 hrs. The reaction mixture was concentrated under reduced pressure to remove solvent. The mixture was adjusted to pH=2 with HCl (1N), then the mixture was extracted with EtOAc. The combined organic layers were dried over Na₂SO₄, filtered and concentrated under reduced pressure to afford a mixture of the title compounds (120 mg, 82%) as a white solid.

### Step 3: rel-(R)-N-(3-(Furan-3-yl)-1H-indazol-5-yl)-1-methyl-4,5,6,7-tetrahydro-1H-indazole-5-carboxamide, rel-(S)-N-(3-(Furan-3-yl)-1H-indazol-5-yl)-1-methyl-4,5,6,7-tetrahydro-1H-indazole-5-carboxamide, rel-(R)-N-(3-(furan-3-yl)-1H-indazol-5-yl)-2-methyl-4,5,6,7-tetrahydro-2H-indazole-5-carboxamide, and rel-(S)-N-(3-(furan-3-yl)-1H-indazol-5-yl)-2-methyl-4,5,6,7-tetrahydro-2H-indazole-5-carboxamide

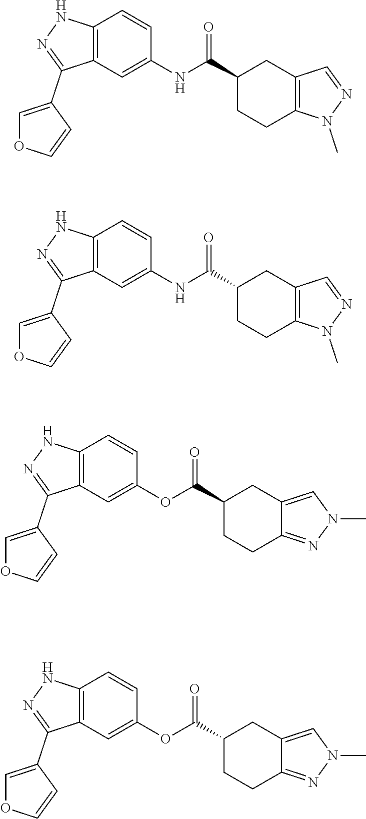 [SMILES: Cn1ncc2c1CC[C@@H](C(=O)Nc1ccc3[nH]nc(-c4ccoc4)c3c1)C2 ; Cn1ncc2c1CC[C@H](C(=O)Nc1ccc3[nH]nc(-c4ccoc4)c3c1)C2 ; Cn1cc2c(n1)CC[C@@H](C(=O)Oc1ccc3[nH]nc(-c4ccoc4)c3c1)C2 ; Cn1cc2c(n1)CC[C@H](C(=O)Oc1ccc3[nH]nc(-c4ccoc4)c3c1)C2]

To a solution of a mixture of 1-methyl-4,5,6,7-tetrahydro-1H-indazole-5-carboxylic acid and 2-methyl-4,5,6,7-tetrahydro-2H-indazole-5-carboxylic acid (90 mg, 499.44 umol) in pyridine (3 mL) was added EDCI (191.49 mg, 998.88 umol) and 3-(furan-3-yl)-1H-indazol-5-amine (129.34 mg, 649.27 umol). The mixture was stirred at 20° C. for 12 hrs. The reaction mixture was concentrated under reduced pressure to remove solvent. A second batch of 50 mg of material that was identically prepared was combined with the concentrated reaction mixture. Purification by preparative HPLC using Method N afforded a product mixture (68 mg) as a gray solid, 9 mg of which was further separated by SFC using Method O. The remaining 59 mg was further separated by SFC using Method P to afford rel-(R)-N-(3-(furan-3-yl)-1H-indazol-5-yl)-1-methyl-4,5,6,7-tetrahydro-1H-indazole-5-carboxamide (Example 161a; 11.17 mg, 6% Ret. Time=2.14) as a white solid. ¹H NMR (400 MHz, DMSO-d₆) δ 13.03 (s, 1H) 10.02 (s, 1H) 8.31 (s, 1H) 8.18 (s, 1H) 7.83 (s, 1H) 7.44-7.57 (m, 2H) 7.17 (s, 1H) 6.97 (s, 1H) 3.67 (s, 3H) 2.54-2.83 (m, 5H) 2.16 (br dd, J=12.84, 4.03 Hz, 1H) 1.82 (qd, J=11.80, 5.81 Hz, 1H). MS-ESI (m/z) calc'd for C₂₀H₂₀N₅O₂ [M+H]⁺: 362.2. Found 362.1. A second fraction was isolated to afford rel-(S)-N-(3-(Furan-3-yl)-1H-indazol-5-yl)-1-methyl-4,5,6,7-tetrahydro-1H-indazole-5-carboxamide (Example 161b; 12.82 mg, 7%, Ret. Time=2.23) as a white solid. ¹H NMR (400 MHz, DMSO-d₆) δ 13.04 (br s, 1H) 10.04 (s, 1H) 8.29-8.34 (m, 1H) 8.15-8.20 (m, 1H) 7.80-7.86 (m, 1H) 7.47-7.56 (m, 2H) 7.17 (s, 1H) 6.97 (d, J=0.73 Hz, 1H) 3.67 (s, 3H) 2.71-2.83 (m, 2H) 2.56-2.68 (m, 3H) 2.10-2.22 (m, 1H) 1.72-1.95 (m, 1H). MS-ESI (m/z) calc'd for C₂₀H₂₀N₅O₂ [M+H]⁺: 362.2. Found 362.1. A third fraction was isolated to afford rel-(R)-N-(3-(furan-3-yl)-1H-indazol-5-yl)-2-methyl-4,5,6,7-tetrahydro-2H-indazole-5-carboxamide (Example 161c; 12.37 mg, 7%, Ret. Time=2.08) as a white solid. ¹H NMR (400 MHz, DMSO-d₆) δ 13.06 (br s, 1H) 10.05 (s, 1H) 8.33 (s, 1H) 8.18 (s, 1H) 7.83 (s, 1H) 7.47-7.59 (m, 2H) 7.31-7.42 (m, 1H) 6.97 (s, 1H) 3.73 (s, 3H) 2.54-2.79 (m, 5H) 2.12 (br d, J=11.37 Hz, 1H) 1.72-1.85 (m, 1H). MS-ESI (m/z) calc'd for C₂₀H₂₀N₅O₂ [M+H]⁺: 362.2. Found 362.0. A fourth fraction was isolated to afford rel-(S)-N-(3-(furan-3-yl)-1H-indazol-5-yl)-2-methyl-4,5,6,7-tetrahydro-2H-indazole-5-carboxamide (Example 161d; 10.18 mg, 6%, Ret. Time=2.48) as a white solid. ¹H NMR (400 MHz, DMSO-d₆) δ 13.04 (s, 1H) 10.04 (s, 1H) 8.32 (s, 1H) 8.18 (s, 1H) 7.83 (s, 1H) 7.44-7.57 (m, 2H) 7.38 (s, 1H) 6.97 (d, J=0.73 Hz, 1H) 3.73 (s, 3H) 2.54-2.82 (m, 5H) 2.07-2.16 (m, 1H) 1.70-1.84 (m, 1H). MS-ESI (m/z) calc'd for C₂₀H₂₀N₅O₂ [M+H]⁺: 362.2. Found 362.0. Absolute stereochemistry and regiochemistry (position of the N-methyl group) arbitrarily assigned.

### Example 162a: rel-(R)-N-(3-(furan-3-yl)-1H-indazol-5-yl)-5,6,7,8-tetrahydro-[1,2,4]triazolo[4,3-a]pyridine-6-carboxamide and Example 162b: rel-(S)-N-(3-(furan-3-yl)-1H-indazol-5-yl)-5,6,7,8-tetrahydro-[1,2,4]triazolo[4,3-a]pyridine-6-carboxamide

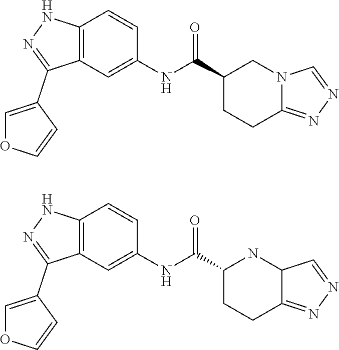 [SMILES: O=C(Nc1ccc2[nH]nc(-c3ccoc3)c2c1)[C@@H]1CCc2nncn2C1 ; O=C(Nc1ccc2[nH]nc(-c3ccoc3)c2c1)[C@H]1CCC2=NN=CC2N1]

### Step 1: 5,6,7,8-tetrahydro-[1,2,4]triazolo[4,3-a]pyridine-6-carboxylic acid

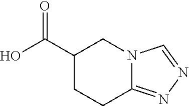 [SMILES: O=C(O)C1CCc2nncn2C1]

To a solution of [1,2,4]triazolo[4,3-a]pyridine-6-carboxylic acid (500 mg, 3.06 mmol) in MeOH (60 mL) was added Pd(OH)₂ (10% on activated charcoal, 500 mg) under Ar. The suspension was degassed under vacuum and purged with H₂ (3×). The mixture was stirred under H₂ (4 Mpa) at 80° C. for 12 hrs. The reaction mixture was filtered and the filtrate was concentrated under reduced pressure to afford the title compound (300 mg, 59%) as a white solid which was used without further purification. ¹H NMR (400 MHz, DMSO-d₆) δ 8.39 (s, 1H), 4.27 (m, 2H), 3.06-2.90 (m, 3H), 2.31 (m, 1H), 2.16 (m, 1H).

### Step 2: rac-3-(Furan-3-yl)-1H-indazol-5-yl 5,6,7,8-tetrahydro-[1,2,4]triazolo[4,3-a]pyridine-6-carboxylate

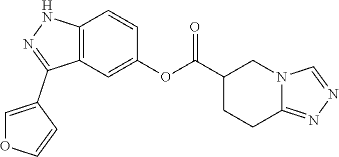 [SMILES: O=C(Oc1ccc2[nH]nc(-c3ccoc3)c2c1)C1CCc2nncn2C1]

To a solution of 5,6,7,8-tetrahydro-[1,2,4]triazolo[4,3-a]pyridine-6-carboxylic acid (200 mg, 1.20 mmol) in pyridine (5 mL) was added 3-(furan-3-yl)-1H-indazol-5-amine (262.17 mg, 1.32 mmol), EDCI (458.71 mg, 2.39 mmol). The mixture was stirred at 20° C. for 12 hrs. The reaction mixture was concentrated and purified by preparative HPLC using Method Q to afford the title compound (180 mg, 43%) as a white solid.

### Step 3: rel-(R)-N-(3-(furan-3-yl)-1H-indazol-5-yl)-5,6,7,8-tetrahydro-[1,2,4]triazolo[4,3-a]pyridine-6-carboxamide and rel-(S)-N-(3-(furan-3-yl)-H-indazol-5-yl)-5,6,7,8-tetrahydro-[1,2,4]triazolo[4,3-a]pyridine-6-carboxamide

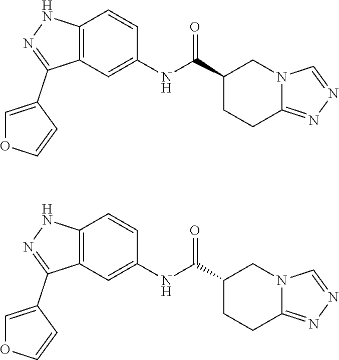 [SMILES: O=C(Nc1ccc2[nH]nc(-c3ccoc3)c2c1)[C@@H]1CCc2nncn2C1 ; O=C(Nc1ccc2[nH]nc(-c3ccoc3)c2c1)[C@H]1CCc2nncn2C1]

A mixture of 3-(furan-3-yl)-1H-indazol-5-yl 5,6,7,8-tetrahydro-[1,2,4]triazolo[4,3-a]pyridine-6-carboxylate (20 mg, 57.41 umol) in MeOH (2 mL) was stirred at 20° C. for 10 min, then it was separated by Method R to afford rel-(R)-N-(3-(furan-3-yl)-1H-indazol-5-yl)-5,6,7,8-tetrahydro-[1,2,4]triazolo[4,3-a]pyridine-6-carboxamide (Example 162a; 4.67 mg, 22%) as a gray solid. ¹H NMR (400 MHz, DMSO-d₆) δ 13.05 (s, 1H) 10.21 (s, 1H) 8.42 (s, 1H) 8.27 (s, 1H) 8.17 (s, 1H) 7.83 (t, J=1.65 Hz, 1H) 7.48-7.56 (m, 2H) 6.97 (dd, J=1.71, 0.73 Hz, 1H) 4.32 (dd, J=12.47, 5.26 Hz, 1H) 4.15 (dd, J=12.53, 8.99 Hz, 1H) 3.06-3.15 (m, 1H) 2.95-3.04 (m, 1H) 2.80-2.91 (m, 1H) 2.24-2.34 (m, 1H) 2.02-2.15 (m, 1H). MS-ESI (m/z) calc'd for C₁₈H₁₆N₅O₃ [M+H]⁺: 349.1. Found 349.1. A second fraction was isolated to afford rel-(S)-N-(3-(furan-3-yl)-1H-indazol-5-yl)-5,6,7,8-tetrahydro-[1,2,4]triazolo[4,3-a]pyridine-6-carboxamide (Example 162b; 3.84 mg, 19%) as a gray solid. ¹H NMR (400 MHz, DMSO-d₆) δ 13.05 (s, 1H) 10.22 (s, 1H) 8.43 (s, 1H) 8.27 (s, 1H) 8.17 (s, 1H) 7.83 (t, J=1.65 Hz, 1H) 7.45-7.55 (m, 2H) 6.96 (d, J=1.10 Hz, 1H) 4.31 (dd, J=12.41, 5.32 Hz, 1H) 4.14 (dd, J=12.47, 8.93 Hz, 1H) 3.05-3.15 (m, 1H) 2.99 (dt, J=16.90, 5.12 Hz, 1H) 2.79-2.90 (m, 1H) 2.23-2.34 (m, 1H) 2.02-2.14 (m, 1H). MS-ESI (m/z) calc'd for C₁₈H₁₆N₅O₃ [M+H]⁺: 349.1. Found 349.1. Absolute stereochemistry arbitrarily assigned.

### Example 163: N-(3-(Furan-3-yl)-1H-indazol-5-yl)-4,6-dimethylpyrazolo[1,5-a]pyrazine-2-carboxamide

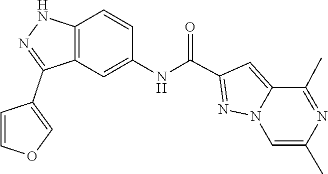 [SMILES: Cc1cn2nc(C(=O)Nc3ccc4[nH]nc(-c5ccoc5)c4c3)cc2c(C)n1]

### Step 1: 4,6-Dimethylpyrazolo[1,5-a]pyrazine-2-carboxylic acid

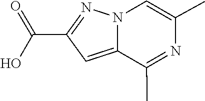

To a solution of ethyl 4,6-dimethylpyrazolo[1,5-a]pyrazine-2-carboxylate (50 mg, 228.06 umol) in THF (1 mL) and H₂O (1 mL) was added LiOH.H₂O (2.5 M, 273.67 uL). The mixture was stirred at 20° C. for 1 hr. The reaction mixture was concentrated under reduced pressure to remove solvent. The residue was diluted with DCM (30 mL) and MeOH (30 mL), then the mixture was filtered and the filtrate was concentrated under reduced pressure to afford the title compound (130 mg) as a white solid which was used without further purification.

### Step 2: N-(3-(Furan-3-yl)-1H-indazol-5-yl)-4,6-dimethylpyrazolo[1,5-a]pyrazine-2-carboxamide

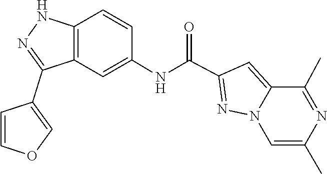 [SMILES: Cc1cn2nc(C(=O)Nc3ccc4[nH]nc(-c5ccoc5)c4c3)cc2c(C)n1]

To a solution of 4,6-dimethylpyrazolo[1,5-a]pyrazine-2-carboxylic acid (130 mg, 679.97 umol) and 3-(furan-3-yl)-1H-indazol-5-amine (176.09 mg, 883.96 umol) in pyridine (4 mL) was added EDCI (260.70 mg, 1.36 mmol) and the mixture was stirred at 20° C. for 12 hrs. The reaction mixture was then concentrated under reduced pressure to remove solvent. The residue was diluted with DMF (1 mL) and acidified with TFA to pH=1. The mixture was purified by preparative HPLC using Method S to afford the title compound (20.72 mg, 6%) as a light red solid, TFA salt. ¹H NMR (400 MHz, DMSO-d₆) δ 13.09 (br s, 1H), 10.48 (s, 1H), 8.52-8.42 (m, 2H), 8.27 (s, 1H), 7.91 (d, J=10.1 Hz, 1H), 7.85 (s, 1H), 7.55 (d, J=8.8 Hz, 1H), 7.52 (s, 1H), 7.01 (s, 1H), 2.75 (s, 3H), 2.47 (s, 3H). MS-ESI (m/z) calc'd for C₂₀H₁₇N₆O₂ [M+H]⁺: 373.1. Found 373.1.

### Example 164: N-(3-(furan-3-yl)-1H-indazol-5-yl)-2-methylpyrazolo[1,5-a]pyrazine-3-carboxamide

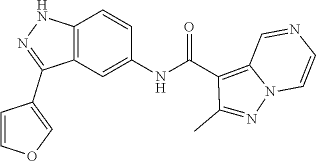 [SMILES: Cc1nn2ccncc2c1C(=O)Nc1ccc2[nH]nc(-c3ccoc3)c2c1]

### Step 1: Potassium Amino Sulfate

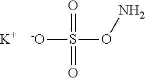

To a solution of (aminooxy)sulfonic acid (56.63 g, 500.74 mmol) in H₂O (250 mL) was added a solution of KOH (28.09 g, 500.74 mmol) in H₂O (156 mL) and the reaction mixture was stirred at 0° C. for 30 min. The crude product in H₂O was used for the next step without monitoring. After warming to 20° C., the colorless solution in water was used for subsequent experiments. The title compound (57 g, 75%) (estimated amount) in H₂O was obtained as a colorless liquid, which was used without further purification.

### Step 2: 1-Aminopyrazin-1-ium Iodide

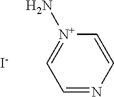

To a solution of pyrazine (30 g, 374.59 mmol) in H₂O (140 mL) was dropwise added potassium amino sulfate (56.63 g, 374.59 mmol) at 60° C. over a period of 15 min. The reaction mixture was stirred at 70° C. for 4 hrs. The solution was made alkaline (pH=8-9) with K₂CO₃. The precipitated K₂SO₄ was filtered off and the filtrate was washed with EtOAc (300 mL×3). The aqueous solution was acidified (pH=3) with a 38% HI aqueous solution and concentrated under reduced pressure at temperatures below 50° C. The residue was washed with EtOH (300 mL) and the insoluble matter was filtered off. The filtrate was evaporated under reduced pressure to afford the title compound (39 g, 47%) as a dark brown solid. See also: Chem. Pharm. Bull. 1974, 22, 1814-1826.

### Step 3: Ethyl 2-methylpyrazolo[1,5-a]pyrazine-3-carboxylate

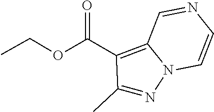

To a solution of 1-aminopyrazin-1-ium iodide (20 g, 89.68 mmol) and ethyl but-2-ynoate (50.28 g, 448.40 mmol, 52.26 mL) in DMF (200 mL) was added K₂CO₃ (30.99 g, 224.20 mmol). The reaction mixture was stirred at 25° C. for 12 hrs. The mixture was poured into water (200 mL) and the aqueous phase was extracted with EtOAc (100 mL×3). The combined organic phases were washed with brine (100 mL×1), dried over Na₂SO₄, filtered and concentrated. The residue was purified by column chromatography on silica gel using a 5-95% gradient of EtOAc in petroleum ether and further purified by preparative HPLC using Method T to afford the title compound (250 mg, 1.36%) as a yellow solid. MS-ESI (m/z) calc'd for C₁₀H₁₂N₃O₂ [M+H]⁺: 205.1. Found 204.8.

### Step 4: N-(3-(Furan-3-yl)-1H-indazol-5-yl)-2-methylpyrazolo[1,5-a]pyrazine-3-carboxamide

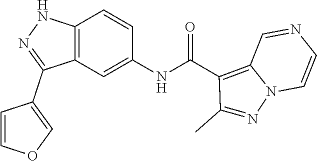 [SMILES: Cc1nn2ccncc2c1C(=O)Nc1ccc2[nH]nc(-c3ccoc3)c2c1]

To a solution of ethyl 2-methylpyrazolo[1,5-a]pyrazine-3-carboxylate (50 mg, 243.65 umol) and 3-(furan-3-yl)-1H-indazol-5-amine (72.81 mg, 365.47 umol) in toluene (2 mL) was added AlMe₃ (2 M, 609.12 uL). The reaction mixture was stirred at 25° C. for 12 hrs. After cooling to 0° C., the reaction mixture was quenched with saturated aqueous NH₄Cl (10 mL) while stirring. The aqueous phase was extracted with EtOAc (10 mL×3) and the combined organic phases were washed with brine (10 mL×1), dried over Na₂SO₄, filtered and concentrated. The residue was purified by preparative HPLC using Method U to afford the title compound (5.69 mg, 6%) as a white solid. ¹H NMR (400 MHz, DMSO-d₆) δ 13.08 (s, 1H), 10.08 (s, 1H), 9.36 (d, J=1.3 Hz, 1H), 8.86-8.78 (m, 1H), 8.35 (d, J=1.1 Hz, 1H), 8.27 (d, J=0.7 Hz, 1H), 8.04 (d, J=4.6 Hz, 1H), 7.85 (t, J=1.7 Hz, 1H), 7.70 (dd, J=1.7, 8.9 Hz, 1H), 7.56 (d, J=9.0 Hz, 1H), 7.01 (d, J=1.1 Hz, 1H), 2.68 (s, 3H). MS-ESI (m/z) calc'd for C₁₉H₁₅N₆O₂ [M+H]⁺: 359.1. Found 359.1.

### Example 165: 1-Methyl-N-(3-(oxazol-5-yl)-1H-indazol-5-yl)-1H-pyrazolo[4,3-b]pyridine-5-carboxamide

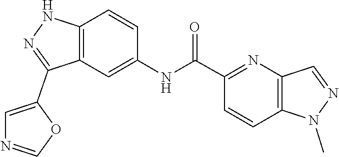 [SMILES: Cn1ncc2nc(C(=O)Nc3ccc4[nH]nc(-c5cnco5)c4c3)ccc21]

### Step 1: 5-Chloro-1-methyl-1H-pyrazolo[4,3-b]pyridine and 5-Chloro-2-methyl-2H-pyrazolo[4,3-b]pyridine

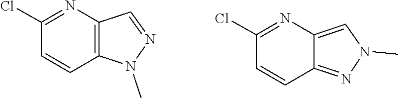

To a solution of 5-chloro-1H-pyrazolo[4,3-b]pyridine (2 g, 13.02 mmol) in acetone (20 mL) was added KOH (2.19 g, 39.07 mmol) at 0° C. for 1 hr. Mel (2.77 g, 19.54 mmol, 1.22 mL) was added and the mixture was stirred at 25° C. for 12 hrs. The reaction mixture was filtered and the filtrate was concentrated. The residue was purified by column chromatography on silica gel using a 0-66% gradient of EtOAc in petroleum ether to afford the title compounds, 5-chloro-1-methyl-1H-pyrazolo[4,3-b]pyridine (1.5 g, 67%) as a white solid and 5-chloro-2-methyl-2H-pyrazolo[4,3-b]pyridin (990 mg, 44%) as a white solid. MS-ESI (m/z) calc'd for C₇H₇ClN₃ [M+H]⁺: 168.0. Found 168.1.

### Step 2: Methyl 1-methyl-1H-pyrazolo[4,3-b]pyridine-5-carboxylate

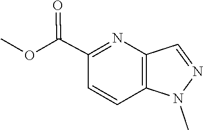

To a solution of 5-chloro-1-methyl-1H-pyrazolo[4,3-b]pyridine (1 g, 5.97 mmol) in MeOH (10 mL) was added Pd(dppf)Cl₂ (436.59 mg, 596.68 umol) and NEt₃ (1.81 g, 17.90 mmol, 2.49 mL) under N₂. The suspension was degassed under vacuum and purged with CO several times. The mixture was stirred under CO (50 psi) at 80° C. for 12 hrs. The reaction mixture was concentrated. The residue was purified by column chromatography on silica gel using a 0-50% gradient of EtOAc in petroleum ether to afford the title compound (550 mg, 48%) as a yellow solid. MS-ESI (m/z) calc'd for C₉H₁₀N₃O₂ [M+H]⁺: 192.1. Found 192.1.

### Step 3: 1-Methyl-1H-pyrazolo[4,3-b]pyridine-5-carboxylic acid

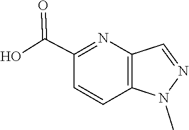

To a solution of methyl 1-methyl-1H-pyrazolo[4,3-b]pyridine-5-carboxylate (300 mg, 1.57 mmol) in THF (3 mL) and H₂O (3 mL) was added LiOH.H₂O (131.69 mg, 3.14 mmol). The mixture was stirred at 25° C. for 12 hrs. The reaction mixture was adjusted to pH 7 with 1 N HCl and the mixture was filtered and the cake was dried to afford the title compound (250 mg, crude) as a white solid which was used without further purification. MS-ESI (m/z) calc'd for C₈H₈N₃O₂ [M+H]⁺: 178.1. Found 178.1.

### Step 4: 1-Methyl-N-(3-(oxazol-5-yl)-1H-indazol-5-yl)-1H-pyrazolo[4,3-b]pyridine-5-carboxamide

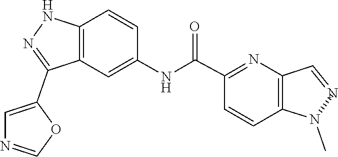

To a solution of 1-methyl-1H-pyrazolo[4,3-b]pyridine-5-carboxylic acid (150 mg, 846.69 umol) and 3-(oxazol-5-yl)-1H-indazol-5-amine (169.50 mg, 846.69 umol) in pyridine (2 mL) was added EDCI (324.63 mg, 1.69 mmol). The mixture was stirred at 25° C. for 2 hrs. The reaction mixture was poured into water (5 mL) and extracted with EtOAc (5 mL×3). The combined organic phases were washed with brine (5 mL×1), dried over anhydrous Na₂SO₄, filtered and concentrated. The residue was purified by preparative HPLC using Method V to afford the title compound (43.09 mg, 14%) as a pale yellow solid. ¹H NMR (400 MHz, DMSO-d₆) δ 13.49 (br s, 1H), 10.81 (s, 1H), 8.70 (s, 1H), 8.59 (s, 1H), 8.49 (s, 1H), 8.39 (d, J=9.5 Hz, 1H), 8.24 (d, J=8.8 Hz, 1H), 8.01 (dd, J=2.0, 9.0 Hz, 1H), 7.70 (s, 1H), 7.63 (d, J=9.0 Hz, 1H), 4.16 (s, 3H). MS-ESI⁻ (m/z) calc'd for C₁₈H₁₂N₇O₂ [M−H]⁻: 358.1. Found 358.1.

### Example 166: 2-Methyl-N-(3-(oxazol-5-yl)-1H-indazol-5-yl)-2H-pyrazolo[4,3-b]pyridine-5-carboxamide

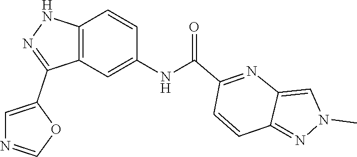

Prepared as described for 1-methyl-N-(3-(oxazol-5-yl)-1H-indazol-5-yl)-1H-pyrazolo[4,3-b]pyridine-5-carboxamide (Example 165) using 5-chloro-2-methyl-2H-pyrazolo[4,3-b]pyridine in place of 5-chloro-1-methyl-1H-pyrazolo[4,3-b]pyridine to afford the title compound (52.59 mg, 25%) as a pale yellow solid. ¹H NMR (400 MHz, DMSO-d₆) δ 10.78 (s, 1H), 8.89 (s, 1H), 8.70 (d, J=1.4 Hz, 1H), 8.59 (s, 1H), 8.31 (dd, J=0.9, 9.0 Hz, 1H), 8.09 (d, J=9.0 Hz, 1H), 8.00 (dd, J=1.9, 9.1 Hz, 1H), 7.70 (s, 1H), 7.62 (d, J=9.1 Hz, 1H), 4.31 (s, 3H). MS-ESI (m/z) calc'd for C₁₈H₁₄N₇O₂ [M+H]⁺: 360.1. Found 360.2.

### Example 167: 1-Methyl-N-(3-(oxazol-5-yl)-1H-indazol-5-yl)-1H-pyrazolo[3,4-c]pyridine-5-carboxamide

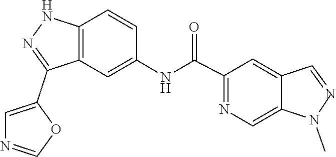

Prepared as described for 1-methyl-N-(3-(oxazol-5-yl)-1H-indazol-5-yl)-1H-pyrazolo[4,3-b]pyridine-5-carboxamide (Example 165) using 5-chloro-1-methyl-1H-pyrazolo[3,4-c]pyridine in place of 5-chloro-1-methyl-1H-pyrazolo[4,3-b]pyridine to afford the title compound (12.23 mg, 5%) as a pale pink solid. ¹H NMR (400 MHz, DMSO-d₆) δ 13.46 (br s, 1H), 10.75 (s, 1H), 9.28 (s, 1H), 8.69 (d, J=1.3 Hz, 1H), 8.62 (d, J=0.9 Hz, 1H), 8.58 (s, 1H), 8.41 (d, J=0.4 Hz, 1H), 8.01 (dd, J=1.8, 9.0 Hz, 1H), 7.71 (s, 1H), 7.62 (d, J=8.8 Hz, 1H), 4.28 (s, 3H). MS-ESI (m/z) calc'd for C₁₈H₁₄N₇O₂ [M+H]⁺: 360.1. Found 360.2.

### Example 168: 5-Methyl-N-(3-(oxazol-5-yl)-1H-indazol-5-yl)isothiazolo[5,4-b]pyridine-6-carboxamide

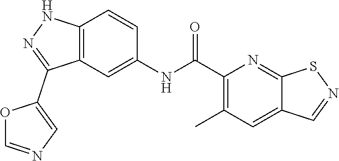 [SMILES: Cc1cc2cnsc2nc1C(=O)Nc1ccc2[nH]nc(-c3cnco3)c2c1]

### Step 1: 5-Bromo-2-(methoxycarbonyl)-3-methylpyridine 1-oxide

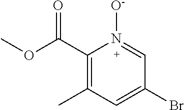

To a solution of methyl 5-bromo-3-methylpicolinate (2.5 g, 10.87 mmol) in DCM (50 mL) was added m-CPBA (4.69 g, 21.73 mmol) (80% purity). The mixture was stirred at 45° C. for 12 hrs. The reaction mixture was quenched with saturated aqueous Na₂SO₃ (100 mL) and stirred at 15° C. for 30 min. The organic layer was separated and the aqueous phase was extracted with DCM (35 mL×3), the combined organic layer was dried over Na₂SO₄, filtered and concentrated to afford the title compound (2.7 g, crude) as a yellow oil.

### Step 2: Methyl 5-bromo-6-chloro-3-methylpicolinate

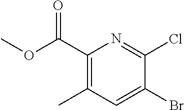

A solution of 5-bromo-2-(methoxycarbonyl)-3-methylpyridine 1-oxide (2.7 g, 10.97 mmol) in POCl₃ (20 mL) was stirred at 90° C. for 3 hrs. The reaction mixture was cooled to 20° C. and poured into H₂O (50 mL). The mixture was basified with saturated aqueous NaHCO₃ to pH=7 and extracted with EtOAc (25 mL×3). The combined organic layer was dried over Na₂SO₄, filtered and concentrated to give a residue. The residue was purified by flash silica gel chromatography (ISCO; 12 g SepaFlash Silica Flash Column, Eluent of 0-5% EtOAc/Petroleum ether gradient @ 100 mL/min) (petroleum ether/EtOAc=5/1, R_{f}=0.43) to afford the title compound (2 g, 69%) as a white solid.

### Step 3: Methyl 6-chloro-3-methyl-5-vinylpicolinate

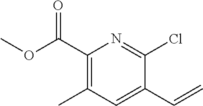

A mixture of methyl 5-bromo-6-chloro-3-methylpicolinate (2 g, 7.56 mmol), potassium trifluoro(vinyl)borate (1.09 g, 8.11 mmol), K₂CO₃ (2.09 g, 15.12 mmol), Pd(dppf)Cl₂ (553.27 mg, 756.13 umol) in THF (60 mL) and H₂O (15 mL) was degassed and purged with N₂ at 20° C. and then the mixture was stirred at 70° C. for 12 hrs under N₂ atmosphere. The reaction mixture was concentrated and purified by silica gel chromatography using a 0-5% EtOAc/petroleum ether gradient eluent (ISCO; 20 g SepaFlash Silica Flash Column) to afford the title compound (1 g, 62%) as a white solid. MS-ESI (m/z) calc'd for C₁₀H₁₀ClNO₂ [M+H]⁺: 212.0. Found 212.0.

### Step 4: Methyl 6-chloro-5-formyl-3-methylpicolinate

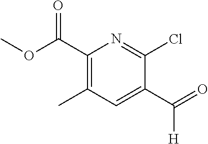

To a solution of methyl 6-chloro-3-methyl-5-vinylpicolinate (1 g, 4.72 mmol) in DCM (50 mL) was bubbled 03 (15 Psi) for 10 min at −78° C., then Me₂S (2.94 g, 47.25 mmol) was added and the mixture was stirred at −78° C. for 20 min. The reaction mixture was quenched with saturated aqueous NaHCO₃ (25 mL) and extracted with DCM (15 mL×3). The combined organic layers were dried over Na₂SO₄, filtered and concentrated to give a residue. The residue was purified by silica gel chromatography using a 0-5% EtOAc/petroleum ether gradient eluent (ISCO®; 12 g SepaFlash® Silica Flash Column) to afford the title compound (930 mg, 92%) as a white solid. ¹H NMR (400 MHz, CDCl₃) δ 10.37 (s, 1H), 8.06 (s, 1H), 3.94 (s, 3H), 2.54 (s, 3H).

### Step 5: Methyl 5-methylisothiazolo[5,4-b]pyridine-6-carboxylate

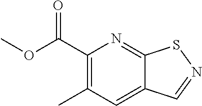

To a solution of NH₃.H₂O (2 mL) (25% purity) in DMF (2 mL) was added methyl 6-chloro-5-formyl-3-methylpicolinate (300 mg, 1.40 mmol) and sulfur (47.29 mg, 1.47 mmol) at 20° C. The mixture was allowed to warm to 90° C. slowly and stirred at 90° C. for 1 hr. The reaction mixture was then diluted with H₂O (15 mL) and extracted with EtOAc (10 mL×5). The combined organic layers were dried over Na₂SO₄, filtered and concentrated to give a residue. The residue was purified by preparative TLC (SiO₂, petroleum ether/EtOAc=1/1, Rf=0.58) to afford the title compound (14 mg, 5%) as a yellow solid, ¹H NMR (400 MHz, CDCl₃) δ 8.88 (s, 1H), 8.18 (s, 1H), 3.99 (s, 3H), 2.65 (s, 3H).

### Step 6: 5-Methylisothiazolo[5,4-b]pyridine-6-carboxylic acid

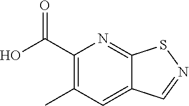

To a solution of methyl 5-methylisothiazolo[5,4-b]pyridine-6-carboxylate (7 mg, 33.62 umol) in THF (0.5 mL) and H₂O (0.5 mL) was added LiOH.H₂O (2.82 mg, 67.23 umol). The mixture was stirred at 20° C. for 1 hr. The reaction mixture was then acidified with 1N HCl to pH=3 and then diluted with H₂O (4 mL) and extracted with EtOAc (3 mL×3). The combined organic layers were dried over Na₂SO₄, filtered and concentrated to afford the title compound (6 mg) as a yellow solid which was used without further purification.

### Step 7: 5-Methyl-N-(3-(oxazol-5-yl)-1H-indazol-5-yl)isothiazolo[5,4-b]pyridine-6-carboxamide

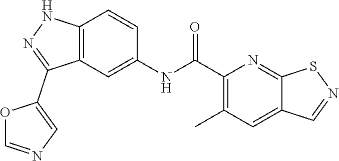 [SMILES: Cc1cc2cnsc2nc1C(=O)Nc1ccc2[nH]nc(-c3cnco3)c2c1]

To a solution of 5-methylisothiazolo[5,4-b]pyridine-6-carboxylic acid (6 mg, 30.89 umol) in pyridine (1 mL) was added EDCI (8.88 mg, 46.34 umol) and 3-(oxazol-5-yl)-1H-indazol-5-amine (7.42 mg, 37.07 umol). The mixture was stirred at 20° C. for 12 hrs. The reaction mixture was concentrated and purified by preparative HPLC using Method W to afford the title compound (1.59 mg, 10%) as a yellow solid. ¹H NMR (400 MHz, DMSO-d₆) δ 13.52 (s, 1H) 10.91 (s, 1H) 9.28 (s, 1H) 8.64 (d, J=6 Hz, 2H) 8.60 (s, 1H) 7.84 (dd, J=9, 2 Hz, 1H) 7.62-7.68 (m, 2H) 2.70 (s, 3H). MS-ESI (m/z) calc'd for C₁₈H₁₃N₆O₂S [M+H]⁺: 377.1. Found 377.1.

### Example 169: 1,4-Dimethyl-N-(3-(oxazol-5-yl)-1H-indazol-5-yl)-1H-pyrazolo[3,4-c]pyridine-5-carboxamide

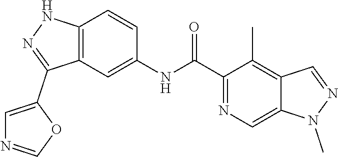 [SMILES: Cc1c(C(=O)Nc2ccc3[nH]nc(-c4cnco4)c3c2)ncc2c1cnn2C]

### Step 1: 2-Bromo-5-fluoro-3-methylisonicotinaldehyde

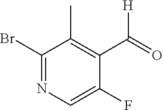

To a solution of 2-bromo-5-fluoro-3-methylpyridine (1 g, 5.26 mmol) in THF (30 mL) was added LDA (2 M in THF, 6.58 mL) at −60° C. The mixture was stirred at −60° C. for 1 hr, then methyl formate (948.12 mg, 15.79 mmol) was added. The mixture was then stirred at −60° C. for another 2 hrs. The reaction mixture was quenched with H₂O (25 mL) and extracted with EtOAc (20 mL×3). The combined organic layers were dried over Na₂SO₄, filtered and concentrated to give a residue. The residue was purified by silica gel chromatography using a 0-93% EtOAc/petroleum ether gradient eluent (ISCO; 3 g SepaFlash Silica Flash Column) to afford the title compound (960 mg, 84%) as a yellow solid. ¹H NMR (400 MHz, CDCl₃) δ 10.42 (s, 1H), 8.26 (s, 1H), 2.60 (s, 3H).

### Step 2: 5-Bromo-4-methyl-1H-pyrazolo[3,4-c]pyridine

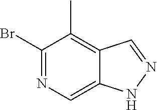

To a solution of 2-bromo-5-fluoro-3-methylisonicotinaldehyde (540 mg, 2.48 mmol) in DME (12 mL) was added NH₂NH₂.H₂O (12 mL). The mixture was stirred at 120° C. for 12 hrs. The reaction mixture was then concentrated under reduced pressure to remove solvent. The residue was diluted with 100 mL of EtOAc, filtered and the filtrate was concentrated under vacuum. The residue was purified by silica gel chromatography using a 0-6% EtOAc/Petroleum ether gradient eluent (ISCO; 12 g SepaFlash Silica Flash Column) to afford the title compound (110 mg, 20%) as a yellow solid. ¹H NMR (400 MHz, CDCl₃) δ 10.65 bs, 1H), 8.70 (s, 1H), 8.15 (s, 1H), 2.67 (s, 3H). MS-ESI (m/z) calc'd for C₇H₇BrN₃ [M+H]⁺: 212.0, 218.0. Found 211.9, 213.9.

### Step 3: 5-Bromo-1,4-dimethyl-1H-pyrazolo[3,4-c]pyridine and 5-Bromo-2,4-dimethyl-2H-pyrazolo[3,4-c]pyridine

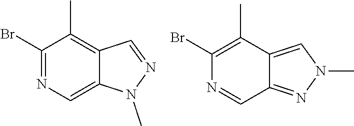

To a stirred solution of 5-bromo-4-methyl-1H-pyrazolo[3,4-c]pyridine (110 mg, 518.75 umol) in acetone (6 mL) was added KOH (87.32 mg, 1.56 mmol) at 0° C. and the reaction mixture was stirred at 0° C. for 1 hr. Mel (147.26 mg, 1.04 mmol) was added and the reaction mixture was warmed to 20° C. and stirred for 3 hrs. The reaction mixture was filtered and the filtrate was concentrated. The residue was purified by preparative TLC (SiO₂, petroleum ether/EtOAc=1:1, P1 R_{f}=0.51, P2 R_{f}=0.38) to afford the 5-bromo-1,4-dimethyl-1H-pyrazolo[3,4-c]pyridine (70 mg, 60%) as a yellow solid, ¹H NMR (400 MHz, CDCl₃) δ 8.57 (s, 1H), 8.01 (s, 1H), 4.14 (s, 3H), 2.62 (s, 3H), and 5-bromo-2,4-dimethyl-2H-pyrazolo[3,4-c]pyridine (58 mg, 49%) as a yellow solid, ¹H NMR (400 MHz, CDCl₃) δ 8.85 (s, 1H), 7.94 (s, 1H), 4.29 (s, 3H), 2.54 (s, 3H).

### Step 4: 5-(1-Ethoxyvinyl)-1,4-dimethyl-1H-pyrazolo[3,4-c]pyridine

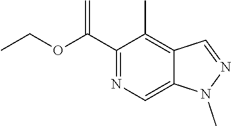 [SMILES: C=C(OCC)c1ncc2c(cnn2C)c1C]

To a solution of 5-bromo-1,4-dimethyl-1H-pyrazolo[3,4-c]pyridine (68 mg, 300.79 umol) in dioxane (3 mL) was added Pd(PPh₃)₂Cl₂ (21.11 mg, 30.08 umol) and tributyl(1-ethoxyvinyl)stannane (130.36 mg, 360.95 umol) at 15° C. The mixture was stirred at 100° C. for 12 hrs under N₂ atmosphere. The reaction mixture was concentrated under reduced pressure to remove the solvent. The residue was purified by preparative TLC (SiO₂, petroleum ether/EtOAc=1:1, Rf=0.16) to afford the title compound (52 mg, 80%) as a yellow oil. MS-ESI (m/z) calc'd for C₁₂H₁₆N₃O [M+H]⁺: 218.1. Found 218.0.

### Step 5: Ethyl 1,4-dimethyl-1H-pyrazolo[3,4-c]pyridine-5-carboxylate

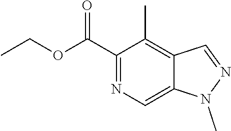 [SMILES: CCOC(=O)c1ncc2c(cnn2C)c1C]

To a solution of 5-(1-ethoxyvinyl)-1,4-dimethyl-1H-pyrazolo[3,4-c]pyridine (52 mg, 239.34 umol) in dioxane (2 mL) was added KMnO₄ (5.67 mg, 35.90 umol) in H₂O (1 mL) and NaIO₄ (102.38 mg, 478.68 umol). The mixture was stirred at 25° C. for 1 hr. The reaction mixture was filtered, the filtrate was diluted with saturated aqueous NaHCO₃ (10 mL) and extracted with EtOAc (8 mL×3). The combined organic layers were dried over Na₂SO₄, filtered and concentrated under reduced pressure to give a residue. The residue was purified by preparative TLC (SiO₂, petroleum ether/EtOAc=1:2, Rf=0.23) to afford the title compound (29 mg, 55%) as a yellow oil. MS-ESI (m/z) calc'd for C₁₁H₁₄N₃O₂ [M+H]⁺: 220.1. Found 220.0.

### Step 6: 1,4-Dimethyl-1H-pyrazolo[3,4-c]pyridine-5-carboxylic acid

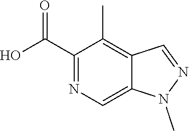

To a solution of ethyl 1,4-dimethyl-1H-pyrazolo[3,4-c]pyridine-5-carboxylate (29 mg, 132.28 umol) in THF (1 mL) and H₂O (1 mL) was added LiOH.H₂O (11.10 mg, 264.55 umol). The mixture was stirred at 25° C. for 12 hrs. The reaction mixture was adjusted to pH 4 with 1 N HCl and concentrated to afford the title compound (25 mg) as a yellow solid which was used without further purification. MS-ESI (m/z) calc'd for C₉H₁₀N₃O₂ [M+H]⁺: 192.1. Found 192.1.

### Step 7: 1,4-Dimethyl-N-(3-(oxazol-5-yl)-1H-indazol-5-yl)-1H-pyrazolo[3,4-c]pyridine-5-carboxamide

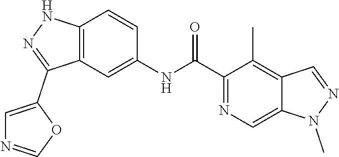 [SMILES: Cc1c(C(=O)Nc2ccc3[nH]nc(-c4cnco4)c3c2)ncc2c1cnn2C]

To a solution of 1,4-dimethyl-1H-pyrazolo[3,4-c]pyridine-5-carboxylic acid (20 mg, 104.61 umol) and 3-(oxazol-5-yl)-1H-indazol-5-amine (41.89 mg, 209.22 umol) in DMF (3 mL) was added EDCI (24.06 mg, 125.53 umol), HOBt (16.96 mg, 125.53 umol) and i-Pr₂NEt (40.56 mg, 313.83 umol). The mixture was stirred at 25° C. for 12 hrs. The reaction mixture was concentrated and purified by preparative HPLC using Method X to afford the title compound (5.26 mg, 10%) as a yellow solid. ¹H NMR (400 MHz, DMSO-d₆) δ 13.46 (br s, 1H), 10.70 (s, 1H), 9.10 (s, 1H), 8.65 (d, J=1.2 Hz, 1H), 8.59 (s, 1H), 8.51 (s, 1H), 7.90 (dd, J=1.7, 9.0 Hz, 1H), 7.69 (s, 1H), 7.61 (d, J=8.9 Hz, 1H), 4.25 (s, 3H), 2.96 (s, 3H). MS-ESI (m/z) calc'd for C₁₉H₁₆N₇O₂ [M+H]⁺: 374.1. Found 374.1.

### Example 170: N-(3-(Furan-3-yl)-1H-indazol-5-yl)thiazolo[4,5-c]pyridine-2-carboxamide

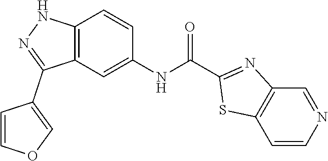 [SMILES: O=C(Nc1ccc2[nH]nc(-c3ccoc3)c2c1)c1nc2cnccc2s1]

To a solution of [1,3]thiazolo[4,5-c]pyridine-2-carboxylic acid (30.07 mg, 0.170 mmol) and 3-(furan-3-yl)-1H-indazol-5-amine (0.05 mL, 0.170 mmol) in DMF (1.5 mL), Et₃N (0.03 mL, 0.250 mmol) and HATU (69.81 mg, 0.180 mmol) were added. The mixture was stirred at r.t. for 18 hrs. The reaction mixture was diluted with water and extracted with EtOAc. The organic phase was separated, dried over Na₂SO₄, filtered and concentrated under reduced pressure. The residue was purified by reversed phase column chromatography using a 2-100% CH₃CN/H₂O (0.1% formic acid) gradient eluent. The product containing fractions were collected and concentrated under reduced pressure. The residue was taken up in MeOH, filtered and concentrated to afford the title compound (17.8 mg, 29%) as a yellow solid. ¹H NMR (400 MHz, DMSO-d₆) δ 13.15 (s, 1H), 11.29 (s, 1H), 9.51 (s, 1H), 8.69 (d, J=5.5 Hz, 1H), 8.56 (d, J=1.9 Hz, 1H), 8.36 (d, J=5.5, 1.0 Hz, 1H), 8.28 (s, 1H), 7.97 (dd, J=8.9, 1.8 Hz, 1H), 7.87 (t, J=1.7 Hz, 1H), 7.60 (d, J=9.0 Hz, 1H), 7.03 (d, J=1.9 Hz, 1H). MS-ESI (m/z) calc'd for C₁₈H₁₂N₅O₂S [M+H]⁺: 362.1. Found 362.1.

### Example 171: N-(3-(Furan-3-yl)-1H-indazol-5-yl)thiazolo[5,4-c]pyridine-2-carboxamide

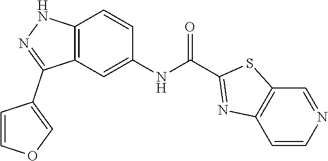 [SMILES: O=C(Nc1ccc2[nH]nc(-c3ccoc3)c2c1)c1nc2ccncc2s1]

To a solution of 3-(furan-3-yl)-1H-indazol-5-amine (49.75 mg, 0.250 mmol) and [1,3]thiazolo[5,4-c]pyridine-2-carboxylic acid (45.0 mg, 0.250 mmol) in DMF (1.5 mL) was added Et₃N (0.05 mL, 0.370 mmol) and HATU (104.46 mg, 0.270 mmol). The mixture was then stirred at r.t. for 18 hrs. The reaction mixture was diluted with water and then extracted with EtOAc. The organic phase was separated, dried over Na₂SO₄, filtered and concentrated under reduced pressure. The residue was purified by reversed phase column chromatography using a 2-100% CH₃CN/H₂O (0.1% formic acid) gradient eluent to afford 35 mg of a product of insufficient purity. The material was then further purified by reversed phase column chromatography using a 2-100% CH₃CN/H₂O (0.1% formic acid) gradient eluent. The product containing fractions were collected and concentrated to give a residue which was taken up in MeOH and filtered. The solid obtained was dried under vacuum to afford the title compound (4.1 mg, 5%) as a yellow solid. ¹H NMR (400 MHz, DMSO-d₆) δ 13.15 (s, 1H), 11.28 (s, 1H), 9.58 (d, J=1.0 Hz, 1H), 8.78 (d, J=5.6 Hz, 1H), 8.56 (s, 1H), 8.29 (d, J=1.4 Hz, 1H), 8.20 (dd, J=5.7, 1.0 Hz, 1H), 7.97 (dd, J=8.9, 1.9 Hz, 1H), 7.87 (t, J=1.7 Hz, 1H), 7.60 (d, J=8.9 Hz, 1H), 7.03 (d, J=1.9 Hz, 1H). MS-ESI (m/z) calc'd for C₁₈H₁₂N₅O₂S [M+H]⁺: 362.1. Found 362.1.

### Example 172: N-(3-(Furan-3-yl)-1H-indazol-5-yl)-8,8-dimethyl-5,6-dihydro-[1,2,4]triazolo[4,3-a]pyrazine-7(8H)-carboxamide

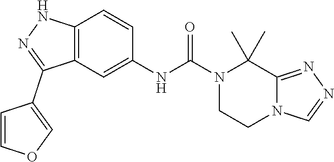 [SMILES: CC1(C)c2nncn2CCN1C(=O)Nc1ccc2[nH]nc(-c3ccoc3)c2c1]

### Step 1: tert-Butyl 2,2-dimethyl-5-oxopiperazine-1-carboxylate

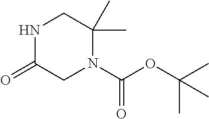 [SMILES: CC(C)(C)OC(=O)N1CC(=O)NCC1(C)C]

To a solution of 3,3-dimethylpiperazin-2-one (500.0 mg, 3.9 mmol) in dry THF (14 mL) was added di-tert-butyl dicarbonate (851.39 mg, 3.9 mmol) at r.t. After stirring for 18 hrs the residue was diluted in EtOAc and washed with saturated aqueous NH₄Cl. The organic phase was dried over Na₂SO₄ and concentrated. The residue was purified by column chromatography on silica gel using a 0-10% gradient of MeOH in DCM to afford the title compound (650 mg, 73%) as a white solid. ¹H NMR (400 MHz, DMSO-d₆) δ 8.02 (br. s., 1H), 3.55-3.44 (m, 2H), 3.35 (m, 1H), 3.10-3.22 (m, 2H), 1.53 (s, 6H), 1.43 (s, 9H). MS-ESI (m/z) calc'd for C₁₁H₂₁N₂O₃ [M+H]⁺: 229.2. Found 229.1.

### Step 2: tert-Butyl 2,2-dimethyl-5-thioxopiperazine-1-carboxylate

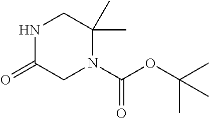 [SMILES: CC(C)(C)OC(=O)N1CC(=O)NCC1(C)C]

To a suspension of 2,4-bis(4-methoxyphenyl)-2,4-dithioxo-1,3,2,4-dithiadiphosphetane (235.64 mg, 0.580 mmol) in toluene (2.2 mL) was added tert-butyl 2,2-dimethyl-3-oxopiperazine-1-carboxylate (133.0 mg, 0.580 mmol). The mixture was heated at 80° C. for 1 hr and then concentrated in vacuo. The residue was dissolved in DCM and purified by column chromatography on silica gel using a 0-50% gradient of EtOAc in cyclohexane to afford the title compound (25.4 mg, 18%) as a white solid. ¹H NMR (400 MHz, CDCl₃) δ 8.43 (br. s., 1H), 3.81-3.63 (m, 2H), 3.53-3.33 (m, 2H), 1.95 (s, 6H), 1.52 (s, 9H). MS-ESI (m/z) calc'd for C₁₁H₂₁N₂O₂S [M+H]⁺: 245.1. Found 245.1.

### Step 3: tert-Butyl 2,2-dimethyl-5-(methylthio)-3,6-dihydropyrazine-1(2H)-carboxylate

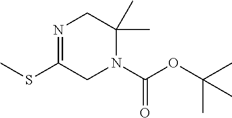 [SMILES: CSC1=NCC(C)(C)N(C(=O)OC(C)(C)C)C1]

To a solution of tert-butyl 2,2-dimethyl-3-sulfanylidenepiperazine-1-carboxylate (25.4 mg, 0.100 mmol) in MeCN (0.500 mL) was added iodomethane (0.01 mL, 0.160 mmol) and the mixture was stirred at r.t. for 2 hrs. The solvent was evaporated to afford the title compound (26.8 mg, 99%) as a pale yellow solid which was used without further purification. MS-ESI (m/z) calc'd for C₁₂H₂₃N₂O₂S [M+H]⁺: 259.1. Found 259.1.

### Step 4: tert-Butyl 8,8-dimethyl-5,6-dihydro-[1,2,4]triazolo[4,3-a]pyrazine-7(8H)-carboxylate

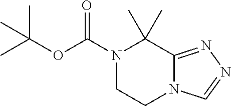 [SMILES: CC(C)(C)OC(=O)N1CCn2cnnc2C1(C)C]

To a solution of tert-butyl 6,6-dimethyl-5-methylsulfanyl-2,3-dihydropyrazine-1-carboxylate (146.0 mg, 0.570 mmol) in EtOH (5.452 mL) was added formylhydrazine (339.37 mg, 5.65 mmol). The resulting mixture was stirred at reflux for 18 hrs. The mixture was concentrated under reduced pressure, diluted with EtOAc and washed with water. The organic phase was separated, dried over Na₂SO₄, filtered and concentrated. The residue was purified by strong cation exchange (first eluting with MeOH and then with a 2 M ammonia solution in MeOH) to afford the title compound (130 mg, 91%) as a white solid. ¹H NMR (400 MHz, DMSO-d₆) δ ppm 8.43 (s, 1H), 4.08 (dd, J=5.72, 4.40 Hz, 2H), 3.62-3.81 (m, 2H), 1.78 (s, 6H), 1.47 (s, 9H). MS-ESI (m/z) calc'd for C₁₂H₂₁N₄O₂ [M+H]⁺: 253.2. Found 253.1.

### Step 5: 8,8-Dimethyl-5,6,7,8-tetrahydro-[1,2,4]triazolo[4,3-a]pyrazine

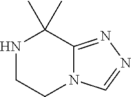

tert-Butyl 8,8-dimethyl-5,6-dihydro-[1,2,4]triazolo[4,3-a]pyrazine-7-carboxylate (130.0 mg, 0.520 mmol) was dissolved in DCM (2 mL). Trifluoroacetic acid (0.16 mL, 2.06 mmol) was added and the resulting solution was stirred at r.t. for 18 hrs. The mixture was concentrated under reduced pressure and the material was purified by strong cation exchange to afford the title compound (70 mg, 89%). ¹H NMR (400 MHz, DMSO-d₆) δ 8.31 (s, 1H), 3.92 (t, J=5.50 Hz, 2H), 3.04 (d, J=3.96 Hz, 2H), 2.57 (br. s., 1H), 1.40 (s, 6H). MS-ESI (m/z) calc'd for C₇H₁₃N₄ [M+H]⁺: 153.1. Found 152.9.

### Step 6: N-(3-(Furan-3-yl)-1H-indazol-5-yl)-8,8-dimethyl-5,6-dihydro-[1,2,4]triazolo[4,3-a]pyrazine-7(8H)-carboxamide

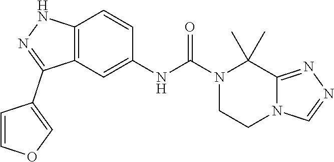 [SMILES: CC1(C)c2nncn2CCN1C(=O)Nc1ccc2[nH]nc(-c3ccoc3)c2c1]

To a solution of 1,1′-carbonyldiimidazole (53.27 mg, 0.330 mmol) in DCM (3.3 mL) was added 3-(furan-3-yl)-1H-indazol-5-amine (0.09 mL, 0.330 mmol) and the suspension was stirred at r.t. for 2 hrs. A solution of 8,8-dimethyl-6,7-dihydro-5H-[1,2,4]triazolo[4,3-a]pyrazine (50.0 mg, 0.330 mmol) in DMF (3.285 mL) was then added and the mixture was stirred at r.t. for 18 hrs and then at 40° C. for 4 hrs. The reaction mixture was concentrated and then partitioned between saturated aqueous NH₄Cl and EtOAc. The phases were separated and the aqueous layer was extracted with EtOAc. The organic phase were concentrated and purified by semi-preparative HPLC using Method Y to afford the title compound (8.2 mg, 7%) as a colorless oil. ¹H NMR (400 MHz, Methanol-d₄) δ 8.45 (s, 1H), 8.20-8.15 (m, 1H), 7.93 (dd, J=1.9, 0.8 Hz, 1H), 7.67 (t, J=1.7 Hz, 1H), 7.51 (dd, J=8.9, 0.8 Hz, 1H), 7.44 (dd, J=8.9, 1.9 Hz, 1H), 7.02 (dd, J=1.9, 0.8 Hz, 1H), 4.33 (dd, J=5.7, 4.4 Hz, 2H), 3.96 (dd, J=5.7, 4.4 Hz, 2H), 1.99 (s, 6H). MS-ESI (m/z) calc'd for C₁₉H₂₀N₇O₂ [M+H]⁺: 378.2. Found 378.2.

### Example 173a: rel-(S)-N-(3-(Furan-3-yl)-1H-indazol-5-yl)-5,6,7,8-tetrahydroimidazo[1,5-a]pyridine-6-carboxamide and Example 173b: rel-(R)-N-(3-(Furan-3-yl)-1H-indazol-5-yl)-5,6,7,8-tetrahydroimidazo[1,5-a]pyridine-6-carboxamide

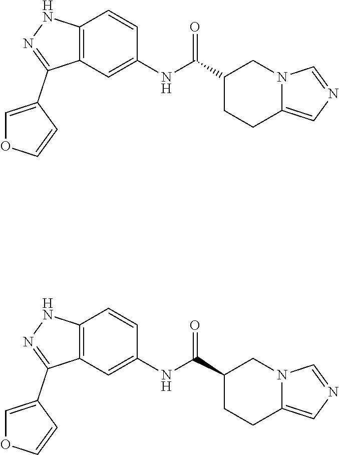

### Step 1: N-(3-(Furan-3-yl)-1H-indazol-5-yl)-5,6,7,8-tetrahydroimidazo[, 5-a]pyridine-6-carboxamide

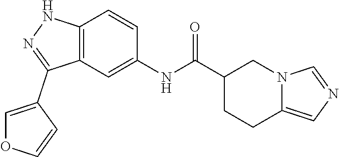 [SMILES: O=C(Nc1ccc2[nH]nc(-c3ccoc3)c2c1)C1CCc2cncn2C1]

To a solution of methyl 5,6,7,8-tetrahydroimidazo[1,5-a]pyridine-6-carboxylate (100 mg, 554.93 umol) in toluene (4 mL) was added Al(CH₃)₃ (2 M, 1.39 mL) and 3-(furan-3-yl)-1H-indazol-5-amine (110.55 mg, 554.93 umol). The mixture was stirred at 100° C. for 2 hrs. The reaction mixture was concentrated under reduced pressure to remove solvent. The residue was purified by preparative HPLC using Method Z to afford the title compound (7.9 mg, 3%) as a yellow solid. ¹H NMR (400 MHz, DMSO-d₆) δ 13.1 (s, 1H) 10.36 (br s, 1H) 9.05 (s, 1H) 8.27 (s, 1H) 8.17 (s, 1H) 7.83 (s, 1H) 7.52 (s, 2H) 7.44 (s, 1H) 6.96 (s, 1H) 4.33-4.53 (m, 2H) 3.19-3.26 (m, 1H) 2.79-2.98 (m, 2H) 2.22-2.32 (m, 1H) 2.03-2.13 (m, 1H). MS-ESI (m/z) calc'd for C₁₉H₁₈N₅O₂ [M+H]⁺: 348.1. Found 348.2.

### Step 2: rel-(S)-N-(3-(Furan-3-yl)-H-indazol-5-yl)-5,6,7,8-tetrahydroimidazo[1,5-a]pyridine-6-carboxamide and rel-(R)-N-(3-(Furan-3-yl)-1H-indazol-5-yl)-5,6,7,8-tetrahydroimidazo[1,5-a]pyridine-6-carboxamide

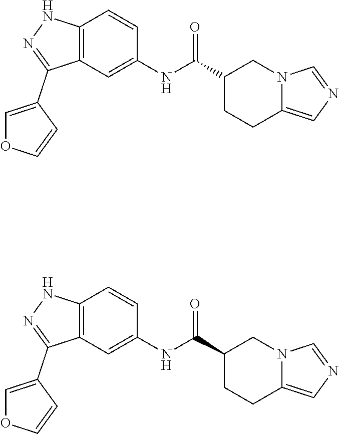

Racemic N-(3-(Furan-3-yl)-1H-indazol-5-yl)-5,6,7,8-tetrahydroimidazo[1,5-a]pyridine-6-carboxamide was subjected to chiral separation using Method AA to afford the first eluting enantiomer, rel-(S)-N-(3-(furan-3-yl)-1H-indazol-5-yl)-5,6,7,8-tetrahydroimidazo[1,5-a]pyridine-6-carboxamide (Example 173a, 1.4 mg, 40%). ¹H NMR (400 MHz, METHANOL-d₄) δ ppm 8.29-8.34 (m, 1H), 8.13-8.19 (m, 1H), 7.64-7.70 (m, 1H), 7.57-7.62 (m, 1H), 7.49-7.56 (m, 2H), 7.00-7.05 (m, 1H), 6.70-6.77 (m, 1H), 4.40-4.48 (m, 1H), 4.18-4.27 (m, 1H), 3.02-3.16 (m, 2H), 2.80-2.91 (m, 1H), 2.28-2.38 (m, 1H), 2.02-2.16 (m, 1H). MS-ESI (m/z) calc'd for C₁₉H₁₈N₅O₂ [M+H]⁺: 348.1. Found 348.2.

A second fraction was isolated to afford the second eluting enantiomer, rel-(R)-N-(3-(furan-3-yl)-1H-indazol-5-yl)-5,6,7,8-tetrahydroimidazo[1,5-a]pyridine-6-carboxamide (Example 173b, 1.4 mg, 40%). ¹H NMR (400 MHz, METHANOL-d₄) δ 8.34-8.29 (m, 1H), 8.20-8.14 (m, 1H), 7.70-7.65 (m, 1H), 7.62-7.57 (m, 1H), 7.56-7.46 (m, 2H), 7.05-7.00 (m, 1H), 6.77-6.71 (m, 1H), 4.47-4.39 (m, 1H), 4.26-4.17 (m, 1H), 3.14-3.03 (m, 2H), 2.91-2.78 (m, 1H), 2.38-2.29 (m, 1H), 2.16-2.03 (m, 1H). MS-ESI (m/z) calc'd for C₁₉H₁₈N₅O₂ [M+H]⁺: 348.1. Found 348.2. Absolute stereochemistry arbitrarily assigned.

### Example A. LRRK2 Kinase Activity

LRRK2 kinase activity was measured using a LanthaScreen™ Kinase Activity Assay from ThermoFisher Scientific. Recombinant wild type or G2019S-LRRK2 protein (Life Technologies, PR8604B or PV4881, respectively), was incubated with a fluorescein-labeled peptide substrate called LRRKtide that is based upon ezrin/radixin/moesin (ERM) (Life Technologies, PV4901) in the presence of ATP and serially diluted compound. After an incubation period of 1 hr, the phosphotransferase activity was stopped and a terbium-labelled anti-pERM antibody (Life Technologies, PV4899) was added to detect the phosphorylation of LRRKtide by measuring the time resolved-Forster resonant energy transfer (TR-FRET) signal from the terbium label on the antibody to the fluorescein tag on LRRKtide, expressed as the 520 nm/495 nm emission ratio. Compound-dependent inhibition of the TR-FRET signal was used to generate a concentration-response curve for IC₅₀ determination.

The assay was carried out under the following protocol conditions: 1 mM compound in DMSO was serially diluted 1:3, 11 points in DMSO with a Biomek FX and 0.1 μL of the diluted compound was subsequently stamped into the assay plate (384-well format Lumitrac 200, Greiner, 781075) with an Echo Labcyte such that the final compound concentration in the assay was 10 μM to 169 μM. Subsequently, 5 μL of 2× kinase solution (2.9 nM final concentration) was added to the assay plate in assay buffer composed of 50 mM Tris pH 8.5 (Sigma, T6791), 5 mM MgCl₂ (Fluka, 63020), 1 mM EGTA (Sigma, E3889), 0.01% BRIJ-35 (Sigma, P1254) and 2 mM DTT. The reaction was started by addition of 2×ATP/LRRKtide solution in assay buffer such that the final concentration was 400 nM LRRKtide and 25 μM ATP. After 60 min incubation at room temperature, the reaction was stopped by addition of μL of 2× stop solution containing a final concentration of 2 nM anti-pERM antibody and 10 mM EDTA. After a 30 min incubation at RT, the TR-FRET signal was measured on a Wallac 2104 EnVision® multilabel reader at an excitation wavelength of 340 nm and reading emission at 520 nm and 495 nm. The ratio of the 520 nm and 495 nm emission was used to analyze the data.

The Results of the LRRK2 kinase activity assay are shown in Table 1. Data is displayed as follows: + is IC₅₀≤100 nM; ++ is 100 nM<IC₅₀≤1,000 nM; and +++ is 1,000 nM<IC₅₀≤10,000 nM.

TABLE 1
LRRK2 Kinase Activity Assay

| | LRRK2 WT IC₅₀ | LRRK2 G2019S |
|---|---|---|
| Example No. | (nM) | IC₅₀ (nM) |
| 1 | ++ | + |
| 2 | + | + |
| 3 | +++ | +++ |
| 5 | +++ | +++ |
| 8 | +++ | +++ |
| 9 | +++ | +++ |
| 10 | >10,000 | +++ |
| 11 | +++ | ++ |
| 12 | ++ | + |
| 13 | ++ | ++ |
| 14 | >10,000 | +++ |
| 15 | +++ | + |
| 18 | ++ | ++ |
| 19 | ++ | + |
| 20 | ++ | + |
| 21 | + | + |
| 22 | +++ | ++ |
| 23 | + | + |
| 24 | + | + |
| 28 | + | + |
| 29 | ++ | + |
| 30 | ++ | + |
| 32 | + | + |
| 34 | ++ | + |
| 35 | + | + |
| 36 | >10,000 | +++ |
| 37 | + | + |
| 38 | +++ | +++ |
| 40 | +++ | ++ |
| 43 | + | + |
| 44 | ++ | + |
| 45 | ++ | + |
| 46 | ++ | + |
| 47 | +++ | ++ |
| 48 | + | + |
| 49 | +++ | + |
| 50 | ++ | + |
| 51 | ++ | + |
| 52 | + | + |
| 55 | +++ | + |
| 56 | ++ | + |
| 57 | + | + |
| 58 | +++ | ++ |
| 59 | + | + |
| 61 | ++ | + |
| 62 | ++ | + |
| 63 | + | + |
| 64 | ++ | + |
| 65 | +++ | ++ |
| 69 | + | + |
| 70 | ++ | + |
| 71 | ++ | + |
| 72 | +++ | ++ |
| 73 | + | + |
| 74 | >10,000 | ++ |
| 75 | +++ | ++ |
| 76 | + | + |
| 77 | +++ | +++ |
| 78 | ++ | + |
| 79 | +++ | ++ |
| 80 | + | + |
| 81 | ++ | + |
| 82 | + | + |
| 83 | + | + |
| 84 | + | + |
| 85 | +++ | + |
| 86 | + | + |
| 87 | + | + |
| 88 | + | + |
| 89 | ++ | + |
| 90 | +++ | ++ |
| 91 | +++ | ++ |
| 92 | ++ | + |
| 93 | >10,000 | +++ |
| 94 | + | + |
| 95 | + | + |
| 96 | ++ | + |
| 97 | + | + |
| 98 | + | + |
| 99 | ++ | + |
| 100 | + | + |
| 101 | ++ | + |
| 102 | ++ | + |
| 103 | ++ | + |
| 104 | +++ | ++ |
| 105 | ++ | + |
| 106 | +++ | + |
| 107 | +++ | ++ |
| 108 | +++ | ++ |
| 109 | ++ | + |
| 110 | ++ | + |
| 111 | + | + |
| 112 | ++ | + |
| 113 | + | + |
| 114 | ++ | + |
| 115 | ++ | + |
| 116 | ++ | + |
| 117 | ++ | + |
| 118 | ++ | ++ |
| 119 | ++ | + |
| 120 | ++ | + |
| 121 | + | + |
| 122 | + | + |
| 123 | ++ | + |
| 124 | ++ | ++ |
| 125 | ++ | + |
| 126 | ++ | + |
| 127 | ++ | + |
| 128 | ++ | ++ |
| 129 | ++ | + |
| 130 | ++ | ++ |
| 131 | + | + |
| 132 | ++ | ++ |
| 133 | + | + |
| 134 | ++ | + |
| 135 | + | + |
| 136 | ++ | + |
| 137 | + | + |
| 138 | ++ | + |
| 139 | +++ | ++ |
| 140 | +++ | ++ |
| 141 | ++ | + |
| 142 | >10,000 | +++ |
| 143 | ++ | + |
| 144 | + | + |
| 145 | ++ | + |
| 146 | + | + |
| 147 | + | + |
| 148 | ++ | + |
| 149 | + | + |
| 150 | + | + |
| 151 | + | + |
| 152 | + | + |
| 153 | + | + |
| 154 | + | + |
| 155 | + | + |
| 156 | + | + |
| 157 | + | + |
| 158 | + | + |
| 159 | + | + |
| 160 | + | + |
| 161a | + | + |
| 161b | ++ | + |
| 161c | ++ | + |
| 161d | ++ | + |
| 162a | + | + |
| 162b | ++ | + |
| 163 | + | + |
| 164 | ++ | ++ |
| 165 | ++ | + |
| 166 | ++ | + |
| 167 | ++ | + |
| 168 | + | + |
| 169 | ++ | + |
| 170 | ++ | + |
| 171 | + | + |
| 172 | ++ | ++ |
| 173a | ++ | + |
| 173b | +++ | +++ |

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A compound of Formula I: 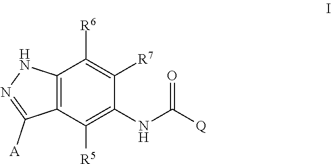
or a pharmaceutically acceptable salt thereof, wherein:
A is C₁₋₆ alkyl, C₁₋₆ haloalkyl, Cy¹, halo, CN, OR^{a}, or NR^{x}R^{y}, wherein said C₁₋₆ alkyl is optionally substituted with Cy¹;
Q is selected from the following groups: 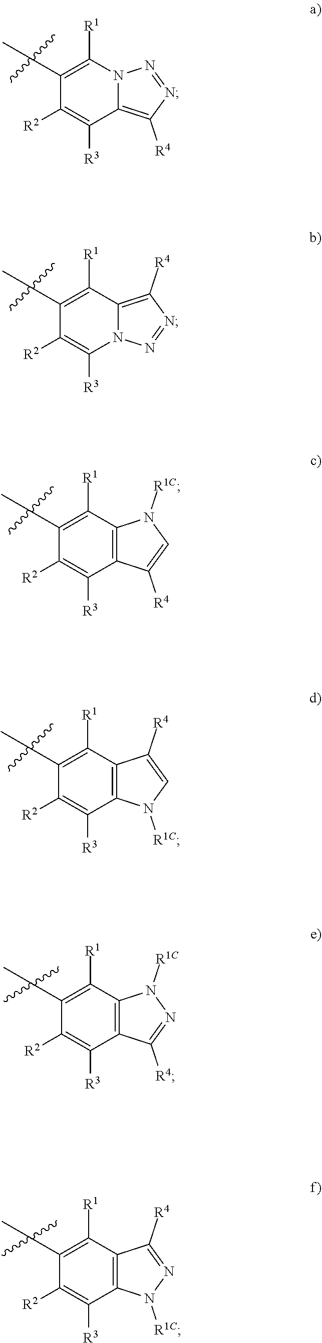 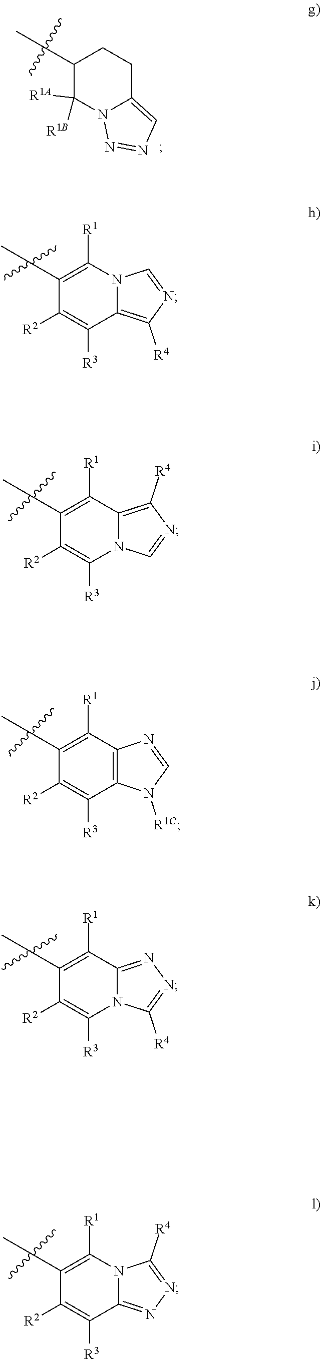 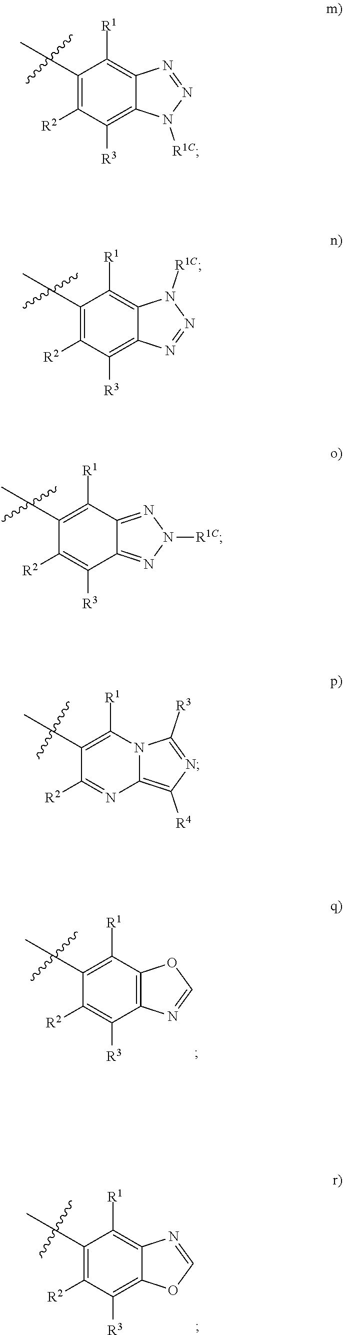 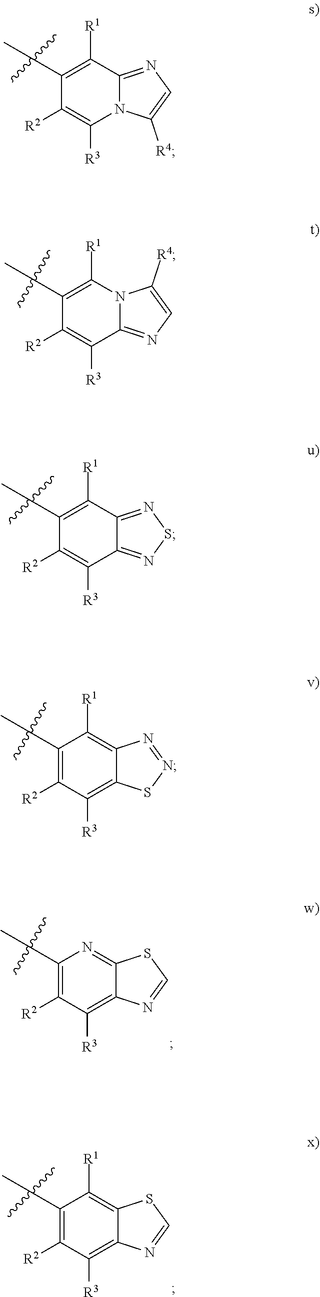 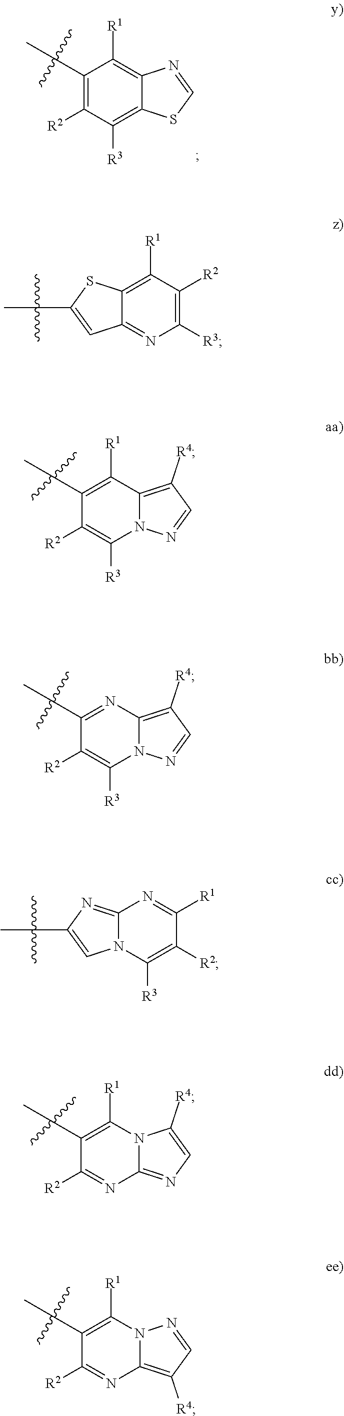 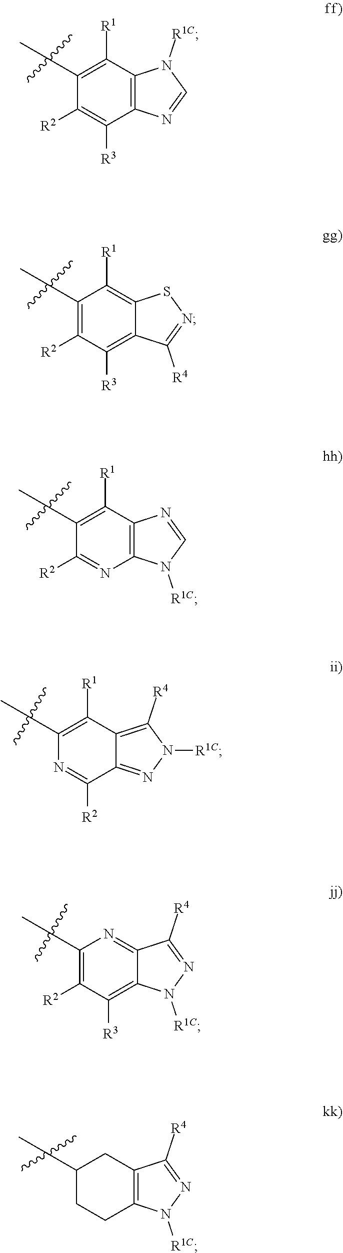 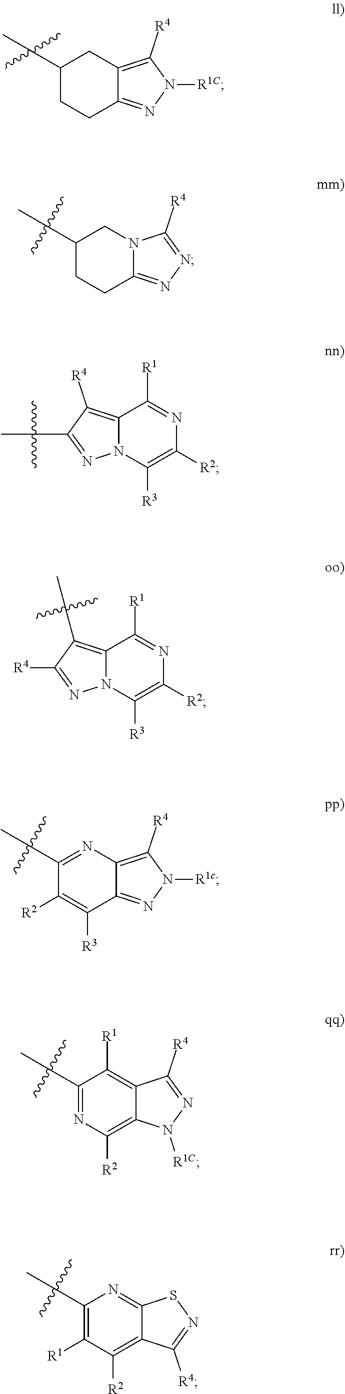 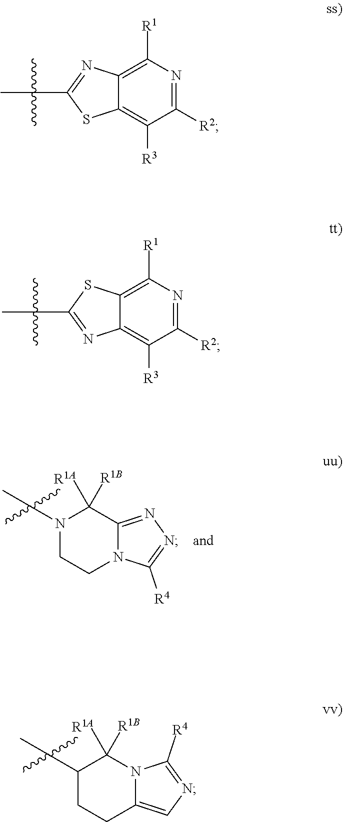
Cy¹ is selected from C₆₋₁₀ aryl, C₃₋₁₀ cycloalkyl, 5-14 membered heteroaryl, and 4-14 membered heterocycloalkyl, each optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR)R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};
R¹, R², R³, R⁴, R⁵, R⁶, and R⁷ are each independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R¹, R², R³, R⁴, R⁵, R⁶, and R⁷ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
R^{1A} and R^{1B} are each independently selected from H, halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR²S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{1A} and R^{1B} are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
or R^{1A} and R^{1B} together form a C₃₋₇ cycloalkyl or 4-10 membered heterocycloalkyl ring, each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
R^{1C} is selected from H and C₁₋₆ alkyl;
each R^{a}, R^{b}, R^{c}, R^{d}, R^{a1}, R^{b1}, R^{c1}, R^{d1}, R^{a2}, R^{b2}, R^{c2}, R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a}, R^{b}, R^{c}, R^{d}, R^{a1}, R^{b1}, R^{c1}, R^{d1}, R^{a2}, R^{b2}, R^{c2}, and R^{d2} is optionally substituted with 1, 2, 3, 4, or 5 substituents independently selected from halo, C₁₋₄ alkyl, C₁₋₄haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
R^{x} is selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of RX is optionally substituted with 1, 2, 3, 4, or 5 substituents independently selected from halo, C₁₋₄ alkyl, C₁₋₄haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR³R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
R^{y} is selected from C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R is optionally substituted with 1, 2, 3, 4, or 5 substituents independently selected from halo, C₁₋₄ alkyl, C₁₋₄haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
each R^{a3}, R^{b3}, R³, and R^{d3} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, 4-7 membered heterocycloalkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, and 4-7 membered heterocycloalkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy; and
each R^{e}, R^{e1}, R^{e2}, and R^{e3} is independently selected from H, C₁₋₄ alkyl, and CN.

2. A compound of Formula I: 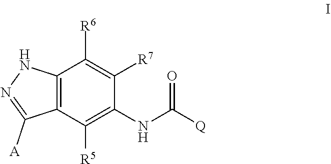
or a pharmaceutically acceptable salt thereof, wherein:
A is C₁₋₆ alkyl, C₁₋₆ haloalkyl, Cy¹, halo, CN, OR^{a}, or NR^{x}R^{y}, wherein said C₁₋₆ alkyl is optionally substituted with Cy¹;
Q is selected from the following groups: 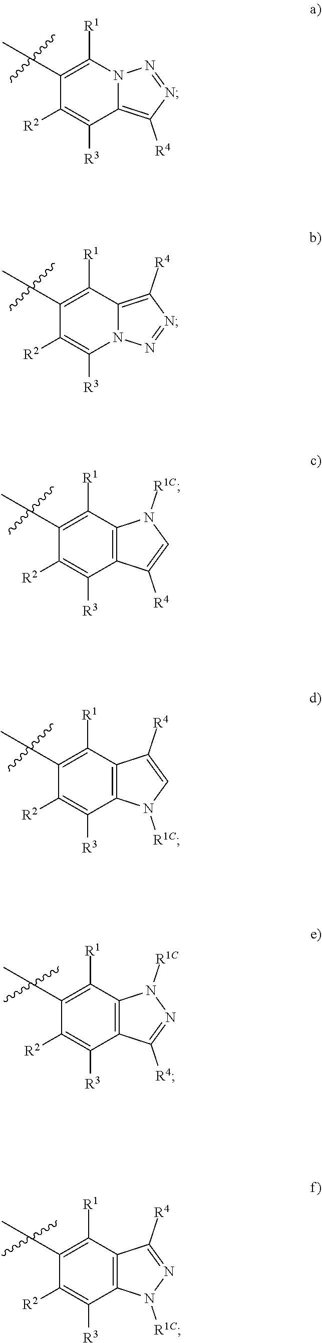 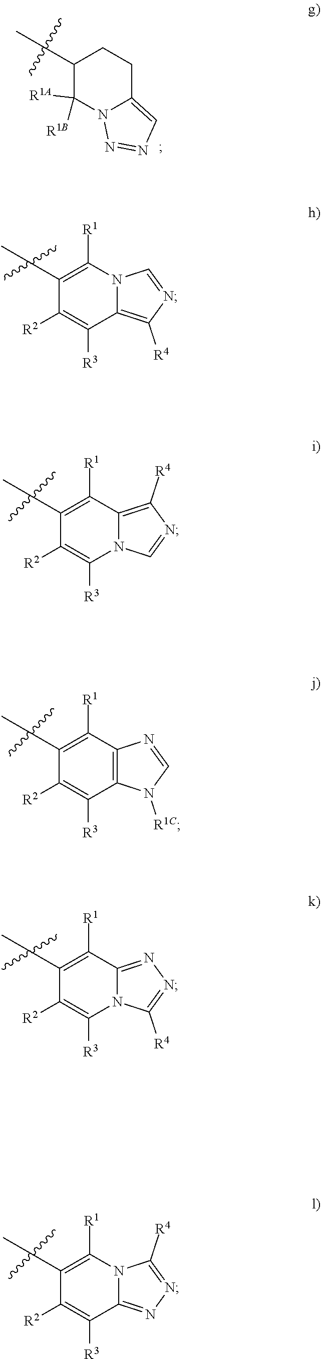 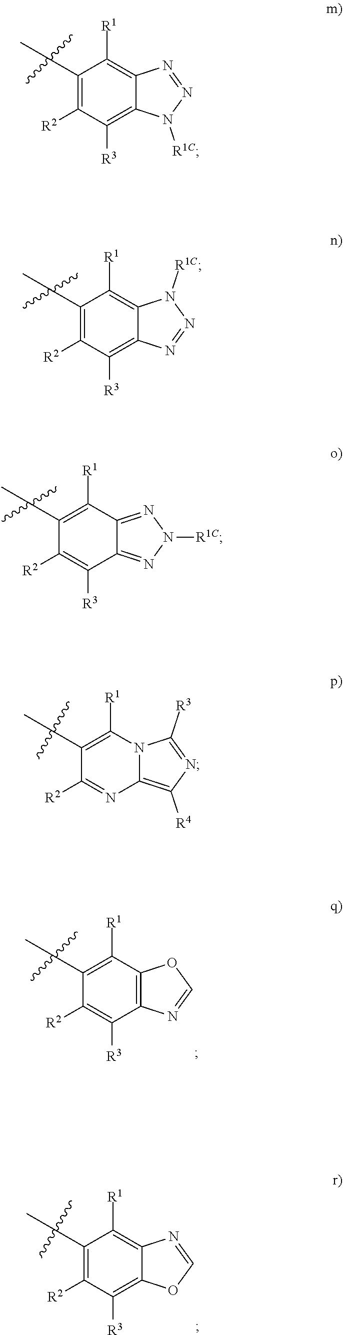 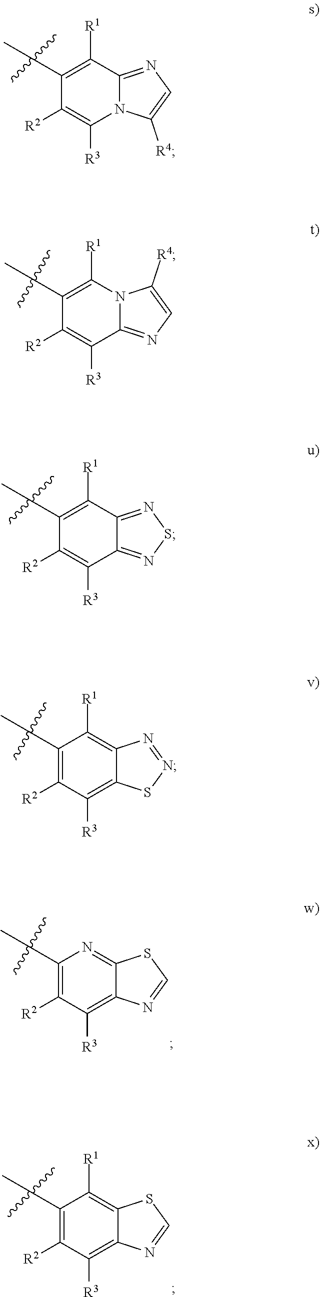 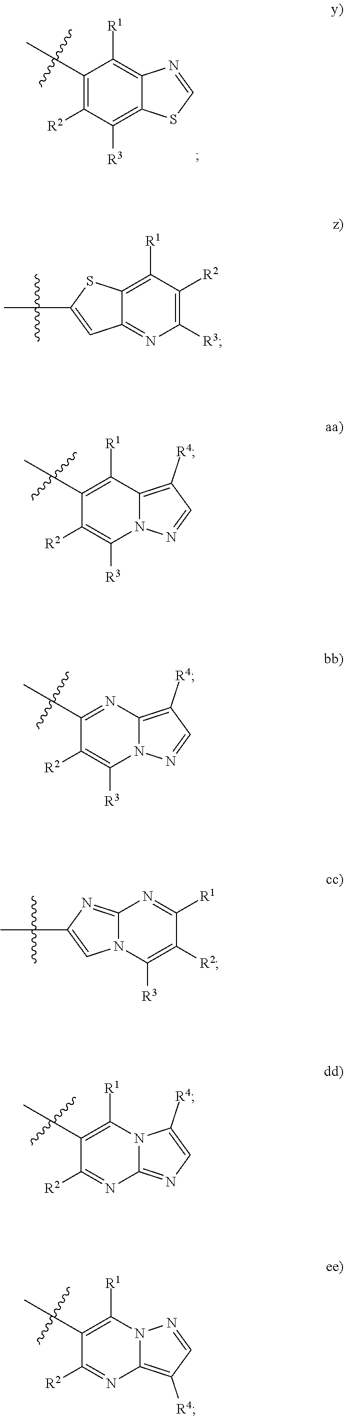 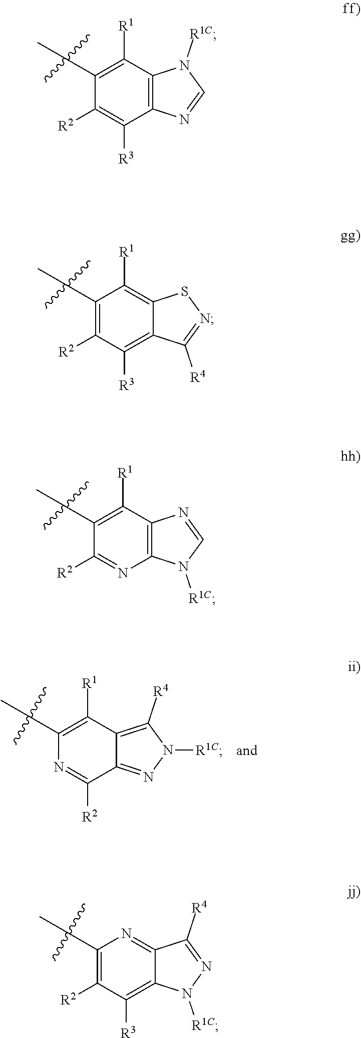
Cy¹ is selected from C₆₋₁₀ aryl, C₃₋₁₀ cycloalkyl, 5-14 membered heteroaryl, and 4-14 membered heterocycloalkyl, each optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR)R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};
R¹, R², R³, R⁴, R⁵, R⁶, and R⁷ are each independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{d1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R¹, R², R³, R⁴, R⁵, R⁶, and R⁷ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
R^{1A} and R^{1B} are each independently selected from H, halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR²S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{1A} and R^{1B} are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
or R^{1A} and R^{1B} together form a C₃₋₇ cycloalkyl or 4-10 membered heterocycloalkyl ring, each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
R^{1C} is selected from H and C₁₋₆ alkyl;
each R^{a}, R^{b}, R^{c}, R^{d}, R^{a1}, R^{b1}, R^{c1}, R^{d1}, R^{a2}, R^{b2}, R^{c2}, R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a}, R^{b}, R^{c}, R^{d}, R^{a1}, R^{b1}, R^{c1}, R^{d1}, R^{a2}, R^{b2}, R^{c2}, and R^{d2} is optionally substituted with 1, 2, 3, 4, or 5 substituents independently selected from halo, C₁₋₄ alkyl, C₁₋₄haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
R^{x} is selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of RX is optionally substituted with 1, 2, 3, 4, or 5 substituents independently selected from halo, C₁₋₄ alkyl, C₁₋₄haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR³R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
R^{y} is selected from C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R is optionally substituted with 1, 2, 3, 4, or 5 substituents independently selected from halo, C₁₋₄ alkyl, C₁₋₄haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
each R^{a3}, R^{b3}, R³, and R^{d3} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, 4-7 membered heterocycloalkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, and 4-7 membered heterocycloalkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy; and
each R^{e}, R^{e1}, R^{e2}, and R^{e3} is independently selected from H, C₁₋₄ alkyl, and CN.

3. A compound of Formula I: 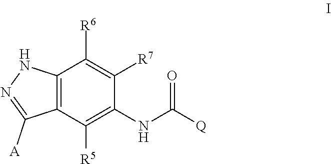
or a pharmaceutically acceptable salt thereof, wherein:
A is C₁₋₆ alkyl, Cy¹, or halo;
Q is selected from the following groups: 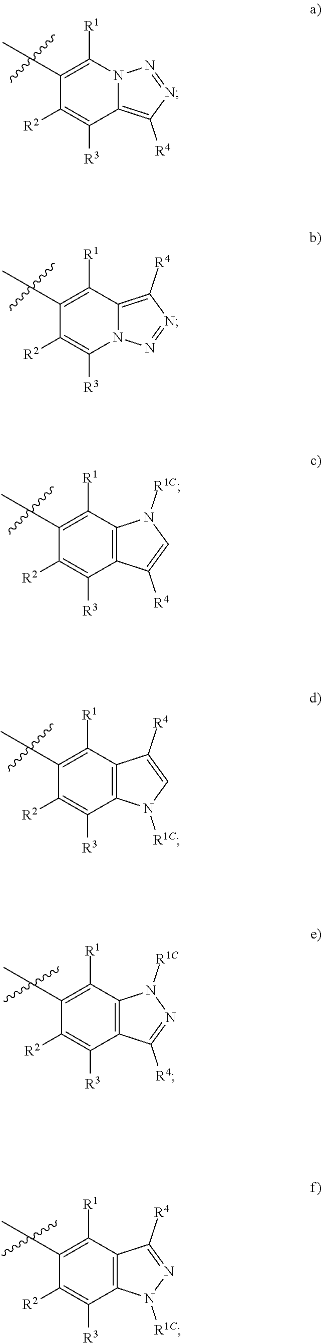 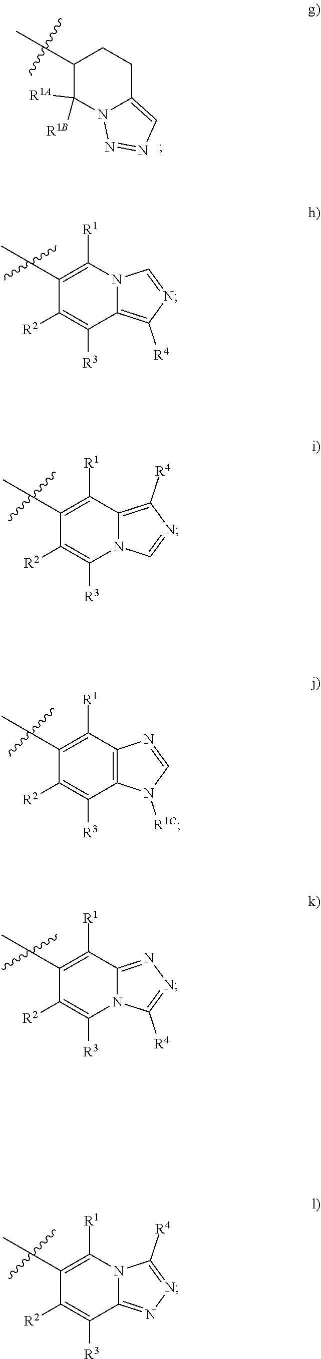 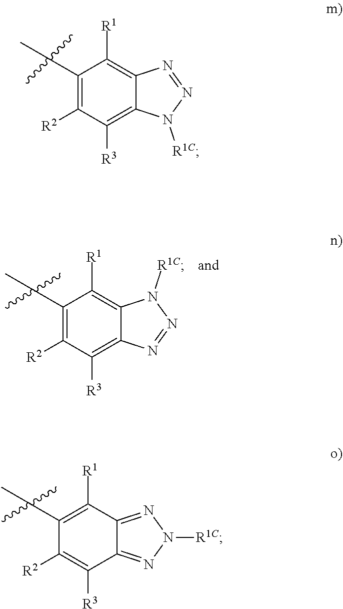
Cy¹ is selected from C₆₋₁₀ aryl, C₃₋₁₀ cycloalkyl, 5-14 membered heteroaryl, and 4-14 membered heterocycloalkyl, each optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR)R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};
R¹, R², R³, R⁴, R⁵, R⁶, and R⁷ are each independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R¹, R², R³, R⁴, R⁵, R⁶, and R⁷ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
R^{1A} and R^{1B} are each independently selected from H, halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{1A} and R^{1B} are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
or R^{1A} and R^{1B} together form a C₃₋₇ cycloalkyl or 4-10 membered heterocycloalkyl ring, each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a2}, SR², C(O)R^{b2}, C(O)NR^{c2}R^{d2}, C(O)OR^{a2}, OC(O)R^{b2}, OC(O)NR^{c2}R^{d2}, NR^{e2}R^{d2} NR^{e2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
R^{1C} is selected from H and C₁₋₆ alkyl;
each R^{a}, R^{b}, R^{c}, R^{d}, R^{a1}, R^{b1}, R^{c1}, R^{d1}, R^{a2}, R^{b2}, R^{c2}, R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a}, R^{b}, R^{c}, R^{d}, R^{a1}, R^{b1}, R^{c1}, R^{d1}, R^{a2}, R^{b2}, R^{c2}, and R^{d2} is optionally substituted with 1, 2, 3, 4, or 5 substituents independently selected from halo, C₁₋₄ alkyl, C₁₋₄haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR³R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
each R^{a3}, R^{b3}, R^{c3}, and R^{d3} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, 4-7 membered heterocycloalkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, and 4-7 membered heterocycloalkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy; and
each R^{e}, R^{e1}, R^{e2}, and R^{e3} is independently selected from H, C₁₋₄ alkyl, and CN.

4. The compound of claim 1, or a pharmaceutically acceptable salt thereof, wherein A is C₁₋₆ alkyl.

5. The compound of claim 1, or a pharmaceutically acceptable salt thereof, wherein A is methyl or propyl.

6. The compound of claim 1, or a pharmaceutically acceptable salt thereof, wherein A is methyl.

7. The compound of claim 1, or a pharmaceutically acceptable salt thereof, wherein A is halo.

8. The compound of claim 1, or a pharmaceutically acceptable salt thereof, wherein A is Br.

9. The compound of claim 1, or a pharmaceutically acceptable salt thereof, wherein A is selected from methyl, propyl, Br, I, CN, methoxy, N(H)CH₂(phenyl), CF₃, and benzyl.

10. The compound of claim 1, or a pharmaceutically acceptable salt thereof, wherein A is Cy¹.

11. The compound of claim 1, or a pharmaceutically acceptable salt thereof, wherein Cy¹ is selected from C₆₋₁₀ aryl and 5-14 membered heteroaryl, each optionally substituted by 1, 2, 3, 4, or 5 substituents independently selected from halo, C₁₋₆ alkyl, C₁₋₆ haloalkyl, C₃₋₁₀ cycloalkyl, 4-14 membered heterocycloalkyl, CN, NO₂, OR^{a}, SR^{a}, NR^{c}R^{d}, NR^{c}C(O)R^{b}, S(O)NR^{c}R^{d}, S(O)₂R^{b}, and S(O)₂NR^{c}R^{d}.

12. The compound of claim 1, or a pharmaceutically acceptable salt thereof, wherein Cy¹ is phenyl, optionally substituted with 1, 2, or 3 substituents independently selected from halo, C₁₋₆ alkyl, and OR^{a}.

13. The compound of claim 1, or a pharmaceutically acceptable salt thereof, wherein Cy¹ is 5-14 membered heteroaryl or 4-14 membered heterocycloalkyl, each of which is optionally substituted with 1, 2, or 3 substituents independently selected from halo, CN, C₁₋₆ alkyl, C₁₋₆ haloalkyl, 4-14 membered heterocycloalkyl, NR^{c}R^{d}, OR^{a}, and C(O)OR^{a}.

14. The compound of claim 1, or a pharmaceutically acceptable salt thereof, wherein Cy¹ is 5-14 membered heteroaryl, optionally substituted with 1, 2, or 3 substituents independently selected from halo, C₁₋₆ alkyl, and OR^{a}.

15. The compound of claim 1, or a pharmaceutically acceptable salt thereof, wherein Cy¹ is selected from pyridyl, phenyl, furanyl, oxazolyl, isoxazolyl, pyrimidinyl, pyrazolyl, thiazolyl, dihydrofuranyl, thiophenyl, tetrahydrofuranyl, pyrrolidinyl, isoindolinyl, azetidinyl, and imidazolyl; each of which is optionally substituted with 1, 2, or 3 substituents independently selected from halo, CN, C₁₋₆ alkyl, C₁₋₆ haloalkyl, 4-14 membered heterocycloalkyl, NR^{c}R^{d}, OR^{a} and C(O)OR^{a}.

16. The compound of claim 1, or a pharmaceutically acceptable salt thereof, wherein Cy¹ is selected from pyridyl, phenyl, furanyl, oxazolyl, isoxazolyl, pyrimidinyl, pyrazolyl, and thiazolyl; each optionally substituted with 1, 2, or 3 substituents independently selected from halo, C₁₋₆ alkyl, and OR^{a}.

17. The compound of claim 1, or a pharmaceutically acceptable salt thereof, wherein Cy¹ is selected from pyridyl, morpholinophenyl, phenyl, furanyl, oxazolyl, isoxazolyl, methylisoxazolyl, dimethylphenyl, methylfuranyl, pyrimidinyl, methylpyrazolyl, dimethylisoxazolyl, methylpyridinyl, thiazolyl, fluorophenyl, and methoxyphenyl.

18. The compound of claim 1, or a pharmaceutically acceptable salt thereof, wherein Cy¹ is selected from pyridyl, morpholinophenyl, phenyl, furanyl, oxazolyl, isoxazolyl, methylisoxazolyl, dimethylphenyl, methylfuranyl, pyrimidinyl, methylpyrazolyl, dimethylisoxazolyl, methylpyridinyl, thiazolyl, fluorophenyl, methoxyphenyl, cyanophenyl, hydroxyphenyl, methylphenyl, dimethylpyrazolyl, methoxypyridinyl, dimethylpyridinyl, (difluoromethyl)pyrazolyl, (dimethylamino)phenyl, methoxymethylphenyl, (trifluoromethyl)pyridinyl, methyl(trifluoromethyl)pyrazol-4-yl, (trifluoromethoxy)phenyl, morpholinophenyl, methylthiazolyl, methylthiophenyl, morpholinopyridinyl, thiophenyl, dimethylfuranyl, tetrahydrofuranyl, pyrrolidinyl, isoindolinyl, azetidinyl, pyrazolyl, imidazolyl, and carboxyazetidinyl.

19. The compound of claim 1, or a pharmaceutically acceptable salt thereof, wherein Q is selected from the following groups:

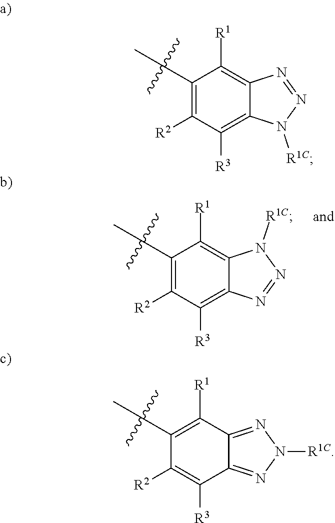

20. The compound of claim 1, or a pharmaceutically acceptable salt thereof, wherein Q is selected from the following groups:

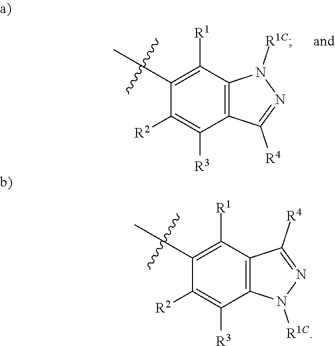

21. The compound of claim 1, or a pharmaceutically acceptable salt thereof, wherein Q is the following group:

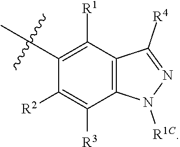

22. The compound of claim 1, or a pharmaceutically acceptable salt thereof, wherein Q is the following group:

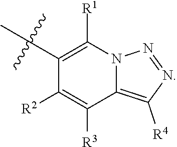

23. The compound of claim 1, or a pharmaceutically acceptable salt thereof, wherein Q is the following group:

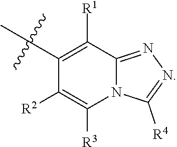

24. The compound of claim 1, or a pharmaceutically acceptable salt thereof, wherein R¹ is selected from H, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl and C₁₋₆ haloalkyl of R¹ are each optionally substituted with 1, 2, 3, 4, or 5 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

25. The compound of claim 1, or a pharmaceutically acceptable salt thereof, wherein R¹ is selected from H, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1}.

26. The compound of claim 1, or a pharmaceutically acceptable salt thereof, wherein R¹ is H, CN, halo, or C₁₋₆ alkyl.

27. The compound of claim 1, or a pharmaceutically acceptable salt thereof, wherein R¹ is H, CN, F, Cl, or methyl.

28. The compound of claim 1, or a pharmaceutically acceptable salt thereof, wherein R² is selected from H, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl and C₁₋₆ haloalkyl of R² are each optionally substituted with 1, 2, 3, 4, or 5 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1} NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

29. The compound of claim 1, or a pharmaceutically acceptable salt thereof, wherein R² is selected from H, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1}.

30. The compound of claim 1, or a pharmaceutically acceptable salt thereof, wherein R² is H, halo, C₁₋₆ alkyl, or C₁₋₆ haloalkyl.

31. The compound of claim 1, or a pharmaceutically acceptable salt thereof, wherein R² is H, Br, F, Cl, methyl, or CF₃.

32. The compound of claim 1, or a pharmaceutically acceptable salt thereof, wherein R³ is selected from H, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl and C₁₋₆ haloalkyl of R³ are each optionally substituted with 1, 2, 3, 4, or 5 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1} NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

33. The compound of claim 1, or a pharmaceutically acceptable salt thereof, wherein R³ is selected from H, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, and NR^{c1}R^{d1}.

34. The compound of claim 1, or a pharmaceutically acceptable salt thereof, wherein R³ is H or C₁₋₆ alkyl.

35. The compound of claim 1, or a pharmaceutically acceptable salt thereof, wherein R³ is H or methyl.

36. The compound of claim 1, or a pharmaceutically acceptable salt thereof, wherein R⁴ is H or C₁₋₆ alkyl.

37. The compound of claim 1, or a pharmaceutically acceptable salt thereof, wherein R⁴ is H or methyl.

38. The compound of claim 1, or a pharmaceutically acceptable salt thereof, wherein R⁵, R⁶, and R⁷ are each independently selected from H, halo, and C₁₋₆ alkyl.

39. The compound of claim 1, or a pharmaceutically acceptable salt thereof, wherein R⁵, R⁶, and R⁷ are each H.

40. The compound of claim 1, or a pharmaceutically acceptable salt thereof, wherein R⁵, R⁶, and R⁷ are each independently H or F.

41. The compound of claim 1, or a pharmaceutically acceptable salt thereof, wherein R^{1A} and R^{1B} are each independently selected from C₁₋₆ alkyl and H.

42. The compound of claim 1, or a pharmaceutically acceptable salt thereof, wherein R^{1A} and RB are each methyl.

43. The compound of claim 1, or a pharmaceutically acceptable salt thereof, wherein R^{1C} is H or C₁₋₆ alkyl.

44. The compound of claim 1, or a pharmaceutically acceptable salt thereof, wherein R^{1C} is H or methyl.

45. The compound of claim 1, wherein the compound is of Formula Ia: 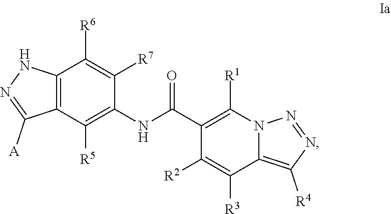
or a pharmaceutically acceptable salt thereof.

46. The compound of claim 1, wherein the compound is of Formula If: 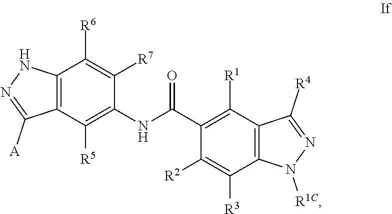
or a pharmaceutically acceptable salt thereof.

47. The compound of claim 1, wherein the compound is of Formula Ik: 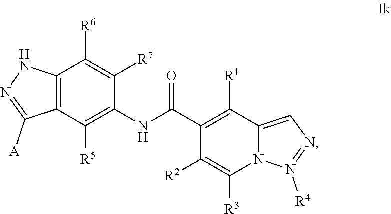
or a pharmaceutically acceptable salt thereof.

48. The compound of claim 1, wherein the compound is of Formula Il: 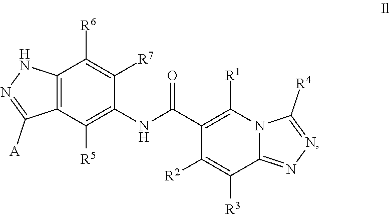
or a pharmaceutically acceptable salt thereof.

49. The compound of claim 1, wherein the compound is of Formula Im: 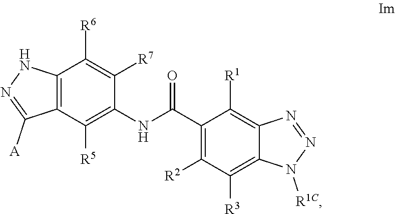
or a pharmaceutically acceptable salt thereof.

50. The compound of claim 1, wherein the compound is of Formula In: 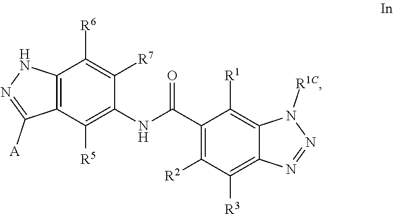
or a pharmaceutically acceptable salt thereof.

51. The compound of claim 1, wherein the compound is of Formula Io: 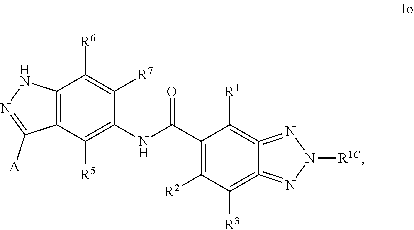
or a pharmaceutically acceptable salt thereof.

52. The compound of claim 1, wherein the compound is of Formula III: 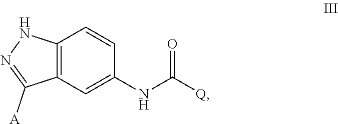
or a pharmaceutically acceptable salt thereof.

53. The compound of claim 1, wherein the compound is of Formula IIIa: 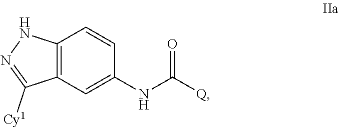
or a pharmaceutically acceptable salt thereof.

54. The compound of claim 1, wherein the compound is of Formula IIIb: 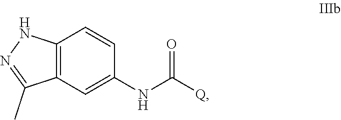
or a pharmaceutically acceptable salt thereof.

55. The compound of claim 1, or a pharmaceutically acceptable salt thereof, wherein:
A is C₁₋₆ alkyl, Cy¹, or halo;
Q is selected from the following groups: 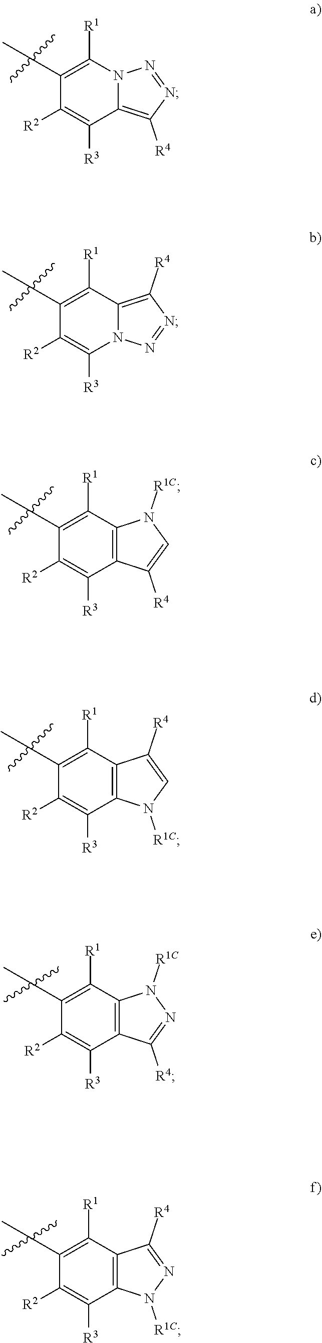 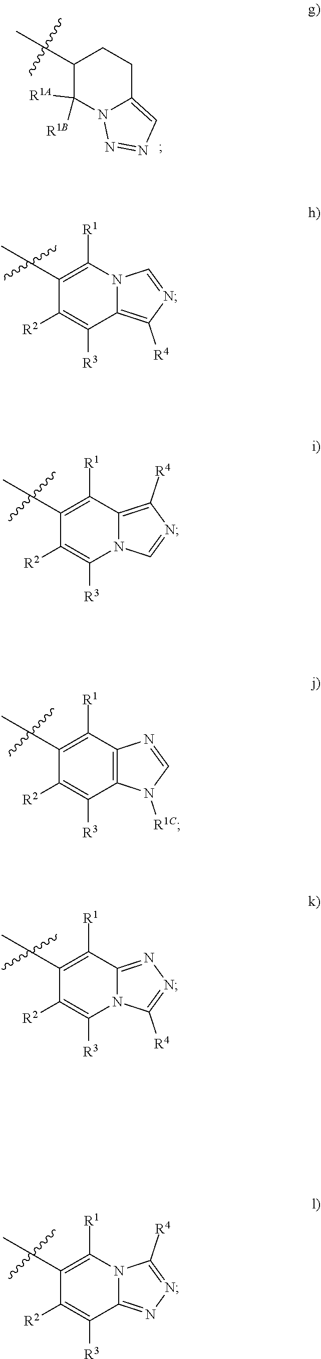 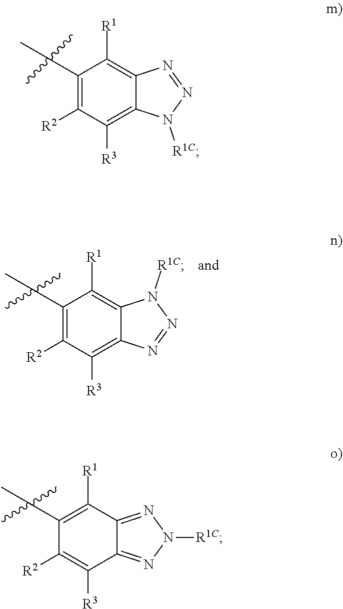
Cy¹ is selected from C₆₋₁₀ aryl and 5-14 membered heteroaryl, each optionally substituted by 1, 2, 3, 4, or 5 substituents independently selected from halo, C₁₋₆ alkyl, C₁₋₆ haloalkyl, C₃₋₁₀ cycloalkyl, 4-14 membered heterocycloalkyl, CN, NO₂, OR^{a}, SR^{a}, NR^{c}R^{d}, NR^{c}C(O)R^{b}, S(O)NR^{c}R^{d}, S(O)₂R^{b}, and S(O)₂NR^{c}R^{d};
R¹, R², R³, R⁴, R⁵, R⁶, and R⁷ are each independently selected from H, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl and C₁₋₆ haloalkyl of R¹, R², R³, R⁴, R⁵, R⁶, and R⁷ are each optionally substituted with 1, 2, 3, 4, or 5 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
R^{1A} and R^{1B} are each independently selected from H, halo, C₁₋₆ alkyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2} and NR²C(O)R^{b2}; wherein said C₁₋₆ alkyl and C₁₋₆ haloalkyl of R^{1A} and R^{1B} are each optionally substituted with 1, 2, 3, 4, or 5 substituents independently selected from CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, and NR^{c2}C(O)R^{b2};
R^{1C} is selected from H and C₁₋₆ alkyl;
each R^{a}, R^{b}, R^{c}, R^{d}, R^{a1}, R^{b1}, R^{c1}, R^{a1}, R^{a2}, R^{b2}, R^{c2}, R^{d2} is independently selected from H, C₁₋₆ alkyl, and C₁₋₆ haloalkyl; and
each R^{e} and R^{e1} is independently selected from H, C₁₋₄ alkyl, and CN.

56. The compound of claim 1, or a pharmaceutically acceptable salt thereof, wherein:
A is C₁₋₆ alkyl, Cy¹, or halo;
Q is selected from the following groups: 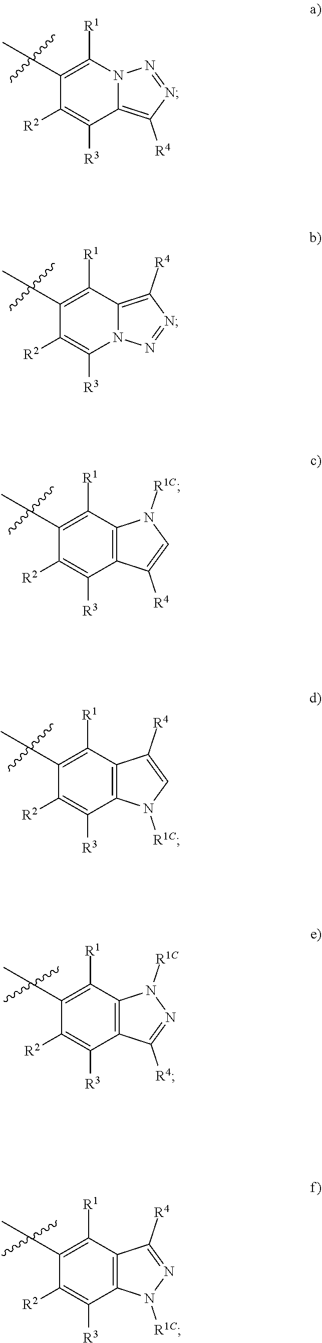 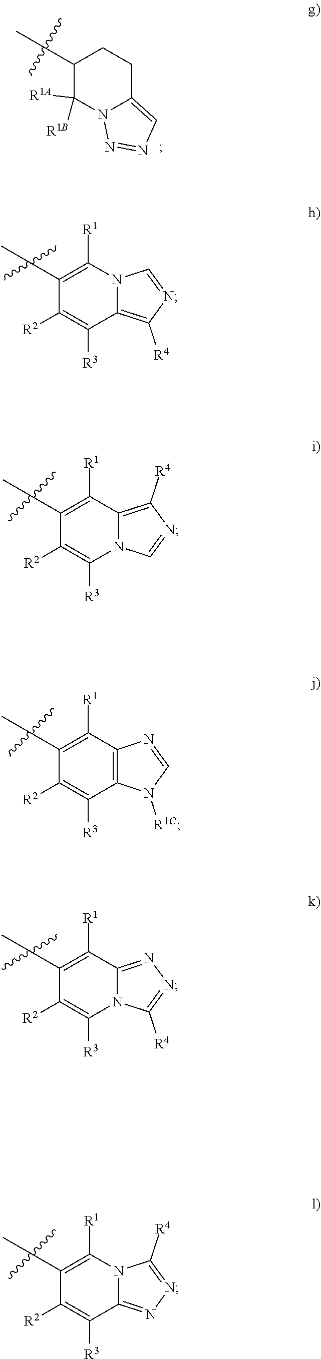 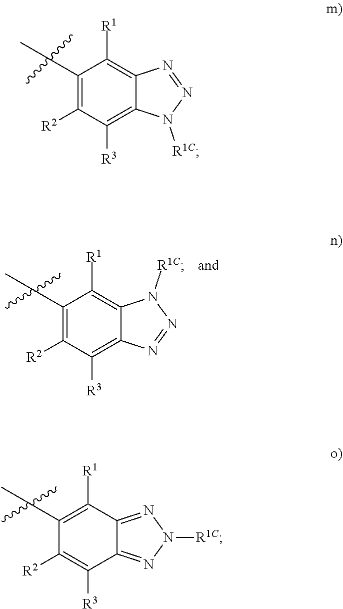
Cy¹ is selected from C₆₋₁₀ aryl and 5-14 membered heteroaryl, each optionally substituted by 1, 2, or 3 substituents independently selected from halo, C₁₋₆ alkyl, 4-14 membered heterocycloalkyl, and OR^{a};
R¹, R², R³, R⁴, R⁵, R⁶, and R⁷ are each independently selected from H, halo, CN, C₁₋₆ alkyl, and C₁₋₆ haloalkyl;
R^{1A} and R^{1B} are each independently selected from H and C₁₋₆ alkyl;
R^{1C} is selected from H and C₁₋₆ alkyl; and
each R^{a} is independently selected from H, C₁₋₆ alkyl, and C₁₋₆ haloalkyl.

57. The compound of claim 1, wherein the compound is selected from:
5-Methyl-N-(3-phenyl-1H-indazol-5-yl)-1H-benzo[d][1,2,3]triazole-6-carboxamide;
5-Methyl-N-(3-(3-morpholinophenyl)-1H-indazol-5-yl)-1H-benzo[d][1,2,3]triazole-6-carboxamide;
2,6-Dimethyl-N-(3-methyl-1H-indazol-5-yl)-2H-benzo[d][1,2,3]triazole-5-carboxamide;
1,6-Dimethyl-N-(3-methyl-1H-indazol-5-yl)-1H-benzo[d][1,2,3]triazole-5-carboxamide;
1,5-dimethyl-N-(3-methyl-1H-indazol-5-yl)-1H-benzo[d][1,2,3]triazole-6-carboxamide;
N-(3-Methyl-1H-indazol-5-yl)-[1,2,4]triazolo[4,3-a]pyridine-7-carboxamide;
3-Methyl-N-(3-methyl-1H-indazol-5-yl)-[1,2,4]triazolo[4,3-a]pyridine-7-carboxamide;
N-(3-Methyl-1H-indazol-5-yl)-[1,2,4]triazolo[4,3-a]pyridine-6-carboxamide;
3-Methyl-N-(3-methyl-1H-indazol-5-yl)-[1,2,4]triazolo[4,3-a]pyridine-6-carboxamide;
N-(3-Methyl-1H-indazol-5-yl)-1H-benzo[d][1,2,3]triazole-6-carboxamide;
N-(3-Methyl-1H-indazol-5-yl)-6-(trifluoromethyl)-1H-benzo[d][1,2,3]triazole-5-carboxamide;
4,6-Dimethyl-N-(3-methyl-1H-indazol-5-yl)-1H-benzo[d][1,2,3]triazole-5-carboxamide;
4-Methyl-N-(3-(pyridin-4-yl)-1H-indazol-5-yl)-1H-benzo[d][1,2,3]triazole-5-carboxamide;
7-Methyl-N-(3-(pyridin-4-yl)-1H-indazol-5-yl)-1H-benzo[d][1,2,3]triazole-5-carboxamide;
4-Cyano-N-(3-methyl-1H-indazol-5-yl)-1H-benzo[d][1,2,3]triazole-5-carboxamide;
N-(3-Bromo-1H-indazol-5-yl)-5-methyl-1H-benzo[d][1,2,3]triazole-6-carboxamide;
5-Methyl-N-(3-methyl-1H-indazol-5-yl)-1H-benzo[d][1,2,3]triazole-6-carboxamide;
6-Methyl-N-(3-phenyl-1H-indazol-5-yl)-1H-benzo[d][1,2,3]triazole-5-carboxamide;
N-(3-Bromo-1H-indazol-5-yl)-6-methyl-1H-benzo[d]imidazole-5-carboxamide;
6-Methyl-N-(3-phenyl-1H-indazol-5-yl)-1H-benzo[d]imidazole-5-carboxamide;
6-Methyl-N-(3-phenyl-1H-indazol-5-yl)-1H-indole-5-carboxamide;
6-Methyl-N-(3-phenyl-1H-indazol-5-yl)-1H-indazole-5-carboxamide;
5-Methyl-N-(3-phenyl-1H-indazol-5-yl)-1H-indole-6-carboxamide;
5-Methyl-N-(3-phenyl-1H-indazol-5-yl)-1H-indazole-6-carboxamide;
5-Bromo-N-(3-phenyl-1H-indazol-5-yl)-1H-indazole-6-carboxamide;
4,6-Difluoro-N-(3-(furan-2-yl)-1H-indazol-5-yl)-1-methyl-1H-indazole-5-carboxamide;
4,6-Difluoro-1-methyl-N-(3-(oxazol-5-yl)-1H-indazol-5-yl)-1H-indazole-5-carboxamide;
4,6-Difluoro-N-(3-(isoxazol-4-yl)-1H-indazol-5-yl)-1-methyl-1H-indazole-5-carboxamide;
4,6-Difluoro-N-(3-(furan-3-yl)-1H-indazol-5-yl)-1-methyl-1H-indazole-5-carboxamide;
4,6-Difluoro-1-methyl-N-(3-(5-methylisoxazol-4-yl)-1H-indazol-5-yl)-1H-indazole-5-carboxamide;
4,6-Difluoro-1-methyl-N-(3-(3-methylisoxazol-4-yl)-1H-indazol-5-yl)-1H-indazole-5-carboxamide;
N-(3-(2,3-Dimethylphenyl)-1H-indazol-5-yl)-4,6-difluoro-1-methyl-1H-indazole-5-carboxamide;
4,6-Difluoro-1-methyl-N-(3-(pyridin-3-yl)-1H-indazol-5-yl)-1H-indazole-5-carboxamide;
4,6-Difluoro-1-methyl-N-(3-(5-methylfuran-2-yl)-1H-indazol-5-yl)-1H-indazole-5-carboxamide;
4,6-Difluoro-1-methyl-N-(3-(pyrimidin-5-yl)-1H-indazol-5-yl)-1H-indazole-5-carboxamide;
4,6-Difluoro-1-methyl-N-(3-(1-methyl-1H-pyrazol-4-yl)-1H-indazol-5-yl)-1H-indazole-5-carboxamide;
N-(3-(3,5-Dimethylisoxazol-4-yl)-1H-indazol-5-yl)-4,6-difluoro-1-methyl-1H-indazole-5-carboxamide;
4,6-Difluoro-1-methyl-N-(3-(2-methylpyridin-4-yl)-1H-indazol-5-yl)-1H-indazole-5-carboxamide;
4,6-Difluoro-1-methyl-N-(3-(4-methylpyridin-3-yl)-1H-indazol-5-yl)-1H-indazole-5-carboxamide;
4,6-Difluoro-1-methyl-N-(3-(2-methylpyridin-3-yl)-1H-indazol-5-yl)-1H-indazole-5-carboxamide;
4,6-Difluoro-1-methyl-N-(3-(3-methylpyridin-4-yl)-1H-indazol-5-yl)-1H-indazole-5-carboxamide;
4,6-Difluoro-1-methyl-N-(3-(pyridin-2-yl)-1H-indazol-5-yl)-1H-indazole-5-carboxamide;
4,6-Difluoro-1-methyl-N-(3-(thiazol-5-yl)-1H-indazol-5-yl)-1H-indazole-5-carboxamide;
5-Methyl-N-(3-phenyl-1H-indazol-5-yl)-[1,2,3]triazolo[1,5-a]pyridine-6-carboxamide;
N-(3-(2-Fluorophenyl)-1H-indazol-5-yl)-5,7-dimethyl-[1,2,3]triazolo[1,5-a]pyridine-6-carboxamide;
N-(3-Bromo-1H-indazol-5-yl)-5,7-dimethyl-[1,2,3]triazolo[1,5-a]pyridine-6-carboxamide;
N-(3-(2-Methoxyphenyl)-1H-indazol-5-yl)-5,7-dimethyl-[1,2,3]triazolo[1,5-a]pyridine-6-carboxamide;
N-(3-(2,3-Dimethylphenyl)-1H-indazol-5-yl)-5,7-dimethyl-[1,2,3]triazolo[1,5-a]pyridine-6-carboxamide;
5,7-Dimethyl-N-(3-phenyl-1H-indazol-5-yl)-[1,2,3]triazolo[1,5-a]pyridine-6-carboxamide;
5,7-Dimethyl-N-(3-methyl-1H-indazol-5-yl)-[1,2,3]triazolo[1,5-a]pyridine-6-carboxamide;
4,6-Difluoro-1-methyl-N-(3-phenyl-1H-indazol-5-yl)-1H-indazole-5-carboxamide;
4,6-Difluoro-1-methyl-N-(3-methyl-1H-indazol-5-yl)-1H-indazole-5-carboxamide;
6-Methyl-N-(3-phenyl-1H-indazol-5-yl)imidazo[1,5-a]pyridine-7-carboxamide;
1-Methyl-N-(3-methyl-1H-indazol-5-yl)-1H-indazole-5-carboxamide;
(R)-7,7-Dimethyl-N-(3-phenyl-1H-indazol-5-yl)-4,5,6,7-tetrahydro-[1,2,3]triazolo[1,5-a]pyridine-6-carboxamide;
5,7-Dimethyl-N-(3-methyl-1H-indazol-5-yl)imidazo[1,5-a]pyridine-6-carboxamide;
6,8-Dichloro-N-(3-methyl-1H-indazol-5-yl)-[1,2,4]triazolo[4,3-a]pyridine-7-carboxamide;
6,8-Dimethyl-N-(3-phenyl-1H-indazol-5-yl)-[1,2,4]triazolo[4,3-a]pyridine-7-carboxamide;
6,8-Dimethyl-N-(3-methyl-1H-indazol-5-yl)-[1,2,4]triazolo[4,3-a]pyridine-7-carboxamide;
6-Chloro-8-methyl-N-(3-phenyl-1H-indazol-5-yl)-[1,2,4]triazolo[4,3-a]pyridine-7-carboxamide;
5,7-Dimethyl-N-(3-phenyl-1H-indazol-5-yl)-[1,2,4]triazolo[4,3-a]pyridine-6-carboxamide;
5,7-Dimethyl-N-(3-methyl-1H-indazol-5-yl)-[1,2,4]triazolo[4,3-a]pyridine-6-carboxamide;
5-Methyl-N-(3-phenyl-1H-indazol-5-yl)imidazo[1,5-a]pyridine-6-carboxamide;
6,8-dichloro-N-(3-phenyl-1H-indazol-5-yl)-[1,2,4]triazolo[4,3-a]pyridine-7-carboxamide;
3,5,7-Trimethyl-N-(3-phenyl-1H-indazol-5-yl)-[1,2,3]triazolo[1,5-a]pyridine-6-carboxamide;
3,5,7-Trimethyl-N-(3-methyl-1H-indazol-5-yl)-[1,2,3]triazolo[1,5-a]pyridine-6-carboxamide;
5,7-Dimethyl-N-(3-(3-morpholinophenyl)-1H-indazol-5-yl)-[1,2,3]triazolo[1,5-a]pyridine-6-carboxamide;
4-Methyl-N-(3-phenyl-1H-indazol-5-yl)-[1,2,3]triazolo[1,5-a]pyridine-5-carboxamide;
N-(3-(Furan-3-yl)-1H-indazol-5-yl)-5,7-dimethyl-[1,2,3]triazolo[1,5-a]pyridine-6-carboxamide; and
5,7-Dimethyl-N-(3-phenyl-1H-indazol-5-yl)imidazo[1,5-a]pyridine-6-carboxamide;
or a pharmaceutically acceptable salt of any of the aforementioned.

58. The compound of claim 1, wherein the compound is selected from:
N-(3-Phenyl-1H-indazol-5-yl)-5-(trifluoromethyl)-1H-indazole-6-carboxamide;
4,6-Difluoro-1-methyl-N-(3-(6-methylpyridin-2-yl)-1H-indazol-5-yl)-1H-indazole-5-carboxamide;
N-(3-(4,5-Dihydrofuran-2-yl)-1H-indazol-5-yl)-4,6-difluoro-1-methyl-1H-indazole-5-carboxamide;
4,6-Difluoro-1-methyl-N-(3-(pyrimidin-4-yl)-1H-indazol-5-yl)-1H-indazole-5-carboxamide;
N-(3-(2-Cyanophenyl)-1H-indazol-5-yl)-4,6-difluoro-1-methyl-1H-indazole-5-carboxamide;
4,6-Difluoro-N-(3-(2-hydroxyphenyl)-1H-indazol-5-yl)-1-methyl-1H-indazole-5-carboxamide;
4,6-Difluoro-1-methyl-N-(3-(m-tolyl)-1H-indazol-5-yl)-1H-indazole-5-carboxamide;
4,6-Difluoro-1-methyl-N-(3-(0-tolyl)-1H-indazol-5-yl)-1H-indazole-5-carboxamide;
4,6-Difluoro-N-(3-(2-fluorophenyl)-1H-indazol-5-yl)-1-methyl-1H-indazole-5-carboxamide;
N-(3-(1,3-Dimethyl-1H-pyrazol-5-yl)-1H-indazol-5-yl)-4,6-difluoro-1-methyl-1H-indazole-5-carboxamide;
N-(3-(1,3-Dimethyl-1H-pyrazol-4-yl)-1H-indazol-5-yl)-4,6-difluoro-1-methyl-1H-indazole-5-carboxamide;
4,6-Difluoro-N-(3-(2-methoxypyridin-3-yl)-1H-indazol-5-yl)-1-methyl-1H-indazole-5-carboxamide;
4,6-Difluoro-N-(3-(5-methoxypyridin-3-yl)-1H-indazol-5-yl)-1-methyl-1H-indazole-5-carboxamide;
N-(3-(2,6-Dimethylpyridin-4-yl)-1H-indazol-5-yl)-4,6-difluoro-1-methyl-1H-indazole-5-carboxamide;
4,6-Difluoro-N-(3-(3-methoxyphenyl)-1H-indazol-5-yl)-1-methyl-1H-indazole-5-carboxamide;
4,6-Difluoro-N-(3-(2-methoxyphenyl)-1H-indazol-5-yl)-1-methyl-1H-indazole-5-carboxamide;
N-(3-(1-(Difluoromethyl)-1H-pyrazol-4-yl)-1H-indazol-5-yl)-4,6-difluoro-1-methyl-1H-indazole-5-carboxamide;
N-(3-(4-(Dimethylamino)phenyl)-1H-indazol-5-yl)-4,6-difluoro-1-methyl-1H-indazole-5-carboxamide;
N-(3-(3-(Dimethylamino)phenyl)-1H-indazol-5-yl)-4,6-difluoro-1-methyl-1H-indazole-5-carboxamide;
4,6-Difluoro-N-(3-(2-methoxy-5-methylphenyl)-1H-indazol-5-yl)-1-methyl-1H-indazole-5-carboxamide;
4,6-Difluoro-N-(3-(5-methoxy-2-methylphenyl)-1H-indazol-5-yl)-1-methyl-1H-indazole-5-carboxamide;
4,6-Difluoro-1-methyl-N-(3-(4-(trifluoromethyl)pyridin-3-yl)-1H-indazol-5-yl)-1H-indazole-5-carboxamide;
4,6-Difluoro-1-methyl-N-(3-(1-methyl-3-(trifluoromethyl)-1H-pyrazol-4-yl)-1H-indazol-5-yl)-1H-indazole-5-carboxamide;
4,6-Difluoro-1-methyl-N-(3-(2-(trifluoromethoxy)phenyl)-1H-indazol-5-yl)-1H-indazole-5-carboxamide;
4,6-Difluoro-1-methyl-N-(3-(4-morpholinophenyl)-1H-indazol-5-yl)-1H-indazole-5-carboxamide;
4,6-Difluoro-1-methyl-N-(3-(2-methylthiazol-5-yl)-1H-indazol-5-yl)-1H-indazole-5-carboxamide;
4,6-Difluoro-1-methyl-N-(3-(1-methyl-1H-pyrazol-5-yl)-1H-indazol-5-yl)-1H-indazole-5-carboxamide;
4,6-Difluoro-1-methyl-N-(3-(5-methylthiophen-2-yl)-1H-indazol-5-yl)-1H-indazole-5-carboxamide;
4,6-Difluoro-1-methyl-N-(3-(5-morpholinopyridin-3-yl)-1H-indazol-5-yl)-1H-indazole-5-carboxamide;
4,6-Difluoro-1-methyl-N-(3-propyl-1H-indazol-5-yl)-1H-indazole-5-carboxamide;
4,6-Difluoro-1-methyl-N-(3-(thiophen-3-yl)-1H-indazol-5-yl)-1H-indazole-5-carboxamide;
N-(3-(2,5-Dimethylfuran-3-yl)-1H-indazol-5-yl)-4,6-difluoro-1-methyl-1H-indazole-5-carboxamide;
4,6-Difluoro-N-(3-(4-methoxypyridin-3-yl)-1H-indazol-5-yl)-1-methyl-1H-indazole-5-carboxamide;
4,6-Difluoro-N-(3-(3-methoxypyridin-4-yl)-1H-indazol-5-yl)-1-methyl-1H-indazole-5-carboxamide;
4,6-Difluoro-1-methyl-N-(3-(tetrahydrofuran-3-yl)-1H-indazol-5-yl)-1H-indazole-5-carboxamide;
N-(3-Cyano-1H-indazol-5-yl)-4,6-difluoro-1-methyl-1H-indazole-5-carboxamide;
4,6-Difluoro-N-(3-methoxy-1H-indazol-5-yl)-1-methyl-1H-indazole-5-carboxamide;
1-Methyl-N-(3-phenyl-1H-indazol-5-yl)-1H-indazole-5-carboxamide;
4,6-Difluoro-1-methyl-N-(3-(pyrrolidin-1-yl)-1H-indazol-5-yl)-1H-indazole-5-carboxamide;
4,6-Difluoro-N-(3-(isoindolin-2-yl)-1H-indazol-5-yl)-1-methyl-1H-indazole-5-carboxamide;
N-(3-(Benzylamino)-1H-indazol-5-yl)-4,6-difluoro-1-methyl-1H-indazole-5-carboxamide;
N-(3-Iodo-1H-indazol-5-yl)-2,4-dimethylimidazo[1,5-a]pyrimidine-3-carboxamide;
N-(3-(Furan-3-yl)-1H-indazol-5-yl)-2,4-dimethylimidazo[1,5-a]pyrimidine-3-carboxamide;
N-(3-(Furan-3-yl)-1H-indazol-5-yl)-4-methylimidazo[1,5-a]pyrimidine-3-carboxamide;
N-(3-(Furan-3-yl)-1H-indazol-5-yl)-4-methylimidazo[1,5-a]pyrimidine-3-carboxamide;
4,6-Difluoro-1-methyl-N-(3-(trifluoromethyl)-1H-indazol-5-yl)-1H-indazole-5-carboxamide;
N-(3-(furan-3-yl)-1H-indazol-5-yl)benzo[d]oxazole-6-carboxamide;
N-(3-(Furan-3-yl)-1H-indazol-5-yl)pyrazolo[1,5-a]pyridine-5-carboxamide;
N-(3-(Furan-3-yl)-1H-indazol-5-yl)imidazo[1,2-a]pyridine-7-carboxamide;
N-(3-(Furan-3-yl)-1H-indazol-5-yl)imidazo[1,2-a]pyridine-6-carboxamide;
N-(3-(Furan-3-yl)-1H-indazol-5-yl)benzo[c][1,2,5]thiadiazole-5-carboxamide;
N-(3-(Furan-3-yl)-1H-indazol-5-yl)benzo[d][1,2,3]thiadiazole-5-carboxamide;
N-(3-(Furan-3-yl)-1H-indazol-5-yl)thiazolo[5,4-b]pyridine-5-carboxamide;
N-(3-(Furan-3-yl)-1H-indazol-5-yl)benzo[d]thiazole-6-carboxamide;
N-(3-(Furan-3-yl)-1H-indazol-5-yl)benzo[d]thiazole-5-carboxamide;
N-(3-(Furan-3-yl)-1H-indazol-5-yl)thieno[3,2-b]pyridine-2-carboxamide;
N-(3-(Furan-3-yl)-1H-indazol-5-yl)benzo[d]oxazole-5-carboxamide;
N-(3-(Furan-3-yl)-1H-indazol-5-yl)pyrazolo[1,5-a]pyrimidine-5-carboxamide;
N-(3-(Furan-3-yl)-1H-indazol-5-yl)imidazo[1,2-a]pyrimidine-2-carboxamide;
N-(3-(Furan-3-yl)-1H-indazol-5-yl)imidazo[1,2-a]pyrimidine-6-carboxamide;
N-(3-(Furan-3-yl)-1H-indazol-5-yl)pyrazolo[1,5-a]pyrimidine-6-carboxamide;
N-(3-(Furan-3-yl)-1H-indazol-5-yl)-1-methyl-1H-benzo[d]imidazole-6-carboxamide;
N-(3-(Furan-3-yl)-1H-indazol-5-yl)-1-methyl-1H-benzo[d]imidazole-5-carboxamide;
N-(3-(Furan-3-yl)-1H-indazol-5-yl)-1-methyl-1H-indazole-5-carboxamide;
N-(3-(Furan-3-yl)-1H-indazol-5-yl)-2-methylbenzo[d]oxazole-6-carboxamide;
4,6-Difluoro-N-(3-(isoxazol-3-yl)-1H-indazol-5-yl)-1-methyl-1H-indazole-5-carboxamide;
4,6-Difluoro-1-methyl-N-(3-(oxazol-4-yl)-1H-indazol-5-yl)-1H-indazole-5-carboxamide;
4,6-Difluoro-N-(3-(isoxazol-5-yl)-1H-indazol-5-yl)-1-methyl-1H-indazole-5-carboxamide;
4,6-Difluoro-1-methyl-N-(3-(oxazol-2-yl)-1H-indazol-5-yl)-1H-indazole-5-carboxamide;
N-(3-(Azetidin-1-yl)-1H-indazol-5-yl)-4,6-difluoro-1-methyl-1H-indazole-5-carboxamide;
N-(3-Benzyl-1H-indazol-5-yl)-4,6-difluoro-1-methyl-1H-indazole-5-carboxamide;
N-(3-(1H-Imidazol-1-yl)-1H-indazol-5-yl)-4,6-difluoro-1-methyl-1H-indazole-5-carboxamide;
1-(5-(4,6-difluoro-1-methyl-1H-indazole-5-carboxamido)-1H-indazol-3-yl)azetidine-3-carboxylic acid;
4,6-Difluoro-N-(6-fluoro-3-phenyl-1H-indazol-5-yl)-1-methyl-1H-indazole-5-carboxamide;
4,6-Difluoro-N-(6-fluoro-3-(furan-3-yl)-1H-indazol-5-yl)-1-methyl-1H-indazole-5-carboxamide;
7-Methyl-N-(3-methyl-1H-indazol-5-yl)imidazo[1,5-a]pyridine-6-carboxamide;
N-(3-(Furan-3-yl)-1H-indazol-5-yl)-7-methylimidazo[1,5-a]pyridine-6-carboxamide;
N-(3-(Furan-3-yl)-1H-indazol-5-yl)-1,5,7-trimethylimidazo[1,5-a]pyridine-6-carboxamide;
1,5,7-Trimethyl-N-(3-methyl-1H-indazol-5-yl)imidazo[1,5-a]pyridine-6-carboxamide;
4-Methyl-N-(3-phenyl-1H-indazol-5-yl)-[1,2,3]triazolo[1,5-a]pyridine-5-carboxamide;
N-(3-(Furan-3-yl)-1H-indazol-5-yl)-5,7-dimethyl-[1,2,3]triazolo[1,5-a]pyridine-6-carboxamide;
N-(3-(Furan-3-yl)-1H-indazol-5-yl)-5,7-dimethylimidazo[1,5-a]pyridine-6-carboxamide;
N-(3-(Furan-3-yl)-1H-indazol-5-yl)-6-methylimidazo[1,5-a]pyridine-7-carboxamide;
N-(3-(Furan-3-yl)-1H-indazol-5-yl)-5,7-dimethyl-[1,2,4]triazolo[4,3-a]pyridine-6-carboxamide;
N-(3-(Furan-2-yl)-1H-indazol-5-yl)-6-methylimidazo[1,5-a]pyridine-7-carboxamide;
N-(3-(Furan-2-yl)-1H-indazol-5-yl)-5,7-dimethyl-[1,2,3]triazolo[1,5-a]pyridine-6-carboxamide;
N-(3-(Furan-3-yl)-1H-indazol-5-yl)benzo[d]isothiazole-6-carboxamide;
N-(3-(Furan-3-yl)-1H-indazol-5-yl)-1,4,6-trimethyl-1H-indazole-5-carboxamide;
N-(3-(Furan-3-yl)-1H-indazol-5-yl)-1,6-dimethyl-1H-indazole-5-carboxamide;
2-Methyl-N-(3-(oxazol-5-yl)-1H-indazol-5-yl)-2H-pyrazolo[3,4-c]pyridine-5-carboxamide; and
1,6-Dimethyl-N-(3-(oxazol-5-yl)-1H-indazol-5-yl)-1H-pyrazolo[4,3-b]pyridine-5-carboxamide,
or a pharmaceutically acceptable salt of any of the aforementioned.

59. The compound of claim 1, wherein the compound is selected from:
(R)-N-(3-(Furan-3-yl)-1H-indazol-5-yl)-1-methyl-4,5,6,7-tetrahydro-1H-indazole-5-carboxamide;
(S)-N-(3-(Furan-3-yl)-1H-indazol-5-yl)-1-methyl-4,5,6,7-tetrahydro-1H-indazole-5-carboxamide;
(R)-N-(3-(furan-3-yl)-1H-indazol-5-yl)-2-methyl-4,5,6,7-tetrahydro-2H-indazole-5-carboxamide;
(S)-N-(3-(furan-3-yl)-1H-indazol-5-yl)-2-methyl-4,5,6,7-tetrahydro-2H-indazole-5-carboxamide;
(R)-N-(3-(furan-3-yl)-1H-indazol-5-yl)-5,6,7,8-tetrahydro-[1,2,4]triazolo[4,3-a]pyridine-6-carboxamide;
(S)-N-(3-(furan-3-yl)-1H-indazol-5-yl)-5,6,7,8-tetrahydro-[1,2,4]triazolo[4,3-a]pyridine-6-carboxamide;
N-(3-(Furan-3-yl)-1H-indazol-5-yl)-4,6-dimethylpyrazolo[1,5-a]pyrazine-2-carboxamide;
N-(3-(furan-3-yl)-1H-indazol-5-yl)-2-methylpyrazolo[1,5-a]pyrazine-3-carboxamide;
1-Methyl-N-(3-(oxazol-5-yl)-1H-indazol-5-yl)-1H-pyrazolo[4,3-b]pyridine-5-carboxamide;
2-Methyl-N-(3-(oxazol-5-yl)-1H-indazol-5-yl)-2H-pyrazolo[4,3-b]pyridine-5-carboxamide;
1-Methyl-N-(3-(oxazol-5-yl)-1H-indazol-5-yl)-1H-pyrazolo[3,4-c]pyridine-5-carboxamide;
5-Methyl-N-(3-(oxazol-5-yl)-1H-indazol-5-yl)isothiazolo[5,4-b]pyridine-6-carboxamide;
1,4-Dimethyl-N-(3-(oxazol-5-yl)-1H-indazol-5-yl)-1H-pyrazolo[3,4-c]pyridine-5-carboxamide;
N-(3-(Furan-3-yl)-1H-indazol-5-yl)thiazolo[4,5-c]pyridine-2-carboxamide;
N-(3-(Furan-3-yl)-1H-indazol-5-yl)thiazolo[5,4-c]pyridine-2-carboxamide;
N-(3-(Furan-3-yl)-1H-indazol-5-yl)-8,8-dimethyl-5,6-dihydro-[1,2,4]triazolo[4,3-a]pyrazine-7(8H)-carboxamide;
(S)-N-(3-(Furan-3-yl)-1H-indazol-5-yl)-5,6,7,8-tetrahydroimidazo[1,5-a]pyridine-6-carboxamide;
(R)-N-(3-(Furan-3-yl)-1H-indazol-5-yl)-5,6,7,8-tetrahydroimidazo[1,5-a]pyridine-6-carboxamide,
or a pharmaceutically acceptable salt of any of the aforementioned.

60. A pharmaceutical composition comprising a compound of claim 1, or a pharmaceutically acceptable salt thereof, and at least one pharmaceutically acceptable carrier.

61. A method of inhibiting LRRK2 activity, said method comprising contacting a compound of claim 1, or a pharmaceutically acceptable salt thereof with LRRK2.

62. The method of claim 61, wherein the LRRK2 is characterized by a G2019S mutation.

63. The method of claim 61, wherein the contacting comprises administering the compound to a patient.

64. A method of treating a disease or disorder associated with elevated expression or activity of LRRK2, or functional variants thereof, said method comprising administering to a patient in need thereof a therapeutically effective amount of a compound of claim 1, or a pharmaceutically acceptable salt thereof.

65. The method of claim 64, wherein the LRRK2 is characterized by a G2019S mutation.

66. A method for treating a neurodegenerative disease in a patient, said method comprising: administering to the patient a therapeutically effective amount of a compound of claim 1, or a pharmaceutically acceptable salt thereof.

67. The method of claim 66, wherein said neurodegenerative disease is selected from Parkinson's disease, Parkinson disease with dementia, Parkinson's disease at risk syndrome, dementia with Lewy bodies, Lewy body variant of Alzheimer's disease, combined Parkinson's disease and Alzheimer's disease, multiple system atrophy, striatonigral degeneration, olivopontocerebellar atrophy, and Shy-Drager syndrome.

68. The method of claim 66, wherein said neurodegenerative disease is Parkinson's disease.

69. The method of claim 66, wherein the Parkinson's disease is characterized by a G2019S mutation in LRRK2.
